SEQUENCE LISTING (988 sequences)

SEQ ID NO: 1 - RNA, 26 nt, Mus musculus
ccugcaugcuuagugccuucagaggu

SEQ ID NO: 2 - RNA, 26 nt, Mus musculus
uaucuguguggcuguguaaagaaaga

SEQ ID NO: 3 - RNA, 29 nt, Mus musculus
uaacaaaccaaccacuaaggaauguagcu

SEQ ID NO: 4 - RNA, 26 nt, Mus musculus
uggaagguaacugcaugcuuagugga

SEQ ID NO: 5 - RNA, 26 nt, Mus musculus
ugaguguggcugucacuggccucugu

SEQ ID NO: 6 - RNA, 31 nt, Mus musculus
ugagauucugcaggcugcugagcuguaaugu

SEQ ID NO: 7 - RNA, 26 nt, Mus musculus
ugacuuagagcccuaucuucugcagg

SEQ ID NO: 8 - RNA, 26 nt, Mus musculus
ucugaguguggcugucacuggccucu

SEQ ID NO: 9 - RNA, 32 nt, Mus musculus
aacacagauuuuaaaaguacacauuuuucugg

SEQ ID NO: 10 - RNA, 31 nt, Mus musculus
caauugcagccuccagaagauagacauggcc

SEQ ID NO: 11 - RNA, 33 nt, Mus musculus
ugaugugcuuuguggguugguugguuuguuugu

SEQ ID NO: 12 - RNA, 26 nt, Mus musculus
ugaaagucccugcagaagauagaggu

SEQ ID NO: 13 - RNA, 29 nt, Mus musculus
uagaagggacagccacacagaugugugua

SEQ ID NO: 14 - RNA, 26 nt, Mus musculus
gaagggacagccacacagaugugugu

SEQ ID NO: 15 - RNA, 29 nt, Mus musculus
uaaaaugcagccgagugaacaggccucuc

SEQ ID NO: 16 - RNA, 35 nt, Mus musculus
gaggcuggugccuaguugguugguugguuugucau

SEQ ID NO: 17 - RNA, 30 nt, Mus musculus
uuuauugcuacaauugucugguugaggaua

SEQ ID NO: 18 - RNA, 26 nt, Mus musculus
ugaagaagagcaagaaggaaagugaa

SEQ ID NO: 19 - RNA, 26 nt, Mus musculus
uauuuaaacugucuuaugugucucca

SEQ ID NO: 20 - RNA, 26 nt, Mus musculus
uuguguucucuuauguucuguaugac

SEQ ID NO: 21 - RNA, 25 nt, Mus musculus
ugguuauuuaaaacuuugucucuga

SEQ ID NO: 22 - RNA, 27 nt, Mus musculus
uacuugguguccuugcucaggaacugg

SEQ ID NO: 23 - RNA, 28 nt, Mus musculus
augaagauuuucaacucaaaggaccagc

SEQ ID NO: 24 - RNA, 26 nt, Mus musculus
uauaacaacuucuguuuccagaagga

SEQ ID NO: 25 - RNA, 26 nt, Mus musculus
uuacugcugccgugaucauagcaauu

SEQ ID NO: 26 - RNA, 30 nt, Mus musculus
uacugcugccgugaucauagcaauuucagc

SEQ ID NO: 27 - RNA, 26 nt, Mus musculus
uuguuggagaagguguuuaagauccu

SEQ ID NO: 28 - RNA, 26 nt, Mus musculus
uuguguucucuuaugguuggugugac

SEQ ID NO: 29 - RNA, 26 nt, Mus musculus
uguguucucuuauggucuguaugaca

SEQ ID NO: 30 - RNA, 27 nt, Mus musculus
ucaaccuucuugaaagaugacaugucu

SEQ ID NO: 31 - RNA, 26 nt, Mus musculus
uuuggugaacagaaaggugaaauaga

SEQ ID NO: 32 - RNA, 26 nt, Mus musculus
uguggaaacugugacuuuuguuuugc

SEQ ID NO: 33 - RNA, 28 nt, Mus musculus
uguaaacugugaaaggucaacccugggc

SEQ ID NO: 34 - RNA, 26 nt, Mus musculus
uagauggaauuucacugcaaaaugcc

SEQ ID NO: 35 - RNA, 26 nt, Mus musculus
cagccucugaugcagauaaucccacc

SEQ ID NO: 36 - RNA, 26 nt, Mus musculus
auucacugagacucuaggcuuucucc

SEQ ID NO: 37 - RNA, 26 nt, Mus musculus
aauuuugagcaguuccuccaggagag

SEQ ID NO: 38 - RNA, 27 nt, Mus musculus
ugaaauuaaggagagagcuacccuccc

SEQ ID NO: 39 - RNA, 26 nt, Mus musculus
cccugugcuugugaaugagaugugag

SEQ ID NO: 40 - RNA, 26 nt, Mus musculus
uaacuuccucuguuaccuggugggga

SEQ ID NO: 41 - RNA, 25 nt, Mus musculus
ugagaagugcucugcagauggcugu

SEQ ID NO: 42 - RNA, 26 nt, Mus musculus
uacuuuuggugaugauagugaugucu

SEQ ID NO: 43 - RNA, 26 nt, Mus musculus
uccuguguuguggcauguuucuaaaa

SEQ ID NO: 44 - RNA, 26 nt, Mus musculus
ugaggaugaagaagauggagagcaga

SEQ ID NO: 45 - RNA, 26 nt, Mus musculus
uucuucuucuucacagaaaggguuug

SEQ ID NO: 46 - RNA, 27 nt, Mus musculus
ugaugaagaagaaggagauguccacga

SEQ ID NO: 47 - RNA, 25 nt, Mus musculus
uacuuauuugaugacuggugcuucc

SEQ ID NO: 48 - RNA, 26 nt, Mus musculus
cuaauucagauacugcuacaagauug

SEQ ID NO: 49 - RNA, 26 nt, Mus musculus
uagaaguggcaccaaaagcucuccac

SEQ ID NO: 50 - RNA, 26 nt, Mus musculus
uaguuccaucucuaacagagcucuau

SEQ ID NO: 51 - RNA, 27 nt, Mus musculus
uguaaucaucagauguauguguggcuu

SEQ ID NO: 52 - RNA, 26 nt, Mus musculus
ugaaaagcaaguugagggggaagagc

SEQ ID NO: 53 - RNA, 28 nt, Mus musculus
uacuuagcugucuuuuaagaaugacucc

SEQ ID NO: 54 - RNA, 27 nt, Mus musculus
ugagauuccuaaagcaguugucccgga

SEQ ID NO: 55 - RNA, 27 nt, Mus musculus
ugaaaagcaaguuggagaggaaagggu

SEQ ID NO: 56 - RNA, 26 nt, Mus musculus
uaccaaaaauaccaguuaagagucac

SEQ ID NO: 57 - RNA, 26 nt, Mus musculus
ugaaagugaugucaaacugaggggca

SEQ ID NO: 58 - RNA, 31 nt, Mus musculus
ucucuggaagaaacuugagucugggagcuca

SEQ ID NO: 59 - RNA, 27 nt, Mus musculus
cucuggaagaaacuugagucugggagc

SEQ ID NO: 60 - RNA, 27 nt, Mus musculus
uaaagcuuugagaaagugucauuugca

SEQ ID NO: 61 - RNA, 27 nt, Mus musculus
uguuucuucugcaugcuccagugucgc

SEQ ID NO: 62 - RNA, 26 nt, Mus musculus
gugccugucugugugguggcccagca

SEQ ID NO: 63 - RNA, 26 nt, Mus musculus
cacaugcagaagaaagagccaucuac

SEQ ID NO: 64 - RNA, 27 nt, Mus musculus
uguguaaaagugccucauuuucuguau

SEQ ID NO: 65 - RNA, 26 nt, Mus musculus
ugcuugcacaugcacagucaccugua

SEQ ID NO: 66 - RNA, 28 nt, Mus musculus
uagauaaccauuauacaaaacauccaau

SEQ ID NO: 67 - RNA, 27 nt, Mus musculus
uaauaaaauguucuuuugacaaaugaa

SEQ ID NO: 68 - RNA, 26 nt, Mus musculus
ugugguaggauuacucagcuguuaau

SEQ ID NO: 69 - RNA, 26 nt, Mus musculus
uguugucuacuguuaggaauagagug

SEQ ID NO: 70 - RNA, 32 nt, Mus musculus
uacaugucaaugacacgucuguguuacauagc

SEQ ID NO: 71 - RNA, 28 nt, Mus musculus
uaauuucuuaagagaacuuuaugguguu

SEQ ID NO: 72 - RNA, 27 nt, Mus musculus
uagaaguuaaaccagaaaugugugccu

SEQ ID NO: 73 - RNA, 26 nt, Mus musculus
uguagagcagguggaagggagcugaa

SEQ ID NO: 74 - RNA, 26 nt, Mus musculus
uacaaaaguuagcaguuuuagguuua

SEQ ID NO: 75 - RNA, 27 nt, Mus musculus
uaaggguguagaagaggauaugugcca

SEQ ID NO: 76 - RNA, 25 nt, Mus musculus
cagagcagguggauuccaaguuuga

SEQ ID NO: 77 - RNA, 26 nt, Mus musculus
uuaaagaaacuuugucugaauaaguu

SEQ ID NO: 78 - RNA, 26 nt, Mus musculus
ugugacuccuguuguguauggucuug

SEQ ID NO: 79 - RNA, 28 nt, Mus musculus
uaaaggugacuccuggcaccccuggccu

SEQ ID NO: 80 - RNA, 26 nt, Mus musculus
uuguuguuuguacagagucucacugu

SEQ ID NO: 81 - RNA, 26 nt, Mus musculus
uaauugguugcucuggagaugagggg

SEQ ID NO: 82 - RNA, 26 nt, Mus musculus
uuuaaaaauuacagaugggaaggaaa

SEQ ID NO: 83 - RNA, 30 nt, Mus musculus
uagaucuguguggccuuggacacucaaacu

SEQ ID NO: 84 - RNA, 26 nt, Mus musculus
uaaaguaaagaaauacagccacuggg

SEQ ID NO: 85 - RNA, 25 nt, Mus musculus
uaguguuugucuuaaacgcucaggg

SEQ ID NO: 86 - RNA, 27 nt, Mus musculus
uaagccaagaauuaugaguguccugcu

SEQ ID NO: 87 - RNA, 26 nt, Mus musculus
uagagcaacaagaguagaugauucuc

SEQ ID NO: 88 - RNA, 26 nt, Mus musculus
gcugcugaaauaaaaagaagaggagg

SEQ ID NO: 89 - RNA, 30 nt, Mus musculus
ugauauggaacguuccugcucuuugguggc

SEQ ID NO: 90 - RNA, 29 nt, Mus musculus
uauacaagcaccaaguaaauccugggagc

SEQ ID NO: 91 - RNA, 30 nt, Mus musculus
gacaaugacaugaagccucagaauuggguu

SEQ ID NO: 92 - RNA, 29 nt, Mus musculus
uuucaagugaaaucaguugauagaagagu

SEQ ID NO: 93 - RNA, 27 nt, Mus musculus
aaaagaugcuucuggaaacaagguuaa

SEQ ID NO: 94 - RNA, 26 nt, Mus musculus
uacaacuuuugauucuugcuuccaaa

SEQ ID NO: 95 - RNA, 26 nt, Mus musculus
uagguucuguuagagaagaugcaucu

SEQ ID NO: 96 - RNA, 30 nt, Mus musculus
uccaaaagcucuucuauuuguggucaaagu

SEQ ID NO: 97 - RNA, 27 nt, Mus musculus
ugauaaaaugucaagaccaaagcuggg

SEQ ID NO: 98 - RNA, 28 nt, Mus musculus
uagaaauguaucuaaggcagaagcaggu

SEQ ID NO: 99 - RNA, 27 nt, Mus musculus
ugagaaaccaccuguggagagaaugga

SEQ ID NO: 100 - RNA, 29 nt, Mus musculus
uagccacaagguuaaugggucuuucuauc

SEQ ID NO: 101 - RNA, 26 nt, Mus musculus
uaaccaccugucuccagcuccgggaa

SEQ ID NO: 102 - RNA, 43 nt, Mus musculus
uggagacagguggucccugaagcucccugguuagauaguccaa

SEQ ID NO: 103 - RNA, 26 nt, Mus musculus
ugcucaaacuggaaaucaagaguucu

SEQ ID NO: 104 - RNA, 26 nt, Mus musculus
uaauuccaguuugaggaaauccaaug

SEQ ID NO: 105 - RNA, 26 nt, Mus musculus
gcugcuguuauaaaguguaauguaaa

SEQ ID NO: 106 - RNA, 26 nt, Mus musculus
cauagccacaaggugucaguguuacc

SEQ ID NO: 107 - RNA, 28 nt, Mus musculus
cuguucuuuucacuggguauguuggugc

SEQ ID NO: 108 - RNA, 26 nt, Mus musculus
ugacacuuaugaaugaccagugcuga

SEQ ID NO: 109 - RNA, 26 nt, Mus musculus
uaugccugcuaguuauguaaccugug

SEQ ID NO: 110 - RNA, 29 nt, Mus musculus
uaaacagagaaaugccuucagcaacaugc

SEQ ID NO: 111 - RNA, 28 nt, Mus musculus
uccacauggaaauggcauuuaaacuccu

SEQ ID NO: 112 - RNA, 27 nt, Mus musculus
uauuuccaugugggcuacguuuaguga

SEQ ID NO: 113 - RNA, 26 nt, Mus musculus
uuguugaguauugccuuuuguagaag

SEQ ID NO: 114 - RNA, 26 nt, Mus musculus
uaacagagauuauaggugugaaccac

SEQ ID NO: 115 - RNA, 27 nt, Mus musculus
uuuucuguuuugcuuuugauugaggua

SEQ ID NO: 116 - RNA, 26 nt, Mus musculus
ugugaauucauauauaugggagcugg

SEQ ID NO: 117 - RNA, 27 nt, Mus musculus
uuuucuguuuugcuuuugauugaggua

SEQ ID NO: 118 - RNA, 26 nt, Mus musculus
uagcauugugggaaauguaguuccua

SEQ ID NO: 119 - RNA, 26 nt, Mus musculus
gaaugcaagagauggaagagagaauc

SEQ ID NO: 120 - RNA, 27 nt, Mus musculus
ugaggccaugcuaaacuuugucgaguc

SEQ ID NO: 121 - RNA, 28 nt, Mus musculus
uccuaaagguccuaaauucaaaucccag

SEQ ID NO: 122 - RNA, 27 nt, Mus musculus
uucuuucugugcuuuggugauuucucc

SEQ ID NO: 123 - RNA, 26 nt, Mus musculus
uuaucuuucugugccugucuuagaga

SEQ ID NO: 124 - RNA, 27 nt, Mus musculus
ucuuucugugcuuuggugauuucuccu

SEQ ID NO: 125 - RNA, 25 nt, Mus musculus
gugugcuuuugcuguguguguauga

SEQ ID NO: 126 - RNA, 27 nt, Mus musculus
uuggcuuccgguguacugcauguaugu

SEQ ID NO: 127 - RNA, 27 nt, Mus musculus
cacacacacugguacauggguguccuu

SEQ ID NO: 128 - RNA, 25 nt, Mus musculus
gucagcugacugaaggggguguuuc

SEQ ID NO: 129 - RNA, 28 nt, Mus musculus
uagagaaagugugucccuggaugugggu

SEQ ID NO: 130 - RNA, 28 nt, Mus musculus
uuguugugaugauguucucuauggaauc

SEQ ID NO: 131 - RNA, 30 nt, Mus musculus
uugacaugguaccaccuugaguuuaguugg

SEQ ID NO: 132 - RNA, 27 nt, Mus musculus
uguuagagaaaguguaccacuaggagc

SEQ ID NO: 133 - RNA, 31 nt, Mus musculus
ugacaugguaccaccuugaguuuaguugguu

SEQ ID NO: 134 - RNA, 30 nt, Mus musculus
ugacaugguaccaccuugaguuuaguugga

SEQ ID NO: 135 - RNA, 27 nt, Mus musculus
uguacugacauuagaggcugccaguug

SEQ ID NO: 136 - RNA, 27 nt, Mus musculus
aaacugcagagucacagcccauaccac

SEQ ID NO: 137 - RNA, 26 nt, Mus musculus
uaggaaguucuguuguccacaucaga

SEQ ID NO: 138 - RNA, 26 nt, Mus musculus
uacaguuugaaaagcugagaucaggg

SEQ ID NO: 139 - RNA, 26 nt, Mus musculus
gaacuuccuuccuucuaggugacccc

SEQ ID NO: 140 - RNA, 26 nt, Mus musculus
uacuagaggagcuacagauaggguuu

SEQ ID NO: 141 - RNA, 30 nt, Mus musculus
ugaccauuucacucagacuguaaccaugac

SEQ ID NO: 142 - RNA, 27 nt, Mus musculus
ugucauguguggcgggugauguggucc

SEQ ID NO: 143 - RNA, 28 nt, Mus musculus
ugacagcuugacaagagugacuuuuaaa

SEQ ID NO: 144 - RNA, 28 nt, Mus musculus
aagaugccacaacuggauccuucugcaa

SEQ ID NO: 145 - RNA, 30 nt, Mus musculus
cacugacuuaaaguuuuaaguagaaaagga

SEQ ID NO: 146 - RNA, 26 nt, Mus musculus
aacuuuaagucagugggguuuacaga

SEQ ID NO: 147 - RNA, 28 nt, Mus musculus
ucaacuuuacuuagggugacugaaggcu

SEQ ID NO: 148 - RNA, 26 nt, Mus musculus
ugugaugucagcacccgagaggcuga

SEQ ID NO: 149 - RNA, 26 nt, Mus musculus
uagagcaagcuguauguacaucugua

SEQ ID NO: 150 - RNA, 26 nt, Mus musculus
ugacagcagauauagggaggucagca

SEQ ID NO: 151 - RNA, 27 nt, Mus musculus
cacagcauucugugaaccucauccaug

SEQ ID NO: 152 - RNA, 28 nt, Mus musculus
uuuuaugugcuuuaauucuauguuaaga

SEQ ID NO: 153 - RNA, 29 nt, Mus musculus
uuucacagaaugcugcuucuaggggcugu

SEQ ID NO: 154 - RNA, 27 nt, Mus musculus
ucugagcccugugaagcugcugccugc

SEQ ID NO: 155 - RNA, 29 nt, Mus musculus
uauaaaacauuuuaccuguuuaaaaugua

SEQ ID NO: 156 - RNA, 26 nt, Mus musculus
uaaggguguuaucaagaaaagugucc

SEQ ID NO: 157 - RNA, 32 nt, Mus musculus
auucacagaaugcuaaaguguccuguaagcuu

SEQ ID NO: 158 - RNA, 26 nt, Mus musculus
uugaauuuagugucauggccuguuuu

SEQ ID NO: 159 - RNA, 26 nt, Mus musculus
uguguguaggugccugaggaugccug

SEQ ID NO: 160 - RNA, 34 nt, Mus musculus
agcuuguaaaguugcuauaaauggcucuuuggca

SEQ ID NO: 161 - RNA, 26 nt, Mus musculus
uaguaagguaaucaaaggaucagugg

SEQ ID NO: 162 - RNA, 27 nt, Mus musculus
uuguagguacagcaauaaauaaauggg

SEQ ID NO: 163 - RNA, 26 nt, Mus musculus
uugaauuuagugucauggccuguuuu

SEQ ID NO: 164 - RNA, 26 nt, Mus musculus
uagugucugaagcaggaaaacacugc

SEQ ID NO: 165 - RNA, 27 nt, Mus musculus
ccugaagcaguggaaggggccucugua

SEQ ID NO: 166 - RNA, 29 nt, Mus musculus
ugucuguagacaucaauuuugcccagccu

SEQ ID NO: 167 - RNA, 26 nt, Mus musculus
uguagauuugacacugaugaccuggc

SEQ ID NO: 168 - RNA, 29 nt, Mus musculus
uucuauuacucugaugugacagagaagga

SEQ ID NO: 169 - RNA, 26 nt, Mus musculus
uaaacaaguagugucuccuagggccu

SEQ ID NO: 170 - RNA, 26 nt, Mus musculus
uaaaagugcuuuugagacaucguugg

SEQ ID NO: 171 - RNA, 26 nt, Mus musculus
guagauuugacacugaugaccuggca

SEQ ID NO: 172 - RNA, 29 nt, Mus musculus
ugucuguagacaucaauuuugcccagccu

SEQ ID NO: 173 - RNA, 28 nt, Mus musculus
uguaaacccacaagguaggaacauggcc

SEQ ID NO: 174 - RNA, 25 nt, Mus musculus
cuuuagcuugugggacugcaggugg

SEQ ID NO: 175 - RNA, 26 nt, Mus musculus
uauuauugaguauuuuaaagaagggc

SEQ ID NO: 176 - RNA, 28 nt, Mus musculus
uugucuuuuuugaugacuuuggguuggg

SEQ ID NO: 177 - RNA, 26 nt, Mus musculus
uauuugagcuuugggauuugaugcua

SEQ ID NO: 178 - RNA, 26 nt, Mus musculus
uuacagaugguuguaagcugucaugu

SEQ ID NO: 179 - RNA, 31 nt, Mus musculus
uacagaugguuguaagccaccauaugggugc

SEQ ID NO: 180 - RNA, 25 nt, Mus musculus
uucuaagugugugugucugucugug

SEQ ID NO: 181 - RNA, 26 nt, Mus musculus
uguguguguucuguuguguaucucaa

SEQ ID NO: 182 - RNA, 26 nt, Mus musculus
uaaaggugauuccuucauaauaaggg

SEQ ID NO: 183 - RNA, 29 nt, Mus musculus
uaucauuaugccuuuucuucccuacagau

SEQ ID NO: 184 - RNA, 26 nt, Mus musculus
uaaaauuauggugauugaugauggua

SEQ ID NO: 185 - RNA, 26 nt, Mus musculus
uacauuuguggguuucuagaaacagc

SEQ ID NO: 186 - RNA, 26 nt, Mus musculus
ugucucugaugcagcuuauacgaaga

SEQ ID NO: 187 - RNA, 27 nt, Mus musculus
uaucaaaguugaaucaggaucaaguug

SEQ ID NO: 188 - RNA, 26 nt, Mus musculus
ugcugaucuuuauaacuggaaaacua

SEQ ID NO: 189 - RNA, 26 nt, Mus musculus
ucugucuugaaagugcucaaaauucu

SEQ ID NO: 190 - RNA, 27 nt, Mus musculus
ugcuuauuagugaugaaguuuuccagg

SEQ ID NO: 191 - RNA, 26 nt, Mus musculus
ugcaguuuuaugaggcuaugggacua

SEQ ID NO: 192 - RNA, 26 nt, Mus musculus
ugauuacagagcaaggguggugaagc

SEQ ID NO: 193 - RNA, 28 nt, Mus musculus
ugaaaguuuacaaccaucuguacagcua

SEQ ID NO: 194 - RNA, 27 nt, Mus musculus
ucagacagauggacaaguuuuuggugc

SEQ ID NO: 195 - RNA, 27 nt, Mus musculus
uacaauuuuuauugcuacaauugucug

SEQ ID NO: 196 - RNA, 27 nt, Mus musculus
uuuuucugaagaagacuggucaguugu

SEQ ID NO: 197 - RNA, 26 nt, Mus musculus
uagaaguaacagagauucuuggacag

SEQ ID NO: 198 - RNA, 29 nt, Mus musculus
uuuccagaguuguuguaccaauuuccaau

SEQ ID NO: 199 - RNA, 27 nt, Mus musculus
ugaaauaacaacucuaauacuuaauua

SEQ ID NO: 200 - RNA, 27 nt, Mus musculus
uauaaagaucagcagaggaaaaugacc

SEQ ID NO: 201 - RNA, 26 nt, Mus musculus
cugcaucagagacaacugaagaugaa

SEQ ID NO: 202 - RNA, 29 nt, Mus musculus
uaucauuaugccuuuucuucccuacagau

SEQ ID NO: 203 - RNA, 27 nt, Mus musculus
uaucaaaguugaaucaggaucaaguug

SEQ ID NO: 204 - RNA, 26 nt, Mus musculus
uaaaggugauuccuucauaauaaggg

SEQ ID NO: 205 - RNA, 31 nt, Mus musculus; identical to SEQ ID NO: 179

SEQ ID NO: 206 - RNA, 26 nt, Mus musculus
uauuauugaguauuuuaaagaagggc

SEQ ID NO: 207 - RNA, 26 nt, Mus musculus
uguuguuuuauuuuuguagcucugga

SEQ ID NO: 208 - RNA, 27 nt, Mus musculus
ccugaagcaguggaaggggccucugua

SEQ ID NO: 209 - RNA, 27 nt, Mus musculus
uuguagguacagcaauaaauaaauggg

SEQ ID NO: 210 - RNA, 27 nt, Mus musculus
uagcacuaacucugacccucuuacccu

SEQ ID NO: 211 - RNA, 26 nt, Mus musculus
uugaauuuagugucauggccuguuuu

SEQ ID NO: 212 - RNA, 26 nt, Mus musculus
uguguguaggugccugaggaugccug

SEQ ID NO: 213 - RNA, 34 nt, Mus musculus; identical to SEQ ID NO: 160

SEQ ID NO: 214 - RNA, 27 nt, Mus musculus
cacagcauucugugaaccucauccaug

SEQ ID NO: 215 - RNA, 27 nt, Mus musculus
ucugagcccugugaagcugcugccugc

SEQ ID NO: 216 - RNA, 29 nt, Mus musculus
uauaaaacauuuuaccuguuuaaaaugua

SEQ ID NO: 217 - RNA, 26 nt, Mus musculus
uagagcaagcuguauguacaucugua

SEQ ID NO: 218 - RNA, 27 nt, Mus musculus
uucaccauagucaccugucaggaaugu

SEQ ID NO: 219 - RNA, 26 nt, Mus musculus
uugagucacaucugcaccacagcucc

SEQ ID NO: 220 - RNA, 26 nt, Mus musculus
uaaguaaaguugagccgccuaucagc

SEQ ID NO: 221 - RNA, 26 nt, Mus musculus
ucuuaaugaagucuuuaaaaaaacca

SEQ ID NO: 222 - RNA, 29 nt, Mus musculus
ucaacauaagaauggauacagaaaaugug

SEQ ID NO: 223 - RNA, 30 nt, Mus musculus; identical to SEQ ID NO: 145

SEQ ID NO: 224 - RNA, 28 nt, Mus musculus
ugacagcuugacaagagugacuuuuaaa

SEQ ID NO: 225 - RNA, 30 nt, Mus musculus; identical to SEQ ID NO: 141

SEQ ID NO: 226 - RNA, 26 nt, Mus musculus
gaacuuccuuccuucuaggugacccc

SEQ ID NO: 227 - RNA, 26 nt, Mus musculus
uuagcucucugaaguugugaguguac

SEQ ID NO: 228 - RNA, 26 nt, Mus musculus
uacaguuugaaaagcugagaucaggg

SEQ ID NO: 229 - RNA, 27 nt, Mus musculus
aagcucucugaaguguuagccaaggag

SEQ ID NO: 230 - RNA, 28 nt, Mus musculus
uagagaaagugugucccuggaugugggu

SEQ ID NO: 231 - RNA, 27 nt, Mus musculus
uguuagagaaaguguaccacuaggagc

SEQ ID NO: 232 - RNA, 25 nt, Mus musculus
gucagcugacugaaggggguguuuc

SEQ ID NO: 233 - RNA, 27 nt, Mus musculus
uuggcuuccgguguacugcauguaugu

SEQ ID NO: 234 - RNA, 26 nt, Mus musculus
uccuuuaauaaagugagaccaggggc

SEQ ID NO: 235 - RNA, 27 nt, Mus musculus
ugaggccaugcuaaacuuugucgaguc

SEQ ID NO: 236 - RNA, 29 nt, Mus musculus
ugaacaacuucagaacagccagccgacuu

SEQ ID NO: 237 - RNA, 28 nt, Mus musculus
uugcccuguuguccugaauggaaggucu

SEQ ID NO: 238 - RNA, 26 nt, Mus musculus
ucuggacacauugaggcugcugcaca

SEQ ID NO: 239 - RNA, 27 nt, Mus musculus
uagcugcauaugaugaaaagauggccu

SEQ ID NO: 240 - RNA, 26 nt, Mus musculus
ugugaauucauauauaugggagcugg

SEQ ID NO: 241 - RNA, 28 nt, Mus musculus
uccacauggaaauggcauuuaaacuccu

SEQ ID NO: 242 - RNA, 26 nt, Mus musculus
uaacagagauuauaggugugaaccac

SEQ ID NO: 243 - RNA, 29 nt, Mus musculus
uaaacagagaaaugccuucagcaacaugc

SEQ ID NO: 244 - RNA, 26 nt, Mus musculus
uaauuccaguuugaggaaauccaaug

SEQ ID NO: 245 - RNA, 28 nt, Mus musculus
cuguucuuuucacuggguauguuggugc

SEQ ID NO: 246 - RNA, 27 nt, Mus musculus
ugagaaaccaccuguggagagaaugga

SEQ ID NO: 247 - RNA, 29 nt, Mus musculus
uagccacaagguuaaugggucuuucuauc

SEQ ID NO: 248 - RNA, 26 nt, Mus musculus
uaaccaccugucuccagcuccgggaa

SEQ ID NO: 249 - RNA, 26 nt, Mus musculus
cauagccacaaggugucaguguuacc

SEQ ID NO: 250 - RNA, 26 nt, Mus musculus
uaagucauugagagcucuagccugga

SEQ ID NO: 251 - RNA, 30 nt, Mus musculus; identical to SEQ ID NO: 96

SEQ ID NO: 252 - RNA, 27 nt, Mus musculus
ugauaaaaugucaagaccaaagcuggg

SEQ ID NO: 253 - RNA, 26 nt, Mus musculus
gaauguugugacuuuuacugcaacaa

SEQ ID NO: 254 - RNA, 28 nt, Mus musculus
uuuguuguuacaaccuucugugucugcu

SEQ ID NO: 255 - RNA, 29 nt, Mus musculus
uauacaagcaccaaguaaauccugggagc

SEQ ID NO: 256 - RNA, 30 nt, Mus musculus; identical to SEQ ID NO: 91

SEQ ID NO: 257 - RNA, 26 nt, Mus musculus
gcugcugaaauaaaaagaagaggagg

SEQ ID NO: 258 - RNA, 26 nt, Mus musculus
uaguaaagauggauguggcaugucaa

SEQ ID NO: 259 - RNA, 27 nt, Mus musculus
uaagccaagaauuaugaguguccugcu

SEQ ID NO: 260 - RNA, 26 nt, Mus musculus
uaaaguaaagaaauacagccacuggg

SEQ ID NO: 261 - RNA, 30 nt, Mus musculus; identical to SEQ ID NO: 83

SEQ ID NO: 262 - RNA, 26 nt, Mus musculus
uaauugguugcucuggagaugagggg

SEQ ID NO: 263 - RNA, 26 nt, Mus musculus
ugugacuccuguuguguauggucuug

SEQ ID NO: 264 - RNA, 28 nt, Mus musculus
uaaaggugacuccuggcaccccuggccu

SEQ ID NO: 265 - RNA, 26 nt, Mus musculus
uguagagcagguggaagggagcugaa

SEQ ID NO: 266 - RNA, 26 nt, Mus musculus
uacaaaaguuagcaguuuuagguuua

SEQ ID NO: 267 - RNA, 25 nt, Mus musculus
cagagcagguggauuccaaguuuga

SEQ ID NO: 268 - RNA, 27 nt, Mus musculus
uagaaguuaaaccagaaaugugugccu

SEQ ID NO: 269 - RNA, 26 nt, Mus musculus
ugugguaggauuacucagcuguuaau

SEQ ID NO: 270 - RNA, 26 nt, Mus musculus
uguugucuacuguuaggaauagagug

SEQ ID NO: 271 - RNA, 28 nt, Mus musculus
uagauaaccauuauacaaaacauccaau

SEQ ID NO: 272 - RNA, 27 nt, Mus musculus
uaauaaaauguucuuuugacaaaugaa

SEQ ID NO: 273 - RNA, 27 nt, Mus musculus
uguuucuucugcaugcuccagugucgc

SEQ ID NO: 274 - RNA, 27 nt, Mus musculus
uguaaucaucagauguauguguggcuu

SEQ ID NO: 275 - RNA, 26 nt, Mus musculus
aauuuugagcaguuccuccaggagag

SEQ ID NO: 276 - RNA, 26 nt, Mus musculus
aaaugacuuuagaucugguaaugagu

SEQ ID NO: 277 - RNA, 26 nt, Mus musculus
ugagggacaaggacuccaugggacac

SEQ ID NO: 278 - RNA, 26 nt, Mus musculus
uucaaaacagaaaguagugaggacuc

SEQ ID NO: 279 - RNA, 27 nt, Mus musculus
gcccucuguagcagccucugaugcaaa

SEQ ID NO: 280 - RNA, 26 nt, Mus musculus
auguuucaaaacagaaaguagugagg

SEQ ID NO: 281 - RNA, 26 nt, Mus musculus
uuggagaggugacucugcaguuaaaa

SEQ ID NO: 282 - RNA, 26 nt, Mus musculus
ugucacaugucuuggacagcaugcgu

SEQ ID NO: 283 - RNA, 50 nt, Mus musculus
ugcuccaguaacaguuacagaaaccccggagccagcaaugcccaguggug

SEQ ID NO: 284 - RNA, 33 nt, Mus musculus
uacccaugugucaaaaucagugcuaacuggaca

SEQ ID NO: 285 - RNA, 28 nt, Mus musculus
uaaaagaauuacuaauaaaacaaaaagg

SEQ ID NO: 286 - RNA, 34 nt, Mus musculus
gccaagucugugaauugcaagguuuuaguucaag

SEQ ID NO: 287 - RNA, 27 nt, Mus musculus
ugaagacccagucccuaccuuagccua

SEQ ID NO: 288 - RNA, 29 nt, Mus musculus
ugugaaggugucuuugucacuaauagaug

SEQ ID NO: 289 - RNA, 27 nt, Mus musculus
uaaaacaggguaauuccugccacggac

SEQ ID NO: 290 - RNA, 27 nt, Mus musculus
ugacuccuggugauuggcaaacgguug

SEQ ID NO: 291 - RNA, 26 nt, Mus musculus
uauuacagaugcugucaaacuuauuc

SEQ ID NO: 292 - RNA, 26 nt, Mus musculus
uucuaagguuggugagaugucuaaac

SEQ ID NO: 293 - RNA, 26 nt, Mus musculus
ugcugacacuacugaacuggacugcu

SEQ ID NO: 294 - RNA, 27 nt, Mus musculus
uaguuaucagacucacuggacuuuugu

SEQ ID NO: 295 - RNA, 26 nt, Mus musculus
uggaaagggcuaucuucucaguguua

SEQ ID NO: 296 - RNA, 26 nt, Mus musculus
ugacuuaugucccuggcccaggguga

SEQ ID NO: 297 - RNA, 30 nt, Mus musculus
ugcaaauuugaugaauucugauggcuuuuu

SEQ ID NO: 298 - RNA, 27 nt, Mus musculus
uagaaaugaucucugcuuuauugucua

SEQ ID NO: 299 - RNA, 26 nt, Mus musculus
uguguguguguguuuaagaauauuau

SEQ ID NO: 300 - RNA, 30 nt, Mus musculus
ugugcuguucuuacugagucuucuugcuca

SEQ ID NO: 301 - RNA, 28 nt, Mus musculus
uggcaucagugucuuuguuucaaaaguu

SEQ ID NO: 302 - RNA, 28 nt, Mus musculus
ugauugcugccagagaccucaucagaau

SEQ ID NO: 303 - RNA, 26 nt, Mus musculus
ugaaaucuaggaagagaaucagcagg

SEQ ID NO: 304 - RNA, 27 nt, Mus musculus
ucagugucuuuguuucaaaaguuguuu

SEQ ID NO: 305 - RNA, 31 nt, Mus musculus
uaccugcaaauuugaugaauucugauggcuu

SEQ ID NO: 306 - RNA, 27 nt, Mus musculus
uaccugauugcugccagagaccucauc

SEQ ID NO: 307 - RNA, 30 nt, Mus musculus
uaaauuuaccugcaaauuugaugaauucug

SEQ ID NO: 308 - RNA, 31 nt, Mus musculus
guagacugugaagucccuguugcuacuccuu

SEQ ID NO: 309 - RNA, 27 nt, Mus musculus
cucagccaggaacaucaccagauguug

SEQ ID NO: 310 - RNA, 31 nt, Mus musculus
aggaacaucaccagauguuggggauccacac

SEQ ID NO: 311 - RNA, 26 nt, Mus musculus
aagauuacaaacuugugccaagagag

SEQ ID NO: 312 - RNA, 29 nt, Mus musculus
ugaagucugccugugaagucugccuguga

SEQ ID NO: 313 - RNA, 30 nt, Mus musculus
uccugaagaagaaucacaaucguucacagu

SEQ ID NO: 314 - RNA, 26 nt, Mus musculus
gugaagucugccugugaagucugccu

SEQ ID NO: 315 - RNA, 27 nt, Mus musculus
uguaugauucuuaaccuaugucuggaa

SEQ ID NO: 316 - RNA, 26 nt, Mus musculus
uggcaggagcaguuccuaacugggag

SEQ ID NO: 317 - RNA, 29 nt, Mus musculus
ugugaucaaccuaucucccuuaauggacu

SEQ ID NO: 318 - RNA, 39 nt, Mus musculus
uaccaugaggugcugagaagaagguguauucuuuuguuu

SEQ ID NO: 319 - RNA, 30 nt, Mus musculus
uaccaugaggugcugagaagaagguauauc

SEQ ID NO: 320 - RNA, 25 nt, Mus musculus
accaugaggugcugagaggaaggua

SEQ ID NO: 321 - RNA, 25 nt, Mus musculus
uaagugcacuugcuguucucgcaga

SEQ ID NO: 322 - RNA, 26 nt, Mus musculus
uuugauggaucuggagauucccggga

SEQ ID NO: 323 - RNA, 31 nt, Mus musculus
uaaauuuguggguaugucagccacagaaggu

SEQ ID NO: 324 - RNA, 26 nt, Mus musculus
gucuuggucauagaaaguaucugaaa

SEQ ID NO: 325 - RNA, 26 nt, Mus musculus
acaaccugguuagaagaugcccguca

SEQ ID NO: 326 - RNA, 27 nt, Mus musculus
auuugagauuagacuuaauacagugug

SEQ ID NO: 327 - RNA, 27 nt, Mus musculus
ggagcgauuugucugguuaauuccgau

SEQ ID NO: 328 - RNA, 29 nt, Mus musculus
gagcgauuugucugguuaauuccgauaac

SEQ ID NO: 329 - RNA, 31 nt, Mus musculus
ugaagaagaaucacaaucguucacagugguc

SEQ ID NO: 330 - RNA, 27 nt, Mus musculus
ugaaaaaugugugccauuugaaacucu

SEQ ID NO: 331 - RNA, 29 nt, Mus musculus
uacacccacaaccagaagugauuguuuuc

SEQ ID NO: 332 - RNA, 29 nt, Mus musculus
uuguugcugauucucuucuccagagguga

SEQ ID NO: 333 - RNA, 29 nt, Mus musculus
uuaguugguggagcgauuugucugguuaa

SEQ ID NO: 334 - RNA, 27 nt, Mus musculus
ugugaagucugccugugaagucugccu

SEQ ID NO: 335 - RNA, 27 nt, Mus musculus
ugugaagucugccugugaagucagucu

SEQ ID NO: 336 - RNA, 27 nt, Mus musculus
ugugaagucugccugugaagucagccu

SEQ ID NO: 337 - RNA, 26 nt, Mus musculus
ugguggcugacagcaucuguaauggg

SEQ ID NO: 338 - RNA, 27 nt, Mus musculus
ugguggagcgauuugucugguuaauuc

SEQ ID NO: 339 - RNA, 29 nt, Mus musculus
ugcuuuucuugugcagggcugauggcugu

SEQ ID NO: 340 - RNA, 32 nt, Mus musculus
ugauagauguucuccugaagaagaaucacaau

SEQ ID NO: 341 - RNA, 30 nt, Mus musculus
ucuuaguugguggagcgauuugucugguua

SEQ ID NO: 342 - RNA, 26 nt, Mus musculus
uaguugguggagcgauuugucugguu

SEQ ID NO: 343 - RNA, 27 nt, Mus musculus
uagcacuuuguuucugagaugagguuc

SEQ ID NO: 344 - RNA, 30 nt, Mus musculus
uagauguucuccugaagaagaaucacaauc

SEQ ID NO: 345 - RNA, 25 nt, Mus musculus
gucagucugugaagucugccuguga

SEQ ID NO: 346 - RNA, 27 nt, Mus musculus
cuuaguugguggagcgauuugucuggu

SEQ ID NO: 347 - RNA, 28 nt, Mus musculus
aguugguggagcgauuugucugguuaau

SEQ ID NO: 348 - RNA, 26 nt, Mus musculus
uaaaaaaugggaauauggaguaacuc

SEQ ID NO: 349 - RNA, 27 nt, Mus musculus
uguugauggucauuguuuguaacaguu

SEQ ID NO: 350 - RNA, 31 nt, Mus musculus
ugucuuuuugaacauguuaccucagcauaca

SEQ ID NO: 351 - RNA, 27 nt, Mus musculus
ugauaaugaggcuuugggaguauaacc

SEQ ID NO: 352 - RNA, 26 nt, Mus musculus
ugauaaugaggcuuugggaauauaac

SEQ ID NO: 353 - RNA, 26 nt, Mus musculus
ugagaguaaaagacugugcugccuua

SEQ ID NO: 354 - RNA, 27 nt, Mus musculus
uaaggacucuucacaauuagaacuguu

SEQ ID NO: 355 - RNA, 29 nt, Mus musculus
uaaauaaagcuuuaauuuaugaugaaccu

SEQ ID NO: 356 - RNA, 26 nt, Mus musculus
guuugcuguuguuguuuaaaugccuu

SEQ ID NO: 357 - RNA, 31 nt, Mus musculus
caggaggaugucuuuuugaacauguuaccuc

SEQ ID NO: 358 - RNA, 31 nt, Mus musculus
aggaggaugucuuuuugaacauguuaccuca

SEQ ID NO: 359 - RNA, 26 nt, Mus musculus
uucaaggccagcagcuacagagugag

SEQ ID NO: 360 - RNA, 26 nt, Mus musculus
ugaaguaacuguguauacuggguaua

SEQ ID NO: 361 - RNA, 26 nt, Mus musculus
uauugugugaauuugguuuuguggug

SEQ ID NO: 362 - RNA, 26 nt, Mus musculus
uauugugugaauuugguuuugucauu

SEQ ID NO: 363 - RNA, 26 nt, Mus musculus
uauugugugaauuugguuuugucagg

SEQ ID NO: 364 - RNA, 26 nt, Mus musculus
auugugugaauuugguuuuguccugg

SEQ ID NO: 365 - RNA, 26 nt, Mus musculus
auugugugaauuugguuuugucaugg

SEQ ID NO: 366 - RNA, 26 nt, Mus musculus
auugugugaauuugguugugucaugg

SEQ ID NO: 367 - RNA, 34 nt, Mus musculus
auguucuucaggcuccccugcagguuuguuuuug

SEQ ID NO: 368 - RNA, 42 nt, Mus musculus
agcuucccucugugguucagugaacacccuuggaaugcccca

SEQ ID NO: 369 - RNA, 26 nt, Mus musculus
uggcauucugguagucauguuguugg

SEQ ID NO: 370 - RNA, 30 nt, Mus musculus
uggaaagauauugugugaauuugguuuugu

SEQ ID NO: 371 - RNA, 26 nt, Mus musculus
ugcugcauacacccuuggagaguuca

SEQ ID NO: 372 - RNA, 27 nt, Mus musculus
ugcauacacccuuggagaguucacugg

SEQ ID NO: 373 - RNA, 27 nt, Mus musculus
ugaaagccaaagccucaauagaaagaa

SEQ ID NO: 374 - RNA, 26 nt, Mus musculus
uauugugugaauuugguuuugucaug

SEQ ID NO: 375 - RNA, 27 nt, Mus musculus
uaagccuugagagucauguucagaaag

SEQ ID NO: 376 - RNA, 26 nt, Mus musculus
aaacaguucuaauugugaagaguccu

SEQ ID NO: 377 - RNA, 26 nt, Mus musculus
uuuuggauugugaagauucugugguu

SEQ ID NO: 378 - RNA, 28 nt, Mus musculus
uuugauugugaagauucuguguucuagu

SEQ ID NO: 379 - RNA, 27 nt, Mus musculus
ugaauguucuucuagagguccugaguu

SEQ ID NO: 380 - RNA, 31 nt, Mus musculus
cgacuguucuuccagaguuccugaguucaau

SEQ ID NO: 381 - RNA, 26 nt, Mus musculus
cagaagaucaggaacuaacaggcaaa

SEQ ID NO: 382 - RNA, 26 nt, Mus musculus
gaagguuuuacaagauaaggggcuuc

SEQ ID NO: 383 - RNA, 27 nt, Mus musculus
ucaggaccucuagaagaacagucagug

SEQ ID NO: 384 - RNA, 26 nt, Mus musculus
ucaggaccucuagaagaacaaucagu

SEQ ID NO: 385 - RNA, 31 nt, Mus musculus
ccaaauuuaagaccagaagaucaggaggauc

SEQ ID NO: 386 - RNA, 27 nt, Mus musculus
uaaacucagaagaucaggaacuaaucg

SEQ ID NO: 387 - RNA, 28 nt, Mus musculus
uugaacucaggaccucuagaagaacagu

SEQ ID NO: 388 - RNA, 25 nt, Mus musculus
ugaacucaggaucucuagaagaaca

SEQ ID NO: 389 - RNA, 29 nt, Mus musculus
ugaacucaggaccucuagaagaacaguca

SEQ ID NO: 390 - RNA, 27 nt, Mus musculus
ugaacucaggaccucuagaagaacaga

SEQ ID NO: 391 - RNA, 28 nt, Mus musculus
gaacugaacucaggaccucuagaagaac

SEQ ID NO: 392 - RNA, 26 nt, Mus musculus
aagaugcucuagaagaauuccugcca

SEQ ID NO: 393 - RNA, 26 nt, Mus musculus
uaguuuuucuguucaaugguucauga

SEQ ID NO: 394 - RNA, 26 nt, Mus musculus
uaguuuuucuguuaagugaagagggg

SEQ ID NO: 395 - RNA, 26 nt, Mus musculus
ugcuuugcuaguucuucuguuuggaa

SEQ ID NO: 396 - RNA, 26 nt, Mus musculus
uugcuuugcuaguucuucuguuugga

SEQ ID NO: 397 - RNA, 26 nt, Mus musculus
uuccaaacagaaaaugcagcuuucga

SEQ ID NO: 398 - RNA, 26 nt, Mus musculus
uccaaacagaagaacuagcaaagcaa

SEQ ID NO: 399 - RNA, 26 nt, Mus musculus
uccaaacagaaaagcuuaaaguuaag

SEQ ID NO: 400 - RNA, 27 nt, Mus musculus
ugcuucuuucagacuucccuucugucu

SEQ ID NO: 401 - RNA, 29 nt, Mus musculus
uggaauguaagcuccuugaacaauggcga

SEQ ID NO: 402 - RNA, 30 nt, Mus musculus
uacauacuguuuccaccgguggaauugcua

SEQ ID NO: 403 - RNA, 26 nt, Mus musculus
ugauguaagguuuuuguauauaagac

SEQ ID NO: 404 - RNA, 27 nt, Mus musculus
uaugcuacuaccuuguuuugugcuugc

SEQ ID NO: 405 - RNA, 26 nt, Mus musculus
uugagauugcuucuuucagaagaugc

SEQ ID NO: 406 - RNA, 38 nt, Mus musculus
cugcugcuucugcaguuuugugcuugcuguauuuuucu

SEQ ID NO: 407 - RNA, 28 nt, Mus musculus
augccuaccacaccguugaagagcuuua

SEQ ID NO: 408 - RNA, 26 nt, Mus musculus
uagcaauuccaccgguggaaacagua

SEQ ID NO: 409 - RNA, 26 nt, Mus musculus
cuguacaagcaaagcucuugggaggu

SEQ ID NO: 410 - RNA, 26 nt, Mus musculus
ccuguaugcagcaaaauguugggucc

SEQ ID NO: 411 - RNA, 27 nt, Mus musculus
uuuccuguaugcagcaaaaugcugugu

SEQ ID NO: 412 - RNA, 29 nt, Mus musculus
ugaguucaaauccuguacaagcaaagcuc

SEQ ID NO: 413 - RNA, 28 nt, Mus musculus
ugagguugguuuccuguaugcagcaaaa

SEQ ID NO: 414 - RNA, 29 nt, Mus musculus
uccuguacaagcaaagcucuugggaggua

SEQ ID NO: 415 - RNA, 29 nt, Mus musculus
ucaaauccuguacaagcaaagcucuuggg

SEQ ID NO: 416 - RNA, 34 nt, Mus musculus
uauuccuguaugcagcaaaaugcuggguccuauu

SEQ ID NO: 417 - RNA, 29 nt, Mus musculus
uagagcaaaaccugaguccugaaagacag

SEQ ID NO: 418 - RNA, 27 nt, Mus musculus
uagaagaacagcaagaccugagucacc

SEQ ID NO: 419 - RNA, 27 nt, Mus musculus
agauggguuuccuguaugcagcaaaau

SEQ ID NO: 420 - RNA, 25 nt, Mus musculus
uuuuaccuggaauggucugucugcc

SEQ ID NO: 421 - RNA, 27 nt, Mus musculus
uuggaagaagacaagaagcaaugaaga

SEQ ID NO: 422 - RNA, 26 nt, Mus musculus
uaugaggacuuugaaaguuggacuaa

SEQ ID NO: 423 - RNA, 26 nt, Mus musculus
uuagguauuuuaugaggacuuuuaag

SEQ ID NO: 424 - RNA, 27 nt, Mus musculus
ugaguaccaauggagauugagacagaa

SEQ ID NO: 425 - RNA, 26 nt, Mus musculus
uagacuaugaggacuuugaaaguugg

SEQ ID NO: 426 - RNA, 26 nt, Mus musculus
uuggacagguaacuauagcagagcca

SEQ ID NO: 427 - RNA, 30 nt, Mus musculus
ugcuggugauucggaucaagauuccaaaua

SEQ ID NO: 428 - RNA, 26 nt, Mus musculus
uguaaggcauuuucucaguuagugau

SEQ ID NO: 429 - RNA, 27 nt, Mus musculus
uaauucaucuaauugaggagcagcauc

SEQ ID NO: 430 - RNA, 26 nt, Mus musculus
aauuugcuuuugcuuuaaucccaggu

SEQ ID NO: 431 - RNA, 26 nt, Mus musculus
uagcaucugugucaucagacaagagg

SEQ ID NO: 432 - RNA, 26 nt, Mus musculus
uuccagaagagccagguucaguuccc

SEQ ID NO: 433 - RNA, 31 nt, Mus musculus
uacacucuugguaguggggagccaugggauc

SEQ ID NO: 434 - RNA, 25 nt, Mus musculus
uuuuagccuuucugccguucugaca

SEQ ID NO: 435 - RNA, 34 nt, Mus musculus
auucuucccugccucuccuauuccuuguuuuagc

SEQ ID NO: 436 - RNA, 25 nt, Mus musculus
uguguauuugugcuuuuuccugaaa

SEQ ID NO: 437 - RNA, 27 nt, Mus musculus
cagaucuuuugguucucuucucuaacc

SEQ ID NO: 438 - RNA, 26 nt, Mus musculus
auguugacuucuauuuggaauccuaa

SEQ ID NO: 439 - RNA, 26 nt, Mus musculus
uaaggaauagcagaaugcuuuaaugc

SEQ ID NO: 440 - RNA, 39 nt, Mus musculus
accagaagucaauuaaugcucugguguugguugguuucu

SEQ ID NO: 441 - RNA, 31 nt, Mus musculus
uagagccuguguaaaaagcacaaaugugugc

SEQ ID NO: 442 - RNA, 25 nt, Mus musculus
uuuuagccuuucugccguucugaca

SEQ ID NO: 443 - RNA, 34 nt, Mus musculus; identical to SEQ ID NO: 435

SEQ ID NO: 444 - RNA, 25 nt, Mus musculus
uguguauuugugcuuuuuccugaaa

SEQ ID NO: 445 - RNA, 27 nt, Mus musculus
cagaucuuuugguucucuucucuaacc

SEQ ID NO: 446 - RNA, 26 nt, Mus musculus
auguugacuucuauuuggaauccuaa

SEQ ID NO: 447 - RNA, 26 nt, Mus musculus
uaaggaauagcagaaugcuuuaaugc

SEQ ID NO: 448 - RNA, 39 nt, Mus musculus; identical to SEQ ID NO: 440

SEQ ID NO: 449 - RNA, 29 nt, Mus musculus
ugaacaaaaaagugagaaccaaacggauu

SEQ ID NO: 450 - RNA, 31 nt, Mus musculus; identical to SEQ ID NO: 441

SEQ ID NO: 451 - RNA, 30 nt, Mus musculus
uuccaggaggcugguucuaaaacaugaugu

SEQ ID NO: 452 - RNA, 26 nt, Mus musculus
ugaaagacuuuuuagaguuuaaugaa

SEQ ID NO: 453 - RNA, 27 nt, Mus musculus
uaaagaaauuaaagacuuuuuagaguu

SEQ ID NO: 454 - RNA, 26 nt, Mus musculus
ugcagcuacaguugugugcuacucuc

SEQ ID NO: 455 - RNA, 28 nt, Mus musculus
ccaggacggaugcagcuacaguugccau

SEQ ID NO: 456 - RNA, 25 nt, Mus musculus
ugugaaacaggaaacugugugguuu

SEQ ID NO: 457 - RNA, 30 nt, Mus musculus
ugucuugagaaaacaagaaaugauuggguc

SEQ ID NO: 458 - RNA, 32 nt, Mus musculus
uggacuucccuauggucgugaccuuucccgcc

SEQ ID NO: 459 - RNA, 34 nt, Mus musculus
cuccaugagcagugcugggaacaguagcaggaac

SEQ ID NO: 460 - RNA, 26 nt, Mus musculus
uaagaugguauuucuagcuguuaggu

SEQ ID NO: 461 - RNA, 25 nt, Mus musculus
acuggugacucuucuuccugcugau

SEQ ID NO: 462 - RNA, 28 nt, Mus musculus
ugaaaggacaaaaagaagaaaggaaggg

SEQ ID NO: 463 - RNA, 26 nt, Mus musculus
uugaacugacagaagaaacagcaagc

SEQ ID NO: 464 - RNA, 30 nt, Mus musculus
ugacagcaaaugaccuacacaggugcaagu

SEQ ID NO: 465 - RNA, 26 nt, Mus musculus
uuguggaagucagagcaaaaugucug

SEQ ID NO: 466 - RNA, 26 nt, Mus musculus
uuauaggcauggaagucacaggaaag

SEQ ID NO: 467 - RNA, 27 nt, Mus musculus
uuaacugaugugggaagaccuuaaauu

SEQ ID NO: 468 - RNA, 27 nt, Mus musculus
uuaaauucaacuccaggggcuggaaac

SEQ ID NO: 469 - RNA, 27 nt, Mus musculus
ugguaucuaaguucaagaaauucaggg

SEQ ID NO: 470 - RNA, 30 nt, Mus musculus
uggacucugaguuuuggggaccaggacacu

SEQ ID NO: 471 - RNA, 28 nt, Mus musculus
ugccagagcaacugagggagccaucuug

SEQ ID NO: 472 - RNA, 26 nt, Mus musculus
ugaggaacaaaaugucugguaagaug

SEQ ID NO: 473 - RNA, 27 nt, Mus musculus
ugaaaaaccagaauggagaaagggccu

SEQ ID NO: 474 - RNA, 26 nt, Mus musculus
ucuaaguucaagaaauucagggcuga

SEQ ID NO: 475 - RNA, 27 nt, Mus musculus
uaugaaaaaccagaauggagaaagggc

SEQ ID NO: 476 - RNA, 26 nt, Mus musculus
uaguucaggacaacagcaaacuguga

SEQ ID NO: 477 - RNA, 26 nt, Mus musculus
uaguguaccaggaacuaaucacccuc

SEQ ID NO: 478 - RNA, 26 nt, Mus musculus
uaggcauggaagucacaggaaagggc

SEQ ID NO: 479 - RNA, 31 nt, Mus musculus
uagcuuuaaauucaacuccaggggcuggaaa

SEQ ID NO: 480 - RNA, 26 nt, Mus musculus
uagagagucagaaagaagaaggcuga

SEQ ID NO: 481 - RNA, 26 nt, Mus musculus
uaauuuccuuggguuuguuuuugguc

SEQ ID NO: 482 - RNA, 26 nt, Mus musculus
ugcagcagauuucuuauuuggguuuu

SEQ ID NO: 483 - RNA, 27 nt, Mus musculus
ucugcagcaggaagagucuuauugucc

SEQ ID NO: 484 - RNA, 27 nt, Mus musculus
ugaaaaucugcagcaggaagaugaggu

SEQ ID NO: 485 - RNA, 25 nt, Mus musculus
uaggaguuuuguuguuguuggccuu

SEQ ID NO: 486 - RNA, 31 nt, Mus musculus
gcuccuguuguuguuggccuuucugccuggu

SEQ ID NO: 487 - RNA, 26 nt, Mus musculus
acuucaaugacuugaucuuuuaaauu

SEQ ID NO: 488 - RNA, 29 nt, Mus musculus
uggacaguguucagaagaggcuugacugu

SEQ ID NO: 489 - RNA, 30 nt, Mus musculus
ugcugccuucuuuuuguccuuucugagcuu

SEQ ID NO: 490 - RNA, 28 nt, Mus musculus
ugcuaaaugaauugaucuuugaaaaguc

SEQ ID NO: 491 - RNA, 28 nt, Mus musculus
ugccuucuuuuuguccuuucugagcuug

SEQ ID NO: 492 - RNA, 25 nt, Mus musculus
ucagaagaggcuugacuguggccug

SEQ ID NO: 493 - RNA, 33 nt, Mus musculus
uucucucuggugggaauguuuucggagauguag

SEQ ID NO: 494 - RNA, 29 nt, Mus musculus
  misc_feature (2)..(2): n is a, c, g, or u
ungguucugauggagcauuguuagcaggc

SEQ ID NO: 495 - RNA, 30 nt, Mus musculus
uggagcauuguuagcaggccacagaaauga

SEQ ID NO: 496 - RNA, 26 nt, Mus musculus
uggaauuuguugccuuguuguuguug

SEQ ID NO: 497 - RNA, 48 nt, Mus musculus
ugcgguuuccccuacugcugcaggaccaggagaggauucguccaacag

SEQ ID NO: 498 - RNA, 31 nt, Mus musculus
uccuguaucagaaauuuggaucuuuucugug

SEQ ID NO: 499 - RNA, 26 nt, Mus musculus
ugccuuguguggugaagggucugcac

SEQ ID NO: 500 - RNA, 26 nt, Mus musculus
ugcaaaccacacaaggcuuuauuccg

SEQ ID NO: 501 - RNA, 26 nt, Mus musculus
ugagcugccuuguguugucuggaauc

SEQ ID NO: 502 - RNA, 26 nt, Mus musculus
uagucaucugcccuguguggucugua

SEQ ID NO: 503 - RNA, 26 nt, Mus musculus
gguguagucuacucuuguuggacaga

SEQ ID NO: 504 - RNA, 30 nt, Mus musculus
uucauucugcacaauguuauuccugugagg

SEQ ID NO: 505 - RNA, 26 nt, Mus musculus
ugugcuauguaguucugacuggugga

SEQ ID NO: 506 - RNA, 26 nt, Mus musculus
uucuggugcaucuaaaguuaacuaca

SEQ ID NO: 507 - RNA, 26 nt, Mus musculus
ugccagggcacaagaguagaccagaa

SEQ ID NO: 508 - RNA, 26 nt, Mus musculus
ugacaagcaaacacaagaguagacuc

SEQ ID NO: 509 - RNA, 26 nt, Mus musculus
uaguguccugaagugagauuaaaguu

SEQ ID NO: 510 - RNA, 26 nt, Mus musculus
ggaaaucuuucauucugcacaauguu

SEQ ID NO: 511 - RNA, 26 nt, Mus musculus
aagugcugagugagauuaaaguugug

SEQ ID NO: 512 - RNA, 26 nt, Mus musculus
ugugauuuuaauaguguggucagugc

SEQ ID NO: 513 - RNA, 29 nt, Mus musculus
uagggagagcugccccuccaguugucugu

SEQ ID NO: 514 - RNA, 27 nt, Mus musculus
ugauuauuccugagagcugccuauauu

SEQ ID NO: 515 - RNA, 26 nt, Mus musculus
uugaauggaagagcccuacacaaaac

SEQ ID NO: 516 - RNA, 43 nt, Mus musculus
cccaggaccauuccgaguguacagugaggauuuugcuucuauc

SEQ ID NO: 517 - RNA, 26 nt, Mus musculus
agaaaaagugguggcucuuuugaagg

SEQ ID NO: 518 - RNA, 26 nt, Mus musculus
uaaaagcuugugaaaauguggugggu

SEQ ID NO: 519 - RNA, 31 nt, Mus musculus
uuugugcauguguuacacacugauggaucuu

SEQ ID NO: 520 - RNA, 27 nt, Mus musculus
uaaugccaagaaaaagugguggcucuu

SEQ ID NO: 521 - RNA, 46 nt, Mus musculus
ccugaugucccaccccaaaaugugguggcuuggaauaagagcuguu

SEQ ID NO: 522 - RNA, 26 nt, Mus musculus
uuaacaguaaucaaauugaaacaccc

SEQ ID NO: 523 - RNA, 27 nt, Mus musculus
uucugcuguaggaaaaggcucaggagu

SEQ ID NO: 524 - RNA, 28 nt, Mus musculus
uuaacaauuuaacaguaaucauaucagg

SEQ ID NO: 525 - RNA, 27 nt, Mus musculus
uguaggaaaaggcucaggagugucccc

SEQ ID NO: 526 - RNA, 30 nt, Mus musculus
ugcuguaggaaaaggcucaggagugucccc

SEQ ID NO: 527 - RNA, 26 nt, Mus musculus
uuuaacaguaaucauaucaggaagcu

SEQ ID NO: 528 - RNA, 27 nt, Mus musculus
ugaaggcucaggagaauggauuugguc

SEQ ID NO: 529 - RNA, 26 nt, Mus musculus
ugucugagcuauucuccugagccuuu

SEQ ID NO: 530 - RNA, 27 nt, Mus musculus
ugacaaaugguugccuagaugaggauu

SEQ ID NO: 531 - RNA, 29 nt, Mus musculus
aguuugaaaucagaggagauuccucugca

SEQ ID NO: 532 - RNA, 26 nt, Mus musculus
ugucuccgagaaguugcagaaguguc

SEQ ID NO: 533 - RNA, 26 nt, Mus musculus
uguccccucuugugccagaaaacaac

SEQ ID NO: 534 - RNA, 26 nt, Mus musculus
uguaagaaaaccgacuccuauaagcu

SEQ ID NO: 535 - RNA, 26 nt, Mus musculus
ugcagaucuaagaggaugcuggcuau

SEQ ID NO: 536 - RNA, 29 nt, Mus musculus
agaagguggacuucagaaaaucaaauaac

SEQ ID NO: 537 - RNA, 27 nt, Mus musculus
agaagguggacuucagaaaaucaaaca

SEQ ID NO: 538 - RNA, 29 nt, Mus musculus
ugagcccaacugcagaucuaagaggaugc

SEQ ID NO: 539 - RNA, 30 nt, Mus musculus
ugacaaaugccugaaaacagacacaaaaaa

SEQ ID NO: 540 - RNA, 27 nt, Mus musculus
uccccucuugugccagaaaacaacguu

SEQ ID NO: 541 - RNA, 27 nt, Mus musculus
ucaagaagguggacuucagaaaaucaa

SEQ ID NO: 542 - RNA, 27 nt, Mus musculus
uagacuugucagaucuaagauccucgu

SEQ ID NO: 543 - RNA, 26 nt, Mus musculus
uagacgacugaggauccaguggacgg

SEQ ID NO: 544 - RNA, 30 nt, Mus musculus
uacacacugacaaaugccugaaaacagaca

SEQ ID NO: 545 - RNA, 26 nt, Mus musculus
uaauacuuaagagcagucauguccuu

SEQ ID NO: 546 - RNA, 28 nt, Mus musculus
cugacaaaugccugaaaacagacacaaa

SEQ ID NO: 547 - RNA, 28 nt, Mus musculus
cacacugacaaaugccugaaaacagaca

SEQ ID NO: 548 - RNA, 29 nt, Mus musculus
uuucuuccucuucugcucagagaacagcc

SEQ ID NO: 549 - RNA, 29 nt, Mus musculus
uuggaggacauuuuugauguugugcuaga

SEQ ID NO: 550 - RNA, 26 nt, Mus musculus
uugagagaugcuuguucaaaaaacau

SEQ ID NO: 551 - RNA, 26 nt, Mus musculus
uaauuguuuccagauucgugugggag

SEQ ID NO: 552 - RNA, 27 nt, Mus musculus
uuguugccuuuguaucagucacugaag

SEQ ID NO: 553 - RNA, 26 nt, Mus musculus
uuccuugcucugguggacgcuuguac

SEQ ID NO: 554 - RNA, 27 nt, Mus musculus
aaaguauuugaacauguggaagaugga

SEQ ID NO: 555 - RNA, 27 nt, Mus musculus
uuacagacucagagcagaagagcuguc

SEQ ID NO: 556 - RNA, 26 nt, Mus musculus
ugaaucuggaaacaauucaguuaguu

SEQ ID NO: 557 - RNA, 29 nt, Mus musculus
uaauggggugauuugauuuucugaagucc

SEQ ID NO: 558 - RNA, 26 nt, Mus musculus
uuucccccuguguucuggaagcaucc

SEQ ID NO: 559 - RNA, 26 nt, Mus musculus
uugacugcucuuuucgaaggucccga

SEQ ID NO: 560 - RNA, 26 nt, Mus musculus
ugagcagaagagcuggauggugugug

SEQ ID NO: 561 - RNA, 28 nt, Mus musculus
uacagacucagagcagaagagcuguccu

SEQ ID NO: 562 - RNA, 29 nt, Mus musculus
uaagacaguguaaagagauguauacucag

SEQ ID NO: 563 - RNA, 27 nt, Mus musculus
gguuauuugauuuucugaagucuuuuu

SEQ ID NO: 564 - RNA, 27 nt, Mus musculus
cagacucagagcagaagagcuguccug

SEQ ID NO: 565 - RNA, 28 nt, Mus musculus
aauggggugauuugauuuucugaagucc

SEQ ID NO: 566 - RNA, 27 nt, Mus musculus
uucugguguaucuggaguguauuuauu

SEQ ID NO: 567 - RNA, 27 nt, Mus musculus
uuaauggggugauuugauuuucugaag

SEQ ID NO: 568 - RNA, 32 nt, Mus musculus
uuaacucugguucuggaagcaaagguaccacu

SEQ ID NO: 569 - RNA, 26 nt, Mus musculus
uguguucuggaagcauccacaagcug

SEQ ID NO: 570 - RNA, 39 nt, Mus musculus
ugugaaccuagcuuugauuuucugaacccauguaaaugc

SEQ ID NO: 571 - RNA, 27 nt, Mus musculus
ugcuccauuguuuugcagaggugucug

SEQ ID NO: 572 - RNA, 25 nt, Mus musculus
ugccucuuagaucugacuucagugu

SEQ ID NO: 573 - RNA, 26 nt, Mus musculus
ucauaaagcaggcacaagagguucag

SEQ ID NO: 574 - RNA, 29 nt, Mus musculus
uaguuaacucugguucuggaagcaaaggu

SEQ ID NO: 575 - RNA, 31 nt, Mus musculus
uaccagcuaguuaacucugguucuggaagca

SEQ ID NO: 576 - RNA, 31 nt, Mus musculus
uaaucccagcaccccagaaguggaagcagga

SEQ ID NO: 577 - RNA, 26 nt, Mus musculus
uaagaguguggaagaacauguggcuu

SEQ ID NO: 578 - RNA, 31 nt, Mus musculus
uaacucugguucuggaagcaaagguaccacu

SEQ ID NO: 579 - RNA, 26 nt, Mus musculus
uaaagcaggcacaagagguucagaac

SEQ ID NO: 580 - RNA, 31 nt, Mus musculus
gcuaguuaacucugguucuggaagcaaaggu

SEQ ID NO: 581 - RNA, 27 nt, Mus musculus
gaguagauugucuucucucuuguuagu

SEQ ID NO: 582 - RNA, 26 nt, Mus musculus
cagaagugagcagaagagcugaagaa

SEQ ID NO: 583 - RNA, 26 nt, Mus musculus
uuuuugugucuaaguagcuguugacu

SEQ ID NO: 584 - RNA, 28 nt, Mus musculus
uuuucuguuuugauuucagcuuaugccc

SEQ ID NO: 585 - RNA, 26 nt, Mus musculus
uuucuuggccuggagaugucucugcu

SEQ ID NO: 586 - RNA, 26 nt, Mus musculus
uuucaugauugauucucgguucaaga

SEQ ID NO: 587 - RNA, 28 nt, Mus musculus
uuguguccaaauguugauuccagaacuc

SEQ ID NO: 588 - RNA, 27 nt, Mus musculus
uucugguggaucugaagacagcuacag

SEQ ID NO: 589 - RNA, 27 nt, Mus musculus
ugacaaaugacccuaacacaugcuggu

SEQ ID NO: 590 - RNA, 26 nt, Mus musculus
uagggucauuugucauguggaaauug

SEQ ID NO: 591 - RNA, 27 nt, Mus musculus
uggugaucugagaauagggaccggggu

SEQ ID NO: 592 - RNA, 29 nt, Mus musculus
ugggauccauggugaucugagaauaggga

SEQ ID NO: 593 - RNA, 31 nt, Mus musculus
gcaacagugggauccauggugaucugagaau

SEQ ID NO: 594 - RNA, 26 nt, Mus musculus
auccauggugaucugagaauagggac

SEQ ID NO: 595 - RNA, 29 nt, Mus musculus
acagugggauccauggugaucugagaaua

SEQ ID NO: 596 - RNA, 28 nt, Mus musculus
uccauggugaucugagaauagggaccgg

SEQ ID NO: 597 - RNA, 27 nt, Mus musculus
uugaugaaccagaaauugaccaauuug

SEQ ID NO: 598 - RNA, 26 nt, Mus musculus
uguuucuucagaggagaauucaucau

SEQ ID NO: 599 - RNA, 26 nt, Mus musculus
ugcagaucagucaaagacauccaugg

SEQ ID NO: 600 - RNA, 28 nt, Mus musculus
gugcccaguggcuuuugaacgcuccugc

SEQ ID NO: 601 - RNA, 30 nt, Mus musculus
cagaggagaauucaucaucugauucagagc

SEQ ID NO: 602 - RNA, 26 nt, Mus musculus
uucuacaucuggauuuucaggcagca

SEQ ID NO: 603 - RNA, 26 nt, Mus musculus
uuccuucucugcaagaaagcaagucc

SEQ ID NO: 604 - RNA, 29 nt, Mus musculus
uucaucucugucgggaguggaugaauggc

SEQ ID NO: 605 - RNA, 26 nt, Mus musculus
uucaaagggaaaacagccugagcucu

SEQ ID NO: 606 - RNA, 26 nt, Mus musculus
uucagcagacucaggagagauagaug

SEQ ID NO: 607 - RNA, 29 nt, Mus musculus
uggagagggccuaguaugcuucugugcac

SEQ ID NO: 608 - RNA, 27 nt, Mus musculus
ugaaucagaugaugaauucuccucuaa

SEQ ID NO: 609 - RNA, 30 nt, Mus musculus
gagggccuaguaugcuucugugcacacagu

SEQ ID NO: 610 - RNA, 27 nt, Mus musculus
ugcuggagugagaggcugaagagggua

SEQ ID NO: 611 - RNA, 50 nt, Mus musculus
ugcagacacacaccugugcagcaaaggagcugccugcuuucuuggguugu

SEQ ID NO: 612 - RNA, 41 nt, Mus musculus
uagaccaaacaucugaggucucugcugcagcugagcucucu

SEQ ID NO: 613 - RNA, 29 nt, Mus musculus
ccucuggcaaugucaggcucccagggauu

SEQ ID NO: 614 - RNA, 27 nt, Mus musculus
ugaaagagggccuaguaugcacuacag

SEQ ID NO: 615 - RNA, 30 nt, Mus musculus
ugaggcuacguucagauucuguguggcaca

SEQ ID NO: 616 - RNA, 30 nt, Mus musculus
ucagaugaugaauucuccucugaagaaaca

SEQ ID NO: 617 - RNA, 26 nt, Mus musculus
uauggaguuugcuucaaaugugcaaa

SEQ ID NO: 618 - RNA, 28 nt, Mus musculus
uacguucagauucuguguggcacagggg

SEQ ID NO: 619 - RNA, 25 nt, Mus musculus
guugcuggagugagaggcugaagag

SEQ ID NO: 620 - RNA, 26 nt, Mus musculus
guuacaggaagucaucugcugccuga

SEQ ID NO: 621 - RNA, 26 nt, Mus musculus
ggcuacguucagauucuguguggcac

SEQ ID NO: 622 - RNA, 30 nt, Mus musculus
gaggcuacguucagauucuguguggcacag

SEQ ID NO: 623 - RNA, 29 nt, Mus musculus
gaaucagaugaugaauucuccucugaaga

SEQ ID NO: 624 - RNA, 38 nt, Mus musculus
cuugaaucaucugccgccugaaccuggcuagcaacaga

SEQ ID NO: 625 - RNA, 29 nt, Mus musculus
cugaaucagaugaugaauucuccucugaa

SEQ ID NO: 626 - RNA, 50 nt, Mus musculus
cugaagauuggcucuaggcuugaaucaucugccgccugaaccuggcuagc

SEQ ID NO: 627 - RNA, 28 nt, Mus musculus
cagaugaugaauucuccucugaagaaac

SEQ ID NO: 628 - RNA, 28 nt, Mus musculus
aucagaugaugaauucuccucugaagaa

SEQ ID NO: 629 - RNA, 27 nt, Mus musculus
aggcuacguucagauucuguguggcac

SEQ ID NO: 630 - RNA, 27 nt, Mus musculus
agaugaugaauucuccucugaagaaac

SEQ ID NO: 631 - RNA, 29 nt, Mus musculus
acaagugaggcuacguucagauucugugu

SEQ ID NO: 632 - RNA, 50 nt, Mus musculus
acaaacccacccaggagaugaggugagcagacaaagccucuggcaauguc

SEQ ID NO: 633 - RNA, 32 nt, Mus musculus
aaucagaugaugaauucuccucugaagaaaca

SEQ ID NO: 634 - RNA, 27 nt, Mus musculus
aagugaggcuacguucagauucugugu

SEQ ID NO: 635 - RNA, 29 nt, Mus musculus
aagcagaugaugaauucuccucugaagaa

SEQ ID NO: 636 - RNA, 26 nt, Mus musculus
uuccucggaugcuucaaauguagaaa

SEQ ID NO: 637 - RNA, 30 nt, Mus musculus
uuccaggugcaguaaacccucuggcacgga

SEQ ID NO: 638 - RNA, 27 nt, Mus musculus
uucagaaucaucugcagcuacauacua

SEQ ID NO: 639 - RNA, 29 nt, Mus musculus
uuauuucuaaugagacauucgucacaggc

SEQ ID NO: 640 - RNA, 27 nt, Mus musculus
aucagaugaugaauucuccucugagga

SEQ ID NO: 641 - RNA, 26 nt, Mus musculus
ucacuuggcaaguguuaucccaaucc

SEQ ID NO: 642 - RNA, 27 nt, Mus musculus
uacaaauauucucuguauagucagcau

SEQ ID NO: 643 - RNA, 26 nt, Mus musculus
uggaacucacucuguagacuaugcug

SEQ ID NO: 644 - RNA, 28 nt, Mus musculus
cuauccucauguuagucuacagagaaua

SEQ ID NO: 645 - RNA, 26 nt, Mus musculus
ugugaucagagcacuucagaagcugg

SEQ ID NO: 646 - RNA, 29 nt, Mus musculus
ugagcacgaaccuggaagcagcuccuacc

SEQ ID NO: 647 - RNA, 26 nt, Mus musculus
ugugcaucagagagaaggccuguguu

SEQ ID NO: 648 - RNA, 26 nt, Mus musculus
ugucacuggaagcagccucaugaggg

SEQ ID NO: 649 - RNA, 26 nt, Mus musculus
ugaauguuuugccugauugucuccau

SEQ ID NO: 650 - RNA, 29 nt, Mus musculus
acaguagaaagguacccauugggaucaca

SEQ ID NO: 651 - RNA, 26 nt, Mus musculus
ugguaccagagcacuucgaaucacag

SEQ ID NO: 652 - RNA, 26 nt, Mus musculus
ugaccagagcacuucaguuccuaaga

SEQ ID NO: 653 - RNA, 30 nt, Mus musculus
cagagagacuggaagcagcucccagaacuc

SEQ ID NO: 654 - RNA, 26 nt, Mus musculus
uguuccuuaaagaaggaggguuaugu

SEQ ID NO: 655 - RNA, 26 nt, Mus musculus
uggucaugagucacuggaaacaucau

SEQ ID NO: 656 - RNA, 28 nt, Mus musculus
ugggagcagaaggccuuaaugaugucua

SEQ ID NO: 657 - RNA, 26 nt, Mus musculus
ugacucuuuacagugaaagaauagcc

SEQ ID NO: 658 - RNA, 26 nt, Mus musculus
uuuggcuucugauuucagaggcuuag

SEQ ID NO: 659 - RNA, 25 nt, Mus musculus
uuuggaagcagcuccucagggaccu

SEQ ID NO: 660 - RNA, 27 nt, Mus musculus
ugauagcacucaauucuuccaaaagga

SEQ ID NO: 661 - RNA, 26 nt, Mus musculus
uuaucaagcucugcuuccagugacuc

SEQ ID NO: 662 - RNA, 28 nt, Mus musculus
ugcuuccagugacucagagcuuaguaau

SEQ ID NO: 663 - RNA, 27 nt, Mus musculus
uuucagguguuucccugccauucggua

SEQ ID NO: 664 - RNA, 26 nt, Mus musculus
uugagaugaccuugaucugccuaugu

SEQ ID NO: 665 - RNA, 26 nt, Mus musculus
uuaccaaaauaggauuuggccagcag

SEQ ID NO: 666 - RNA, 26 nt, Mus musculus
uguaaaacucugaaaucagaaacugu

SEQ ID NO: 667 - RNA, 27 nt, Mus musculus
ugaguggccuuuagaucuaauucaauc

SEQ ID NO: 668 - RNA, 31 nt, Mus musculus
ugggccuucucucugaagggagaugcuaagu

SEQ ID NO: 669 - RNA, 28 nt, Mus musculus
ucuuuauguaaaacucugaaaucagaaa

SEQ ID NO: 670 - RNA, 26 nt, Mus musculus
ucuggaauccaggacccacagagguu

SEQ ID NO: 671 - RNA, 26 nt, Mus musculus
ucauugugagcucaaggucaaccugg

SEQ ID NO: 672 - RNA, 26 nt, Mus musculus
ucaggauuccgauguacauggaagac

SEQ ID NO: 673 - RNA, 28 nt, Mus musculus
uaguuaccaaaauaggauuuggccagca

SEQ ID NO: 674 - RNA, 27 nt, Mus musculus
uaggcaucugaguauguucccuccugc

SEQ ID NO: 675 - RNA, 29 nt, Mus musculus
uaccaaaauaggauuuggccagcaggaga

SEQ ID NO: 676 - RNA, 31 nt, Mus musculus
uaaauaugaauuagauuagaagaacaguugg

SEQ ID NO: 677 - RNA, 25 nt, Mus musculus
uaaaacucugaaaucagaaacugug

SEQ ID NO: 678 - RNA, 31 nt, Mus musculus
gacuugggccuucucucugaagggagaugcu

SEQ ID NO: 679 - RNA, 27 nt, Mus musculus
aaguuucccugccauucagaaggacaa

SEQ ID NO: 680 - RNA, 27 nt, Mus musculus
uuucugaaagaauccaggaccaccagc

SEQ ID NO: 681 - RNA, 29 nt, Mus musculus
uguuacccacuuccagugacucacagcaa

SEQ ID NO: 682 - RNA, 27 nt, Mus musculus
ugugagcucaaucggaggaucauggaa

SEQ ID NO: 683 - RNA, 27 nt, Mus musculus
ugguuaugaagaauccagaugccuuuu

SEQ ID NO: 684 - RNA, 33 nt, Mus musculus
ugagggaguauggucauugugagacuaagaucc

SEQ ID NO: 685 - RNA, 26 nt, Mus musculus
ugaagugcucuggucuagcucguuac

SEQ ID NO: 686 - RNA, 26 nt, Mus musculus
uccuaguggccuuuagaugaagaugu

SEQ ID NO: 687 - RNA, 26 nt, Mus musculus
uuuaaaagaugccuuuuucugccuga

SEQ ID NO: 688 - RNA, 30 nt, Mus musculus
ugguucaggcauucuuuaaggaacuaaguc

SEQ ID NO: 689 - RNA, 28 nt, Mus musculus
uggauuuaaaagaugccuuuuucugucu

SEQ ID NO: 690 - RNA, 27 nt, Mus musculus
ugauuuaaaagaugccuuuuucugccu

SEQ ID NO: 691 - RNA, 27 nt, Mus musculus
ugagaggucugaagugcucugaggucc

SEQ ID NO: 692 - RNA, 26 nt, Mus musculus
ugaaagaugccuuuuucugucucaga

SEQ ID NO: 693 - RNA, 30 nt, Mus musculus
uuagcuagucuaacuuccuugaugaguucc

SEQ ID NO: 694 - RNA, 29 nt, Mus musculus
uagcuagucuaacuuccuugaugaguucc

SEQ ID NO: 695 - RNA, 27 nt, Mus musculus
agcuagucuaacuuccuugaugaguuc

SEQ ID NO: 696 - RNA, 27 nt, Mus musculus
uggcuagcuucugaacagcaggauggg

SEQ ID NO: 697 - RNA, 30 nt, Mus musculus
ugccacuucaaaugaacagcaagagcuuuu

SEQ ID NO: 698 - RNA, 26 nt, Mus musculus
ugaguuguacuguugguaaacugagc

SEQ ID NO: 699 - RNA, 27 nt, Mus musculus
ucgcuccucaugguuuacucagucugc

SEQ ID NO: 700 - RNA, 31 nt, Mus musculus
aacuugcugguuagcuagucuaacuuccuug

SEQ ID NO: 701 - RNA, 29 nt, Mus musculus
gcucaaagaugccacuucaaaugaacagc

SEQ ID NO: 702 - RNA, 25 nt, Mus musculus
ugaggagagacugaguaguguugga

SEQ ID NO: 703 - RNA, 26 nt, Mus musculus
uucauuugaaguggcaucuuugagcu

SEQ ID NO: 704 - RNA, 30 nt, Mus musculus
ugaauagaaaagacagguguugcagcugua

SEQ ID NO: 705 - RNA, 26 nt, Mus musculus
ugaaccaggagcuagaaguuagagau

SEQ ID NO: 706 - RNA, 26 nt, Mus musculus
ucagaagcuagaagagaguguccgau

SEQ ID NO: 707 - RNA, 29 nt, Mus musculus
aauagaaaagacagguguugcagcuguaa

SEQ ID NO: 708 - RNA, 26 nt, Mus musculus
uagguucagaagcuggaaguucagga

SEQ ID NO: 709 - RNA, 26 nt, Mus musculus
uaggagagaaagaaaagacaggaucu

SEQ ID NO: 710 - RNA, 26 nt, Mus musculus
uaagagagaaagaaaagacaggaucu

SEQ ID NO: 711 - RNA, 26 nt, Mus musculus
uaaaggcaguaggagagacugagccu

SEQ ID NO: 712 - RNA, 27 nt, Mus musculus
aagucaugaauagaaaagacagguguu

SEQ ID NO: 713 - RNA, 26 nt, Mus musculus
cuggaauuaggagauaaaccucuggu

SEQ ID NO: 714 - RNA, 26 nt, Mus musculus
uuuucugugauaaaggccaagaagga

SEQ ID NO: 715 - RNA, 27 nt, Mus musculus
uagagcacucugauugacagcagucug

SEQ ID NO: 716 - RNA, 26 nt, Mus musculus
cgugugcagugaaaaccagagcuagc

SEQ ID NO: 717 - RNA, 30 nt, Mus musculus
uggagaaaggcagcccugguuccuggauga

SEQ ID NO: 718 - RNA, 29 nt, Mus musculus
ugauaaaggccaagaagugcaggcccaga

SEQ ID NO: 719 - RNA, 27 nt, Mus musculus
ucaacaaaggaugucaggacuggaacu

SEQ ID NO: 720 - RNA, 26 nt, Mus musculus
uagcugugucaccuaaggguguuggg

SEQ ID NO: 721 - RNA, 26 nt, Mus musculus
uagaagaaaaaucaaagagaucagga

SEQ ID NO: 722 - RNA, 25 nt, Mus musculus
uaauuuaugaaggaugucagguggu

SEQ ID NO: 723 - RNA, 28 nt, Mus musculus
uuugcagauugugcccugguuccugggc

SEQ ID NO: 724 - RNA, 29 nt, Mus musculus
uuucugucaaaaccaucuuccagggagac

SEQ ID NO: 725 - RNA, 27 nt, Mus musculus
uuucuaguuccauuuuuugauccugga

SEQ ID NO: 726 - RNA, 26 nt, Mus musculus
uuggcagugcguugcuagucaauaug

SEQ ID NO: 727 - RNA, 26 nt, Mus musculus
ucuaagcucauguuguuccauuccac

SEQ ID NO: 728 - RNA, 26 nt, Mus musculus
ucucaagauguugggguguuggccag

SEQ ID NO: 729 - RNA, 25 nt, Mus musculus
uaagauggaagcguuuugggaguca

SEQ ID NO: 730 - RNA, 26 nt, Mus musculus
ugccacuucaaaaaauggaaccccgg

SEQ ID NO: 731 - RNA, 27 nt, Mus musculus
ugagcauaauuuuuggaaacaaacaug

SEQ ID NO: 732 - RNA, 26 nt, Mus musculus
ugagauguugggguguuggccagcuc

SEQ ID NO: 733 - RNA, 26 nt, Mus musculus
uuguuccaggaaccacggagacuguc

SEQ ID NO: 734 - RNA, 26 nt, Mus musculus
uucccaaugauuugauugcccuccca

SEQ ID NO: 735 - RNA, 26 nt, Mus musculus
uuaugaagaaguguuccaggcuggaa

SEQ ID NO: 736 - RNA, 26 nt, Mus musculus
uggaugagggucccaguaagcuauuc

SEQ ID NO: 737 - RNA, 27 nt, Mus musculus
uggaucucucugauuucaaggucagcc

SEQ ID NO: 738 - RNA, 26 nt, Mus musculus
ugcuaugaagaaguguuuaggaacca

SEQ ID NO: 739 - RNA, 25 nt, Mus musculus
ucccaaugauuugauugcccuccca

SEQ ID NO: 740 - RNA, 26 nt, Mus musculus
uaugaagaaguguuccaggcuggaau

SEQ ID NO: 741 - RNA, 28 nt, Mus musculus
uaacaguuucccaaugauuugauugccc

SEQ ID NO: 742 - RNA, 29 nt, Mus musculus
gcuaacaguuucccaaugauuugauugcc

SEQ ID NO: 743 - RNA, 26 nt, Mus musculus
uuguuugaaauaggaucucugugugu

SEQ ID NO: 744 - RNA, 34 nt, Mus musculus
uugcuguccaggccugaggaucagaauagcugag

SEQ ID NO: 745 - RNA, 27 nt, Mus musculus
gcuuuggucaugguaucucuucauagc

SEQ ID NO: 746 - RNA, 36 nt, Mus musculus
ugugauaccaugcugggcuccaugcaguguuuuugu

SEQ ID NO: 747 - RNA, 26 nt, Mus musculus
ugagagcagcucaaagaggccuauga

SEQ ID NO: 748 - RNA, 28 nt, Mus musculus
caugauaccaugcucaugugaacucuga

SEQ ID NO: 749 - RNA, 26 nt, Mus musculus
gcagcucaaagaggccuacgaaccag

SEQ ID NO: 750 - RNA, 26 nt, Mus musculus
ccugagagcagcucaaagaggccuau

SEQ ID NO: 751 - RNA, 27 nt, Mus musculus
ugugguauuuuggugaagaaucuggcu

SEQ ID NO: 752 - RNA, 27 nt, Mus musculus
ugagcaugguaucauggaguuccccag

SEQ ID NO: 753 - RNA, 26 nt, Mus musculus
uccugguucguaggccucuuugagcu

SEQ ID NO: 754 - RNA, 27 nt, Mus musculus
augagcaugguaucauggaguucccca

SEQ ID NO: 755 - RNA, 26 nt, Mus musculus
aggccucuuugagcccuucauggcuu

SEQ ID NO: 756 - RNA, 26 nt, Mus musculus
uggugaagaaucuggcugcuuuuugc

SEQ ID NO: 757 - RNA, 27 nt, Mus musculus
ucagaguucacaugagcaugguaucau

SEQ ID NO: 758 - RNA, 30 nt, Mus musculus
uaggccucuuugagcugcucucaggcggua

SEQ ID NO: 759 - RNA, 28 nt, Mus musculus
uaaaagcacaaggccucuuugagcccua

SEQ ID NO: 760 - RNA, 29 nt, Mus musculus
gaguucacaugagcaugguaucauggagu

SEQ ID NO: 761 - RNA, 30 nt, Mus musculus
cagcuggacugcacaggccucuuugagccc

SEQ ID NO: 762 - RNA, 28 nt, Mus musculus
aggccucuuugagcugcucucaggcggu

SEQ ID NO: 763 - RNA, 26 nt, Mus musculus
cagaguucacaugagcaugguaucau

SEQ ID NO: 764 - RNA, 31 nt, Mus musculus
agcuggacugcacaggccucuuugagcccuu

SEQ ID NO: 765 - RNA, 26 nt, Mus musculus
ugaggcaguauuaugguuauucucag

SEQ ID NO: 766 - RNA, 26 nt, Mus musculus
uaauauagggaaacuucauuuggcuc

SEQ ID NO: 767 - RNA, 30 nt, Mus musculus
ccuuucaccauguggaucugggagaucaga

SEQ ID NO: 768 - RNA, 27 nt, Mus musculus
cagggaaugcaacuugaagcugacgga

SEQ ID NO: 769 - RNA, 27 nt, Mus musculus
uuggaaagucagagcacugcagcacug

SEQ ID NO: 770 - RNA, 28 nt, Mus musculus
uggaacggaaucagguugggcagcguga

SEQ ID NO: 771 - RNA, 26 nt, Mus musculus
ugcgcagauauguuguauuuggaccu

SEQ ID NO: 772 - RNA, 26 nt, Mus musculus
ugauugauugugaggaguggauucuu

SEQ ID NO: 773 - RNA, 26 nt, Mus musculus
ugaggaaaucagacauggaaagcaua

SEQ ID NO: 774 - RNA, 26 nt, Mus musculus
ugaccacuggacuucuugaguuguug

SEQ ID NO: 775 - RNA, 26 nt, Mus musculus
ugaaggcagaaauaacauugagcauu

SEQ ID NO: 776 - RNA, 28 nt, Mus musculus
uccuagguacacugggacauuugcuaga

SEQ ID NO: 777 - RNA, 27 nt, Mus musculus
ucacgugccagcagcauugaccaggua

SEQ ID NO: 778 - RNA, 25 nt, Mus musculus
uagguacaggaagacauacaugugc

SEQ ID NO: 779 - RNA, 26 nt, Mus musculus
uaggcaaagucagaagacagauguga

SEQ ID NO: 780 - RNA, 25 nt, Mus musculus
uaggaaugaggaaaucagacaugga

SEQ ID NO: 781 - RNA, 26 nt, Mus musculus
uaagaugaggaguggggugggaaggc

SEQ ID NO: 782 - RNA, 33 nt, Mus musculus
cguccugacagaugcuucuaacugcagucauuu

SEQ ID NO: 783 - RNA, 32 nt, Mus musculus
uuucaaugaagaugagaaguauuucugagagc

SEQ ID NO: 784 - RNA, 27 nt, Mus musculus
uuggugccuaaauguaaagguuugaac

SEQ ID NO: 785 - RNA, 30 nt, Mus musculus
uuggaguaguucuuaugacuugaugguccu

SEQ ID NO: 786 - RNA, 47 nt, Mus musculus
cugcgcugucaggaacaucuugacaacagguugucuauucuguuuug

SEQ ID NO: 787 - RNA, 26 nt, Mus musculus
uucugacuuugccuuucugccauaga

SEQ ID NO: 788 - RNA, 28 nt, Mus musculus
uggcuaacagacuauugaaacaaugguu

SEQ ID NO: 789 - RNA, 25 nt, Mus musculus
uggauuccaccucagagaccucgga

SEQ ID NO: 790 - RNA, 32 nt, Mus musculus
ugaucccagggguaaccaguagauugagucuc

SEQ ID NO: 791 - RNA, 29 nt, Mus musculus
ucccaugcugccuguugguccuccucugc

SEQ ID NO: 792 - RNA, 26 nt, Mus musculus
uagcuucuguuuguauccuccuuccc

SEQ ID NO: 793 - RNA, 26 nt, Mus musculus
gacaaagagguuuccucauguaggga

SEQ ID NO: 794 - RNA, 28 nt, Mus musculus
cugccucccaaguccuggggucacaggu

SEQ ID NO: 795 - RNA, 26 nt, Mus musculus
uuucugaacauguccacagccgcccu

SEQ ID NO: 796 - RNA, 27 nt, Mus musculus
uugauugcuggauaaccuauuggggaa

SEQ ID NO: 797 - RNA, 26 nt, Mus musculus
ugagaagaaggaguuggcugcugcca

SEQ ID NO: 798 - RNA, 29 nt, Mus musculus
uuugugacauucuggcugcuaacaaagcu

SEQ ID NO: 799 - RNA, 26 nt, Mus musculus
ugaggagacuccaaagaggagacaau

SEQ ID NO: 800 - RNA, 29 nt, Mus musculus
uagaugguuuuguggucuuaauuacaccu

SEQ ID NO: 801 - RNA, 29 nt, Mus musculus
uacacuagcaaaauuuugcugaaaggagc

SEQ ID NO: 802 - RNA, 26 nt, Mus musculus
uuuugcugaaaggauccagauauagc

SEQ ID NO: 803 - RNA, 27 nt, Mus musculus
uuuugcugaaaggacccagauauggcu

SEQ ID NO: 804 - RNA, 29 nt, Mus musculus
uuuugcugaaaggacccagauauagcuug

SEQ ID NO: 805 - RNA, 28 nt, Mus musculus
uuuugcugaaaggacccagauauagcug

SEQ ID NO: 806 - RNA, 26 nt, Mus musculus
uuuugcugaaaggacccagauagaac

SEQ ID NO: 807 - RNA, 25 nt, Mus musculus
uuuugcugaaaggacccagauaaag

SEQ ID NO: 808 - RNA, 29 nt, Mus musculus
uuuugcugaaaggacccagagauagcugu

SEQ ID NO: 809 - RNA, 28 nt, Mus musculus
uuugcugaaaggagccagauauagcugu

SEQ ID NO: 810 - RNA, 26 nt, Mus musculus
ugugaauuuuuuuggucuucgugggc

SEQ ID NO: 811 - RNA, 39 nt, Mus musculus
ugucacaggguggcugcuuggggccucuaauugaaccca

SEQ ID NO: 812 - RNA, 26 nt, Mus musculus
uggcugaaaggaggaagacuaugcgu

SEQ ID NO: 813 - RNA, 25 nt, Mus musculus
uggaaaaguagauggcuaagggaga

SEQ ID NO: 814 - RNA, 26 nt, Mus musculus
uggcuuugaacucuuggagagagagu

SEQ ID NO: 815 - RNA, 26 nt, Mus musculus
uagugagaugagagggucuuaaggag

SEQ ID NO: 816 - RNA, 27 nt, Mus musculus
ugugccuggcuaacuagggauguguuu

SEQ ID NO: 817 - RNA, 27 nt, Mus musculus
ugucucacugagccugggagcuugcug

SEQ ID NO: 818 - RNA, 26 nt, Mus musculus
ugggugcagaucugagccuggaaagu

SEQ ID NO: 819 - RNA, 26 nt, Mus musculus
ugaucugagccugggauggggaagac

SEQ ID NO: 820 - RNA, 27 nt, Mus musculus
uuugaauuagucaguguggagaggagu

SEQ ID NO: 821 - RNA, 27 nt, Mus musculus
uuguugaagaugcuuaagccucaaaga

SEQ ID NO: 822 - RNA, 26 nt, Mus musculus
uuggaucugagccuggguaugagcug

SEQ ID NO: 823 - RNA, 26 nt, Mus musculus
uuauccaugagccugggagcuucucu

SEQ ID NO: 824 - RNA, 29 nt, Mus musculus
uuaaagaacccacugcuuauggccuagac

SEQ ID NO: 825 - RNA, 27 nt, Mus musculus
ugugugacucugguguccccugcugcu

SEQ ID NO: 826 - RNA, 26 nt, Mus musculus
ugcaagagcucucuggcuaggugguu

SEQ ID NO: 827 - RNA, 25 nt, Mus musculus
ugaugucugucaguguggaaaaaaa

SEQ ID NO: 828 - RNA, 29 nt, Mus musculus
ugaagaccagaucugaguucagagagcuu

SEQ ID NO: 829 - RNA, 31 nt, Mus musculus
uccaggcuuacauuaauugguuagaccagau

SEQ ID NO: 830 - RNA, 27 nt, Mus musculus
gugugugacucugguguccccugcugu

SEQ ID NO: 831 - RNA, 26 nt, Mus musculus
ugucugucaguguggaaaaaaaaaaa

SEQ ID NO: 832 - RNA, 31 nt, Mus musculus
ugcugaugucugucaguguggaaaaaaaaaa

SEQ ID NO: 833 - RNA, 27 nt, Mus musculus
uagugugugacucugguguccccugcu

SEQ ID NO: 834 - RNA, 29 nt, Mus musculus
uacaucuuaaagaacccacugcuuauggc

SEQ ID NO: 835 - RNA, 26 nt, Mus musculus
uacaucauccugaucccucagaccca

SEQ ID NO: 836 - RNA, 27 nt, Mus musculus
uacagaaguagugugugacucuggugu

SEQ ID NO: 837 - RNA, 28 nt, Mus musculus
uaaagaacccacugcuuauggccuagac

SEQ ID NO: 838 - RNA, 30 nt, Mus musculus
guagugugugacucugguguccccugcugc

SEQ ID NO: 839 - RNA, 27 nt, Mus musculus
guacagaaguagugugugacucuggug

SEQ ID NO: 840 - RNA, 27 nt, Mus musculus
gguacagaaguagugugugacucuggu

SEQ ID NO: 841 - RNA, 27 nt, Mus musculus
gaaguagugugugacucuggugucccc

SEQ ID NO: 842 - RNA, 27 nt, Mus musculus
cugaugucugucaguguggaaaaaaaa

SEQ ID NO: 843 - RNA, 29 nt, Mus musculus
  misc_feature (2)..(2): n is a, c, g, or u
cngaaguagugugugacucuggugucccc

SEQ ID NO: 844 - RNA, 27 nt, Mus musculus
cagaaguagugugugacucuggugucc

SEQ ID NO: 845 - RNA, 28 nt, Mus musculus
aguagugugugacucugguguccccugc

SEQ ID NO: 846 - RNA, 27 nt, Mus musculus
agaaguagugugugacucugguguccc

SEQ ID NO: 847 - RNA, 28 nt, Mus musculus
acgugcuugcugugugacucuggugacc

SEQ ID NO: 848 - RNA, 26 nt, Mus musculus
acagaaguagugugugacucuggugu

SEQ ID NO: 849 - RNA, 32 nt, Mus musculus
aaguagugugugacucugguguccccugcugc

SEQ ID NO: 850 - RNA, 26 nt, Mus musculus
acagaaguagugugugacucuggugu

SEQ ID NO: 851 - RNA, 26 nt, Mus musculus
uuuuguucccuuuccacacugacugu

SEQ ID NO: 852 - RNA, 26 nt, Mus musculus
uagcucagaucuggucacuccuccaa

SEQ ID NO: 853 - RNA, 26 nt, Mus musculus
uaacuucaguuaccagagucaaaggu

SEQ ID NO: 854 - RNA, 27 nt, Mus musculus
uaaaagggucugagcucccuaauggca

SEQ ID NO: 855 - RNA, 26 nt, Mus musculus
gauaacuucaguuaccagagucaaag

SEQ ID NO: 856 - RNA, 26 nt, Mus musculus
uagaaggagagagaucuugccccacc

SEQ ID NO: 857 - RNA, 26 nt, Mus musculus
ucuaaugaaggagagagaugcucuca

SEQ ID NO: 858 - RNA, 28 nt, Mus musculus
ucccagagagaugggugcucggucuaca

SEQ ID NO: 859 - RNA, 26 nt, Mus musculus
uguaaucuggugacccaucucucucc

SEQ ID NO: 860 - RNA, 26 nt, Mus musculus
ucugaugcaacucacaguagggucuu

SEQ ID NO: 861 - RNA, 26 nt, Mus musculus
uaacagcagcaggaagcacuucacac

SEQ ID NO: 862 - RNA, 25 nt, Mus musculus
uucaaucgccagcagcagaacaaua

SEQ ID NO: 863 - RNA, 26 nt, Mus musculus
ucuuuaguucuugggagcagccagcc

SEQ ID NO: 864 - RNA, 26 nt, Mus musculus
uaagagaguuccugggagcagcagga

SEQ ID NO: 865 - RNA, 27 nt, Mus musculus
uaaagccuuugcaacucacagucaucu

SEQ ID NO: 866 - RNA, 26 nt, Mus musculus
ugcccuaaaggaucaacugagucagg

SEQ ID NO: 867 - RNA, 26 nt, Mus musculus
ugauggagcagcaggaaggauuacag

SEQ ID NO: 868 - RNA, 29 nt, Mus musculus
ugaggcuagagcagcuccaggcagcaugg

SEQ ID NO: 869 - RNA, 25 nt, Mus musculus
uaauacaguuccuuggguucuaguu

SEQ ID NO: 870 - RNA, 26 nt, Mus musculus
uaaaccuggcuguggaaacggagcaa

SEQ ID NO: 871 - RNA, 30 nt, Mus musculus
gucucuguuagcucuggcuguggaaaggca

SEQ ID NO: 872 - RNA, 27 nt, Mus musculus
cuuacaggccagacaaauuuucaguug

SEQ ID NO: 873 - RNA, 35 nt, Mus musculus
ccagugaaauuuccucacagucuggggaacagugu

SEQ ID NO: 874 - RNA, 35 nt, Mus musculus
cacuggggcaucaagccuucacaggaccaagggac

SEQ ID NO: 875 - RNA, 26 nt, Mus musculus
uucaaaggaucaacagcuggaauaca

SEQ ID NO: 876 - RNA, 28 nt, Mus musculus
ucaaaggaucaacagcuggaauacacuu

SEQ ID NO: 877 - RNA, 27 nt, Mus musculus
ugaagaagagcagcaggaagcuggggu

SEQ ID NO: 878 - RNA, 27 nt, Mus musculus
uccaagaacccaagaaacaagagagga

SEQ ID NO: 879 - RNA, 26 nt, Mus musculus
gacagaugcuguugccuguaugaaac

SEQ ID NO: 880 - RNA, 36 nt, Mus musculus
ugucugcugcugcacuaguggccauucuucuaauau

SEQ ID NO: 881 - RNA, 33 nt, Mus musculus
ugaaaucgugucugcugcugcacuaguggccau

SEQ ID NO: 882 - RNA, 50 nt, Mus musculus
cagagccugcugcucccaaggaugcagaggccucagccgaagauucacca

SEQ ID NO: 883 - RNA, 39 nt, Mus musculus
aagaggucaccaugucauauggucuuccugcugcucccu

SEQ ID NO: 884 - RNA, 26 nt, Mus musculus
uuguacuuccugcugcuccuggacuu

SEQ ID NO: 885 - RNA, 31 nt, Mus musculus
uuccacagccaggagaguacauuaggugccu

SEQ ID NO: 886 - RNA, 26 nt, Mus musculus
uguauguauggcugccuggagcugcu

SEQ ID NO: 887 - RNA, 26 nt, Mus musculus
uguauggcugccuggagcugcucgaa

SEQ ID NO: 888 - RNA, 26 nt, Mus musculus
uggugaagccuaguugcaacagauga

SEQ ID NO: 889 - RNA, 26 nt, Mus musculus
ugcgccugggugucuggccuguacuc

SEQ ID NO: 890 - RNA, 27 nt, Mus musculus
ucuuugucuggccuguagguuaucguc

SEQ ID NO: 891 - RNA, 28 nt, Mus musculus
ucuguauguauggcugccuggagcugcu

SEQ ID NO: 892 - RNA, 25 nt, Mus musculus
ucacaucuuugucuggccuguaggu

SEQ ID NO: 893 - RNA, 26 nt, Mus musculus
uaucaucggggugcugcuugaugccc

SEQ ID NO: 894 - RNA, 29 nt, Mus musculus
uagugcuuccugcuuucugauucagucag

SEQ ID NO: 895 - RNA, 26 nt, Mus musculus
uaguagagguccaggaacaaagcucc

SEQ ID NO: 896 - RNA, 42 nt, Mus musculus
uaauuugcugcugcacuauguaccccuggcuacccagccugu

SEQ ID NO: 897 - RNA, 26 nt, Mus musculus
uaagggaacagaugcuguuggcuagc

SEQ ID NO: 898 - RNA, 27 nt, Mus musculus
gaagaccauuguauugccuggagcugc

SEQ ID NO: 899 - RNA, 26 nt, Mus musculus
ccugcugugaguugcaucagaucggc

SEQ ID NO: 900 - RNA, 29 nt, Mus musculus
ccaucacaucuuugucuggccuguagguu

SEQ ID NO: 901 - RNA, 26 nt, Mus musculus
cacugacccugacugcugcugcacua

SEQ ID NO: 902 - RNA, 27 nt, Mus musculus
auccacuaugucuggccuguacagucu

SEQ ID NO: 903 - RNA, 41 nt, Mus musculus
agauagccaagguuguacuuccugcugcuccuggacuuggu

SEQ ID NO: 904 - RNA, 27 nt, Mus musculus
ugggcugagggaacuggagcugcuuga

SEQ ID NO: 905 - RNA, 26 nt, Mus musculus
ucauagugcuuccuggcagagagugu

SEQ ID NO: 906 - RNA, 27 nt, Mus musculus
uagugauuucccccaaggaacaaaggc

SEQ ID NO: 907 - RNA, 34 nt, Mus musculus
uagccgaggcuggccaugagcuguugauccuccu

SEQ ID NO: 908 - RNA, 31 nt, Mus musculus
agaaguugcaacagaugucagacuauugccu

SEQ ID NO: 909 - RNA, 26 nt, Mus musculus
uaggucuauauaagcagcaccaguuc

SEQ ID NO: 910 - RNA, 25 nt, Mus musculus
uaacagaguggggauuuccaagucu

SEQ ID NO: 911 - RNA, 31 nt, Mus musculus
uggaacacuacuacuguccauagaagacaca

SEQ ID NO: 912 - RNA, 26 nt, Mus musculus
uguugauuuuggugccauucugaccu

SEQ ID NO: 913 - RNA, 26 nt, Mus musculus
ugcuuauauagaccuaggagugacgu

SEQ ID NO: 914 - RNA, 26 nt, Mus musculus
uagagauguugauguacugccaacuu

SEQ ID NO: 915 - RNA, 27 nt, Mus musculus
ggcugagguuguacugccaaguggacc

SEQ ID NO: 916 - RNA, 46 nt, Mus musculus
ugguggagacuuggauuuaaaaggcugcguccagugugaugaccca

SEQ ID NO: 917 - RNA, 32 nt, Mus musculus
ugcuaagugauguacugcaaaaaccaggcagu

SEQ ID NO: 918 - RNA, 27 nt, Mus musculus
ugauguacugggcauauucugacuuuc

SEQ ID NO: 919 - RNA, 26 nt, Mus musculus
ugagacuuggaaaucuagggaggugg

SEQ ID NO: 920 - RNA, 27 nt, Mus musculus
uaugcuaagugauguacugcaaaaacc

SEQ ID NO: 921 - RNA, 26 nt, Mus musculus
uagcugauguacugcaaaguggaaaa

SEQ ID NO: 922 - RNA, 32 nt, Mus musculus
uaagugauguacugcaaaaaccaggcaguugu

SEQ ID NO: 923 - RNA, 26 nt, Mus musculus
uagcagaacacccccaggucuauggg

SEQ ID NO: 924 - RNA, 35 nt, Mus musculus
accagcagaacacccuugcugcucauugaauccag

SEQ ID NO: 925 - RNA, 27 nt, Mus musculus
uguuagaugacgucaucgagcugaagg

SEQ ID NO: 926 - RNA, 45 nt, Mus musculus
ugggcugugguccugcuggagggcugguugcuaaaaccagcaccg

SEQ ID NO: 927 - RNA, 26 nt, Mus musculus
ugcugcucccugacaaccacuaagug

SEQ ID NO: 928 - RNA, 26 nt, Mus musculus
ugacacaagcuggaguaaucacugag

SEQ ID NO: 929 - RNA, 25 nt, Mus musculus
ugaagcugcacgacuucuccucucc

SEQ ID NO: 930 - RNA, 25 nt, Mus musculus
ucucugaaacccuggugaaccgagc

SEQ ID NO: 931 - RNA, 31 nt, Mus musculus
uccgaccacaugaaggugcacagccagggcc

SEQ ID NO: 932 - RNA, 29 nt, Mus musculus
ucaagugugcagugcuucagucacacagu

SEQ ID NO: 933 - RNA, 33 nt, Mus musculus
uaccugagcacccugucccaggucacagaagac

SEQ ID NO: 934 - RNA, 30 nt, Mus musculus
uacaaaguguacugaagcugcacgacuucu

SEQ ID NO: 935 - RNA, 26 nt, Mus musculus
ggccgugcugcugcccaagaagacgg

SEQ ID NO: 936 - RNA, 50 nt, Mus musculus
gcugcccugagcuggcaguaccugagcacccugucccaggucacagaaga

SEQ ID NO: 937 - RNA, 50 nt, Mus musculus
gcguccugcccaacauccaggccgugcugcugcccaagaagacggagagc

SEQ ID NO: 938 - RNA, 27 nt, Mus musculus
gccgugcugcugcccaagaagacggag

SEQ ID NO: 939 - RNA, 38 nt, Mus musculus
caguaccugagcacccugucccaggucacagaagacua

SEQ ID NO: 940 - RNA, 50 nt, Mus musculus
cagggcggcguccugcccaacauccaggccgugcugcugcccaagaagac

SEQ ID NO: 941 - RNA, 31 nt, Mus musculus
caggccgugcugcugcccaagaagacggaga

SEQ ID NO: 942 - RNA, 29 nt, Mus musculus
accaucuguaacuccagcugaagggcauc

SEQ ID NO: 943 - RNA, 27 nt, Mus musculus
aaguguacugaagcugcacgacuucuc

SEQ ID NO: 944 - RNA, 26 nt, Mus musculus
aacauccaggccgugcugcugcccaa

SEQ ID NO: 945 - RNA, 26 nt, Mus musculus
ugggauacuucaaggaggacuuccug

SEQ ID NO: 946 - RNA, 26 nt, Mus musculus
ugcugaacauccuggggcagagugcc

SEQ ID NO: 947 - RNA, 28 nt, Mus musculus
uucucugugauaacuccagcuugugcca

SEQ ID NO: 948 - RNA, 26 nt, Mus musculus
ugugcugcuucauguguagaauucag

SEQ ID NO: 949 - RNA, 28 nt, Mus musculus
uaacuacuguugcugcuucauguggcgc

SEQ ID NO: 950 - RNA, 31 nt, Mus musculus
agaaacugcugaugaacuucagggugaagua

SEQ ID NO: 951 - RNA, 30 nt, Mus musculus
acugcugaugaacuucagggugaaguaucu

SEQ ID NO: 952 - RNA, 29 nt, Mus musculus
aagaaacugcugaugaacuucagggugaa

SEQ ID NO: 953 - RNA, 28 nt, Mus musculus
uuucucagugauuacuccagcuuguguc

SEQ ID NO: 954 - RNA, 30 nt, Mus musculus
uuucaucagucuugaagaagauggaaauac

SEQ ID NO: 955 - RNA, 31 nt, Mus musculus
uucaucagucuugaagaagauggaaauacag

SEQ ID NO: 956 - RNA, 26 nt, Mus musculus
ugagggaacgcugcuucauguggagu

SEQ ID NO: 957 - RNA, 26 nt, Mus musculus
ugaagaagauggugucuguccaaaca

SEQ ID NO: 958 - RNA, 30 nt, Mus musculus
ugaacuccagcaggaagaggagauagcucu

SEQ ID NO: 959 - RNA, 31 nt, Mus musculus
ucaaacagauggugcgcuccgagcgacgagc

SEQ ID NO: 960 - RNA, 29 nt, Mus musculus
uaucaaacagauggugcgcuccgagcgac

SEQ ID NO: 961 - RNA, 27 nt, Mus musculus
cugaacuccagcaggaagaggagauag

SEQ ID NO: 962 - RNA, 27 nt, Mus musculus
ccuugaagaagauguugguauucuugc

SEQ ID NO: 963 - RNA, 31 nt, Mus musculus
caucagucuugaagaagauggaaauacagga

SEQ ID NO: 964 - RNA, 30 nt, Mus musculus
cagucuugaagaagauggaaauacaggaua

SEQ ID NO: 965 - RNA, 27 nt, Mus musculus
caggacaggaacuccagcaggagcuga

SEQ ID NO: 966 - RNA, 32 nt, Mus musculus
aucaaacagauggugcgcuccgagcgacgagc

SEQ ID NO: 967 - RNA, 26 nt, Mus musculus
uaugaagaagauggugggaaagaucu

SEQ ID NO: 968 - RNA, 28 nt, Mus musculus
uaggacugggugcucacugcugaggcua

SEQ ID NO: 969 - RNA, 26 nt, Mus musculus
uuuggcucugaagacaagaucuguag

SEQ ID NO: 970 - RNA, 25 nt, Mus musculus
ugaagacaagaucuguagccauccc

SEQ ID NO: 971 - DNA, 20 bp, Artificial Sequence
  primer
tatgtacacaccgcatacag

SEQ ID NO: 972 - DNA, 20 bp, Artificial Sequence
  primer
cgcgggtgataaacatgtta

SEQ ID NO: 973 - DNA, 20 bp, Artificial Sequence
  primer
acccaggagtcaaatggaaa

SEQ ID NO: 974 - DNA, 20 bp, Artificial Sequence
  primer
actgactagagactagtggc

SEQ ID NO: 975 - DNA, 20 bp, Artificial Sequence
  primer
gaggctggatttttggtact

SEQ ID NO: 976 - DNA, 20 bp, Artificial Sequence
  primer
catgaatagcaacagggact

SEQ ID NO: 977 - DNA, 20 bp, Artificial Sequence
  primer
aataggggctgaacatgtca

SEQ ID NO: 978 - DNA, 20 bp, Artificial Sequence
  primer
atctgaaggagtagcatcct

SEQ ID NO: 979 - DNA, 20 bp, Artificial Sequence
  primer
aaagagatggcaactagcac

SEQ ID NO: 980 - DNA, 20 bp, Artificial Sequence
  primer
aggacaagcaaaagcaaatt

SEQ ID NO: 981 - DNA, 21 bp, Artificial Sequence
  primer
aacactttgcttcacactcaa

SEQ ID NO: 982 - DNA, 20 bp, Artificial Sequence
  primer
cacatggggatagcactact

SEQ ID NO: 983 - RNA, 2060 nt, SARS-CoV-2
uauguacacaccgcauacagucuuacaggcuguuggggcuuguguucuuugcaauucacagacuucauuaagauguggugcuugcauacguagaccauucuuauguuguaaaugcuguuacgaccaugucauaucaacaucacauaaauuagucuugucuguuaauccguauguuugcaaugcuccagguugugaugucacagaugugacucaacuuuacuuaggagguaugagcuauuauuguaaaucacauaaaccacccauuaguuuuccauugugugcuaauggacaaguuuuugguuuauauaaaaauacauguguugguagcgauaauguuacugacuuuaaugcaauugcaacaugugacuggacaaaugcuggugauuacauuuuagcuaacaccuguacugaaagacucaagcuuuuugcagcagaaacgcucaaagcuacugaggagacauuuaaacugucuuaugguauugcuacuguacgugaagugcugucugacagagaauuacaucuuucaugggaaguugguaaaccuagaccaccacuuaaccgaaauuaugucuuuacugguuaucguguaacuaaaaacaguaaaguacaaauaggagaguacaccuuugaaaaaggugacuauggugaugcuguuguuuaccgagguacaacaacuuacaaauuaaauguuggugauuauuuugugcugacaucacauacaguaaugccauuaagugcaccuacacuagugccacaagagcacuauguuagaauuacuggcuuauacccaacacucaauaucucagaugaguuuucuagcaauguugcaaauuaucaaaagguugguaugcaaaaguauucuacacuccagggaccaccugguacugguaagagucauuuugcuauuggccuagcucucuacuacccuucugcucgcauaguguauacagcuugcucucaugccgcuguugaugcacuaugugagaaggcauuaaaauauuugccuauagauaaauguaguagaauuauaccugcacgugcucguguagaguguuuugauaaauucaaagugaauucaacauuagaacaguaugucuuuuguacuguaaaugcauugccugagacgacagcagauauaguugucuuugaugaaauuucaauggccacaaauuaugauuugaguguugucaaugccagauuacgugcuaagcacuauguguacauuggcgacccugcucaauuaccugcaccacgcacauugcuaacuaagggcacacuagaaccagaauauuucaauucaguguguagacuuaugaaaacuauagguccagacauguuccucggaacuugucggcguuguccugcugaaauuguugacacugugagugcuuugguuuaugauaauaagcuuaaagcacauaaagacaaaucagcucaaugcuuuaaaauguuuuauaaggguguuaucacgcaugauguuucaucugcaauuaacaggccacaaauaggcgugguaagagaauuccuuacacguaacccugcuuggagaaaagcugucuuuauuucaccuuauaauucacagaaugcuguagccucaaagauuuugggacuaccaacucaaacuguugauucaucacagggcucagaauaugacuaugucauauucacucaaaccacugaaacagcucacucuuguaauguaaacagauuuaauguugcuauuaccagagcaaaaguaggcauacuuugcauaaugucugauagagaccuuuaugacaaguugcaauuuacaagucuugaaauuccacguaggaauguggcaacuuuacaagcugaaaauguaacaggacucuuuaaagauuguaguaagguaaucacuggguuacauccuacacaggcaccuacacaccucaguguugacacuaaauucaaaacugaagguuuauguguugacauaccuggcauaccuaaggacaugaccuauagaagacucaucucuaugauggguuuuaaaaugaauuaucaaguuaaugguuacccuaacauguuuaucacccgcg

SEQ ID NO: 984 - RNA, 1367 nt, SARS-CoV-2
acccaggagucaaauggaaauugauuucuuagaauuagcuauggaugaauucauugaacgguauaaauuagaaggcuaugccuucgaacauaucguuuauggagauuuuagucauagucaguuaggugguuuacaucuacugauuggacuagcuaaacguuuuaaggaaucaccuuuugaauuagaagauuuuauuccuauggacaguacaguuaaaaacuauuucauaacagaugcgcaaacagguucaucuaaguguguguguucuguuauugauuuauuacuugaugauuuuguugaaauaauaaaaucccaagauuuaucuguaguuucuaagguugucaaagugacuauugacuauacagaaauuucauuuaugcuuugguguaaagauggccauguagaaacauuuuacccaaaauuacaaucuagucaagcguggcaaccggguguugcuaugccuaaucuuuacaaaaugcaaagaaugcuauuagaaaagugugaccuucaaaauuauggugauagugcaacauuaccuaaaggcauaaugaugaaugucgcaaaauauacucaacugugucaauauuuaaacacauuaacauuagcuguacccuauaauaugagaguuauacauuuuggugcugguucugauaaaggaguugcaccagguacagcuguuuuaagacagugguugccuacggguacgcugcuugucgauucagaucuuaaugacuuugucucugaugcagauucaacuuugauuggugauugugcaacuguacauacagcuaauaaaugggaucucauuauuagugauauguacgacccuaagacuaaaaauguuacaaaagaaaaugacucuaaagaggguuuuuucacuuacauuuguggguuuauacaacaaaagcuagcucuuggagguuccguggcuauaaagauaacagaacauucuuggaaugcugaucuuuauaagcucaugggacacuucgcaugguggacagccuuuguuacuaaugugaaugcgucaucaucugaagcauuuuuaauuggauguaauuaucuuggcaaaccacgcgaacaaauagaugguuaugucaugcaugcaaauuacauauuuuggaggaauacaaauccaauucaguugucuuccuauucuuuauuugacaugaguaaauuuccccuuaaauuaagggguacugcuguuaugucuuuaaaagaaggucaaaucaaugauaugauuuuaucucuucuuaguaaagguagacuuauaauuagagaaaacaacagaguuguuauuucuagugauguucuuguuaacaacuaaauguuuguuuuucuuguuuuauugccacuagucucuagucagu

SEQ ID NO: 985 - RNA, 1572 nt, SARS-CoV-2
gaggcuggauuuuugguacuacuuuagauucgaagacccagucccuacuuauuguuaauaacgcuacuaauguuguuauuaaagucugugaauuucaauuuuguaaugauccauuuuuggguguuuauuaccacaaaaacaacaaaaguuggauggaaagugaguucagaguuuauucuagugcgaauaauugcacuuuugaauaugucucucagccuuuucuuauggaccuugaaggaaaacaggguaauuucaaaaaucuuagggaauuuguguuuaagaauauugaugguuauuuuaaaauauauucuaagcacacgccuauuaauuuagugcgugaucucccucaggguuuuucggcuuuagaaccauugguagauuugccaauagguauuaacaucacuagguuucaaacuuuacuugcuuuacauagaaguuauuugacuccuggugauucuucuucagguuggacagcuggugcugcagcuuauuauguggguuaucuucaaccuaggacuuuucuauuaaaauauaaugaaaauggaaccauuacagaugcuguagacugugcacuugacccucucucagaaacaaaguguacguugaaauccuucacuguagaaaaaggaaucuaucaaacuucuaacuuuagaguccaaccaacagaaucuauuguuagauuuccuaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucagacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucaggccgguagcacaccuuguaaugguguugaagguuuuaauuguuacuuuccuuuacaaucauaugguuuccaacccacuaaugguguugguuaccaaccauacagaguaguaguacuuucuuuugaacuucuacaugcaccagcaacuguuuguggaccuaaaaagucuacuaauuugguuaaaaacaaaugugucaauuucaacuucaaugguuuaacaggcacagguguucuuacugagucuaacaaaaaguuucugccuuuccaacaauuuggcagagacauugcugacacuacugaugcuguccgugauccacagacacuugagauucuugacauuacaccauguucuuuugguggugucaguguuauaacaccaggaacaaauacuucuaaccagguugcuguucuuuaucaggauguuaacugcacagaagucccuguugcuauucaug

SEQ ID NO: 986 - RNA, 1954 nt, SARS-CoV-2
aauaggggcugaacaugucaacaacucauaugagugugacauacccauuggugcagguauaugcgcuaguuaucagacucagacuaauucuccucggcgggcacguaguguagcuagucaauccaucauugccuacacuaugucacuuggugcagaaaauucaguugcuuacucuaauaacucuauugccauacccacaaauuuuacuauuaguguuaccacagaaauucuaccagugucuaugaccaagacaucaguagauuguacaauguacauuuguggugauucaacugaaugcagcaaucuuuuguugcaauauggcaguuuuuguacacaauuaaaccgugcuuuaacuggaauagcuguugaacaagacaaaaacacccaagaaguuuuugcacaagucaaacaaauuuacaaaacaccaccaauuaaagauuuuggugguuuuaauuuuucacaaauauuaccagauccaucaaaaccaagcaagaggucauuuauugaagaucuacuuuucaacaaagugacacuugcagaugcuggcuucaucaaacaauauggugauugccuuggugauauugcugcuagagaccucauuugugcacaaaaguuuaacggccuuacuguuuugccaccuuugcucacagaugaaaugauugcucaauacacuucugcacuguuagcggguacaaucacuucugguuggaccuuuggugcaggugcugcauuacaaauaccauuugcuaugcaaauggcuuauagguuuaaugguauuggaguuacacagaauguucucuaugagaaccaaaaauugauugccaaccaauuuaauagugcuauuggcaaaauucaagacucacuuucuuccacagcaagugcacuuggaaaacuucaagauguggucaaccaaaaugcacaagcuuuaaacacgcuuguuaaacaacuuagcuccaauuuuggugcaauuucaaguguuuuaaaugauauccuuucacgucuugacaaaguugaggcugaagugcaaauugauagguugaucacaggcagacuucaaaguuugcagacauaugugacucaacaauuaauuagagcugcagaaaucagagcuucugcuaaucuugcugcuacuaaaaugucagaguguguacuuggacaaucaaaaagaguugauuuuuguggaaagggcuaucaucuuauguccuucccucagucagcaccucaugguguagucuucuugcaugugacuuaugucccugcacaagaaaagaacuucacaacugcuccugccauuugucaugauggaaaagcacacuuuccucgugaaggugucuuuguuucaaauggcacacacugguuuguaacacaaaggaauuuuuaugaaccacaaaucauuacuacagacaacacauuugugucugguaacugugauguuguaauaggaauugucaacaacacaguuuaugauccuuugcaaccugaauuagacucauucaaggaggaguuagauaaauauuuuaagaaucauacaucaccagauguugauuuaggugacaucucuggcauuaaugcuucaguuguaaacauucaaaaagaaauugaccgccucaaugagguugccaagaauuuaaaugaaucucucaucgaucuccaagaacuuggaaaguaugagcaguauauaaaauggccaugguacauuuggcuagguuuuauagcuggcuugauugccauaguaauggugacaauuaugcuuugcuguaugaccaguugcuguaguugucucaagggcuguuguucuuguggauccugcugcaaauuugaugaagacgacucugagccagugcucaaaggagucaaauuacauuacacauaaauggauuuguuuaugagaaucuucacaauuggaacuguaacuuugaagcaaggugaaaucaaggaugcuacuccuucagau

SEQ ID NO: 987 - RNA, 1968 nt, SARS-CoV-2
aaagagauggcaacuagcacucuccaaggguguucacuuuguuugcaacuugcuguuguuguuuguaacaguuuacucacaccuuuugcucguugcugcuggccuugaagccccuuuucucuaucuuuaugcuuuagucuacuucuugcagaguauaaacuuuguaagaauaauaaugaggcuuuggcuuugcuggaaaugccguuccaaaaacccauuacuuuaugaugccaacuauuuucuuugcuggcauacuaauuguuacgacuauuguauaccuuacaauaguguaacuucuucaauugucauuacuucaggugauggcacaacaaguccuauuucugaacaugacuaccagauuggugguuauacugaaaaaugggaaucuggaguaaaagacuguguuguauuacacaguuacuucacuucagacuauuaccagcuguacucaacucaauugaguacagacacugguguugaacauguuaccuucuucaucuacaauaaaauuguugaugagccugaagaacauguccaaauucacacaaucgacgguucauccggaguuguuaauccaguaauggaaccaauuuaugaugaaccgacgacgacuacuagcgugccuuuguaaauguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuucuuuuuacguuuacucucguguuaaaaaucugaauucuucuagaguuccugaucuucuggucuaaacgaacuaaauauuauauuaguuuuucuguuuggaacuuuaauuuuagccauggcagauuccaacgguacuauuaccguugaagagcuuaaaaagcuccuugaacaauggaaccuaguaauagguuuccuauuccuuacauggauuugucuucuacaauuugccuaugccaacaggaauagguuuuuguauauaauuaaguuaauuuuccucuggcuguuauggccaguaacuuuagcuuguuuugugcuugcugcuguuuacagaauaaauuggaucaccgguggaauugcuaucgcaauggcuugucuuguaggcuugauguggcucagcuacuucauugcuucuuucagacuguuugcgcguacgcguuccauguggucauucaauccagaaacuaacauucuucucaacgugccacuccauggcacuauucugaccagaccgcuucuagaaagugaacucguaaucggagcugugauccuucguggacaucuucguauugcuggacaccaucuaggacgcugugacaucaaggaccugccuaaagaaaucacuguugcuacaucacgaacgcuuucuuauuacaaauugggagcuucgcagcguguagcaggugacucagguuuugcugcauacagucgcuacaggauuggcaacuauaaauuaaacacagaccauuccaguagcagugacaauauugcuuugcuuguacaguaaauguuucaucucguugacuuucagguuacuauagcagagauauuacuaauuauuaugaggacuuuuaaaguuuccauuuggaaucuugauuacaucauaaaccucauaauuaaaaauuuaucuaagucacuaacugagaauaaauauucucaauuagaugaagagcaaccaauggagauugauuaaaugaaaauuauucuuuucuuggcacugauaacacucgcuacuugugagcuuuaucacuaccaagaguguguuagagguacaacaguacuuuuaaaagaaccuugcucuucuggaacauacgagggcaauucaccauuucauccucuagcugauaacaaauuugcacugacuugcuuuagcacucaauuugcuuuugcuuguccu

SEQ ID NO: 988 - RNA, 2201 nt, SARS-CoV-2
aacacuuugcuucacacucaaaagaaagacagaaugauugaacuuucauuaauugacuucuauuugugcuuuuuagccuuucugcuauuccuuguuuuaauuaugcuuauuaucuuuugguucucacuugaacaugauugaacuuucauuaauugacuucuauuugugcuuuuuagccuuucugcuauuccuuguuuuaauuaugcuuauuaucuuuugguucucacuugaacugcaagaucauaaugaaacuugucacgccuaaaugaaauuucuuguuuucuuaggaaucaucacaacuguagcugcauuucaccaagaauguaguuuacagucauguacucaacaucaaccauauguaguugaugacccguguccuauucacuucuauucuaaaugguauauuagaguaggagcuagaaaaucagcaccuuuaauugaauugugcguggaugaggcugguucuaaaucacccauucaguacaucgauaucgguaauuauacaguuuccuguuuaccuuuuacaauuaauugccaggaaccuaaauuggguagucuuguagugcguuguucguucuaugaagacuuuuuagaguaucaugacguucguguuguuuuagauuucaucuaaaugucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaaacucaugcagaccacacaaggcagaugggcuauauaaacguuuucgcuuuuccguuuacgauauauagucuacucuugugcagaaugaauucucguaacuacauagcacaaguagauguaguuaacuuuaaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugug

(19) United States
(12) Patent Application Publication
(10) Pub. No.: US 2023/0374515 A1
(43) Pub. Date: Nov. 23, 2023

(54) METHODS OF TREATING VIRAL INFECTIONS WITH PiRNAS OR EXTRACELLULAR VESSICLES RELEASED FROM NEURAL STEM CELLS OR NEURAL PROGENITOR CELLS

(71) Applicant: Albert Einstein College of Medicine, Bronx, NY (US)

(72) Inventor: Dongsheng Cai, Rye, NY (US)

(21) Appl. No.: 18/247,152

(22) PCT Filed: Oct. 5, 2021
(86) PCT No.: PCT/US21/53580

§ 371 Date: Mar. 29, 2023

Related U.S. Application Data
(60) Provisional application No. 63/087,590, filed on Oct. 5, 2020.

(51) Int. Cl. C12N 15/113; A61P 31/14

(52) U.S. Cl. CPC C12N 15/1131; A61P 31/14; C12N 2310/11

(57) ABSTRACT

Described herein are methods of treating an individual in need of treatment or prevention of infection with a virus including administering to the individual a therapeutically effective amount of extracellular vesicles released from neural stem cells and/or neural progenitor cells, or administering to the individual a therapeutically effective amount of a composition including one or more piRNAs, wherein the piRNAs have a perfect match with a sense or antisense sequence of the genome of the virus in a primary lead sequence at positions 2-8. Also included are compositions including the piRNAs.

### CROSS REFERENCE TO RELATED APPLICATION

This application claims the benefit of U.S. Provisional Patent Application No. 63/087,590, filed Oct. 5, 2020, which is incorporated herein in its entirety.

### STATEMENT REGARDING FEDERALLY SPONSORED RESEARCH & DEVELOPMENT

This invention was made with government support under AG031774 awarded by the National Institutes of Health. The government has certain rights in the invention.

### BACKGROUND

RNA viruses represent an enormous public health problem in the U.S. and worldwide. Well-known RNA viruses include influenza virus (including the avian and swine isolates; also referred to as flu), Hepatitis C virus (HCV), Dengue virus (DNV), West Nile virus (WNV), SARS-coronavirus (SARS), MERS-coronavirus (MERS), respiratory syncytial virus (RSV), and human immunodeficiency virus (HIV). These viruses are responsible for pandemic outbreaks and threats to public health that have occurred throughout history. Flaviviruses, Henipaviruses, Filoviruses, and Arenaviruses are among emerging RNA viruses that pose significant public health and biodefense risks. These viruses collectively place hundreds of millions of people at risk of infection throughout the world. Many of the emerging RNA viruses cause viral hemorrhagic fever and can result in significant morbidity and mortality. Dengue virus (DNV) and West Nile virus (WNV) are both Flaviviruses (positive strand RNA virus) and Arboviruses, transmitted through mosquitoes; thus these viruses represent a potent potential biological threat through their ability to transmit readily among insects or animals and humans, high infectivity, and their potential to be weaponized in bioterror events.

SARS-CoV-2 is the name given to the coronavirus that causes the respiratory disease called coronavirus disease 19 (COVID-19). SARS-CoV-2 was first known to infect humans in 2019. Despite efforts to contain the disease in China, the virus has spread around the world, and COVID-19 was thus declared to be a pandemic by the World Health Organization (WHO) in March 2020. COVID-19 primarily spreads through the respiratory tract, by droplets, respiratory secretions, and direct contact. SARS-CoV-2 is highly transmissible in humans, particularly in the elderly and people with underlying chronic diseases.

Currently, Currently RNA vaccines have been developed but no specific antiviral treatment for COVID-19 is available. What is needed are novel therapies for the treatment and/or prevention of SARS-CoV-2 infection, in addition to other viral infections such as HIV infection.

### BRIEF SUMMARY

In an aspect, a method of treating an individual in need of treatment or prevention of infection with a virus comprises administering to the individual a therapeutically effective amount of extracellular vesicles released from neural stem cells and/or neural progenitor cells.

In another aspect, a method of treating an individual in need of treatment or prevention of infection with a virus comprises administering to the individual a therapeutically effective amount of a composition comprising one or more piRNAs, wherein the piRNAs have a perfect match with a sense or antisense sequence of the genome of the virus in a primary lead sequence at positions 2-8.

In another aspect, a composition comprises a polymeric particle, liposome, micelle, lipid-polymer particle, dendrimer, inorganic nanoparticle, hydrogel, or a combination thereof, and a population of piRNAs, wherein the population of piRNAs have a perfect match with a sense or antisense sequence of the genome of a virus in a primary lead sequence at positions 2-8.

### BRIEF DESCRIPTION OF THE DRAWINGS

FIG. 1 is a schematic diagram depicting mouse piRNAs which potentially target the sense or antisense sequence of the SARS-CoV-2 genome. Small bars indicate piRNAs that have the matched sequences with the adjacent viral RNA strand and thus have a complementary match with the other RNA strand, showing piRNAs fitting with Criteria 1 and piRNAs fitting with Criteria 2. The drawn lengths of viral genomic segments are not proportional to their actual sizes. For Orf1ab and spike sequences, the presentation includes only piRNAs which met Criteria 1.

FIG. 2 shows that exosomes and microvesicles (labelled as “Ex/Mv” thereafter) were isolated and purified from htNSCPGHM (labelled as htNSCP), hpNSC and MSC, and the same amount of total small RNA from these Ex/Mv were examined by qPCR for a list of piRNAs which could potentially target SARS-CoV-2 genome. htNSCPGHM was a subtype of htNSC. Data for each piRNA are expressed as fold changes compared to the values of MSC (the values of MSC were all adjusted as 1). Information on sequences and labels of these piRNAs are detailed in Table 1. *p<0.05, **p<0.01, ***p<0.001; ANOVA/post-hoc, compared between indicated groups, n=3 independent biological samples per group, values represent the mean±s.e.m.

FIG. 3 shows htNSC vs. htNSCPGHM Ex/Mv piRNAs against SARS-CoV-2 genome. Ex/Mv were isolated and purified from htNSCPGHM (labelled as htNSCP) and htNSC, and the same amount of total small RNA from these Ex/Mv were examined by qPCR for a list of piRNAs which could potentially target SARS-CoV-2 genome. Data of each piRNA are expressed as fold changes compared to the values of htNSC (the values of htNSC were all adjusted as 1). Table 1 has detailed information of these piRNAs. *p<0.05, **p<0.01, ***p<0.001; 2-tailed Student's t-test, n=4 independent biological samples per group, values represent the mean±s.e.m.

FIG. 4 shows the presence of PIWIL2 in NSC-released Ex/Mv-. The result is a Western blot of PIWIL2 in Ex/Mv isolated from mouse htNSC, htNSC^{PGHM} (labelled as htNSC^{P}), hpNSC and MSC. β-actin blot of the same membrane was included as a reference.

FIG. 5 shows immunostaining of PIWIL2 for cultured htNSC, htNSC^{PGHM} (labelled as htNSC^{P}) and hpNSC compared to MSC. The panel only presented the images of htNSCP and MSC (images of htNSC^{P}, htNSC and hpNSC were all similar). DAPI nuclear staining was included as a reference. Scale bar 20 μm.

FIG. 6 shows co-immunostaining of PIWIL2 (middle) and exosomal marker TSG101 (top) for Ex/Mv isolated from cultured htNSC^{PGHM} (labelled as htNSC^{P}). Right panel shows a higher-magnification view from an Ex/Mv cropped from the left panel. DAPI nuclear staining was included as a reference. Scale bars, 500 nm.

FIG. 7 is a schematic diagram depicting mouse piRNAs which potentially target the sense or antisense sequence of pseudotyped SARS-CoV-2 genome (spike 2 enveloped ΔG-luc-VSV). Small bars indicate piRNAs that have the matched sequences with the adjacent viral RNA strand and thus complementary match with the other RNA strand), showing piRNAs fitting with Criteria 1 and piRNAs with Criteria 2. The drawn lengths of viral genomic segments are not proportional to their actual sizes.

FIG. 8 shows Ex/Mv were isolated and purified from htNSCPGHM (labelled as htNSC^{P}), hpNSC and MSC, and the same amount of total small RNA from these Ex/Mv were examined by qPCR for a list of piRNAs which could potentially target pseudotyped SARS-CoV-2 genome. Data of each piRNA are expressed as fold changes compared to the values of MSC (the values of MSC were all adjusted as 1). Information on sequences and labels of these piRNAs are detailed in Table 2. *p<0.05, **p<0.01, ***p<0.001; ANOVA/post-hoc, compared between indicated groups, n=3 independent biological samples per group, values represent the mean±s.e.m.

FIG. 9 shows additional NSC Ex/Mv piRNAs against pseudotyped SARS-CoV-2. Refer to Table 2 for detailed information of these piRNAs. *p<0.05, **p<0.01, ***p<0.001; ANOVA/post-hoc, compared between indicated groups, n=3 independent biological samples per group, values represent the mean±s.e.m.

FIG. 10 shows htNSC^{PGHM} vs. htNSC Ex/Mv piRNAs against pseudotyped SARS-CoV-2. Ex/Mv were isolated and purified from htNSC^{PGHM} (labelled as htNSC^{P}) and htNSC, and the same amount of total small RNA from these Ex/Mv were examined by qPCR for a list of piRNAs which could potentially target pseudotyped SARS-CoV-2 genome. Data of each piRNA are expressed as fold changes compared to the values of htNSC (the values of htNSC were all adjusted as 1). Refer to Table 2 for detailed information of these piRNAs. *p<0.05, **p<0.01, ***p<0.001; two-tailed Student's t test, n=4 independent biological samples per group, values represent the mean±s.e.m.

FIG. 11 shows evaluation of hACE2-expressing NSC via immunostaining NSC were induced with hACE2-expressing lentivirus (CMV promoter) and selected through an antibiotic marker of this lentivirus to make stable cell lines. These cells were evaluated through immunostaining via hACE2 antibody. DAPI nuclear staining was included to provide a technical reference. Scale bar, 50 μm.

FIG. 12 shows induced NSC Ex/Mv piRNAs by pseudotyped SARS-CoV-2 stimulation. Cultured hACE2-expressing htNSC were incubated with a pseudotyped SARS-CoV-2 (spike 2 enveloped ΔG-luc-VSV) for 2 generations and then returned to normal culture without virus for 5 generations before experiments (labelled as “induced”). VSV: vesicular stomatitis virus. These cells under the same procedure except viral infection were used as the baseline control (labelled as “basal”). Ex/Mv were isolated and purified from these basal and induced NSCs, and the same amount of total small RNA from these Ex/Mv were examined by qPCR for a list of piRNAs which could potentially target this pseudotyped SARS-CoV-2 genome. Data of each piRNA after normalization with U6 were calculated as fold changes compared to the values of basal group (the values of the basal group were all adjusted as 1). Refer to Table 2 for detailed information of these piRNAs. *p<0.05, **p<0.01, ***p<0.001; 2-tailed Student's t-test, compared between induced and basal groups, n=4 independent biological samples per group, values represent the mean±s.e.m.

FIG. 13 shows information on piRNAs that potentially target the sense or antisense of luciferase RNA sequence which was contained in the genome of pseudotyped SARS-CoV-2. Comparisons are among Ex/Mv of htNSC^{PGHM} (labelled as htNSC^{P}), hpNSC and MSC.

FIG. 14 shows comparison between htNSC^{PGHM} Ex/Mv vs. htNSC Ex/Mv for the levels of basal and virus stimulated-induced piRNAs against luciferase RNA sequence in the genome of pseudotyped SARS-CoV-2.

FIG. 15 shows additional comparison between htNSC^{PGHM} Ex/Mv vs. htNSC Ex/Mv for the levels of basal and virus stimulation-induced piRNAs against luciferase RNA sequence in the genome of pseudotyped SARS-CoV-2.

FIG. 16 shows a schematic diagram depicting mouse piRNAs which potentially target against the sense or antisense sequence of recombinant lentiviral genome. Small bars indicate piRNAs that have the matched sequences with the adjacent viral RNA strand and thus complementary match with the other RNA strand), showing piRNAs fitting with Criteria land piRNAs fitting with Criteria 2. The drawn lengths of viral genomic segments are not proportional to their actual sizes. 5′-LTR and 3′-LTR are based on the same genomic sequences and piRNAs.

FIG. 17 shows Ex/Mv were isolated and purified from htNSC^{PGHM} (labelled as htNSC^{P}), hpNSC and MSC, and the same amount of total small RNA from these Ex/Mv were examined by qPCR for a list of piRNAs which could potentially target a lentiviral genome. Data of each piRNA are expressed as fold changes compared to the values of MSC (the values of MSC were all adjusted as 1).—Information on sequences and labels of these piRNAs are detailed in—Table 3. *p<0.05, **p<0.01, ***p<0.001; ANOVA/post-hoc, compared between indicated groups, n=3 independent biological samples per group. Values represent the mean±s.e.m.

FIG. 18 shows additional piRNAs for FIG. 17. Refer to Table 3 for detailed information of these piRNAs. *p<0.05, **p<0.01, ***p<0.001; ANOVA/post-hoc, compared between indicated groups, n=3 independent biological samples per group, values represent the mean±s.e.m.

FIGS. 19 and 20 show cultured htNSC^{P} or hpNSC were incubated with a VSVG-enveloped GFP lentivirus for 2 generations and then returned to normal culture for 5 generations before experiments (labelled as “i-” followed by NSC type). These cells under the same procedure except viral infection were used as the basal control (labelled as “b-” followed NSC type). Ex/Mv were isolated and purified from these basal and induced NSCs, and the same amount of total small RNA from these Ex/Mv were examined by qPCR for a list of piRNAs which could potentially target this lentiviral RNA genome (FIG. 20) or GFP RNA (FIG. 21). Data of each piRNA after normalization with U6 were calculated as fold changes compared to the values of basal group (the values of the basal group were all adjusted as 1).

FIG. 21 shows additional information on NSC Ex/Mv piRNAs against lentivirus.

FIG. 22 shows antiviral effects of NSC vs. MSC Ex/Mv for VSVG-incorporated lentivirus. Cultured A549 cells were infected with VSVG-incorporated GFP lentivirus in the presence or absence of Ex/Mv that were isolated from NSC types and MSC as indicated, and 2 days later, these cells were fixed for GFP immunostaining. DAPI nuclear staining was included as a technical control. Scale bar, 100 μm.

FIGS. 23 and 24 show cultured A549 cells were infected with a low (FIG. 23) or high (FIG. 24) dose of VSVG-enveloped GFP lentivirus in the presence or absence of Ex/Mv that were isolated from basal vs. induced NSC types as indicated, and 2 days later, these cells were harvested and lysed for western blot for GFP. Blot for beta tubulin or beta actin for the same membrane was included to provide a reference. n.s.: non-specific.

FIG. 25 shows VSVG-enveloped GFP lentiviruses were mixed with basal Ex/Mv from indicated NSC type for 0.5 hour at room temperature and then 4-24 hours at 4° C. These lentiviruses or Ex/Mv alone under the same conditions were included as controls. These samples were then lysed and processed for western blot using an antibody against VSVG. Ponceau staining of duplicated gels under the same procedure was include as a technical control.

FIGS. 26 and 27 show antiviral effects of NSC Ex/Mv for pseudotyped SARS-CoV-2. Cultured A549, HepG2 and Calu3 cells were infected with pseudotyped SARS-CoV-2 (spike 2 incorporated ΔG-luc-VSV) in the presence or absence of purified Ex/Mv (labelled as “Ex”) that were isolated from indicated NSC types as indicated, and 2 days later, these cells were harvested for the measurement of luciferase activities (FIG. 26) or fixed for immunostaining of luciferase protein (FIG. 27) Immunostaining images were based on htNSC group, while images of all NSC groups were quantitatively analyzed in right panels (randomized 90 cells per group were analyzed, each dot represented the average fluorescence intensity of a single cell). Scale bar 100 μm. ***p<0.001; ANOVA/post-hoc, compared between indicated groups, n=3 independent biological samples per group, values represent the mean±s.e.m.

FIG. 28 shows cultured Calu3 cells were infected with pseudotyped SARS-CoV-2 in the presence or absence of purified Ex/Mv from a basal (labelled as “b-”) or induced (labelled as “i-”) NSC types versus vehicle, and 2 days later, these cells were harvested for the measurement of luciferase activities. *p<0.05, **p<0.01; ANOVA/post-hoc, compared between indicated groups, n=3 independent biological samples per group, values represent the mean±s.e.

FIGS. 29 and 30 shows cell-free antiviral effects of NSC Ex/Mv for pseudotyped SARS-CoV-2. Pseudotyped SARS-CoV-2 (spike 2 enveloped ΔG-luc-VSV) were mixed with indicated purified exosomes (without including microvesicles) vs. vehicle control and incubated for 0.5 hour at room temperature and then overnight at 4° C. Samples were employed to do uranyl acetate negative staining and TEM on grids. E: exosome, V: virus. Arrows in FIG. 29 point to virus or exosome. Arrows in FIG. 30 point to exosome-virus interaction or damaged virus. Scale bar 100 μm.

FIG. 31 shows pseudotyped SARS-CoV-2 were mixed with Ex/Mv vs. vehicle control and incubated for 0.5 hour at room temperature and then overnight at 4° C. Samples were then lysed and processed for western blot using an antibody against spike 2 or exosomal markers CD81 and TSG101. Ponceau staining of the gels was included as a technical control.

FIG. 32 shows pseudotyped SARS-CoV-2 (spike 2 enveloped ΔG-luc-VSV) and indicated Ex/Mv with overnight pre-mixture (labelled as “V & Ex pre-mixture”) or alone (labeled as “V & Ex alone”) under the same conditions were used to infect HepG2 cells for 24 hours. Subsequently these cells were harvested and lysed for measurement of luciferase activities. *p<0.05, **p<0.01; ANOVA/post-hoc, compared between indicated groups, n=3 independent biological samples per group, values represent the mean±s.e.m.

FIG. 33A-E shows antiviral effects of htNSC Ex/Mv against SARS-CoV-2. (A-C) hACE2-A549 cells were evaluated for (A) hACE2 mRNA via qPCR, (B) hACE2 immunostaining, and (C) hACE2 protein via Western blot. A: hACE2 mRNA levels in hACE2-A549 cells as fold change compared to the levels in A549 cells of which the average was adjusted as 1. AU: arbitrary unit. B: hACE2 staining is shown in bottom panel while DAPI staining in top panel reveals nuclei of all cells in slides. Scale bar, 50 μm. C: blotting for GAPDH in the same membrane was used as a technical control. (D-E) hACE2-A549 cells were infected with (D) 0.1 MOI and (E) 1.0 MOI of SARS-CoV-2 virus USA-WA1/2020 and treated with htNSC Ex/Mv (17.5 μg per106 cells) or vehicle control, and 2 days later these cells were lysed for measuring the levels of N1, N2, N3, S and E segments as well as sub-genomic E region (Sg-E) of SARS-CoV-2 RNA genome via qPCR. Data are expressed as fold changes with respect to control group of which the average was adjusted as 1. ***p<0.001, ****p<0.0001, 2-tailed unpaired student t-test was applied between groups as indicated (A, D, E); data represent mean±SEM.

FIG. 34A-F shows Piwil2 knockout in htNSC leading to decreased Ex/Mv piRNAs. (A-B) Knockout of piwil2 gene in htNSC was done using mammalian CRISPRlentiviral transduction followed by blasticidin selection to create stable htNSC-PIWIL2 KO cell lines. These htNSC-PIWIL2 KO cells were evaluated in comparison with Control htNSC using (A) Western blot for Piwil2 protein with β-actin of the same membrane as a technical control, and (B) Piwil2 immunostaining (left panel) of neurospheres while DAPI nuclear staining (right panel) was used as a reference. Scale bars, 50 μm. (C-F) Equal amount of Ex/Mv from htNSC-PIWIL2 KO (labelled as PIWIL2 KO Ex/Mv) versus control htNSC (labelled as Control Ex/Mv) were used to extract small RNAs for quantitation of (C) total small RNAs, (D) small RNAs at the size of 10-40 nt via small RNA bioanalyzer assay, (E) total protein levels, and (F) a list of piRNAs by qPCR. F: abbreviations of piRNA species corresponded to a region in SARS-CoV-2 genome including regions for making spike protein (S), membrane protein (M) and nucleocapsid (N), open reading frames (O), UTR (U), and gap sequences which linked some of these regions (G). Data of each piRNA is expressed as fold changes relative to the average values of control adjusted as 1. *p<0.05, **p<0.01, ***p<0.001, ****p<0.0001, 2-tailed unpaired student t-test was applied between groups as indicated, data represent mean±SEM.

FIG. 35A-E shows loss of antiviral effects by htNSC Ex/Mv due to Piwil2 knockout. (A) hACE2-A549 cells were infected with 1.0 MOI SARS-CoV-2 virus and treated with treated with Ex/Mv (17.5 μg per 106 cells) isolated from htNSC PIWIL2 KO (labelled as “PIWIL2 KO Ex/Mv”—rightmost bar) versus control htNSC (labelled as “Control Ex/Mv”—center bar) or vehicle control (labelled as “Vehicle Control”—leftmost bar) for 2 days. These cells were harvested and analyzed for N1, N2, N3, S and E gene segments as well as sub-genomic E region (Sg-E) of SARS-CoV-2. Data are represented as fold change with respect to control whose average was adjusted as 1. (B-E) Immunostaining for viral luciferase was used to confirm the infection models of (B) pseudotyped SARS-CoV-2 and (D) pseudotyped SARS-CoV-1 in hACE2-A549 cells, and using these infection models, hACE2-A549 cells were infected with (C) pseudotyped SARS-CoV-2 or (E) pseudotyped SARS-CoV-1 and were treated with the same amount of Ex/Mv from htNSC P1WIL2 KO versus Control htNSC for 3 days. These cells were then harvested for measuring luciferase activities to quantitively report the infection levels of these pseudotyped viruses Immunostaining for uninfected hACE2-A549 cells were included as control references (B, D). *p<0.05, **p<0.01, ***p<0.001, ****p<0.0001, one-way ANOVA followed by Tukey's post-hoc test; data represent mean±SEM.

FIG. 36A-B shows induced Ex/Mv in htNSC via SARS-CoV-2 RNA fragmentstimulation(A) Schematic of SARS-CoV-2 genomic fragments (F1-F6) which were cloned into pCR-Blunt II-TOPO vector leading to productions of F1-6 RNA fragments via in vitro transcription. Small bars above F1-6 indicate piRNA species with the sequences related to one of these fragments. (B) htNSC were transfected with a mixture of F1-6 RNA fragments, this process was repeatedly 2 weeks later, and these cells were established as induced htNSC. Small RNAs were extracted from an equal amount of Ex/Mv from induced htNSC (labelled as induced Ex/Mv) versus control htNSC (labelled as Control Ex/Mv) and measured for piRNAs which could potentially target SARS-CoV-2 genome. Data of each piRNA in induced htNSC Ex/Mv are expressed as fold changes relative to the average values of control htNSC Ex/Mv adjusted as 1. Please refer to FIG. 35 regarding abbreviations of these piRNA species. *p<0.05, **p<0.01, ***p<0.001, ****p<0.0001, 2-tailed unpaired student t-test was applied between groups as indicated; data represent mean±SEM. FIG. 37 A-B shows Piwil2-dependent enhanced antiviral effects of induced htNSC Ex/Mv. (A) hACE2-A549 cells were infected with SARS-CoV-2 and treated with Ex/Mv from induced htNSC (labelled as induced Ex/Mv—rightmost bar) versus control htNSC (labelled as Control Ex/Mv—center bar) for 2 days, and cells were lysed and examined for N1, N2, N3, S and E gene segments along with sub-genomic E region (Sg-E) of SARS-CoV-2 using qPCR. (B) htNSC-PIWIL2 KO versus control htNSC were subjected to twice exposures of F1-6 through transfection as described in FIG. 37A. Same amount Ex/Mv from htNSC-PIWIL2 KO (rightmost bar) versus control htNSC (center bar) or vehicle (leftmost bar) were used to treat hACE2-A549 cells upon infection of MOI 1.0 SARS-CoV-2 for 2 days. Then, cells were lysed and examined for N1, N2, N3, S and E gene segments along with sub-genomic E region (Sg-E) of SARS-CoV-2 using qPCR using primers. Data of expression is represented as fold change with respect to control whose average was adjusted as 1. *p<0.05, **p<0.01, ***p<0.001, ****p<0.0001; ANOVA/post-hoc was applied between the indicated groups, values represent mean±SEM.

The above-described and other features will be appreciated and understood by those skilled in the art from the following detailed description, drawings, and appended claims.

### DETAILED DESCRIPTION

The brain is the “headquarters” of the body and should be strictly protected from infections, especially infections by viruses which are small and can penetrate the brain parenchyma relatively more easily than other pathogens such as bacteria. This antiviral protection requires brain immunity which has been appreciated at the level of innate immunity. Recently, with the increasing knowledge regarding the neural control of immunity, research further suggests that the brain has its own lymphatic system and thus likely its own adaptive immunity. Of interest, it was recently reported that NSCs in the olfactory bulb play an important role in the brain's immune defense. As unexpectedly shown herein, extracellular vesicles released from neural stem cells or neural progenitor cells can be used as an effective antiviral therapy.

In addition, the hypothalamus, for example, has a role in regulating adaptive immunity, and hypothalamic neural stem/progenitor cells (htNSC) abundantly produce exosomal miRNAs which circulate throughout the cerebrospinal fluid (CSF). In view of these results, htNSC exosomes/microvesicles (henceforth referred to as Ex/Mv) were studied for P-element induced wimpy testis (PIWI)-interacting RNAs (piRNAs), a very large family of small RNAs which is increasingly gaining attention and has been linked to viral immunity, although mainly in insect models. Without being held to theory, it is believed that the piRNAs in extracellular vesicles released from neural stem cells or neural progenitor cells are at least partially responsible for the observed antiviral effects. An antiviral extracellular vesicle and/or piRNA strategy could be complementary to vaccine development, as the latter is challenged by the hurdle of targeting an RNA virus, partially because variants and mutations of RNA viruses are high, for instance, systematic-level mutational and protein profile analyses revealed a large number of amino acid substitutions in SARS-CoV-2 indicating that the viral proteins are heterogeneous.

In an aspect, a method of treating an individual in need of treatment or prevention of infection with a virus comprises administering to the individual a therapeutically effective amount of extracellular vesicles released from neural stem cells and/or neural progenitor cells.

As used herein the term NSC includes both neural stem cells and neural progenitor cells. Neural stem cells are multipotent cells which are able to self-renew and proliferate without limit, to produce progeny cells which terminally differentiate into neurons, astrocytes and oligodendrocytes. The non-stem cell progeny of NSCs are referred to as neural progenitor cells. NSCs release vesicles which contain cargo including, but not limited to, non-coding RNAs, DNA, and proteins such as heat shock proteins and tetraspanins.

As used herein, the term extracellular vesicles includes exosomes, microvesicles, or both. The extracellular vesicles can be isolated from any animal with NSCs that release vesicles, such as mouse, human, rat, hamster, guinea pig, rabbit, pig, goat, cow, dog, cat, non-human primates, and combinations thereof.

In an aspect, the neural stem cells and/or neural progenitor cells originate from a brain region such as the hypothalamus or hippocampus or subventricular zone (SVZ). The term originate includes cells released from neural stem cells and/or neural progenitor cells, as well as clones or subclones derived from neural stem cells and/or neural progenitor cells.

Also included herein is a method of treating an individual in need of treatment or prevention of infection with a virus, the method comprising administering to the individual a therapeutically effective amount of a composition comprising one or more piRNAs, wherein the piRNAs have a perfect match with a sense or antisense sequence of the genome of the virus in a primary lead sequence at positions 2-8. In a more specific aspect, the piRNAs have a perfect match with a sense or antisense sequence of the genome of the virus in a primary lead sequence at positions 2-11, and no more than 5 mismatches in a secondary lead sequence at nucleotides 12-21.

piRNAs are 24-32 nucleotides, slightly longer than miRNAs (21-24 nucleotides), but differ from miRNAs in many ways including that piRNAs lack a conserved sequence and their biogenesis does not require the Dicer machinery. Functionally, piRNAs induce genic and intergenic silencing and particularly transposon silencing and splicing, and this regulation on transposons represents an immunity-like action against transposon invasion. Mammalian piRNAs are present in germ cells, and the action of silencing transposons by piRNAs is crucial for spermatogenesis and embryonic development, agreeing with the appreciated predominant levels of piRNAs in testes and ovaries. Recently, evidence suggests that piRNAs are present in the nervous system in significant amounts and play roles in neural development, neurobehavior, and memory formation. The activities of piRNAs are mediated by PIWI proteins, mainly PIWI1 and PIWI2 (PIWIL1 and PIWIL2 in mammals, respectively), and while both PIWIL1 and 2 are important for germline piRNAs, PIWIL2 can also mediate the functions of piRNAs in the brain. Described herein is evidence indicating that murine NSCs produce Ex/Mv that contain large antiviral piRNA libraries and the immunity against viruses including HIV-based lentivirus and VSV-based pseudotyped SARS-CoV-2, which could be developed as a strategy for combating SARS-CoV-2.

In an aspect, the extracellular vesicles comprise piRNAs, wherein the piRNAs have a perfect match with a sense or antisense sequence of the genome of the virus in a primary lead sequence at positions 2-8.

In an aspect, the extracellular vesicles or composition comprise a population of 1, 2, 3, 4, 5, 6, 7, 8, 9, 10, 11, 12, 13, 14, 15, 16, 17, 18, 19, 20, 50, 100, 200, 500, 1000 or more distinct piRNA sequences.

The methods described herein can be used to treat infections caused by both RNA and DNA viruses, because both need to generate RNAs for infections. In an aspect, the viral infection is caused by a virus of the family Arbovirus, Arenaviridae, Arterivirus, Astroviridae, Birnaviridae, Bromoviridae, Bunyaviridae, Caliciviridae, Circoviridae, Closteroviridae, Comoviridae, Coronaviridae, Cystoviridae, Filoviridae, Flaviviridae, Flexiviridae, Hepadnaviridae, Hepevirus, Herpesviridae, Leviviridae, Luteoviridae, Mesoniviridae, Mononegavirales, Mosaic Viruses, Nidovirales, Nodaviridae, Orthomyxoviridae, Papillomaviridae, Papovaviridae, Parvoviridae, Paramyxoviridae, Picobirnaviridae, Picobirnavirus, Picornaviridae, Potyviridae, Poxviridae, Reoviridae, Retroviridae, Roniviridae, Sequiviridae, Tenuivirus, Togaviridae, Tombusviridae, Totiviridae, or Tymoviridae.

Specific viruses include Alfuy virus, Banzi virus, bovine diarrhea virus, Chikungunya virus, Dengue virus (DNV), Epstein Barr Virus (EBV), Hepatitis B virus (HBV), Hepatitis C virus (HCV), herpes simplex virus type 1 (HSV-1), herpes simplex virus type 2 (HSV-2), human cytomegalovirus (hCMV), human immunodeficiency virus (HIV), Ilheus virus, influenza virus (including avian and swine isolates), rhinovirus, norovirus, adenovirus, Japanese encephalitis virus, Kaposi's sarcoma associated herpesvirus (KSHV), Kokobera virus, Kunjin virus, Kyasanur forest disease virus, louping-ill virus, measles virus, MERS-coronavirus (MERS), metapneumovirus, any of the Mosaic Viruses, Murray Valley virus, parainfluenza virus, poliovirus, Powassan virus, respiratory syncytial virus (RSV), Rocio virus, SARS-coronavirus (SARS), St. Louis encephalitis virus, tick-borne encephalitis virus, West Nile virus (WNV), Ebola virus, Nipah virus, Lassa virus, Tacaribe virus, Junin virus, yellow fever virus, Varicella zoster virus (VZV), or vesicular stomatitis virus.

In a specific aspect, the virus is HIV, VSV, or SARS-CoV-2.

When the virus is HIV, the piRNA sequences are homologous with a sense or antisense sequence of long-terminal repeat (LTR), Rev response element (RRE), psi, U3, or a combination thereof. Exemplary piRNAs include SEQ ID NOs. 816-970 (Table 3).

When the virus is VSV, and wherein the piRNA sequences are homologous with a sense or antisense sequence of the RNA sequences encoding nucleocapsid protein (N sequence), phosphoprotein (P sequence), matrix protein (M sequence), RNA polymerase (R sequence), a non-encoding structural sequence, or a combination thereof. Exemplary piRNAs include any of SEQ ID NOS. 522-815.

When the virus is SARS-CoV-2, the piRNA sequences are homologous with a sense or antisense sequence of spike protein, envelope protein, membrane protein, nucleocapsid protein, open reading frame sequence 1ab (Orf1ab), Orf 3a, Orf 6, Orf 7a, Orf 7b, Orf 8, Orf 10, 5′ end untranslated region (UTR) sequence, 3′ end UTR sequence, a non-encoding sequence, or a combination thereof. Exemplary piRNAs include SEQ ID NOs. 1-521 (Table 1).

Exemplary individuals for the methods described herein include mammals and non-mammals. “Mammals” means a member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “individual” does not denote a particular age or sex.

The phrase “effective amount,” as used herein, means an amount of an agent which is sufficient enough to significantly and positively modify symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

Exemplary modes of administration include parenteral, intravenous, subcutaneous, intranasal, oral, pulmonary, ocular, vaginal, rectal, or intrathecal administration.

As used herein, “pharmaceutical composition” means therapeutically effective amounts of the compound together with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

For topical application to the eye, the inhibitor may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anaesthetic, preservative and buffering agents can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

Exemplary compositions comprising the piRNAs include compositions in the form of in the form of polymeric particles, liposomes, micelles, lipid-polymer particles, dendrimers, inorganic nanoparticles, hydrogels, or a combination thereof.

Synthetic nanoparticles for piRNA delivery include polymeric nanoparticles, micelles, lipid micelles, liposomes, and hybrid lipid-polymer nanoparticles.

Polymeric particles typically include biodegradable, biocompatible polymers. Biocompatible polymers include, but are not limited to, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polylactides, polyglycolides, polysiloxanes, polyurethanes and copolymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt; polyacrylic acid polymers such as polymers of acrylic and methacrylic esters such as poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyalkylenes such as polyethylene, polypropylene poly(ethylene glycol), poly(ethylene oxide), and poly(ethylene terephthalate), poly(vinyl alcohols), poly(vinyl acetate), poly vinyl chloride polystyrene and polyvinylpryrrolidone, derivatives thereof, linear and branched copolymers and block copolymers thereof, and blends thereof.

Exemplary biodegradable polymers include, but are not limited to, polyesters, poly(ortho esters), poly(ethylene imines), poly(caprolactones), poly(hydroxybutyrates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In particularly preferred embodiments the polymeric core contains biodegradable hydrophobic polyesters such as poly(lactic acid), poly(glycolic acid), and poly(lactic-co-glycolic acid), and/or these polymers conjugated to polyalkylene oxides such as polyethylene glycol or block copolymers such as the polypropylene oxide-polyethylene oxide PLURONICs®.

A mixture or blend of polymers, copolymer or block copolymers may be employed, such as copolymers of modified polyethylene glycol (PEG) and polyesters, such as various forms of PLGA-PEG or PLA-PEG copolymers, collectively referred to herein as “PEGylated polymers.

The piRNAs may be delivered in the form of a liposome, such as a lipid monolayer or bilayer. In some embodiments, nanoparticles including the piRNAs include a polymeric core or a non-polymeric core (e.g., metal particle, quantum dot, ceramic particle, bone particle, etc.) surrounded by a lipid layer.

Exemplary lipids are biocompatible oils such as plant oils and butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Exemplary oils include a fatty acid group such as of butyric, caproic, caprylic, capric, lauric, myristic, palmitic, stearic, arachidic, behenic, or lignoceric acid, palmitoleic, oleic, vaccenic, linoleic, alpha-linolenic, gamma-linoleic, arachidonic, gadoleic, arachidonic, eicosapentaenoic, docosahexaenoic, or erucic acid.

Additional lipids include a steroid (e.g., cholesterol, bile acid), vitamin (e g vitamin E), phospholipid (e.g. phosphatidyl choline), sphingolipid (e.g. ceramides), or lipoprotein (e.g. apolipoprotein).

In certain embodiments, the lipid is phosphatidylcholine, lipid A, cholesterol, dolichol, sphingosine, sphingomyelin, ceramide, glycosylceramide, cerebroside, sulfatide, phytosphingosine, phosphatidyl-ethanolamine, phosphatidylglycerol, phosphatidylinositol, phosphatidylserine, cardiolipin, phosphatidic acid, and/or lyso-phophatides.

In another aspect, the piRNA composition comprises nanoparticles include one or more polymers associated covalently, or non-covalently with one or more lipids, such as phospholipids.

Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and beta-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine.

Exemplary dendrimers for piRNA compositions include, poly(amido-amine) (PAMAM), poly(ethyleneimine) (PEI), polyester, polylysine, polypropyleneimine (PPI), diaminobutane amine polypropylenimine tetramine (DAB-Am 4), polypropylamine (POPAM), polylysine, polyester, iptycene, aliphatic poly(ether), and/or aromatic polyether dendrimers. The dendrimers can have carboxylic, amine and hydroxyl terminations and can be of any generation including, but not limited to, generation 1 dendrimers (G1), generation 2 dendrimers (G2), generation 3 dendrimers (G3), generation 4 dendrimers (G4), generation 5 dendrimers (G5), generation 6 dendrimers (G6), generation 7 dendrimers (G7), generation 8 dendrimers (G8), generation 9 dendrimers (G9), or generation 10 dendrimers (G10).

Exemplary hydrogels for piRNA compositions include an alginate or a derivative thereof, gelatin, collagen, agarose, a natural or synthetic polysaccharide, polylactic acid, polyglycolic acid, poly(lysine), a polyanhydride; a poly(lactide-co-glycolide) (PLGA) polymer, a polyamino acid, a poly(alkylene oxide), a poly(ethylene oxide), a poly(allylamine)(PAM), a poly(acrylate), a polyester, polyhydroxybutyrate and poly-epsilon-caprolactone, a polyphosphazine, a poly(vinyl alcohol), a modified styrene polymer, poly(4-aminomethylstyrene), a pluronic polyol, a polyoxamer, a poly(uronic acid), a poly(vinylpyrrolidone), and/or a copolymer comprising one or more of an alginate or a derivative thereof, gelatin, collagen, agarose, a natural or synthetic polysaccharide, polylactic acid, polyglycolic acid, poly(lysine), a polyanhydride; a poly(lactide-co-glycolide) (PLGA) polymer, a polyamino acid, a poly(alkylene oxide), a poly(ethylene oxide), a poly(allylamine)(PAM), a poly(acrylate), a polyester, polyhydroxybutyrate and poly-epsilon-caprolactone, a polyphosphazine, a poly(vinyl alcohol), a modified styrene polymer, poly(4-aminomethylstyrene), a pluronic polyol, a polyoxamer, a poly(uronic acid), and a poly(vinylpyrrolidone).

In addition to the therapeutic methods described herein, the extracellular vesicles and piRNAs can be used in decontamination of a substrate, such as hospital instruments or work surfaces. In order to treat a contaminated substrate, the extracellular vesicles and piRNAs may be applied to the site of such contamination in an amount sufficient to kill viruses.

The invention is further illustrated by the following non-limiting examples.

### EXAMPLES

### Methods

Primary NSC models and culture: Primary culture of NSCs was performed as described in the art. In brief, the hypothalamus or hippocampus was dissected from newborn C57BL/6 mice, cut into small pieces of approximately 1 mm3, followed by digestion using TrypLE™ Express enzyme (Life Technologies™/ThermoFisher Scientific™) for 10 min at 37° C. After centrifugation, cells were suspended in NSC medium composed of Neurobasal™-A (Life Technologies™/ThermoFisher Scientific™), 0.25% GlutaMAX™ supplement (Life Technologies™/ThermoFisher Scientific™), 2% B27 without vitamin A (Life Technologies™/ThermoFisher Scientific™), 10 ng ml-1 EGF (Sigma-Aldrich™) 10 ng ml-1 bFGF (Life Technologies™/ThermoFisher Scientific™) and 1% penicillin— streptomycin and seeded in ultralow-adhesion 6-well plates (Corning). One week later, neurospheres were collected by centrifugation and trypsinized with TrypLE™Express enzyme into single cells, passaged and maintained in neurosphere culture. All procedures of involving animal uses were approved by the Institutional Care and Use Committee of Albert Einstein College of Medicine.

NSC lines: The line of htNSC^{PGHM} was established previously. hACE2 overexpressing stable NSC cell lines were generated by hACE2 lentivirus transduction for two days and puromycin-based sections for one week. Lentivirus or pseudotyped SARS-CoV-2 infected stem cell lines were generated by persistent infection with 2 μl virus per 106 cells for 1 week, followed by normal NSC media culture.

Other cell lines: Mouse mesenchymal stem cells (MUBMX-01001, OriCell™ Cyagen) were cultured in MSC medium (MUXMX-90011, OriCell™ Cyagen). HEK293T (CRL-3216, ATCC®), HepG2 (HB-8065, ATCC®) and BHK21 (EH1011, Kerafast™) cells were incubated in Dulbecco's modified Eagle medium (ThermoFisher Scientific™). A549 (CCL-185, ATCC®) was cultured in DMEM/F-12 Medium (ATCC®). Calu3 (HTB-55, ATCC®) was cultured in Minimum Essential Medium (ATCC®). Except NSC culture medium which was serum-free, all other culture media were supplemented with 5-10% heat-inactivated fetal bovine serum (Sigma Aldrich™) or MSC specific fetal bovine serum (Cyagen™), 100 U/mL of penicillin and streptomycin (Gibco™). For seeding and subcultivation, cells were first washed with phosphate buffered saline (PBS) and then incubated in the presence of trypsin/EDTA solution (Life Technologies™/ThermoFisher Scientific™) until cells detached. BHK21 was cultured at 37° C. and 7% CO2 in a humidified atmosphere, while all other cell lines were cultured at 37° C. and 5% CO2 in a humidified atmosphere.

The model of htNSC-PIWIL2 KO was generated by subjecting htNSC to mammalian CRISPR lentiviral infection followed by blasticidin selection and were then maintained as a stable htNSC cell line. The model of hACE2-A549 cells was generated through infecting A549 cells (CCL-185, ATCC®) with hACE2-expressing lentiviruses for two days followed by puromycin selection leading to a stable cell line. A549 and hACE2-A549 cells were cultured using F-12K nutrient mixture (Gibco™). HEK293T (CRL-3216, ATCC®) and BHK21 (EH1011, Kerafast™) cell cultures were done using Dulbecco's modified Eagle medium (Gibco™). HEK293T, BHK21, A549, hACE2-A549 cell culture media was supplemented with 5-10% heat-inactivated fetal bovine serum, FBS (Gibco™) and 1% Penicillin-Streptomycin (Gibco™) BHK21 cell culture was maintained at 37° C. and 7% CO2 humidified atmosphere, and all other cell models were maintained at 37° C. and 5% CO2 humidified atmosphere. 0.25% Trypsin-EDTA (Gibco™) was used for trypsinization of attached culture for passaging. Lipofectamine 3000 (Life Technologies™/ThermoFisher Scientific™) was used for cell transfection per the manufacturer's instruction.

Ex/Mv isolation and purification: Ex/Mv in MSC, NSC, and htNSC cell culture media were purified by differential centrifugation as described in the art. In brief, culture medium was processed by ultracentrifugation at 100,000 g, 4° C. overnight to remove particles to generate exosome-free medium. Cells were cultured in exosome-free medium for two days, after which the medium was collected, centrifuged to remove cells and large debris. The supernatant was collected and centrifuged at 2000 g for 10 min to remove small debris, immediately followed by Ex/Mv isolation in 4° C., then filtered through 0.8 μm pore-size filter for Ex/Mv or 0.2 μm pore-size filter for exosomes only. The filtrate was collected and ultra-centrifuged at 100,000g for 70 min to pellet down particles and re-suspended with PBS. Fresh Ex/Mv particles were made within 2-3 weeks before an experiment for its use, while repeated freezing and thawing were avoided.

Plasmids and virus production: All procedures related to virus production were handled under biosafety level 2 and approved by institutional biosafety committee. The lentiviral vector of CMV-promoter-driven GFP was generated in the art. The lentiviral vector of EF1A-promoter-driven hACE2 (VB200000-2751mcf) was purchased from Vector Builder™. Vector pCAGGS containing the SARS-Related Coronavirus 2, Wuhan-Hu-1 spike glycoprotein gene (NR-52310) was obtained from BEI Resources. pCAGGS-G-Kan plasmid (EH1017) was purchased from Kerafast. Vector pCAGGS containing SARS-CoV-1 spike glycoprotein was gifted by Whittaker Lab, Robert Frederick Smith School of Chemical & Biomolecular Engineering, Cornell University. Lentiviral plasmid vector EF1A-promoter-driven hACE2 (VB200000-2751mcf) and mammalian CRISPR lentiviral plasmid vector for PIWIL2 knockout (VB201202-1202gqa) were purchased from Vector Builder™. Lentiviruses were produced by transfecting HEK293T cells with corresponding viral and packaging plasmids, purified by ultracentrifugation as described previously. Pseudotyped ΔG-luciferase rVSV (EH1025-PM) was purchased from Kerafast™. Generation of pseudotyped ΔG-luciferase rVSV or pseudotypes with SARS-CoV-2 glycoprotein was performed as described in the art. In brief, BHK-21 cells were first transfected with pCAGGS-G-Kan or SARS-CoV-2 spike glycoprotein plasmid, then 24 hrs later infected with ΔG-luciferase rVSV. Culture supernatants were harvested at 24 hrs post-infection and ultracentrifuged with a 20% sucrose cushion at 100,000 g for 35 min. The recombinant lentiviruses, rVSV or pseudotyped SARS-CoV-2 viruses were reconstituted in a small volume of PBS to achieve a 1000× concentration.

Cell lysate was prepared for Western blot by sonication in ice-cold RIPA lysis buffer (20 mM Tris-HCl, pH 7.4, 10 mM NaCl, 1 mM EDTA, 0.01% SDS, 1% Triton X-100 and 1× protease inhibitor cocktail). After protein concentration was determined, cell lysate samples were denatured in sample buffer at 95° C. for 5 min and prepared for loading.

Ex/Mv preparation for Western blot: 25 μg of Ex/Mv that isolated from NSC-secreted media were mixed with 1 μl lentivirus or 5 μl pseudovirus SARS2-luciferaseVSV. The mixtures incubated for designed time and at designed temperature, were the incubated mixture was mixed with sample buffer and boiled at 95° C. for 5 min, then used for western blot to analyze VSV-G or SAR-CoV-2 glycoprotein level.

Western blotting: The boiled protein samples were separated by 10% SDS-PAGE and were transferred onto 0.2 μm PVDF membrane. After blocking by 5% non-fat milk in TBST, the membranes were blotted with anti-PIWIL2 rabbit pAb (ab36764, ABCAM™), anti-CD81 mouse mAb (sc-23962, Santa Cruz Biotechnology®), anti-TSG101 mouse mAb (sc-7964, Santa Cruz Biotechnology®), anti-GFP rabbit pAb (NB600-308, Novus Biologicals™), anti-β-Actin rabbit pAb (4967S, Cell signaling Technology®), anti-Tubulin mouse mAb (4466, Cell signaling Technology®) anti-VSV-G rabbit pAb (NB1002485, Novus Biologicals™) or anti-SAR2-CoV-2 S human mAb (NR-52392, BEI™) overnight at 4° C., followed by treatment with HRP-conjugated goat anti-rabbit (7074, Cell Signaling Technology®), anti-mouse (7076, Cell Signaling Technology®) or anti-human (PI31410, Invitrogen™, Thermo Fisher Scientific™) secondary antibody. Ponceau S staining (Sigma Aldrich™, #P7170) was used as total protein loading control. Image J software was used for quantitatively analysis.

piRNA analysis: Different sense and antisense segments of viral RNA sequence were based on lentiviral CMV-promoter-driven-GFP vector (Thermo Scientific Fisher™), pseudotyped ΔG-luciferase rVSV genome (Karafast™, Thermo Scientific Fisher™), and SARS2-CoV-2, Wuhan-Hu-1 genome (NC_045512.2). The mouse piRNA database (piRNAQuest) was used for screening according to methods known in the art. Continuous alignment of 14-16 nucleotides and above with target RNA was set up to generate an initial list of piRNAs, which were then further aligned against a target RNA sequence to manually examine the total number of nucleotide matches as well as position-wide nucleotide matches. Criteria #1 required perfect match for the primary seed sequence (piRNA nucleotides 2-11) and allowed no more than 4 mismatches in the secondary seed sequence (piRNA nucleotides 12-21), and Criteria #2 allowed 5 mismatches in the secondary lead sequence but still required perfect matches for primary seed sequence.

Exosomal small RNA isolation and qPCR: The Ambion mirVana™ miRNA isolation kit (AM1560) were used to isolate small RNA from purified Ex/Mv, following manufacturer's guidelines. RNA concentration and purity were measured at 260 nm and 280 nm absorbance using a Nanodrop™ spectrophotometer. Extracted small RNAs were polyadenylated and reverse transcribed to cDNA using the Lucigen poly(A) polymerase tailing kit (PAP5104H) and RevertAid RT reverse transcription kit (K1691), then subjected to real-time qPCR with specific primers, universal primer and SYBR® Green PCR Master Mix. Specific primers were designed using sRNAPrimerDB module. All qPCR measurements were based on using the same amount of Ex/Mv total small RNA among groups of comparison, and U6 was used as a technical control.

Cell preparation for immunostaining: NSCs were seeded on laminin-coated coverslips. After reaching 70% confluence, cells were fixed for 15 min at room temperature with 4% PFA. A549 cells were seeded on polylysine-coated coverslips in 24-well plates and treated with 0.02-0.1 μl lentivirus and 3-5 μg exosomes or PBS, 72 hrs after seeding, cells were fixed with 4% PFA for 15 min at room temperature before immunostaining. A549 or Calu3 were seeded on polylysine-coated coverslips in 24-well plates, and treated with 1 μl pseudotyped SARS-CoV-2 virus and 3 μg exosomes or PBS, 24 hrs after seeding, cells were fixed with 4% PFA for 15 min at room temperature before immunostaining.

Exosome preparation for immunostaining: Purified exosomes isolated from NSCs or MSC were seeded on polylysine-coated coverslips and fixed after 24 hrs.

Western blotting: Cells were lysed on ice using RIPA buffer (Alfa Aesar™) with protease inhibitor cocktail (ThermoFisher Scientific™). Following protein quantification, equal quantities of cell lysate samples were mixed with laemmli buffer (Pierce™) and boiled at 95° C. for 5 min. Samples were run on SDS-PAGE gel and were transferred onto a 0.2 μm PVDF membrane (Bio-Rad®). The membranes were blocked using 5% non-fat milk in TBST and were blotted with anti-ACE2 rabbit mAb (SN0754, Invitrogen), anti-PIWIL2 mouse mAb (sc-377258, Santa Cruz Biotechnology®), anti-GAPDH mouse mAb (GA1R, Invitrogen™) or anti-β-actin rabbit pAb (4967S, Cell Signaling Technology™) overnight at 4° C. in 5% non-fat milk in TBST. Secondary HRP-conjugated antibodies subsequently applied including anti-mouse (7076P2, Cell Signaling Technology™) or anti-rabbit (7074P2, Cell Signaling Technology™). The membranes were probed using ECL system (Bio-Rad®) and Images were developed on the Image Studio software.

Immunofluorescence staining: Cell and exosome immunofluorescence staining were conducted as described in the art. In brief, specimens were washed three times in cold PBS and incubated 30 min in permeabilization buffer (Triton™ X-100 0.1%, Goat serum 5%, PBS). Cells were incubated overnight at 4° C. with primary antibodies against PIWIL2 (ab36764, ABCAM™), CD81 (sc-23962, Santa Cruz Biotechnology®), TSG101 (sc-7964, Santa Cruz Biotechnology®), GFP (NB600-308, Novus Biologicals™), Luciferase (NB600307, Novus Biologicals™), anti-ACE2 rabbit mAb (SN0754, Invitrogen™), anti-PIWIL2 mouse mAb (sc-377258, Santa Cruz Biotechnology®), or anti-luciferase, firefly rabbit pAb (AB3256, EMD Milipore®) in 5% Bovine serum albumin (BSA). and human ACE2 (MA5-32307). Cells or exosomes were incubated in secondary antibodies (Goat anti-rabbit, goat anti-mouse conjugated with Alexa Fluor® 488 or 633 and 555, respectively, Invitrogen™, ThermoFisher Scientific™) and after 3 times wash with cold PBS, cells were mounted (VECTA-SHIELD® mounting media with DAPI). Images were captured using Leica SP8 confocal microscope and analyzed by FIJI software.

Luciferase assay: A549, HepG2 or Calu3 cells seeded in 24-well plates (5×104 cells per well), cells were incubated with 1 μl pseudotyped SARS-CoV-2 virus (spike 2 incorporated ΔG-luciferase-VSV) and 3 μg exosomes, or the mixture of pseudotyped SARS-CoV-2 virus and exosomes which were pre-incubated at 4° C. overnight. 24 hrs after infection, cells were harvested and washed 3 times with PBS. The expression of firefly luciferase was quantitatively measured by Roche Luciferase reporter gene assay (11669893001). Light emission within lOs after adding luciferase assay reagent was measured by Monolight 3010 luminometer.

Alternatively, Firefly luciferase activities in cultured cells were analyzed through applying equal amount of protein lysate to One-Glo™ Luciferase assay system (Promega™). The light emissions were measured using SpectraMax® iD3. Small RNA isolated from purified Ex/Mv were quantified via spectrophotometer and subjected to small RNA chip assay with 2100 Agilent Bioanalyzer (Agilent technologies™) for quantification of small RNA species at 10-40 nt (via the Molecular Pathology Platform, Herbert Irving Comprehensive Cancer Center, Columbia University).

Electron microscopy: Exosomes were filtered (for smaller than 200 nm in diameter) and purified by ultracentrifuge with a 30% sucrose cushion at 100,000 g for 90 min, followed by washing the exosome containing sucrose layer to pellet down the exosomes by diluting with phosphate-buffered saline and ultracentrifugation at 100,000 g for 90 min. 30 μl exosomes extracted from NSCs supernatant, pseudotyped SARS-CoV-2 virus, or the mixture of exosomes and virus that was pre-incubated for overnight was used for structural analysis by transmission electron microscope. Grid preparation, uranyl acetate negative staining and transmission electron microscopy were performed at the Microscopy Core and Advanced Bioimaging Center, Icahn School of medicine at Mount Sinai.

Statistics and reproducibility: All measured data were presented as mean±SEM. Sample sizes with sufficient power were designed according to published studies, relevant literature and preliminary studies. The majority of experiments in this study were repeated independently. A two-tailed unpaired Student's t-test was used for analyses which involved only two groups for comparison, and ANOVA and Tukey's post-hoc tests were used for analyses which involved more than two groups for comparisons. The software for performing statistics included Prism and Matlab, and p values of less than 0.05 were considered statistically significant.

SARS-CoV-2 and cell treatment: SARS-CoV-2 viruses (USA-WA1/2020, BEI Resources™, NR-52281) were generated as previously described. Briefly, Vero E6 monolayer was infected with serial dilutions of viruses and then cytopathic effects for these cells were scored. The dilution at which half of cells showed cytopathic effects was calculated using Reed and Muench method as TCID50 per ml. This was used to calculate MOI values for further infections. For testing the effect of htNSC Ex/Mv, SARS-CoV-2 was mixed with Ex/Mv or vehicle in reduced serum media and added to about 80% confluent hACE2-A549 cells to achieve the final indicated MOI. Cells were maintained in these conditions for 2 days before they were treated with Trizol for RNA analysis. All these procedures were performed in Columbia Aaron Diamond AIDS Center BSL3 facility per the guidelines of the high containment BSL3 laboratory for Columbia University.

Pseudotyped SARS-CoV and cell treatment: Pseudotyped ΔG-luciferase rVSV with SARS-CoV-2 glycoprotein or SARS-CoV-1 glycoprotein was performed as similarly described in recent publication 3. In brief, 80% confluent BHK-21 were transfected with SARS-CoV-2 or SARS-CoV-1 spike glycoprotein expression plasmid. Transfected cells were culture at 37° C. with 7% CO2 in reduced serum DMEM for 30 hrs. Next day, transfection media was removed, and cells were infected with pseudotyped ΔG-luciferase rVSV in serum and antibiotic free DMEM at an MOI of 3. After 4 hrs of infection, cells were washed with 1×DPBS, reduced serum DMEM was added, and cells were further cultured for another 24 hrs. On the following day, pseudotyped viruses in culture medium were collected and filtered using 0.45 μm pore-size filter (Corning®); the filtrate was ultracentrifuged with 20% sucrose cushion at 100,000 g for 35 min at 4° C. and reconstituted in PBS for experimental use. Cultured hACE2-A549 cells at an appropriate density were added in the medium with a dose of pseudotyped SARS-CoV-2 or pseudotyped SARS-CoV-1 and about 1 hour later were added with a dose of Ex/Mv or vehicle in the medium. Cells were maintained under these conditions for 3 days before fixed or lysed for subsequent assays.

RNA in vitro transcription and cell transfection: DNA products of different SARS-CoV-2 genomic fragments were generated by the method of PCR using the cDNAs of SARS-CoV-2 (USA-WA1/2020) viral genome as the template and specific primers listed in Table 5. These PCR products were each cloned into pCR-Blunt II-TOPO vector with T7 promoter using Zero Blunt™ TOPO™ PCR Cloning Kit (Invitrogen™). Restriction enzyme digestion was carried out to screen the correct orientation of each PCR fragment containing T7 promoter. The clones containing each PCR product were linearized for in vitro transcription followed by poly (A) tailing with HiScribe™ T7 ARCA mRNA Kit (New England Biolabs™). The resulting RNA fragments were purified and quantitated for the use of cell transfection.

RNA assays by qPCR: Total RNA from cells infected with SARS-CoV-2 or variants were extracted and reverse transcribed to cDNA. Real-time qPCR assays for SARS-CoV-2 genomic sequences in N1, N2, N3, S and E region as well as sub-genomic E region (sg-E) were performed using power-up SYBR green master mix (Thermo Fisher Scientific™). All these qPCR results were normalized using the expression of house-keeping β-actin as an internal control. Primers used for these assays are described above. Total small RNAs from purified Ex/Mv were isolated using Ambion mirVana™ miRNA isolation kit (AM1560). The concentration and purity of total small RNAs were measured at 260 nm and 280 nm absorbance. Extracted small RNAs were polyadenylated by Lucigen poly(A) polymerase tailing kit (PAP5104H) and reverse transcribed by SuperScript™ III First-Strand Synthesis System (Invitrogen™, 18080-051) with universal RT primer to produce cDNAs for real-time qPCR via SYBR™ Green PCR Master Mix (Applied Biosystems™, 4309155). All piRNA qPCR results were normalized according to house-keeping U6 levels which were stable among different experimental conditions. Specific primers and the universal reverse primer used for piRNA qPCR assays. ACE2 mRNA levels in A549 and hACE2-A549 cells was also evaluated using subjecting total RNAs isolated from these cells to OCR, and these OCR results were also normalized via the expression levels of house-keeping β-actin as an internal control.

### Example 1: Antiviral piRNA Library of Murine NSC Ex/Mv Against SARS-CoV-2 Virus

According to piRNA database disposition, there are hundreds of millions of piRNA species. The numbers of unique piRNA species in mammals are also huge, for example, a mouse has at least 68 million piRNA species. The potential role of piRNAs in antiviral immunity has recently been suggested based on insect models such as mosquitoes, but piRNAs in mammals are much less studied and it is unclear if they could play a critical antiviral role and if so, where in the body and how. It has previously been shown that htNSCs abundantly produce exosomal miRNAs, and notably, as shown in exosomal RNA profiles, htNSC exosomal small RNAs contained a large number of species which were slightly bigger than miRNAs. Coincidently, the sizes of piRNAs are often 24-32 nucleotides, which are a bit longer compared to the length of miRNAs (21-24 nucleotides), provoking the hypothesis that these murine exosomal small RNAs might comprise piRNA libraries against certain viruses, as they could have been established evolutionarily. Indeed, endogenous viral elements are widely present in arthropod genomes and commonly give rise to piRNAs. Given the pandemic of SARS-CoV-2 and considering that the mouse species is resistant to this infection, the whole genome of this SARS-CoV-2 was analyzed against mouse piRNA species in piRNAQuest, a piRNA database which provides the option of searching piRNAs against a target RNA sequence.

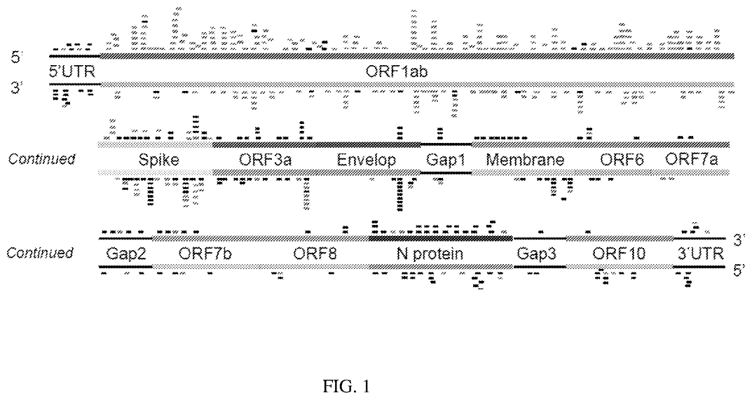

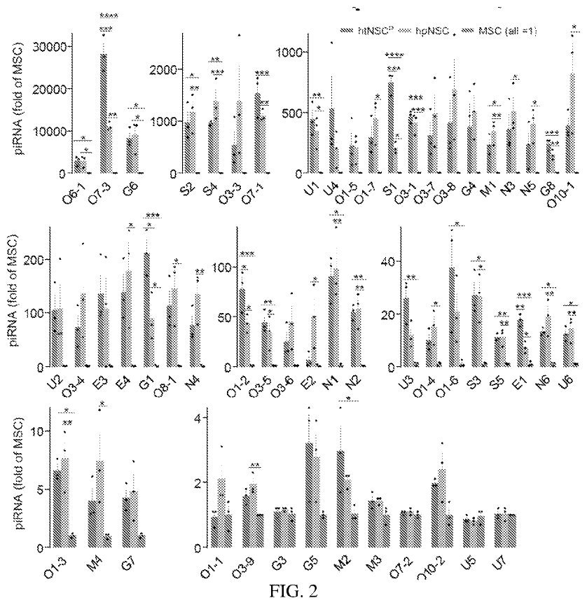

More specifically, the genomic RNA sequence of SARS-CoV-2 virus was examined, including the coding sequences for spike protein, envelope protein, membrane protein, nucleocapsid protein, open reading frame (Orf) sequences Orf1ab, 3a, 6, 7a, 7b, 8 and 10, untranslated region (UTR) sequences at 5′ end and 3′ end, and 3 gap structural sequences (gap 1-3), as shown in FIG. 1. Although SARS-CoV-2 is single-stranded RNA virus, both sense and antisense sequences were analyzed since production and replication of RNA viruses in host cells involves the synthesis of both strands. A piRNA has a primary lead sequence made of nucleotides from position 2 to 11 and a secondary lead sequence made of nucleotides from position 12 to 21. While the general rules of piRNAs in targeting RNAs are still not well established, it has recently been described in C. elegans research that matching of primary lead and particularly nucleotides 2-8 with target RNA is important, and the secondary lead sequence could have a few mismatches with target RNA. piRNAs which have at least 16 nucleotides matching with SARS-CoV-2 RNA were screened and over a thousand unique piRNA species were identified which met this requirement. The diagram in FIG. 1. presents only a portion of the identified piRNAs and their sequence information is detailed in Table 1. For piRNAs in Table 1, a perfect match of the primary lead nucleotides 2-11 was subsequently required and no more than 4 mismatches for the secondary lead nucleotides 12-21 (Criteria #1). About 170 piRNA species met this criteria (FIG. 1, Table 1). Given that SARS-CoV-2 viruses have many variants and mutations, the criteria were adjusted by allowing 5 mismatches in the secondary lead sequence, while the primary lead sequence was still required for perfect matches (Criteria #2). This led to identification of about 150 additional piRNA species that could also target SARS-CoV-2 (FIG. 1, Table 1). Over 80% of these piRNAs aligned with Orflab sequence (21kb) and spike encoding sequence (3.8kb), since these two genomic regions are much larger than other genomic fragments of this virus. Clearly, mouse species has unique piRNAs against both the sense and antisense sequence of SARS-CoV-2 genome. These piRNAs might not be the entire collection, since the piRNAQuest database is incomplete (for providing analytical options). Regardless, the experiment was repeated for human piRNAs and it was noted that there are many fewer human piRNA species meeting this criteria of targeting SARS-CoV-2.

TABLE 1
MOUSE PIRNAS POTENTIALLY AGAINST SARS-COV-2

| | | | | | SEQ |
|---|---|---|---|---|---|
| piRNA ID | piRNA sequence | qPCR | Criteria | Criteria | ID |
| (piRNAQuest) | (5′ to 3′) | label | #1 | #2 | NO: |

piRNA matching 5′ UTR (sense)

| | | | | | |
|---|---|---|---|---|---|
| mmu_piRNA_ | CCUGCAUGCUUAGUGCCUUCAGAGGU | U1 | ✓ | | 1 |
| 141534 | | | | | |
| mmu_piRNA_ | UAUCUGUGUGGCUGUGUAAAGAAAGA | U2 | | ✓ | 2 |
| 429488 | | | | | |
| mmu_piRNA_ | UAACAAACCAACCACUAAGGAAUGUAGCU | U3 | | | 3 |
| 266543 | | | | | |
| mmu_piRNA_ | UGGAAGGUAACUGCAUGCUUAGUGGA | | | | 4 |
| 711652 | | | | | |
| mmu_piRNA_ | UGAGUGUGGCUGUCACUGGCCUCUGU | | | | 5 |
| 621776 | | | | | |
| mmu_piRNA_ | UGAGAUUCUGCAGGCUGCUGAGCUGUAAUGU | | | | 6 |
| 602217 | | | | | |
| mmu_piRNA_ | UGACUUAGAGCCCUAUCUUCUGCAGG | | | | 7 |
| 585955 | | | | | |
| mmu_piRNA_ | UCUGAGUGUGGCUGUCACUGGCCUCU | | | | 8 |
| 519806 | | | | | |

piRNA matching 5′ UTR (antisense)

| | | | | |
|---|---|---|---|---|
| mmu_piRNA_ | AACACAGAUUUUAAAAGUACACAUUUUUCUGG | U4 | ✓ | 9 |
| 6868 | | | | |
| mmu_piRNA_ | CAAUUGCAGCCUCCAGAAGAUAGACAUGGCC | | | 10 |
| 95511 | | | | |
| mmu_piRNA_ | UGAUGUGCUUUGUGGGUUGGUUGGUUUGUUU | | | 11 |
| 640897 | GU | | | |
| mmu_piRNA_ | UGAAAGUCCCUGCAGAAGAUAGAGGU | | | 12 |
| 544141 | | | | |
| mmu_piRNA_ | UAGAAGGGACAGCCACACAGAUGUGUGUA | | | 13 |
| 362959 | | | | |
| mmu_piRNA_ | GAAGGGACAGCCACACAGAUGUGUGU | | | 14 |
| 181190 | | | | |
| mmu_piRNA_ | UAAAAUGCAGCCGAGUGAACAGGCCUCUC | | | 15 |
| 251265 | | | | |
| mmu_piRNA_ | GAGGCUGGUGCCUAGUUGGUUGGUUGGUUUG | | | 16 |
| 195325 | UCAU | | | |

piRNA matching ORF1ab (sense)

| | | | | | |
|---|---|---|---|---|---|
| mmu_piRNA_ | UUUAUUGCUACAAUUGUCUGGUUGAGGAUA | O1-1 | ✓ | | 17 |
| 921631 | | | | | |
| mmu_piRNA_ | UGAAGAAGAGCAAGAAGGAAAGUGAA | O1-2 | ✓ | | 18 |
| 553635 | | | | | |
| mmu_piRNA_ | UAUUUAAACUGUCUUAUGUGUCUCCA | O1-3 | ✓ | | 19 |
| 443751 | | | | | |
| mmu_piRNA_ | UUGUGUUCUCUUAUGUUCUGUAUGAC | O1-4 | ✓ | | 20 |
| 916068 | | | | | |
| mmu_piRNA_ | UGGUUAUUUAAAACUUUGUCUCUGA | O1-5 | ✓ | | 21 |
| 776097 | | | | | |
| mmu_piRNA_ | UACUUGGUGUCCUUGCUCAGGAACUGG | | | ✓ | 22 |
| 354467 | | | | | |
| mmu_piRNA_ | AUGAAGAUUUUCAACUCAAAGGACCAGC | | | ✓ | 23 |
| 80156 | | | | | |
| mmu_piRNA_ | UAUAACAACUUCUGUUUCCAGAAGGA | | ✓ | | 24 |
| 420243 | | | | | |
| mmu_piRNA_ | UUACUGCUGCCGUGAUCAUAGCAAUU | | ✓ | | 25 |
| 848541 | | | | | |
| mmu_piRNA_ | UACUGCUGCCGUGAUCAUAGCAAUUUCAGC | | ✓ | | 26 |
| 350007 | | | | | |
| mmu_piRNA_ | UUGUUGGAGAAGGUGUUUAAGAUCCU | | ✓ | | 27 |
| 917189 | | | | | |
| mmu_piRNA_ | UUGUGUUCUCUUAUGGUUGGUGUGAC | | ✓ | | 28 |
| 916067 | | | | | |
| mmu_piRNA_ | UGUGUUCUCUUAUGGUCUGUAUGACA | | | ✓ | 29 |
| 822152 | | | | | |
| mmu_piRNA_ | UCAACCUUCUUGAAAGAUGACAUGUCU | | ✓ | | 30 |
| 449701 | | | | | |
| mmu_piRNA_ | UUUGGUGAACAGAAAGGUGAAAUAGA | | | ✓ | 31 |
| 931796 | | | | | |
| mmu_piRNA_ | UGUGGAAACUGUGACUUUUGUUUUGC | | | ✓ | 32 |
| 812772 | | | | | |
| mmu_piRNA_ | UGUAAACUGUGAAAGGUCAACCCUGGGC | | ✓ | | 33 |
| 780078 | | | | | |
| mmu_piRNA_ | UAGAUGGAAUUUCACUGCAAAAUGCC | | | ✓ | 34 |
| 376501 | | | | | |
| mmu_piRNA_ | CAGCCUCUGAUGCAGAUAAUCCCACC | | | ✓ | 35 |
| 113412 | | | | | |
| mmu_piRNA_ | AUUCACUGAGACUCUAGGCUUUCUCC | | | ✓ | 36 |
| 87149 | | | | | |
| mmu_piRNA_ | AAUUUUGAGCAGUUCCUCCAGGAGAG | | ✓ | | 37 |
| 25793 | | | | | |
| mmu_piRNA_ | UGAAAUUAAGGAGAGAGCUACCCUCCC | | ✓ | | 38 |
| 546757 | | | | | |
| mmu_piRNA_ | CCCUGUGCUUGUGAAUGAGAUGUGAG | | | ✓ | 39 |
| 137643 | | | | | |
| mmu_piRNA_ | UAACUUCCUCUGUUACCUGGUGGGGA | | ✓ | | 40 |
| 277495 | | | | | |
| mmu_piRNA_ | UGAGAAGUGCUCUGCAGAUGGCUGU | | ✓ | | 41 |
| 590983 | | | | | |
| mmu_piRNA_ | UACUUUUGGUGAUGAUAGUGAUGUCU | | ✓ | | 42 |
| 355460 | | | | | |
| mmu_piRNA_ | UCCUGUGUUGUGGCAUGUUUCUAAAA | | | ✓ | 43 |
| 502000 | | | | | |
| mmu_piRNA_ | UGAGGAUGAAGAAGAUGGAGAGCAGA | | ✓ | | 44 |
| 612330 | | | | | |
| mmu_piRNA_ | UUCUUCUUCUUCACAGAAAGGGUUUG | | | ✓ | 45 |
| 887325 | | | | | |
| mmu_piRNA_ | UGAUGAAGAAGAAGGAGAUGUCCACGA | | ✓ | | 46 |
| 634017 | | | | | |
| mmu_piRNA_ | UACUUAUUUGAUGACUGGUGCUUCC | | | ✓ | 47 |
| 353300 | | | | | |
| mmu_piRNA_ | CUAAUUCAGAUACUGCUACAAGAUUG | | ✓ | | 48 |
| 148282 | | | | | |
| mmu_piRNA_ | UAGAAGUGGCACCAAAAGCUCUCCAC | | ✓ | | 49 |
| 363728 | | | | | |
| mmu_piRNA_ | UAGUUCCAUCUCUAACAGAGCUCUAU | | ✓ | | 50 |
| 417232 | | | | | |
| mmu_piRNA_ | UGUAAUCAUCAGAUGUAUGUGUGGCUU | | | ✓ | 51 |
| 783080 | | | | | |
| mmu_piRNA_ | UGAAAAGCAAGUUGAGGGGGAAGAGC | | | ✓ | 52 |
| 537175 | | | | | |
| mmu_piRNA_ | UACUUAGCUGUCUUUUAAGAAUGACUCC | | ✓ | | 53 |
| 353093 | | | | | |
| mmu_piRNA_ | UGAGAUUCCUAAAGCAGUUGUCCCGGA | | ✓ | | 54 |
| 602155 | | | | | |
| mmu_piRNA_ | UGAAAAGCAAGUUGGAGAGGAAAGGGU | | ✓ | | 55 |
| 537176 | | | | | |
| mmu_piRNA_ | UACCAAAAAUACCAGUUAAGAGUCAC | | ✓ | | 56 |
| 333044 | | | | | |
| mmu_piRNA_ | UGAAAGUGAUGUCAAACUGAGGGGCA | | ✓ | | 57 |
| 544287 | | | | | |
| mmu_piRNA_ | UCUCUGGAAGAAACUUGAGUCUGGGAGCUCA | | ✓ | | 58 |
| 514850 | | | | | |
| mmu_piRNA_ | CUCUGGAAGAAACUUGAGUCUGGGAGC | | | ✓ | 59 |
| 155276 | | | | | |
| mmu_piRNA_ | UAAAGCUUUGAGAAAGUGUCAUUUGCA | | | ✓ | 60 |
| 259267 | | | | | |
| mmu_piRNA_ | UGUUUCUUCUGCAUGCUCCAGUGUCGC | | ✓ | | 61 |
| 835335 | | | | | |
| mmu_piRNA_ | GUGCCUGUCUGUGUGGUGGCCCAGCA | | ✓ | | 62 |
| 235261 | | | | | |
| mmu_piRNA_ | CACAUGCAGAAGAAAGAGCCAUCUAC | | ✓ | | 63 |
| 98313 | | | | | |
| mmu_piRNA_ | UGUGUAAAAGUGCCUCAUUUUCUGUAU | | | ✓ | 64 |
| 816246 | | | | | |
| mmu_piRNA_ | UGCUUGCACAUGCACAGUCACCUGUA | | ✓ | | 65 |
| 700479 | | | | | |
| mmu_piRNA_ | UAGAUAACCAUUAUACAAAACAUCCAAU | | | ✓ | 66 |
| 374498 | | | | | |
| mmu_piRNA_ | UAAUAAAAUGUUCUUUUGACAAAUGAA | | | ✓ | 67 |
| 298991 | | | | | |
| mmu_piRNA_ | UGUGGUAGGAUUACUCAGCUGUUAAU | | | ✓ | 68 |
| 815366 | | | | | |
| mmu_piRNA_ | UGUUGUCUACUGUUAGGAAUAGAGUG | | ✓ | | 69 |
| 833014 | | | | | |
| mmu_piRNA_ | UACAUGUCAAUGACACGUCUGUGUUACAUAGC | | | ✓ | 70 |
| 332065 | | | | | |
| mmu_piRNA_ | UAAUUUCUUAAGAGAACUUUAUGGUGUU | | | ✓ | 71 |
| 312136 | | | | | |
| mmu_piRNA_ | UAGAAGUUAAACCAGAAAUGUGUGCCU | | ✓ | | 72 |
| 363830 | | | | | |
| mmu_piRNA_ | UGUAGAGCAGGUGGAAGGGAGCUGAA | | | ✓ | 73 |
| 785707 | | | | | |
| mmu_piRNA_ | UACAAAAGUUAGCAGUUUUAGGUUUA | | ✓ | | 74 |
| 313042 | | | | | |
| mmu_piRNA_ | UAAGGGUGUAGAAGAGGAUAUGUGCCA | | ✓ | | 75 |
| 294481 | | | | | |
| mmu_piRNA_ | CAGAGCAGGUGGAUUCCAAGUUUGA | | ✓ | | 76 |
| 108079 | | | | | |
| mmu_piRNA_ | UUAAAGAAACUUUGUCUGAAUAAGUU | | | ✓ | 77 |
| 840877 | | | | | |
| mmu_piRNA_ | UGUGACUCCUGUUGUGUAUGGUCUUG | | | ✓ | 78 |
| 803608 | | | | | |
| mmu_piRNA_ | UAAAGGUGACUCCUGGCACCCCUGGCCU | | | ✓ | 79 |
| 260546 | | | | | |
| mmu_piRNA_ | UUGUUGUUUGUACAGAGUCUCACUGU | | ✓ | | 80 |
| 917476 | | | | | |
| mmu_piRNA_ | UAAUUGGUUGCUCUGGAGAUGAGGGG | | | ✓ | 81 |
| 311506 | | | | | |
| mmu_piRNA_ | UUUAAAAAUUACAGAUGGGAAGGAAA | | ✓ | | 82 |
| 918178 | | | | | |
| mmu_piRNA_ | UAGAUCUGUGUGGCCUUGGACACUCAAACU | | | ✓ | 83 |
| 375660 | | | | | |
| mmu_piRNA_ | UAAAGUAAAGAAAUACAGCCACUGGG | | ✓ | | 84 |
| 260804 | | | | | |
| mmu_piRNA_ | UAGUGUUUGUCUUAAACGCUCAGGG | | | ✓ | 85 |
| 416377 | | | | | |
| mmu_piRNA_ | UAAGCCAAGAAUUAUGAGUGUCCUGCU | | | ✓ | 86 |
| 289984 | | | | | |
| mmu_piRNA_ | UAGAGCAACAAGAGUAGAUGAUUCUC | | ✓ | | 87 |
| 370808 | | | | | |
| mmu_piRNA_ | GCUGCUGAAAUAAAAAGAAGAGGAGG | | | ✓ | 88 |
| 213317 | | | | | |
| mmu_piRNA_ | UGAUAUGGAACGUUCCUGCUCUUUGGUGGC | | | ✓ | 89 |
| 630132 | | | | | |
| mmu_piRNA_ | UAUACAAGCACCAAGUAAAUCCUGGGAGC | | | ✓ | 90 |
| 421880 | | | | | |
| mmu_piRNA_ | GACAAUGACAUGAAGCCUCAGAAUUGGGUU | | | ✓ | 91 |
| 184083 | | | | | |
| mmu_piRNA_ | UUUCAAGUGAAAUCAGUUGAUAGAAGAGU | | ✓ | | 92 |
| 922044 | | | | | |
| mmu_piRNA_ | AAAAGAUGCUUCUGGAAACAAGGUUAA | | ✓ | | 93 |
| 692 | | | | | |
| mmu_piRNA_ | UACAACUUUUGAUUCUUGCUUCCAAA | | ✓ | | 94 |
| 316091 | | | | | |
| mmu_piRNA_ | UAGGUUCUGUUAGAGAAGAUGCAUCU | | | ✓ | 95 |
| 406329 | | | | | |
| mmu_piRNA_ | UCCAAAAGCUCUUCUAUUUGUGGUCAAAGU | | ✓ | | 96 |
| 475314 | | | | | |
| mmu_piRNA_ | UGAUAAAAUGUCAAGACCAAAGCUGGG | | ✓ | | 97 |
| 625217 | | | | | |
| mmu_piRNA_ | UAGAAAUGUAUCUAAGGCAGAAGCAGGU | | ✓ | | 98 |
| 358462 | | | | | |
| mmu_piRNA_ | UGAGAAACCACCUGUGGAGAGAAUGGA | | | ✓ | 99 |
| 587682 | | | | | |
| mmu_piRNA_ | UAGCCACAAGGUUAAUGGGUCUUUCUAUC | | | ✓ | 100 |
| 383720 | | | | | |
| mmu_piRNA_ | UAACCACCUGUCUCCAGCUCCGGGAA | | ✓ | | 101 |
| 272491 | | | | | |
| mmu_piRNA_ | UGGAGACAGGUGGUCCCUGAAGCUCCCUGGUU | | ✓ | | 102 |
| 719288 | AGAUAGUCCAA | | | | |
| mmu_piRNA_ | UGCUCAAACUGGAAAUCAAGAGUUCU | | | ✓ | 103 |
| 686332 | | | | | |
| mmu_piRNA_ | UAAUUCCAGUUUGAGGAAAUCCAAUG | | ✓ | | 104 |
| 310483 | | | | | |
| mmu_piRNA_ | GCUGCUGUUAUAAAGUGUAAUGUAAA | | | ✓ | 105 |
| 213392 | | | | | |
| mmu_piRNA_ | CAUAGCCACAAGGUGUCAGUGUUACC | | ✓ | | 106 |
| 125873 | | | | | |
| mmu_piRNA_ | CUGUUCUUUUCACUGGGUAUGUUGGUGC | | | ✓ | 107 |
| 170256 | | | | | |
| mmu_piRNA_ | UGACACUUAUGAAUGACCAGUGCUGA | | ✓ | | 108 |
| 572764 | | | | | |
| mmu_piRNA_ | UAUGCCUGCUAGUUAUGUAACCUGUG | | ✓ | | 109 |
| 434258 | | | | | |
| mmu_piRNA_ | UAAACAGAGAAAUGCCUUCAGCAACAUGC | | ✓ | | 110 |
| 253112 | | | | | |
| mmu_piRNA_ | UCCACAUGGAAAUGGCAUUUAAACUCCU | | ✓ | | 111 |
| 478866 | | | | | |
| mmu_piRNA_ | UAUUUCCAUGUGGGCUACGUUUAGUGA | | ✓ | | 112 |
| 444455 | | | | | |
| mmu_piRNA_ | UUGUUGAGUAUUGCCUUUUGUAGAAG | | ✓ | | 113 |
| 916988 | | | | | |
| mmu_piRNA_ | UAACAGAGAUUAUAGGUGUGAACCAC | | | ✓ | 114 |
| 269188 | | | | | |
| mmu_piRNA_ | UUUUCUGUUUUGCUUUUGAUUGAGGUA | | ✓ | | 115 |
| 936321 | | | | | |
| mmu_piRNA_ | UGUGAAUUCAUAUAUAUGGGAGCUGG | | | ✓ | 116 |
| 802313 | | | | | |
| mmu_piRNA_ | UUUUCUGUUUUGCUUUUGAUUGAGGUA | | ✓ | | 117 |
| 936321 | | | | | |
| mmu_piRNA_ | UAGCAUUGUGGGAAAUGUAGUUCCUA | | ✓ | | 118 |
| 383295 | | | | | |
| mmu_piRNA_ | GAAUGCAAGAGAUGGAAGAGAGAAUC | | ✓ | | 119 |
| 182620 | | | | | |
| mmu_piRNA_ | UGAGGCCAUGCUAAACUUUGUCGAGUC | | ✓ | | 120 |
| 613254 | | | | | |
| mmu_piRNA_ | UCCUAAAGGUCCUAAAUUCAAAUCCCAG | | ✓ | | 121 |
| 493329 | | | | | |
| mmu_piRNA_ | UUCUUUCUGUGCUUUGGUGAUUUCUCC | | ✓ | | 122 |
| 888456 | | | | | |
| mmu_piRNA_ | UUAUCUUUCUGUGCCUGUCUUAGAGA | | ✓ | | 123 |
| 854568 | | | | | |
| mmu_piRNA_ | UCUUUCUGUGCUUUGGUGAUUUCUCCU | | | ✓ | 124 |
| 534319 | | | | | |
| mmu_piRNA_ | GUGUGCUUUUGCUGUGUGUGUAUGA | | ✓ | | 125 |
| 238083 | | | | | |
| mmu_piRNA_ | UUGGCUUCCGGUGUACUGCAUGUAUGU | | | ✓ | 126 |
| 907162 | | | | | |
| mmu_piRNA_ | CACACACACUGGUACAUGGGUGUCCUU | | | ✓ | 127 |
| 96243 | | | | | |
| mmu_piRNA_ | GUCAGCUGACUGAAGGGGGUGUUUC | | ✓ | | 128 |
| 229916 | | | | | |
| mmu_piRNA_ | UAGAGAAAGUGUGUCCCUGGAUGUGGGU | | ✓ | | 129 |
| 369010 | | | | | |
| mmu_piRNA_ | UUGUUGUGAUGAUGUUCUCUAUGGAAUC | | | ✓ | 130 |
| 917373 | | | | | |
| mmu_piRNA_ | UUGACAUGGUACCACCUUGAGUUUAGUUGG | | ✓ | | 131 |
| 893132 | | | | | |
| mmu_piRNA_ | UGUUAGAGAAAGUGUACCACUAGGAGC | | ✓ | | 132 |
| 825168 | | | | | |
| mmu_piRNA_ | UGACAUGGUACCACCUUGAGUUUAGUUGGUU | | | ✓ | 133 |
| 577193 | | | | | |
| mmu_piRNA_ | UGACAUGGUACCACCUUGAGUUUAGUUGGA | | | ✓ | 134 |
| 577192 | | | | | |
| mmu_piRNA_ | UGUACUGACAUUAGAGGCUGCCAGUUG | | | ✓ | 135 |
| 784960 | | | | | |
| mmu_piRNA_ | AAACUGCAGAGUCACAGCCCAUACCAC | | | ✓ | 136 |
| 2239 | | | | | |
| mmu_piRNA_ | UAGGAAGUUCUGUUGUCCACAUCAGA | | | ✓ | 137 |
| 392625 | | | | | |
| mmu_piRNA_ | UACAGUUUGAAAAGCUGAGAUCAGGG | | | ✓ | 138 |
| 329597 | | | | | |
| mmu_piRNA_ | GAACUUCCUUCCUUCUAGGUGACCCC | | | ✓ | 139 |
| 178724 | | | | | |
| mmu_piRNA_ | UACUAGAGGAGCUACAGAUAGGGUUU | | | ✓ | 140 |
| 344270 | | | | | |
| mmu_piRNA_ | UGACCAUUUCACUCAGACUGUAACCAUGAC | | | ✓ | 141 |
| 578951 | | | | | |
| mmu_piRNA_ | UGUCAUGUGUGGCGGGUGAUGUGGUCC | | ✓ | | 142 |
| 793638 | | | | | |
| mmu_piRNA_ | UGACAGCUUGACAAGAGUGACUUUUAAA | | ✓ | | 143 |
| 574567 | | | | | |
| mmu_piRNA_ | AAGAUGCCACAACUGGAUCCUUCUGCAA | | | ✓ | 144 |
| 15478 | | | | | |
| mmu_piRNA_ | CACUGACUUAAAGUUUUAAGUAGAAAAGGA | | | ✓ | 145 |
| 100949 | | | | | |
| mmu_piRNA_ | AACUUUAAGUCAGUGGGGUUUACAGA | | | ✓ | 146 |
| 12512 | | | | | |
| mmu_piRNA_ | UCAACUUUACUUAGGGUGACUGAAGGCU | | ✓ | | 147 |
| 450047 | | | | | |
| mmu_piRNA_ | UGUGAUGUCAGCACCCGAGAGGCUGA | | | ✓ | 148 |
| 808299 | | | | | |
| mmu_piRNA_ | UAGAGCAAGCUGUAUGUACAUCUGUA | | ✓ | | 149 |
| 370842 | | | | | |
| mmu_piRNA_ | UGACAGCAGAUAUAGGGAGGUCAGCA | | | ✓ | 150 |
| 574048 | | | | | |
| mmu_piRNA_ | CACAGCAUUCUGUGAACCUCAUCCAUG | | ✓ | | 151 |
| 97296 | | | | | |
| mmu_piRNA_ | UUUUAUGUGCUUUAAUUCUAUGUUAAGA | | ✓ | | 152 |
| 935175 | | | | | |
| mmu_piRNA_ | UUUCACAGAAUGCUGCUUCUAGGGGCUGU | | | ✓ | 153 |
| 922160 | | | | | |
| mmu_piRNA_ | UCUGAGCCCUGUGAAGCUGCUGCCUGC | | | ✓ | 154 |
| 518852 | | | | | |
| mmu_piRNA_ | UAUAAAACAUUUUACCUGUUUAAAAUGUA | | | ✓ | 155 |
| 419404 | | | | | |
| mmu_piRNA_ | UAAGGGUGUUAUCAAGAAAAGUGUCC | | | ✓ | 156 |
| 294496 | | | | | |
| mmu_piRNA_ | AUUCACAGAAUGCUAAAGUGUCCUGUAAGCUU | | ✓ | | 157 |
| 87122 | | | | | |
| mmu_piRNA_ | UUGAAUUUAGUGUCAUGGCCUGUUUU | | | ✓ | 158 |
| 892841 | | | | | |
| mmu_piRNA_ | UGUGUGUAGGUGCCUGAGGAUGCCUG | | ✓ | | 159 |
| 820166 | | | | | |
| mmu_piRNA_ | AGCUUGUAAAGUUGCUAUAAAUGGCUCUUUGG | | ✓ | | 160 |
| 60952 | CA | | | | |
| mmu_piRNA_ | UAGUAAGGUAAUCAAAGGAUCAGUGG | | ✓ | | 161 |
| 407491 | | | | | |
| mmu_piRNA_ | UUGUAGGUACAGCAAUAAAUAAAUGGG | | | ✓ | 162 |
| 911730 | | | | | |
| mmu_piRNA_ | UUGAAUUUAGUGUCAUGGCCUGUUUU | | | ✓ | 163 |
| 892841 | | | | | |
| mmu_piRNA_ | UAGUGUCUGAAGCAGGAAAACACUGC | | ✓ | | 164 |
| 415573 | | | | | |
| mmu_piRNA_ | CCUGAAGCAGUGGAAGGGGCCUCUGUA | | | ✓ | 165 |
| 140834 | | | | | |
| mmu_piRNA_ | UGUCUGUAGACAUCAAUUUUGCCCAGCCU | | | ✓ | 166 |
| 798648 | | | | | |
| mmu_piRNA_ | UGUAGAUUUGACACUGAUGACCUGGC | | | ✓ | 167 |
| 785981 | | | | | |
| mmu_piRNA_ | UUCUAUUACUCUGAUGUGACAGAGAAGGA | | | ✓ | 168 |
| 877814 | | | | | |
| mmu_piRNA_ | UAAACAAGUAGUGUCUCCUAGGGCCU | | ✓ | | 169 |
| 252706 | | | | | |
| mmu_piRNA_ | UAAAAGUGCUUUUGAGACAUCGUUGG | | | ✓ | 170 |
| 249700 | | | | | |
| mmu_piRNA_ | GUAGAUUUGACACUGAUGACCUGGCA | | | ✓ | 171 |
| 228639 | | | | | |
| mmu_piRNA_ | UGUCUGUAGACAUCAAUUUUGCCCAGCCU | | | ✓ | 172 |
| 798648 | | | | | |
| mmu_piRNA_ | UGUAAACCCACAAGGUAGGAACAUGGCC | | | ✓ | 173 |
| 779977 | | | | | |
| mmu_piRNA_ | CUUUAGCUUGUGGGACUGCAGGUGG | | ✓ | | 174 |
| 173866 | | | | | |
| mmu_piRNA_ | UAUUAUUGAGUAUUUUAAAGAAGGGC | | ✓ | | 175 |
| 440102 | | | | | |
| mmu_piRNA_ | UUGUCUUUUUUGAUGACUUUGGGUUGGG | | | ✓ | 176 |
| 913615 | | | | | |
| mmu_piRNA_ | UAUUUGAGCUUUGGGAUUUGAUGCUA | | ✓ | | 177 |
| 444862 | | | | | |
| mmu_piRNA_ | UUACAGAUGGUUGUAAGCUGUCAUGU | | | | 178 |
| 846200 | | | | | |
| mmu_piRNA_ | UACAGAUGGUUGUAAGCCACCAUAUGGGUGC | | | | 179 |
| 325150 | | | | | |
| mmu_piRNA_ | UUCUAAGUGUGUGUGUCUGUCUGUG | | | ✓ | 180 |
| 875670 | | | | | |
| mmu_piRNA_ | UGUGUGUGUUCUGUUGUGUAUCUCAA | | ✓ | | 181 |
| 821440 | | | | | |
| mmu_piRNA_ | UAAAGGUGAUUCCUUCAUAAUAAGGG | | ✓ | | 182 |
| 260567 | | | | | |
| mmu_piRNA_ | UAUCAUUAUGCCUUUUCUUCCCUACAGAU | | | ✓ | 183 |
| 427145 | | | | | |
| mmu_piRNA_ | UAAAAUUAUGGUGAUUGAUGAUGGUA | | ✓ | | 184 |
| 252053 | | | | | |
| mmu_piRNA_ | UACAUUUGUGGGUUUCUAGAAACAGC | | ✓ | | 185 |
| 332967 | | | | | |
| mmu_piRNA_ | UGUCUCUGAUGCAGCUUAUACGAAGA | | ✓ | | 186 |
| 797426 | | | | | |
| mmu_piRNA_ | UAUCAAAGUUGAAUCAGGAUCAAGUUG | | ✓ | | 187 |
| 424779 | | | | | |
| mmu_piRNA_ | UGCUGAUCUUUAUAACUGGAAAACUA | | | ✓ | 188 |
| 691842 | | | | | |
| mmu_piRNA_ | UCUGUCUUGAAAGUGCUCAAAAUUCU | | | | 189 |
| 527011 | | | | | |
| mmu_piRNA_ | UGCUUAUUAGUGAUGAAGUUUUCCAGG | | | | 190 |
| 698758 | | | | | |
| mmu_piRNA_ | UGCAGUUUUAUGAGGCUAUGGGACUA | | | | 191 |
| 661477 | | | | | |
| mmu_piRNA_ | UGAUUACAGAGCAAGGGUGGUGAAGC | | | | 192 |
| 642102 | | | | | |
| mmu_piRNA_ | UGAAAGUUUACAACCAUCUGUACAGCUA | | | | 192 |
| 544637 | | | | | |
| mmu_piRNA_ | UCAGACAGAUGGACAAGUUUUUGGUGC | | | | 194 |
| 460924 | | | | | |
| mmu_piRNA_ | UACAAUUUUUAUUGCUACAAUUGUCUG | | | | 195 |
| 318705 | | | | | |
| mmu_piRNA_ | UUUUUCUGAAGAAGACUGGUCAGUUGU | | | | 196 |
| 938315 | | | | | |

piRNA matching ORF1ab (antisense)

| | | | | | |
|---|---|---|---|---|---|
| mmu_piRNA_ | UAGAAGUAACAGAGAUUCUUGGACAG | O1-6 | ✓ | | 197 |
| 363350 | | | | | |
| mmu_piRNA_ | UUUCCAGAGUUGUUGUACCAAUUUCCAAU | O1-7 | ✓ | | 198 |
| 923720 | | | | | |
| mmu_piRNA_ | UGAAAUAACAACUCUAAUACUUAAUUA | | ✓ | | 199 |
| 544742 | | | | | |
| mmu_piRNA_ | UAUAAAGAUCAGCAGAGGAAAAUGACC | | | ✓ | 200 |
| 419795 | | | | | |
| mmu_piRNA_ | CUGCAUCAGAGACAACUGAAGAUGAA | | | ✓ | 201 |
| 160853 | | | | | |
| mmu_piRNA_ | UAUCAUUAUGCCUUUUCUUCCCUACAGAU | | | ✓ | 202 |
| 427145 | | | | | |
| mmu_piRNA_ | UAUCAAAGUUGAAUCAGGAUCAAGUUG | | ✓ | | 203 |
| 424779 | | | | | |
| mmu_piRNA_ | UAAAGGUGAUUCCUUCAUAAUAAGGG | | ✓ | | 204 |
| 260567 | | | | | |
| mmu_piRNA_ | UACAGAUGGUUGUAAGCCACCAUAUGGGUGC | | ✓ | | 205 |
| 325150 | | | | | |
| mmu_piRNA_ | UAUUAUUGAGUAUUUUAAAGAAGGGC | | ✓ | | 206 |
| 440102 | | | | | |
| mmu_piRNA_ | UGUUGUUUUAUUUUUGUAGCUCUGGA | | ✓ | | 207 |
| 833864 | | | | | |
| mmu_piRNA_ | CCUGAAGCAGUGGAAGGGGCCUCUGUA | | | ✓ | 208 |
| 140834 | | | | | |
| mmu_piRNA_ | UUGUAGGUACAGCAAUAAAUAAAUGGG | | | ✓ | 209 |
| 911730 | | | | | |
| mmu_piRNA_ | UAGCACUAACUCUGACCCUCUUACCCU | | | ✓ | 210 |
| 379919 | | | | | |
| mmu_piRNA_ | UUGAAUUUAGUGUCAUGGCCUGUUUU | | | ✓ | 211 |
| 892841 | | | | | |
| mmu_piRNA_ | UGUGUGUAGGUGCCUGAGGAUGCCUG | | ✓ | | 212 |
| 820166 | | | | | |
| mmu_piRNA_ | AGCUUGUAAAGUUGCUAUAAAUGGCUCUUUGG | | ✓ | | 213 |
| 60952 | CA | | | | |
| mmu_piRNA_ | CACAGCAUUCUGUGAACCUCAUCCAUG | | ✓ | | 214 |
| 97296 | | | | | |
| mmu_piRNA_ | UCUGAGCCCUGUGAAGCUGCUGCCUGC | | | ✓ | 215 |
| 518852 | | | | | |
| mmu_piRNA_ | UAUAAAACAUUUUACCUGUUUAAAAUGUA | | | ✓ | 216 |
| 419404 | | | | | |
| mmu_piRNA_ | UAGAGCAAGCUGUAUGUACAUCUGUA | | ✓ | | 217 |
| 370842 | | | | | |
| mmu_piRNA_ | UUCACCAUAGUCACCUGUCAGGAAUGU | | ✓ | | 218 |
| 860010 | | | | | |
| mmu_piRNA_ | UUGAGUCACAUCUGCACCACAGCUCC | | | ✓ | 219 |
| 895245 | | | | | |
| mmu_piRNA_ | UAAGUAAAGUUGAGCCGCCUAUCAGC | | ✓ | | 220 |
| 295549 | | | | | |
| mmu_piRNA_ | UCUUAAUGAAGUCUUUAAAAAAACCA | | ✓ | | 221 |
| 529249 | | | | | |
| mmu_piRNA_ | UCAACAUAAGAAUGGAUACAGAAAAUGUG | | | ✓ | 222 |
| 449448 | | | | | |
| mmu_piRNA_ | CACUGACUUAAAGUUUUAAGUAGAAAAGGA | | | | 223 |
| 100949 | | | | | |
| mmu_piRNA_ | UGACAGCUUGACAAGAGUGACUUUUAAA | | | | 224 |
| 574567 | | | | | |
| mmu_piRNA_ | UGACCAUUUCACUCAGACUGUAACCAUGAC | | | ✓ | 225 |
| 578951 | | | | | |
| mmu_piRNA_ | GAACUUCCUUCCUUCUAGGUGACCCC | | | ✓ | 226 |
| 178724 | | | | | |
| mmu_piRNA_ | UUAGCUCUCUGAAGUUGUGAGUGUAC | | ✓ | | 227 |
| 851406 | | | | | |
| mmu_piRNA_ | UACAGUUUGAAAAGCUGAGAUCAGGG | | | ✓ | 228 |
| 329597 | | | | | |
| mmu_piRNA_ | AAGCUCUCUGAAGUGUUAGCCAAGGAG | | ✓ | | 229 |
| 17193 | | | | | |
| mmu_piRNA_ | UAGAGAAAGUGUGUCCCUGGAUGUGGGU | | ✓ | | 230 |
| 369010 | | | | | |
| mmu_piRNA_ | UGUUAGAGAAAGUGUACCACUAGGAGC | | ✓ | | 231 |
| 825168 | | | | | |
| mmu_piRNA_ | GUCAGCUGACUGAAGGGGGUGUUUC | | ✓ | | 232 |
| 229916 | | | | | |
| mmu_piRNA_ | UUGGCUUCCGGUGUACUGCAUGUAUGU | | | ✓ | 233 |
| 907162 | | | | | |
| mmu_piRNA_ | UCCUUUAAUAAAGUGAGACCAGGGGC | | | ✓ | 234 |
| 503976 | | | | | |
| mmu_piRNA_ | UGAGGCCAUGCUAAACUUUGUCGAGUC | | ✓ | | 235 |
| 613254 | | | | | |
| mmu_piRNA_ | UGAACAACUUCAGAACAGCCAGCCGACUU | | ✓ | | 236 |
| 547453 | | | | | |
| mmu_piRNA_ | UUGCCCUGUUGUCCUGAAUGGAAGGUCU | | ✓ | | 237 |
| 899693 | | | | | |
| mmu_piRNA_ | UCUGGACACAUUGAGGCUGCUGCACA | | ✓ | | 238 |
| 523683 | | | | | |
| mmu_piRNA_ | UAGCUGCAUAUGAUGAAAAGAUGGCCU | | ✓ | | 239 |
| 388255 | | | | | |
| mmu_piRNA_ | UGUGAAUUCAUAUAUAUGGGAGCUGG | | | ✓ | 240 |
| 802313 | | | | | |
| mmu_piRNA_ | UCCACAUGGAAAUGGCAUUUAAACUCCU | | ✓ | | 241 |
| 478866 | | | | | |
| mmu_piRNA_ | UAACAGAGAUUAUAGGUGUGAACCAC | | | ✓ | 242 |
| 269188 | | | | | |
| mmu_piRNA_ | UAAACAGAGAAAUGCCUUCAGCAACAUGC | | ✓ | | 243 |
| 253112 | | | | | |
| mmu_piRNA_ | UAAUUCCAGUUUGAGGAAAUCCAAUG | | ✓ | | 244 |
| 310483 | | | | | |
| mmu_piRNA_ | CUGUUCUUUUCACUGGGUAUGUUGGUGC | | | ✓ | 245 |
| 170256 | | | | | |
| mmu_piRNA_ | UGAGAAACCACCUGUGGAGAGAAUGGA | | | ✓ | 246 |
| 587682 | | | | | |
| mmu_piRNA_ | UAGCCACAAGGUUAAUGGGUCUUUCUAUC | | | ✓ | 247 |
| 383720 | | | | | |
| mmu_piRNA_ | UAACCACCUGUCUCCAGCUCCGGGAA | | ✓ | | 248 |
| 272491 | | | | | |
| mmu_piRNA_ | CAUAGCCACAAGGUGUCAGUGUUACC | | ✓ | | 249 |
| 125873 | | | | | |
| mmu_piRNA_ | UAAGUCAUUGAGAGCUCUAGCCUGGA | | ✓ | | 250 |
| 296384 | | | | | |
| mmu_piRNA_ | UCCAAAAGCUCUUCUAUUUGUGGUCAAAGU | | ✓ | | 251 |
| 475314 | | | | | |
| mmu_piRNA_ | UGAUAAAAUGUCAAGACCAAAGCUGGG | | ✓ | | 252 |
| 625217 | | | | | |
| mmu_piRNA_ | GAAUGUUGUGACUUUUACUGCAACAA | | | ✓ | 253 |
| 183035 | | | | | |
| mmu_piRNA_ | UUUGUUGUUACAACCUUCUGUGUCUGCU | | | ✓ | 254 |
| 933405 | | | | | |
| mmu_piRNA_ | UAUACAAGCACCAAGUAAAUCCUGGGAGC | | | ✓ | 255 |
| 421880 | | | | | |
| mmu_piRNA_ | GACAAUGACAUGAAGCCUCAGAAUUGGGUU | | | ✓ | 256 |
| 184083 | | | | | |
| mmu_piRNA_ | GCUGCUGAAAUAAAAAGAAGAGGAGG | | | ✓ | 257 |
| 213317 | | | | | |
| mmu_piRNA_ | UAGUAAAGAUGGAUGUGGCAUGUCAA | | | ✓ | 258 |
| 406971 | | | | | |
| mmu_piRNA_ | UAAGCCAAGAAUUAUGAGUGUCCUGCU | | | ✓ | 259 |
| 289984 | | | | | |
| mmu_piRNA_ | UAAAGUAAAGAAAUACAGCCACUGGG | | ✓ | | 260 |
| 260804 | | | | | |
| mmu_piRNA_ | UAGAUCUGUGUGGCCUUGGACACUCAAACU | | | ✓ | 261 |
| 375660 | | | | | |
| mmu_piRNA_ | UAAUUGGUUGCUCUGGAGAUGAGGGG | | | ✓ | 262 |
| 311506 | | | | | |
| mmu_piRNA_ | UGUGACUCCUGUUGUGUAUGGUCUUG | | | ✓ | 263 |
| 803608 | | | | | |
| mmu_piRNA_ | UAAAGGUGACUCCUGGCACCCCUGGCCU | | | ✓ | 264 |
| 260546 | | | | | |
| mmu_piRNA_ | UGUAGAGCAGGUGGAAGGGAGCUGAA | | | ✓ | 265 |
| 785707 | | | | | |
| mmu_piRNA_ | UACAAAAGUUAGCAGUUUUAGGUUUA | | ✓ | | 266 |
| 313042 | | | | | |
| mmu_piRNA_ | CAGAGCAGGUGGAUUCCAAGUUUGA | | | | 267 |
| 108079 | | | | | |
| mmu_piRNA_ | UAGAAGUUAAACCAGAAAUGUGUGCCU | | | | 268 |
| 363830 | | | | | |
| mmu_piRNA_ | UGUGGUAGGAUUACUCAGCUGUUAAU | | | ✓ | 269 |
| 815366 | | | | | |
| mmu_piRNA_ | UGUUGUCUACUGUUAGGAAUAGAGUG | | ✓ | | 270 |
| 833014 | | | | | |
| mmu_piRNA_ | UAGAUAACCAUUAUACAAAACAUCCAAU | | | ✓ | 271 |
| 374498 | | | | | |
| mmu_piRNA_ | UAAUAAAAUGUUCUUUUGACAAAUGAA | | | ✓ | 272 |
| 298991 | | | | | |
| mmu_piRNA_ | UGUUUCUUCUGCAUGCUCCAGUGUCGC | | ✓ | | 273 |
| 835335 | | | | | |
| mmu_piRNA_ | UGUAAUCAUCAGAUGUAUGUGUGGCUU | | | ✓ | 274 |
| 783080 | | | | | |
| mmu_piRNA_ | AAUUUUGAGCAGUUCCUCCAGGAGAG | | ✓ | | 275 |
| 25793 | | | | | |
| mmu_piRNA_ | AAAUGACUUUAGAUCUGGUAAUGAGU | | | ✓ | 276 |
| 5453 | | | | | |
| mmu_piRNA_ | UGAGGGACAAGGACUCCAUGGGACAC | | ✓ | | 277 |
| 614043 | | | | | |
| mmu_piRNA_ | UUCAAAACAGAAAGUAGUGAGGACUC | | | | 278 |
| 856650 | | | | | |
| mmu_piRNA_ | GCCCUCUGUAGCAGCCUCUGAUGCAAA | | | | 279 |
| 208638 | | | | | |
| mmu_piRNA_ | AUGUUUCAAAACAGAAAGUAGUGAGG | | | | 280 |
| 86536 | | | | | |
| mmu_piRNA_ | UUGGAGAGGUGACUCUGCAGUUAAAA | | | | 281 |
| 904466 | | | | | |
| mmu_piRNA_ | UGUCACAUGUCUUGGACAGCAUGCGU | | | | 282 |
| 790579 | | | | | |
| mmu_piRNA_ | UGCUCCAGUAACAGUUACAGAAACCCCGGAGC | | | | 283 |
| 687591 | CAGCAAUGCCCAGUGGUG | | | | |
| mmu_piRNA_ | UACCCAUGUGUCAAAAUCAGUGCUAACUGGACA | | | | 284 |
| 337571 | | | | | |
| mmu_piRNA_ | UAAAAGAAUUACUAAUAAAACAAAAAGG | | | | 285 |
| 247270 | | | | | |
| mmu_piRNA_ | GCCAAGUCUGUGAAUUGCAAGGUUUUAGUUCA | | | | 286 |
| 207153 | AG | | | | |

piRNA matching Spike (sense)

| | | | | | |
|---|---|---|---|---|---|
| mmu_piRNA_ | UGAAGACCCAGUCCCUACCUUAGCCUA | S1 | ✓ | | 287 |
| 555154 | | | | | |
| mmu_piRNA_ | UGUGAAGGUGUCUUUGUCACUAAUAGAUG | S2 | ✓ | | 288 |
| 801725 | | | | | |
| mmu_piRNA_ | UAAAACAGGGUAAUUCCUGCCACGGAC | | | ✓ | 289 |
| 245678 | | | | | |
| mmu_piRNA_ | UGACUCCUGGUGAUUGGCAAACGGUUG | | | ✓ | 290 |
| 583180 | | | | | |
| mmu_piRNA_ | UAUUACAGAUGCUGUCAAACUUAUUC | | | ✓ | 291 |
| 438728 | | | | | |
| mmu_piRNA_ | UUCUAAGGUUGGUGAGAUGUCUAAAC | | | ✓ | 292 |
| 875617 | | | | | |
| mmu_piRNA_ | UGCUGACACUACUGAACUGGACUGCU | | | ✓ | 293 |
| 690421 | | | | | |
| mmu_piRNA_ | UAGUUAUCAGACUCACUGGACUUUUGU | | | ✓ | 294 |
| 416863 | | | | | |
| mmu_piRNA_ | UGGAAAGGGCUAUCUUCUCAGUGUUA | | ✓ | | 295 |
| 705519 | | | | | |
| mmu_piRNA_ | UGACUUAUGUCCCUGGCCCAGGGUGA | | ✓ | | 296 |
| 586053 | | | | | |
| mmu_piRNA_ | UGCAAAUUUGAUGAAUUCUGAUGGCUUUUU | | | ✓ | 297 |
| 648660 | | | | | |
| mmu_piRNA_ | UAGAAAUGAUCUCUGCUUUAUUGUCUA | | | | 298 |
| 358318 | | | | | |
| mmu_piRNA_ | UGUGUGUGUGUGUUUAAGAAUAUUAU | | | | 299 |
| 821383 | | | | | |
| mmu_piRNA_ | UGUGCUGUUCUUACUGAGUCUUCUUGCUCA | | | | 300 |
| 812229 | | | | | |
| mmu_piRNA_ | UGGCAUCAGUGUCUUUGUUUCAAAAGUU | | | | 301 |
| 733364 | | | | | |
| mmu_piRNA_ | UGAUUGCUGCCAGAGACCUCAUCAGAAU | | | | 302 |
| 644234 | | | | | |
| mmu_piRNA_ | UGAAAUCUAGGAAGAGAAUCAGCAGG | | | | 303 |
| 545483 | | | | | |
| mmu_piRNA_ | UCAGUGUCUUUGUUUCAAAAGUUGUUU | | | | 304 |
| 469620 | | | | | |
| mmu_piRNA_ | UACCUGCAAAUUUGAUGAAUUCUGAUGGCUU | | | | 305 |
| 339934 | | | | | |
| mmu_piRNA_ | UACCUGAUUGCUGCCAGAGACCUCAUC | | | | 306 |
| 339923 | | | | | |
| mmu_piRNA_ | UAAAUUUACCUGCAAAUUUGAUGAAUUCUG | | | | 307 |
| 266133 | | | | | |
| mmu_piRNA_ | GUAGACUGUGAAGUCCCUGUUGCUACUCCUU | | | | 308 |
| 228562 | | | | | |
| mmu_piRNA_ | CUCAGCCAGGAACAUCACCAGAUGUUG | | | | 309 |
| 151798 | | | | | |
| mmu_piRNA_ | AGGAACAUCACCAGAUGUUGGGGAUCCACAC | | | | 310 |
| 61587 | | | | | |
| mmu_piRNA_ | AAGAUUACAAACUUGUGCCAAGAGAG | | | | 311 |
| 15664 | | | | | |

piRNA matching Spike (antisense)

| | | | | | |
|---|---|---|---|---|---|
| mmu_piRNA_ | UGAAGUCUGCCUGUGAAGUCUGCCUGUGA | S3 | | ✓ | 312 |
| 562740 | | | | | |
| mmu_piRNA_ | UCCUGAAGAAGAAUCACAAUCGUUCACAGU | S4 | ✓ | | 313 |
| 498273 | | | | | |
| mmu_piRNA_ | GUGAAGUCUGCCUGUGAAGUCUGCCU | S5 | ✓ | | 314 |
| 233057 | | | | | |
| mmu_piRNA_ | UGUAUGAUUCUUAACCUAUGUCUGGAA | | ✓ | | 315 |
| 788249 | | | | | |
| mmu_piRNA_ | UGGCAGGAGCAGUUCCUAACUGGGAG | | ✓ | | 316 |
| 732409 | | | | | |
| mmu_piRNA_ | UGUGAUCAACCUAUCUCCCUUAAUGGACU | | | ✓ | 317 |
| 807222 | | | | | |
| mmu_piRNA_ | UACCAUGAGGUGCUGAGAAGAAGGUGUAUUCU | | | | 318 |
| 336565 | UUUGUUU | | | | |
| mmu_piRNA_ | UACCAUGAGGUGCUGAGAAGAAGGUAUAUC | | | | 319 |
| 336564 | | | | | |
| mmu_piRNA_ | ACCAUGAGGUGCUGAGAGGAAGGUA | | | | 320 |
| 33842 | | | | | |
| mmu_piRNA_ | UAAGUGCACUUGCUGUUCUCGCAGA | | ✓ | | 321 |
| 297079 | | | | | |
| mmu_piRNA_ | UUUGAUGGAUCUGGAGAUUCCCGGGA | | | ✓ | 322 |
| 929508 | | | | | |
| mmu_piRNA_ | UAAAUUUGUGGGUAUGUCAGCCACAGAAGGU | | ✓ | | 323 |
| 266288 | | | | | |
| mmu_piRNA_ | GUCUUGGUCAUAGAAAGUAUCUGAAA | | | ✓ | 324 |
| 232630 | | | | | |
| mmu_piRNA_ | ACAACCUGGUUAGAAGAUGCCCGUCA | | | ✓ | 325 |
| 26348 | | | | | |
| mmu_piRNA_ | AUUUGAGAUUAGACUUAAUACAGUGUG | | ✓ | | 326 |
| 89235 | | | | | |
| mmu_piRNA_ | GGAGCGAUUUGUCUGGUUAAUUCCGAU | | | ✓ | 327 |
| 218123 | | | | | |
| mmu_piRNA_ | GAGCGAUUUGUCUGGUUAAUUCCGAUAAC | | | ✓ | 328 |
| 193194 | | | | | |
| mmu_piRNA_ | UGAAGAAGAAUCACAAUCGUUCACAGUGGUC | | | ✓ | 329 |
| 553610 | | | | | |
| mmu_piRNA_ | UGAAAAAUGUGUGCCAUUUGAAACUCU | | | | 330 |
| 536042 | | | | | |
| mmu_piRNA_ | UACACCCACAACCAGAAGUGAUUGUUUUC | | | | 331 |
| 319947 | | | | | |
| mmu_piRNA_ | UUGUUGCUGAUUCUCUUCUCCAGAGGUGA | | | | 332 |
| 917127 | | | | | |
| mmu_piRNA_ | UUAGUUGGUGGAGCGAUUUGUCUGGUUAA | | | | 333 |
| 852931 | | | | | |
| mmu_piRNA_ | UGUGAAGUCUGCCUGUGAAGUCUGCCU | | | | 334 |
| 801808 | | | | | |
| mmu_piRNA_ | UGUGAAGUCUGCCUGUGAAGUCAGUCU | | | | 335 |
| 801807 | | | | | |
| mmu_piRNA_ | UGUGAAGUCUGCCUGUGAAGUCAGCCU | | | | 336 |
| 801806 | | | | | |
| mmu_piRNA_ | UGGUGGCUGACAGCAUCUGUAAUGGG | | | | 337 |
| 772789 | | | | | |
| mmu_piRNA_ | UGGUGGAGCGAUUUGUCUGGUUAAUUC | | | | 338 |
| 772370 | | | | | |
| mmu_piRNA_ | UGCUUUUCUUGUGCAGGGCUGAUGGCUGU | | | | 339 |
| 702969 | | | | | |
| mmu_piRNA_ | UGAUAGAUGUUCUCCUGAAGAAGAAUCACAAU | | | | 340 |
| 628384 | | | | | |
| mmu_piRNA_ | UCUUAGUUGGUGGAGCGAUUUGUCUGGUUA | | | | 341 |
| 530057 | | | | | |
| mmu_piRNA_ | UAGUUGGUGGAGCGAUUUGUCUGGUU | | | | 342 |
| 418225 | | | | | |
| mmu_piRNA_ | UAGCACUUUGUUUCUGAGAUGAGGUUC | | | | 343 |
| 380254 | | | | | |
| mmu_piRNA_ | UAGAUGUUCUCCUGAAGAAGAAUCACAAUC | | | | 344 |
| 377095 | | | | | |
| mmu_piRNA_ | GUCAGUCUGUGAAGUCUGCCUGUGA | | | | 345 |
| 230097 | | | | | |
| mmu_piRNA_ | CUUAGUUGGUGGAGCGAUUUGUCUGGU | | | | 346 |
| 171138 | | | | | |
| mmu_piRNA_ | AGUUGGUGGAGCGAUUUGUCUGGUUAAU | | | | 347 |
| 74145 | | | | | |

piRNA matching ORF3a (sense)

| | | |
|---|---|---|
| mmu_piRNA_ | UAAAAAAUGGGAAUAUGGAGUAACUC | 348 |
| 242723 | | |
| mmu_piRNA_ | UGUUGAUGGUCAUUGUUUGUAACAGUU | 349 |
| 830907 | | |
| mmu_piRNA_ | UGUCUUUUUGAACAUGUUACCUCAGCAUACA | 350 |
| 799983 | | |
| mmu_piRNA_ | UGAUAAUGAGGCUUUGGGAGUAUAACC | 351 |
| 626566 | | |
| mmu_piRNA_ | UGAUAAUGAGGCUUUGGGAAUAUAAC | 352 |
| 626565 | | |
| mmu_piRNA_ | UGAGAGUAAAAGACUGUGCUGCCUUA | 353 |
| 597767 | | |
| mmu_piRNA_ | UAAGGACUCUUCACAAUUAGAACUGUU | 354 |
| 292581 | | |
| mmu_piRNA_ | UAAAUAAAGCUUUAAUUUAUGAUGAACCU | 355 |
| 262253 | | |
| mmu_piRNA_ | GUUUGCUGUUGUUGUUUAAAUGCCUU | 356 |
| 241636 | | |
| mmu_piRNA_ | CAGGAGGAUGUCUUUUUGAACAUGUUACCUC | 357 |
| 117204 | | |
| mmu_piRNA_ | AGGAGGAUGUCUUUUUGAACAUGUUACCUCA | 358 |
| 63822 | | |

piRNA matching ORF3a (antisense)

| | | | | |
|---|---|---|---|---|
| mmu_piRNA_ | UUCAAGGCCAGCAGCUACAGAGUGAG | O3-1 | ✓ | 359 |
| 858506 | | | | |
| mmu_piRNA_ | UGAAGUAACUGUGUAUACUGGGUAUA | | | 360 |
| 561925 | | | | |
| mmu_piRNA_ | UAUUGUGUGAAUUUGGUUUUGUGGUG | O3-3 | ✓ | 361 |
| 443463 | | | | |
| mmu_piRNA_ | UAUUGUGUGAAUUUGGUUUUGUCAUU | O3-4 | ✓ | 362 |
| 443462 | | | | |
| mmu_piRNA_ | UAUUGUGUGAAUUUGGUUUUGUCAGG | O3-5 | ✓ | 363 |
| 443460 | | | | |
| mmu_piRNA_ | AUUGUGUGAAUUUGGUUUUGUCCUGG | O3-6 | ✓ | 364 |
| 88635 | | | | |
| mmu_piRNA_ | AUUGUGUGAAUUUGGUUUUGUCAUGG | O3-7 | ✓ | 365 |
| 88634 | | | | |
| mmu_piRNA_ | AUUGUGUGAAUUUGGUUGUGUCAUGG | O3-8 | ✓ | 366 |
| 88633 | | | | |
| mmu_piRNA_ | AUGUUCUUCAGGCUCCCCUGCAGGUUUGUUUU | O3-9 | ✓ | 367 |
| 86326 | UG | | | |
| mmu_piRNA_ | AGCUUCCCUCUGUGGUUCAGUGAACACCCUUG | | | 368 |
| 60782 | GAAUGCCCCA | | | |
| mmu_piRNA_ | UGGCAUUCUGGUAGUCAUGUUGUUGG | | | 369 |
| 734206 | | | | |
| mmu_piRNA_ | UGGAAAGAUAUUGUGUGAAUUUGGUUUUGU | | | 370 |
| 705032 | | | | |
| mmu_piRNA_ | UGCUGCAUACACCCUUGGAGAGUUCA | | | 371 |
| 692502 | | | | |
| mmu_piRNA_ | UGCAUACACCCUUGGAGAGUUCACUGG | | | 372 |
| 661667 | | | | |
| mmu_piRNA_ | UGAAAGCCAAAGCCUCAAUAGAAAGAA | | | 373 |
| 542392 | | | | |
| mmu_piRNA_ | UAUUGUGUGAAUUUGGUUUUGUCAUG | | | 374 |
| 443461 | | | | |
| mmu_piRNA_ | UAAGCCUUGAGAGUCAUGUUCAGAAAG | | | 375 |
| 290569 | | | | |
| mmu_piRNA_ | AAACAGUUCUAAUUGUGAAGAGUCCU | | | 376 |
| 1660 | | | | |
| mmu_piRNA_ | UUUUGGAUUGUGAAGAUUCUGUGGUU | | | 377 |
| 937254 | | | | |
| mmu_piRNA_ | UUUGAUUGUGAAGAUUCUGUGUUCUAGU | | | 378 |
| 929801 | | | | |

piRNA matching Envelope (sense)

| | | |
|---|---|---|
| mmu_piRNA_ | UGAAUGUUCUUCUAGAGGUCCUGAGUU | 379 |
| 567861 | | |
| mmu_piRNA_ | CGACUGUUCUUCCAGAGUUCCUGAGUUCAAU | 380 |
| 144588 | | |

piRNA matching Envelope (anti-sense)

| | | | | |
|---|---|---|---|---|
| mmu_piRNA_ | CAGAAGAUCAGGAACUAACAGGCAAA | E1 | ✓ | 381 |
| 104250 | | | | |
| mmu_piRNA_ | GAAGGUUUUACAAGAUAAGGGGCUUC | E2 | ✓ | 382 |
| 181390 | | | | |
| mmu_piRNA_ | UCAGGACCUCUAGAAGAACAGUCAGUG | E3 | | 383 |
| 466371 | | | | |
| mmu_piRNA_ | UCAGGACCUCUAGAAGAACAAUCAGU | E4 | | 384 |
| 466370 | | | | |
| mmu_piRNA_ | CCAAAUUUAAGACCAGAAGAUCAGGAGGAUC | | | 385 |
| 129308 | | | | |
| mmu_piRNA_ | UAAACUCAGAAGAUCAGGAACUAAUCG | | | 386 |
| 254896 | | | | |
| mmu_piRNA_ | UUGAACUCAGGACCUCUAGAAGAACAGU | | | 387 |
| 890311 | | | | |
| mmu_piRNA_ | UGAACUCAGGAUCUCUAGAAGAACA | | | 388 |
| 551245 | | | | |
| mmu_piRNA_ | UGAACUCAGGACCUCUAGAAGAACAGUCA | | | 389 |
| 551174 | | | | |
| mmu_piRNA_ | UGAACUCAGGACCUCUAGAAGAACAGA | | | 390 |
| 551173 | | | | |
| mmu_piRNA_ | GAACUGAACUCAGGACCUCUAGAAGAAC | | | 391 |
| 178401 | | | | |
| mmu_piRNA_ | AAGAUGCUCUAGAAGAAUUCCUGCCA | | | 392 |
| 15493 | | | | |

piRNA matching Gap1 (sense)

| | | | |
|---|---|---|---|
| mmu_piRNA_ | UAGUUUUUCUGUUCAAUGGUUCAUGA | G1 | 393 |
| 419319 | | | |
| mmu_piRNA_ | UAGUUUUUCUGUUAAGUGAAGAGGGG | | 394 |
| 419318 | | | |
| mmu_piRNA_ | UGCUUUGCUAGUUCUUCUGUUUGGAA | | 395 |
| 702426 | | | |
| mmu_piRNA_ | UUGCUUUGCUAGUUCUUCUGUUUGGA | | 396 |
| 902355 | | | |

piRNA matching Gap1 (antisense)

| | | | | | |
|---|---|---|---|---|---|
| mmu_piRNA_ | UUCCAAACAGAAAAUGCAGCUUUCGA | G3 | | | 397 |
| 865863 | | | | | |
| mmu_piRNA_ | UCCAAACAGAAGAACUAGCAAAGCAA | G4 | ✓ | | 398 |
| 475430 | | | | | |
| mmu_piRNA_ | UCCAAACAGAAAAGCUUAAAGUUAAG | G5 | | ✓ | 399 |
| 475429 | | | | | |

piRNA matching Membrane (sense)

| | | | |
|---|---|---|---|
| mmu_piRNA_ | UGCUUCUUUCAGACUUCCCUUCUGUCU | M1 | 400 |
| 700126 | | | |
| mmu_piRNA_ | UGGAAUGUAAGCUCCUUGAACAAUGGCGA | | 401 |
| 713942 | | | |
| mmu_piRNA_ | UACAUACUGUUUCCACCGGUGGAAUUGCUA | | 402 |
| 330072 | | | |
| mmu_piRNA_ | UGAUGUAAGGUUUUUGUAUAUAAGAC | | 403 |
| 639782 | | | |
| mmu_piRNA_ | UAUGCUACUACCUUGUUUUGUGCUUGC | | 404 |
| 434372 | | | |
| mmu_piRNA_ | UUGAGAUUGCUUCUUUCAGAAGAUGC | | 405 |
| 894353 | | | |
| mmu_piRNA_ | CUGCUGCUUCUGCAGUUUUGUGCUUGCUGUAU | | 406 |
| 162352 | UUUUCU | | |
| mmu_piRNA_ | AUGCCUACCACACCGUUGAAGAGCUUUA | | 407 |
| 82028 | | | |

piRNA matching Membrane (antisense)

| | | | | | |
|---|---|---|---|---|---|
| mmu_piRNA_ | UAGCAAUUCCACCGGUGGAAACAGUA | M2 | ✓ | | 408 |
| 379382 | | | | | |
| mmu_piRNA_ | CUGUACAAGCAAAGCUCUUGGGAGGU | M3 | | ✓ | 409 |
| 167046 | | | | | |
| mmu_piRNA_ | CCUGUAUGCAGCAAAAUGUUGGGUCC | M4 | ✓ | | 410 |
| 142622 | | | | | |
| mmu_piRNA_ | UUUCCUGUAUGCAGCAAAAUGCUGUGU | | | | 411 |
| 924299 | | | | | |
| mmu_piRNA_ | UGAGUUCAAAUCCUGUACAAGCAAAGCUC | | | | 412 |
| 622566 | | | | | |
| mmu_piRNA_ | UGAGGUUGGUUUCCUGUAUGCAGCAAAA | | | | 413 |
| 616766 | | | | | |
| mmu_piRNA_ | UCCUGUACAAGCAAAGCUCUUGGGAGGUA | | | | 414 |
| 501196 | | | | | |
| mmu_piRNA_ | UCAAAUCCUGUACAAGCAAAGCUCUUGGG | | | | 415 |
| 448760 | | | | | |
| mmu_piRNA_ | UAUUCCUGUAUGCAGCAAAAUGCUGGGUCCUA | | | | 416 |
| 440874 | UU | | | | |
| mmu_piRNA_ | UAGAGCAAAACCUGAGUCCUGAAAGACAG | | | | 417 |
| 370786 | | | | | |
| mmu_piRNA_ | UAGAAGAACAGCAAGACCUGAGUCACC | | | | 418 |
| 360587 | | | | | |
| mmu_piRNA_ | AGAUGGGUUUCCUGUAUGCAGCAAAAU | | | | 419 |
| 53513 | | | | | |
| mmu_piRNA_ | UUUUACCUGGAAUGGUCUGUCUGCC | | | | 420 |
| 934375 | | | | | |
| mmu_piRNA_ | UUGGAAGAAGACAAGAAGCAAUGAAGA | | | | 421 |
| 903067 | | | | | |

piRNA matching ORF6 (sense)

| | | | | |
|---|---|---|---|---|
| mmu_piRNA_ | UAUGAGGACUUUGAAAGUUGGACUAA | O6-1 | ✓ | 422 |
| 432244 | | | | |
| mmu_piRNA_ | UUAGGUAUUUUAUGAGGACUUUUAAG | | | 423 |
| 852106 | | | | |
| mmu_piRNA_ | UGAGUACCAAUGGAGAUUGAGACAGAA | | | 424 |
| 617347 | | | | |
| mmu_piRNA_ | UAGACUAUGAGGACUUUGAAAGUUGG | | | 425 |
| 367911 | | | | |
| mmu_piRNA_ | UUGGACAGGUAACUAUAGCAGAGCCA | | | 426 |
| 903825 | | | | |

piRNA matching ORF6 (antisense)

| | | |
|---|---|---|
| mmu_piRNA_ | UGCUGGUGAUUCGGAUCAAGAUUCCAAAUA | 427 |
| 695121 | | |
| mmu_piRNA_ | UGUAAGGCAUUUUCUCAGUUAGUGAU | 428 |
| 782416 | | |
| mmu_piRNA_ | UAAUUCAUCUAAUUGAGGAGCAGCAUC | 429 |
| 310351 | | |

piRNA matching ORF7a (sense)

| | | | |
|---|---|---|---|
| mmu_piRNA_ | AAUUUGCUUUUGCUUUAAUCCCAGGU | O7-1 | 430 |
| 25718 | | | |
| mmu_piRNA_ | UAGCAUCUGUGUCAUCAGACAAGAGG | | 431 |
| 382741 | | | |

piRNA matching ORF7a (antisense)

| | | | | |
|---|---|---|---|---|
| mmu_piRNA_ | UUCCAGAAGAGCCAGGUUCAGUUCCC | O7-2 | ✓ | 432 |
| 867226 | | | | |
| mmu_piRNA_ | UACACUCUUGGUAGUGGGGAGCCAUGGGAUC | O7-3 | ✓ | 433 |
| 320536 | | | | |

piRNA matching Gap2 (sense)

| | | | | |
|---|---|---|---|---|
| mmu_piRNA_ | UUUUAGCCUUUCUGCCGUUCUGACA | G6 | ✓ | 434 |
| 934618 | | | | |
| mmu_piRNA_ | AUUCUUCCCUGCCUCUCCUAUUCCUUGUUUUA | | | 435 |
| 87778 | GC | | | |
| mmu_piRNA_ | UGUGUAUUUGUGCUUUUUCCUGAAA | | | 436 |
| 817362 | | | | |
| mmu_piRNA_ | CAGAUCUUUUGGUUCUCUUCUCUAACC | | | 437 |
| 110096 | | | | |
| mmu_piRNA_ | AUGUUGACUUCUAUUUGGAAUCCUAA | | | 438 |
| 86376 | | | | |

piRNA matching Gap2 (antisense)

| | | | | |
|---|---|---|---|---|
| mmu_piRNA_ | UAAGGAAUAGCAGAAUGCUUUAAUGC | G7 | ✓ | 439 |
| 292200 | | | | |
| mmu_piRNA_ | ACCAGAAGUCAAUUAAUGCUCUGGUGUUGGUU | | | 440 |
| 33139 | GGUUUCU | | | |
| mmu_piRNA_ | UAGAGCCUGUGUAAAAAGCACAAAUGUGUGC | | | 441 |
| 371473 | | | | |

piRNA matching ORF7b (sense)

| | | | | |
|---|---|---|---|---|
| mmu_piRNA_ | UUUUAGCCUUUCUGCCGUUCUGACA | O7-4 | ✓ | 442 |
| 934618 | | | | |
| mmu_piRNA_ | AUUCUUCCCUGCCUCUCCUAUUCCUUGUUUUA | | | 443 |
| 87778 | GC | | | |
| mmu_piRNA_ | UGUGUAUUUGUGCUUUUUCCUGAAA | | | 444 |
| 817362 | | | | |
| mmu_piRNA_ | CAGAUCUUUUGGUUCUCUUCUCUAACC | | | 445 |
| 110096 | | | | |
| mmu_piRNA_ | AUGUUGACUUCUAUUUGGAAUCCUAA | | | 446 |
| 86376 | | | | |

piRNA matching ORF7b (antisense)

| | | | | |
|---|---|---|---|---|
| mmu_piRNA_ | UAAGGAAUAGCAGAAUGCUUUAAUGC | O7-5 | ✓ | 447 |
| 292200 | | | | |
| mmu_piRNA_ | ACCAGAAGUCAAUUAAUGCUCUGGUGUUGGUU | | | 448 |
| 33139 | GGUUUCU | | | |
| mmu_piRNA_ | UGAACAAAAAAGUGAGAACCAAACGGAUU | | | 449 |
| 547248 | | | | |
| mmu_piRNA_ | UAGAGCCUGUGUAAAAAGCACAAAUGUGUGC | | | 450 |
| 371473 | | | | |

piRNA matching ORF8 (sense)

| | | |
|---|---|---|
| mmu_piRNA_ | UUCCAGGAGGCUGGUUCUAAAACAUGAUGU | 451 |
| 868325 | | |
| mmu_piRNA_ | UGAAAGACUUUUUAGAGUUUAAUGAA | 452 |
| 541469 | | |
| mmu_piRNA_ | UAAAGAAAUUAAAGACUUUUUAGAGUU | 453 |
| 256076 | | |

piRNA matching ORF8 (antisense)

| | | | | |
|---|---|---|---|---|
| mmu_piRNA_ | UGCAGCUACAGUUGUGUGCUACUCUC | O8-1 | ✓ | 454 |
| 658285 | | | | |
| mmu_piRNA_ | CCAGGACGGAUGCAGCUACAGUUGCCAU | | | 455 |
| 132725 | | | | |
| mmu_piRNA_ | UGUGAAACAGGAAACUGUGUGGUUU | | | 456 |
| 800123 | | | | |
| mmu_piRNA_ | UGUCUUGAGAAAACAAGAAAUGAUUGGGUC | | | 457 |
| 799553 | | | | |

piRNA matching N protein (sense)

| | | | | | |
|---|---|---|---|---|---|
| mmu_piRNA_ | UGGACUUCCCUAUGGUCGUGACCUUUCCCGCC | N1 | ✓ | | 458 |
| 718264 | | | | | |
| mmu_piRNA_ | CUCCAUGAGCAGUGCUGGGAACAGUAGCAGGA | N2 | ✓ | | 459 |
| 153125 | AC | | | | |
| mmu_piRNA_ | UAAGAUGGUAUUUCUAGCUGUUAGGU | N3 | | ✓ | 460 |
| 288153 | | | | | |
| mmu_piRNA_ | ACUGGUGACUCUUCUUCCUGCUGAU | | | | 461 |
| 41434 | | | | | |
| mmu_piRNA_ | UGAAAGGACAAAAAGAAGAAAGGAAGGG | | | | 462 |
| 542972 | | | | | |
| mmu_piRNA_ | UUGAACUGACAGAAGAAACAGCAAGC | | | | 463 |
| 890495 | | | | | |
| mmu_piRNA_ | UGACAGCAAAUGACCUACACAGGUGCAAGU | | | | 464 |
| 573940 | | | | | |
| mmu_piRNA_ | UUGUGGAAGUCAGAGCAAAAUGUCUG | | | | 465 |
| 914889 | | | | | |
| mmu_piRNA_ | UUAUAGGCAUGGAAGUCACAGGAAAG | | | | 466 |
| 853564 | | | | | |
| mmu_piRNA_ | UUAACUGAUGUGGGAAGACCUUAAAUU | | | | 467 |
| 843058 | | | | | |
| mmu_piRNA_ | UUAAAUUCAACUCCAGGGGCUGGAAAC | | | | 468 |
| 842426 | | | | | |
| mmu_piRNA_ | UGGUAUCUAAGUUCAAGAAAUUCAGGG | | | | 469 |
| 764789 | | | | | |
| mmu_piRNA_ | UGGACUCUGAGUUUUGGGGACCAGGACACU | | | | 470 |
| 717578 | | | | | |
| mmu_piRNA_ | UGCCAGAGCAACUGAGGGAGCCAUCUUG | | | | 471 |
| 668105 | | | | | |
| mmu_piRNA_ | UGAGGAACAAAAUGUCUGGUAAGAUG | | | | 472 |
| 610030 | | | | | |
| mmu_piRNA_ | UGAAAAACCAGAAUGGAGAAAGGGCCU | | | | 473 |
| 535593 | | | | | |
| mmu_piRNA_ | UCUAAGUUCAAGAAAUUCAGGGCUGA | | | | 474 |
| 507595 | | | | | |
| mmu_piRNA_ | UAUGAAAAACCAGAAUGGAGAAAGGGC | | | | 475 |
| 429955 | | | | | |
| mmu_piRNA_ | UAGUUCAGGACAACAGCAAACUGUGA | | | | 476 |
| 417083 | | | | | |
| mmu_piRNA_ | UAGUGUACCAGGAACUAAUCACCCUC | | | | 477 |
| 415183 | | | | | |
| mmu_piRNA_ | UAGGCAUGGAAGUCACAGGAAAGGGC | | | | 478 |
| 400154 | | | | | |
| mmu_piRNA_ | UAGCUUUAAAUUCAACUCCAGGGGCUGGAAA | | | | 479 |
| 389835 | | | | | |
| mmu_piRNA_ | UAGAGAGUCAGAAAGAAGAAGGCUGA | | | | 480 |
| 370285 | | | | | |

piRNA matching N protein (antisense)

| | | | | | |
|---|---|---|---|---|---|
| mmu_piRNA_ | UAAUUUCCUUGGGUUUGUUUUUGGUC | N4 | ✓ | | 481 |
| 312079 | | | | | |
| mmu_piRNA_ | UGCAGCAGAUUUCUUAUUUGGGUUUU | N5 | | ✓ | 482 |
| 657627 | | | | | |
| mmu_piRNA_ | UCUGCAGCAGGAAGAGUCUUAUUGUCC | N6 | | ✓ | 483 |
| 521705 | | | | | |
| mmu_piRNA_ | UGAAAAUCUGCAGCAGGAAGAUGAGGU | | | | 484 |
| 538219 | | | | | |
| mmu_piRNA_ | UAGGAGUUUUGUUGUUGUUGGCCUU | | | | 485 |
| 397118 | | | | | |
| mmu_piRNA_ | GCUCCUGUUGUUGUUGGCCUUUCUGCCUGGU | | | | 486 |
| 212085 | | | | | |
| mmu_piRNA_ | ACUUCAAUGACUUGAUCUUUUAAAUU | | | | 487 |
| 42341 | | | | | |
| mmu_piRNA_ | UGGACAGUGUUCAGAAGAGGCUUGACUGU | | | | 488 |
| 715953 | | | | | |
| mmu_piRNA_ | UGCUGCCUUCUUUUUGUCCUUUCUGAGCUU | | | | 489 |
| 692980 | | | | | |
| mmu_piRNA_ | UGCUAAAUGAAUUGAUCUUUGAAAAGUC | | | | 490 |
| 681775 | | | | | |
| mmu_piRNA_ | UGCCUUCUUUUUGUCCUUUCUGAGCUUG | | | | 491 |
| 678904 | | | | | |
| mmu_piRNA_ | UCAGAAGAGGCUUGACUGUGGCCUG | | | | 492 |
| 459959 | | | | | |
| mmu_piRNA_ | UUCUCUCUGGUGGGAAUGUUUUCGGAGAUGUA | | | | 493 |
| 879409 | G | | | | |
| mmu_piRNA_ | UNGGUUCUGAUGGAGCAUUGUUAGCAGGC | | | | 494 |
| 839180 | | | | | |
| mmu_piRNA_ | UGGAGCAUUGUUAGCAGGCCACAGAAAUGA | | | | 495 |
| 721075 | | | | | |
| mmu_piRNA_ | UGGAAUUUGUUGCCUUGUUGUUGUUG | | | | 496 |
| 714805 | | | | | |
| mmu_piRNA_ | UGCGGUUUCCCCUACUGCUGCAGGACCAGGAG | | | | 497 |
| 680846 | AGGAUUCGUCCAACAG | | | | |
| mmu_piRNA_ | UCCUGUAUCAGAAAUUUGGAUCUUUUCUGUG | | | | 498 |
| 501344 | | | | | |

piRNA matching Gap3 (sense)

| | | | |
|---|---|---|---|
| mmu_piRNA_ | UGCAAACCACACAAGGCUUUAUUCCG | G8 | 499 |
| 647225 | | | |

piRNA matching Gap3 (antisense)

| | | |
|---|---|---|
| mmu_piRNA_ | UGCCUUGUGUGGUGAAGGGUCUGCAC | 500 |
| 679242 | | |
| mmu_piRNA_ | UGAGCUGCCUUGUGUUGUCUGGAAUC | 501 |
| 608498 | | |
| mmu_piRNA_ | UAGUCAUCUGCCCUGUGUGGUCUGUA | 502 |
| 410547 | | |

piRNA matching ORF10 (sense)

| | | |
|---|---|---|
| mmu_piRNA_ | GGUGUAGUCUACUCUUGUUGGACAGA | 503 |
| 226542 | | |

piRNA matching ORF10 (antisense)

| | | | | |
|---|---|---|---|---|
| mmu_piRNA_ | UUCAUUCUGCACAAUGUUAUUCCUGUGAGG | O10-1 | ✓ | 504 |
| 865467 | | | | |
| mmu_piRNA_ | UGUGCUAUGUAGUUCUGACUGGUGGA | O10-2 | ✓ | 505 |
| 811305 | | | | |
| mmu_piRNA_ | UUCUGGUGCAUCUAAAGUUAACUACA | | | 506 |
| 884237 | | | | |
| mmu_piRNA_ | UGCCAGGGCACAAGAGUAGACCAGAA | | | 507 |
| 669260 | | | | |
| mmu_piRNA_ | UGACAAGCAAACACAAGAGUAGACUC | | | 508 |
| 570804 | | | | |
| mmu_piRNA_ | UAGUGUCCUGAAGUGAGAUUAAAGUU | | | 509 |
| 415484 | | | | |
| mmu_piRNA_ | GGAAAUCUUUCAUUCUGCACAAUGUU | | | 510 |
| 215860 | | | | |
| mmu_piRNA_ | AAGUGCUGAGUGAGAUUAAAGUUGUG | | | 511 |
| 21371 | | | | |

piRNA matching 3′UTR (sense)

| | | | | |
|---|---|---|---|---|
| mmu_piRNA_ | UGUGAUUUUAAUAGUGUGGUCAGUGC | U5 | ✓ | 512 |
| 808977 | | | | |
| mmu_piRNA_ | UAGGGAGAGCUGCCCCUCCAGUUGUCUGU | U6 | ✓ | 513 |
| 402005 | | | | |
| mmu_piRNA_ | UGAUUAUUCCUGAGAGCUGCCUAUAUU | | | 514 |
| 642703 | | | | |
| mmu_piRNA_ | UUGAAUGGAAGAGCCCUACACAAAAC | | | 515 |
| 892510 | | | | |
| mmu_piRNA_ | CCCAGGACCAUUCCGAGUGUACAGUGAGGAUU | | | 516 |
| 135551 | UUGCUUCUAUC | | | |

piRNA matching 3′UTR (antisense)

| | | | |
|---|---|---|---|
| mmu_piRNA_ | AGAAAAAGUGGUGGCUCUUUUGAAGG | U7 | 517 |
| 43308 | | | |
| mmu_piRNA_ | UAAAAGCUUGUGAAAAUGUGGUGGGU | | 518 |
| 248529 | | | |
| mmu_piRNA_ | UUUGUGCAUGUGUUACACACUGAUGGAUCUU | | 519 |
| 932777 | | | |
| mmu_piRNA_ | UAAUGCCAAGAAAAAGUGGUGGCUCUU | | 520 |
| 306892 | | | |
| mmu_piRNA_ | CCUGAUGUCCCACCCCAAAAUGUGGUGGCUUG | | 521 |
| 141378 | GAAUAAGAGCUGUU | | |

This table contains piRNA species having at least 16 nt matches with SARS-CoV-2 sense or antisense RNA sequence; among them, piRNAs which meet criteria 1 or 2 are indicated by a check. SEQ ID NOs. 1, 2, 3, 9, 17-21, 197, 198, 287, 288, 312-314, 359-366, 380-383, 392, 394-397, 405-407, 419, 427, 429-431, 436, 439, 444, 451, 455-457, 481-483, 499, 504, 505, 512, 513, and 517 are piRNAs for qPCR.

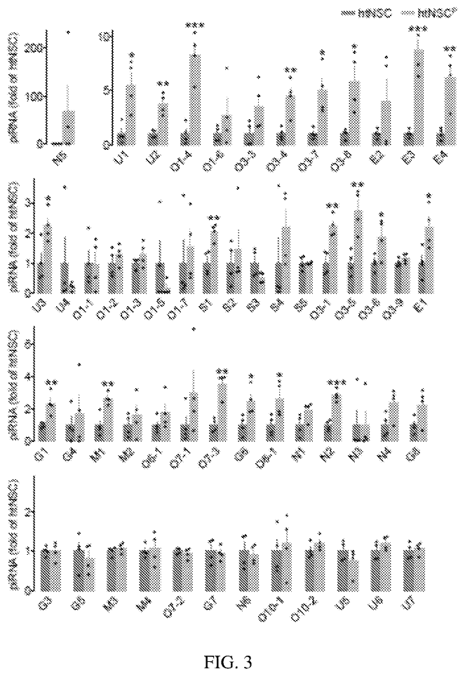

Some of these identified piRNAs were then quantified in NSC Ex/Mv, conceptually in line with previous work showing that htNSC abundantly produce exosomal miRNAs. To do so, Ex/Mv from NSCs including htNSC and hpNSC were comparatively studied versus Ex/Mv from mesenchymal stem cells (MSC), a non-neural stem cell model which has gained clinical attention during recent years. In light of htNSC, the experiments started with htNSC^{PGHM}, a htNSC recently established subpopulation, because Ex/Mv from this htNSC subtype have important pro-survival functions and also because this NSC subtype can be applied peripherally. In the procedure, Ex/Mv that were released from cultured htNSC^{PGHM}, hpNSC and MSC were isolated and cultures, then total small RNAs were extracted from these Ex/Mv using a standard kit, and finally the same quantity of these small RNAs were subjected to qPCR using specific primers against candidate piRNAs. About 60 piRNA species were selected that could target different regions of this genome under Criteria 1 or 2. The results showed that most of these piRNAs were present in Ex/Mv from htNSC^{PGHM} and hpNSC; in contrast, these piRNAs were negligible or weak in MSC Ex/Mv. Relatively, the levels of many piRNAs were 100 to 30,000-fold higher in NSC Ex/Mv than in MSC Ex/Mv, based on the same quantity of Ex/Mv total small RNA (FIG. 2). NSC are >10 times stronger than MSC in producing Ex/Mv and further, an NSC Ex/Mv produces total small RNA 5-10 times more than an MSC Ex/Mv does; hence, Ex/Mv piRNAs from NSC are 10⁴ to 10⁶ more abundant than MSC per cell. The piRNA levels in htNSC^{PGHM} and hpNSC were usually in the same order of magnitude (FIG. 2), as also observed in our experiment for comparing Ex/Mv piRNAs from htNSC^{PGHM} versus htNSC (FIG. 3), although htNSC^{PGHM} were slightly stronger for producing some piRNAs. Therefore, murine NSCs evidently produce Ex/Mv piRNAs which could potentially target against SARS-CoV-2.

### Example 2: The Presence of PIWI Protein Machinery in NSC Ex/Mv

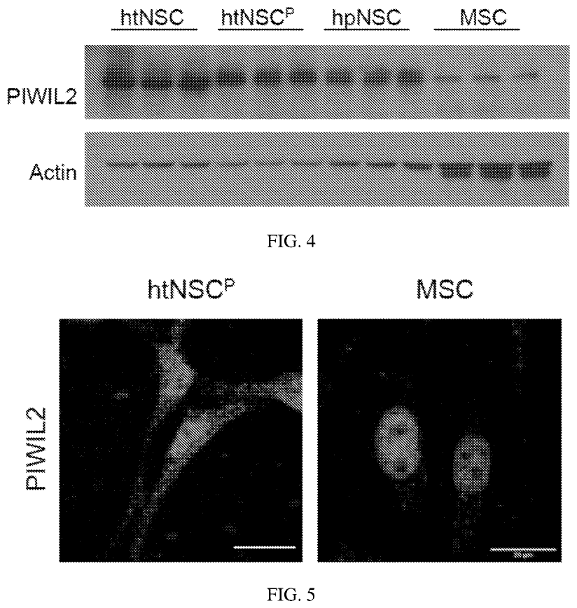

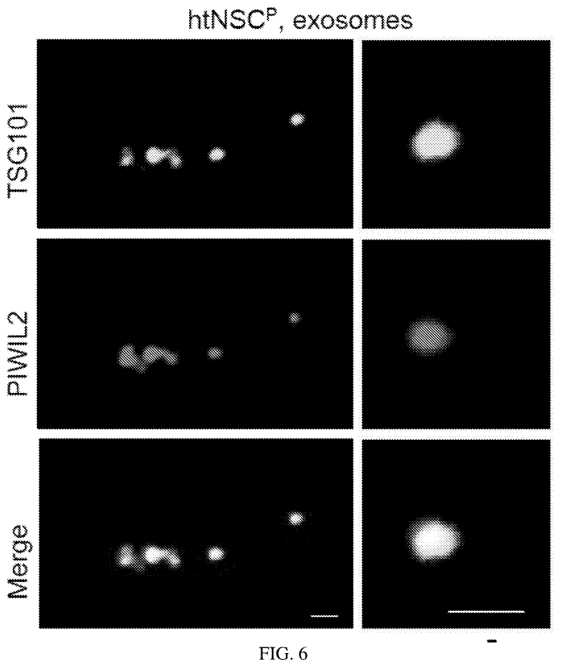

PIWI proteins, a family of highly conserved RNA-binding proteins, were initially identified in Drosophila and are also present in plants and animals including rodents and humans. PIWI proteins are typically nuclear proteins with slicer activity and the function to control piRNA biogenesis. Studies have revealed that PIWI proteins are important for regulating germ cell biology and stem cell differentiation. In mammals, humans have four PIWI homologs (PIWIL1-4), while mouse species have three homologs (PIWIL1, 2 and 4); among them, PIWIL1 and 2 are the major isoforms and have mostly been described for their expression and function in spermatocyte formation. Recent research has begun to recognize that some PIWI proteins are present in a few somatic tissues and particularly in the nervous system. Based on the finding that NSCs produce piRNAs abundantly in FIG. 1, it was predicted that some PIWI isoform(s) should be present in these murine cells. Therefore, the protein levels of PIWIL1 and 2 in NSCs were compared to MSC through western blotting. While PIWIL1 western blotting hardly gave signals, PIWIL2 was strongly present in htNSC and htNSC^{PGHM} as well as hpNSC, but much less present in MSC (FIG. 4) Immunostaining was performed for these cells and it was found that while PIWIL2 was present typically in the nuclei of MSC, it was strongly present in the nuclei as well as the cytoplasm of all NSC types (FIG. 5). The cytoplasmic distribution of PIWIL2 in NSCs agrees with the need of cytoplasmic PIWI protein for making and assembling exosomal piRNA machinery. In addition, PIWIL2 immunostaining was performed directly for isolated and purified NSC Ex/Mv and it was further confirmed that PIWIL2 was present in NSC Ex/Mv (FIG. 6). Hence, NSC Ex/Mv contain piRNAs as well as PIWIL2 protein, further suggesting that these particles have piRNA biological functions.

### Example 3: Antiviral piRNA Library of NSC Ex/Mv for VSV-Based Pseudotyped SARS-CoV-2 Virus

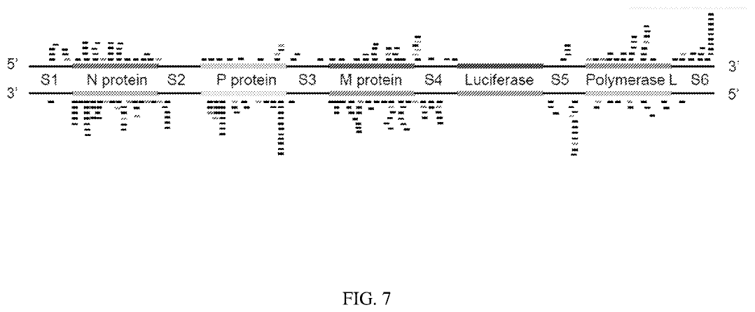

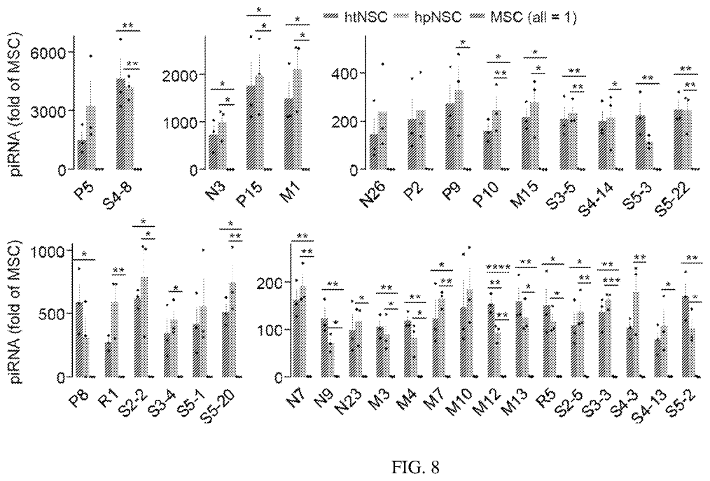

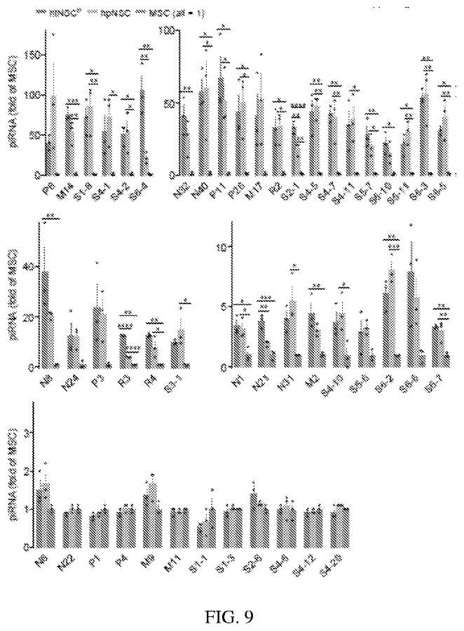

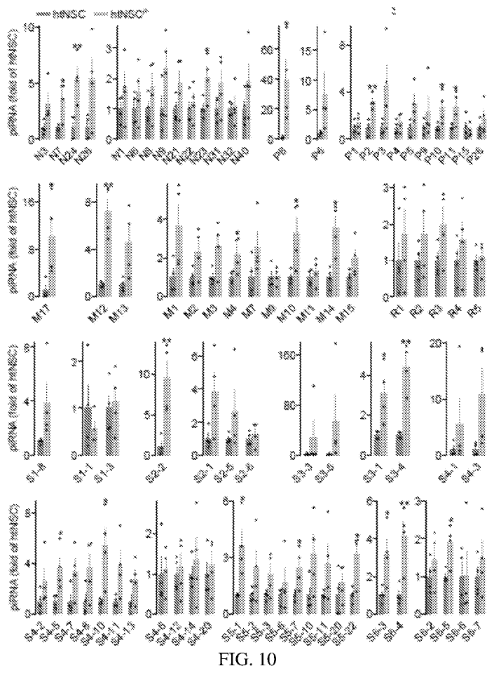

Glycoprotein-deficient VSV (ΔG-VSV) is a standard tool to create pseudotyped viruses such as pseudotyped SARS-CoV-2 virus. Because ΔG-VSV was designed to accurately report viral infection rate, SARS-CoV-2 spike protein (S2) was incorporated into luciferase-expressing ΔG-VSV to generate a pseudotyped SARS-CoV-2 virus. This viral system is used to mimic S2 glycoprotein-mediated SARS-CoV-2 infection, but the genome of this pseudotyped virus was made of VSV elements, including the RNA sequences encoding nucleocapsid protein (N sequence), phosphoprotein (P sequence), matrix protein (M sequence), RNA polymerase (R sequence) and structural non-encoding sequences (S1-6), as shown in FIG. 6. Sense and antisense sequences were applied to screen for piRNA species through the mouse piRNAQuest database and a long list of piRNAs was identified with at least 15-16 nucleotides matching with the viral sequence, and among them, about 40 piRNA species were identified to meet Criteria 1 or 2 (FIG. 7, Table 2). Some of these piRNAs were quantitated in Ex/Mv from htNSC^{PGHM} and hpNSC, and compared to the levels in MSC Ex/Mv. As shown in FIGS. 8 and 9, most of these piRNAs were present in Ex/Mv from htNSC^{PGHM} and hpNSC but were absent or negligible in MSC Ex/Mv. Additional assays showed that htNSC^{PGHM} were comparable or slightly stronger than htNSC in producing these piRNAs (FIG. 10). All these results were very similar to the data pattern for SARS-CoV-2, and thus the description for FIG. 2 applies here without need to repeat the discussion. Thus, based on the information from wildtype and pseudotyped SARS-CoV-2, NSC Ex/Mv contain piRNAs against the genomic sequences of both viruses, although these NSC were not previously exposed to either virus, suggesting that this mouse species has evolved to establish large piRNA libraries in NSC Ex/Mv potentially against viruses.

TABLE 2
INFORMATION ON MOUSE PIRNAS POTENTIALLY AGAINST PSEUDOTYPED SARS-COV-2

| piRNA ID | piRNA sequence | qPCR | Criteria | Criteria | SEQ |
|---|---|---|---|---|---|
| (piRNAQuest) | (5′ to 3′) | label | #1 | #2 | ID NO: |

piRNA matching structural non-encoding 1 (antisense)

| | | | | |
|---|---|---|---|---|
| mmu_piRNA_ | UUAACAGUAAUCAAAUUGAAACACCC | S1-1 | ✓ | 522 |
| 842651 | | | | |
| mmu_piRNA_ | UUCUGCUGUAGGAAAAGGCUCAGGAGU | S1-3 | | 523 |
| 882797 | | | | |
| mmu_piRNA_ | UUAACAAUUUAACAGUAAUCAUAUCAGG | | | 524 |
| 842553 | | | | |
| mmu_piRNA_ | UGUAGGAAAAGGCUCAGGAGUGUCCCC | | | 525 |
| 786539 | | | | |
| mmu_piRNA_ | UGCUGUAGGAAAAGGCUCAGGAGUGUCCC | | | 526 |
| 695588 | C | | | |
| mmu_piRNA_ | UUUAACAGUAAUCAUAUCAGGAAGCU | | | 527 |
| 918745 | | | | |
| mmu_piRNA_ | UGAAGGCUCAGGAGAAUGGAUUUGGUC | | | 528 |
| 560626 | | | | |

piRNA matching structural non-encoding 1 (sense)

| | | | |
|---|---|---|---|
| mmu_piRNA_ | UGUCUGAGCUAUUCUCCUGAGCCUUU | S1-8 | 529 |
| 797912 | | | |

piRNA matching Nucleoprotein (antisense)

| | | | | |
|---|---|---|---|---|
| mmu_piRNA_ | UGACAAAUGGUUGCCUAGAUGAGGAUU | N1 | ✓ | 530 |
| 570091 | | | | |
| mmu_piRNA_ | AGUUUGAAAUCAGAGGAGAUUCCUCUGCA | N3 | | 531 |
| 74437 | | | | |
| mmu_piRNA_ | UGUCUCCGAGAAGUUGCGAAGUGUC | N6 | | 532 |
| 797183 | | | | |
| mmu_piRNA_ | UGUCCCCUCUUGUGCCAGAAAACAAC | N7 | | 533 |
| 794717 | | | | |
| mmu_piRNA_ | UGUAAGAAAACCGACUCCUAUAAGCU | N8 | | 534 |
| 781539 | | | | |
| mmu_piRNA_ | UGCAGAUCUAAGAGGAUGCUGGCUAU | N9 | | 535 |
| 657091 | | | | |
| mmu_piRNA_ | AGAAGGUGGACUUCAGAAAAUCAAAUAAC | | | 536 |
| 46100 | | | | |
| mmu_piRNA_ | AGAAGGUGGACUUCAGAAAAUCAAACA | | | 537 |
| 46099 | | | | |
| mmu_piRNA_ | UGAGCCCAACUGCAGAUCUAAGAGGAUGC | | | 538 |
| 605761 | | | | |
| mmu_piRNA_ | UGACAAAUGCCUGAAAACAGACACAAAA | | | 539 |
| 570058 | AA | | | |
| mmu_piRNA_ | UCCCCUCUUGUGCCAGAAAACAACGUU | | | 540 |
| 489286 | | | | |
| mmu_piRNA_ | UCAAGAAGGUGGACUUCAGAAAAUCAA | | | 541 |
| 450172 | | | | |
| mmu_piRNA_ | UAGACUUGUCAGAUCUAAGAUCCUCGU | | | 542 |
| 368825 | | | | |
| mmu_piRNA_ | UAGACGACUGAGGAUCCAGUGGACGG | | | 543 |
| 367596 | | | | |
| mmu_piRNA_ | UACACACUGACAAAUGCCUGAAAACAGA | | | 544 |
| 319043 | CA | | | |
| mmu_piRNA_ | UAAUACUUAAGAGCAGUCAUGUCCUU | | | 545 |
| 300775 | | | | |
| mmu_piRNA_ | CUGACAAAUGCCUGAAAACAGACACAAA | | | 546 |
| 157583 | | | | |
| mmu_piRNA_ | CACACUGACAAAUGCCUGAAAACAGACA | | | 547 |
| 96594 | | | | |
| mmu_piRNA_ | UUUCUUCCUCUUCUGCUCAGAGAACAGCC | | | 548 |
| 926357 | | | | |
| mmu_piRNA_ | UUGGAGGACAUUUUUGAUGUUGUGCUAGA | | | 549 |
| 904942 | | | | |
| mmu_piRNA_ | UUGAGAGAUGCUUGUUCAAAAAACAU | | | 550 |
| 894148 | | | | |
| mmu_piRNA_ | UAAUUGUUUCCAGAUUCGUGUGGGAG | | | 551 |
| 311706 | | | | |
| mmu_piRNA_ | UUGUUGCCUUUGUAUCAGUCACUGAAG | | | 552 |
| 917104 | | | | |

piRNA matching Nucleoprotein (sense)

| | | | | | |
|---|---|---|---|---|---|
| mmu_piRNA_ | UUCCUUGCUCUGGUGGACGCUUGUAC | N21 | ✓ | | 553 |
| 873994 | | | | | |
| mmu_piRNA_ | AAAGUAUUUGAACAUGUGGAAGAUGGA | N22 | | | 554 |
| 4406 | | | | | |
| mmu_piRNA_ | UUACAGACUCAGAGCAGAAGAGCUGUC | N23 | | | 555 |
| 845981 | | | | | |
| mmu_piRNA_ | UGAAUCUGGAAACAAUUCAGUUAGUU | N24 | | ✓ | 556 |
| 565798 | | | | | |
| mmu_piRNA_ | UAAUGGGGUGAUUUGAUUUUCUGAAGUCC | N26 | | | 557 |
| 308033 | | | | | |
| mmu_piRNA_ | UUUCCCCCUGUGUUCUGGAAGCAUCC | N31 | | | 558 |
| 923923 | | | | | |
| mmu_piRNA_ | UUGACUGCUCUUUUCGAAGGUCCCGA | N32 | ✓ | | 559 |
| 893620 | | | | | |
| mmu_piRNA_ | UGAGCAGAAGAGCUGGAUGGUGUGUG | N40 | | ✓ | 560 |
| 603751 | | | | | |
| mmu_piRNA_ | UACAGACUCAGAGCAGAAGAGCUGUCCU | | | | 561 |
| 322409 | | | | | |
| mmu_piRNA_ | UAAGACAGUGUAAAGAGAUGUAUACUCAG | | | | 562 |
| 282018 | | | | | |
| mmu_piRNA_ | GGUUAUUUGAUUUUCUGAAGUCUUUUU | | | | 563 |
| 226988 | | | | | |
| mmu_piRNA_ | CAGACUCAGAGCAGAAGAGCUGUCCUG | | | | 564 |
| 106770 | | | | | |
| mmu_piRNA_ | AAUGGGGUGAUUUGAUUUUCUGAAGUCC | | | | 565 |
| 24333 | | | | | |
| mmu_piRNA_ | UUCUGGUGUAUCUGGAGUGUAUUUAUU | | | | 566 |
| 884428 | | | | | |
| mmu_piRNA_ | UUAAUGGGGUGAUUUGAUUUUCUGAAG | | | | 567 |
| 844857 | | | | | |
| mmu_piRNA_ | UUAACUCUGGUUCUGGAAGCAAAGGUAC | | | | 568 |
| 843021 | CACU | | | | |
| mmu_piRNA_ | UGUGUUCUGGAAGCAUCCACAAGCUG | | | | 569 |
| 822191 | | | | | |
| mmu_piRNA_ | UGUGAACCUAGCUUUGAUUUUCUGAACC | | | | 570 |
| 800769 | CAUGUAAAUGC | | | | |
| mmu_piRNA_ | UGCUCCAUUGUUUUGCAGAGGUGUCUG | | | | 571 |
| 687643 | | | | | |
| mmu_piRNA_ | UGCCUCUUAGAUCUGACUUCAGUGU | | | | 572 |
| 676208 | | | | | |
| mmu_piRNA_ | UCAUAAAGCAGGCACAAGAGGUUCAG | | | | 573 |
| 470402 | | | | | |
| mmu_piRNA_ | UAGUUAACUCUGGUUCUGGAAGCAAAGGU | | | | 574 |
| 416458 | | | | | |
| mmu_piRNA_ | UACCAGCUAGUUAACUCUGGUUCUGGAA | | | | 575 |
| 335320 | GCA | | | | |
| mmu_piRNA_ | UAAUCCCAGCACCCCAGAAGUGGAAGCA | | | | 576 |
| 303661 | GGA | | | | |
| mmu_piRNA_ | UAAGAGUGUGGAAGAACAUGUGGCUU | | | | 577 |
| 286146 | | | | | |
| mmu_piRNA_ | UAACUCUGGUUCUGGAAGCAAAGGUAC | | | | 578 |
| 275786 | CACU | | | | |
| mmu_piRNA_ | UAAAGCAGGCACAAGAGGUUCAGAAC | | | | 579 |
| 258498 | | | | | |
| mmu_piRNA_ | GCUAGUUAACUCUGGUUCUGGAAGCAA | | | | 580 |
| 211561 | AGGU | | | | |
| mmu_piRNA_ | GAGUAGAUUGUCUUCUCUCUUGUUAGU | | | | 581 |
| 196372 | | | | | |
| mmu_piRNA_ | CAGAAGUGAGCAGAAGAGCUGAAGAA | | | | 582 |
| 105137 | | | | | |
| mmu_piRNA_ | UUUUUGUGUCUAAGUAGCUGUUGACU | | | | 583 |
| 938577 | | | | | |
| mmu_piRNA_ | UUUUCUGUUUUGAUUUCAGCUUAUGCCC | | | | 584 |
| 936320 | | | | | |
| mmu_piRNA_ | UUUCUUGGCCUGGAGAUGUCUCUGCU | | | ✓ | 585 |
| 926469 | | | | | |
| mmu_piRNA_ | UUUCAUGAUUGAUUCUCGGUUCAAGA | | | | 586 |
| 923376 | | | | | |
| mmu_piRNA_ | UUGUGUCCAAAUGUUGAUUCCAGAACUC | | | | 587 |
| 915534 | | | | | |
| mmu_piRNA_ | UUCUGGUGGAUCUGAAGACAGCUACAG | | ✓ | | 588 |
| 884372 | | | | | |

piRNA matching structural non-encoding 2 (antisense)

| | | | | |
|---|---|---|---|---|
| mmu_piRNA_ | UGACAAAUGACCCUAACACAUGCUGGU | S2-1 | ✓ | 589 |
| 403023 | | | | |

piRNA matching structural non-encoding 2 (sense)

| | | | | |
|---|---|---|---|---|
| mmu_piRNA_ | UAGGGUCAUUUGUCAUGUGGAAAUUG | S2-2 | ✓ | 590 |
| 403023 | | | | |
| mmu_piRNA_ | UGGUGAUCUGAGAAUAGGGACCGGGGU | S2-5 | | 591 |
| 770774 | | | | |
| mmu_piRNA_ | UGGGAUCCAUGGUGAUCUGAGAAUAGGGA | S2-6 | | 592 |
| 748594 | | | | |
| mmu_piRNA_ | GCAACAGUGGGAUCCAUGGUGAUCUGAG | | | 593 |
| 203277 | AAU | | | |
| mmu_piRNA_ | AUCCAUGGUGAUCUGAGAAUAGGGAC | | | 594 |
| 77628 | | | | |
| mmu_piRNA_ | ACAGUGGGAUCCAUGGUGAUCUGAGAAUA | | | 595 |
| 31198 | | | | |
| mmu_piRNA_ | UCCAUGGUGAUCUGAGAAUAGGGACCGG | | | 596 |
| 484615 | | | | |

piRNA matching Phosphoprotein (antisense)

| | | | | |
|---|---|---|---|---|
| mmu_piRNA_ | UUGAUGAACCAGAAAUUGACCAAUUUG | P1 | | 597 |
| 896254 | | | | |
| mmu_piRNA_ | UGUUUCUUCAGAGGAGAAUUCAUCAU | P2 | | 598 |
| 835319 | | | | |
| mmu_piRNA_ | UGCAGAUCAGUCAAAGACAUCCAUGG | P3 | | 599 |
| 657042 | | | | |
| mmu_piRNA_ | GUGCCCAGUGGCUUUUGAACGCUCCUGC | P4 | | 600 |
| 235149 | | | | |
| mmu_piRNA_ | CAGAGGAGAAUUCAUCAUCUGAUUCA | P5 | ✓ | 601 |
| 108650 | GAGC | | | |
| mmu_piRNA_ | UUCUACAUCUGGAUUUUCAGGCAGCA | | | 602 |
| 876030 | | | | |
| mmu_piRNA_ | UUCCUUCUCUGCAAGAAAGCAAGUCC | | | 603 |
| 873856 | | | | |
| mmu_piRNA_ | UUCAUCUCUGUCGGGAGUGGAUGAAUGGC | | ✓ | 604 |
| 864606 | | | | |
| mmu_piRNA_ | UUCAAAGGGAAAACAGCCUGAGCUCU | | | 605 |
| 857329 | | | | |
| mmu_piRNA_ | UUCAGCAGACUCAGGAGAGAUAGAUG | | | 606 |
| 862016 | | | | |

piRNA matching Phosphoprotein (sense)

| | | | | | |
|---|---|---|---|---|---|
| mmu_piRNA_ | UGGAGAGGGCCUAGUAUGCUUCUGUGCAC | P6 | | | 607 |
| 719978 | | | | | |
| mmu_piRNA_ | UGAAUCAGAUGAUGAAUUCUCCUCUAA | P8 | | | 608 |
| 565212 | | | | | |
| mmu_piRNA_ | GAGGGCCUAGUAUGCUUCUGUGCACACAG | P9 | ✓ | | 609 |
| 195514 | U | | | | |
| mmu_piRNA_ | UGCUGGAGUGAGAGGCUGAAGAGGGUA | P10 | | | 610 |
| 694131 | | | | | |
| mmu_piRNA_ | UGCAGACACACACCUGUGCAGCAAAGG | P11 | | | 611 |
| 655758 | AGCUGCCUGCUUUCUUGGGUUGU | | | | |
| mmu_piRNA_ | UAGACCAAACAUCUGAGGUCUCUGCUG | P15 | | ✓ | 612 |
| 367015 | CAGCUGAGCUCUCU | | | | |
| mmu_piRNA_ | CCUCUGGCAAUGUCAGGCUCCCAGGGAUU | P26 | | ✓ | 613 |
| 140476 | | | | | |
| mmu_piRNA_ | UGAAAGAGGGCCUAGUAUGCACUACAG | | | | 614 |
| 541749 | | | | | |
| mmu_piRNA_ | UGAGGCUACGUUCAGAUUCUGUGUGG | | | | 615 |
| 613507 | CACA | | | | |
| mmu_piRNA_ | UCAGAUGAUGAAUUCUCCUCUGAA | | | | 616 |
| 463637 | GAAACA | | | | |
| mmu_piRNA_ | UAUGGAGUUUGCUUCAAAUGUGCAAA | | | | 617 |
| 435175 | | | | | |
| mmu_piRNA_ | UACGUUCAGAUUCUGUGUGGCACAGGGG | | | | 618 |
| 342551 | | | | | |
| mmu_piRNA_ | GUUGCUGGAGUGAGAGGCUGAAGAG | | | | 619 |
| 240635 | | | | | |
| mmu_piRNA_ | GUUACAGGAAGUCAUCUGCUGCCUGA | | | | 620 |
| 239140 | | | | | |
| mmu_piRNA_ | GGCUACGUUCAGAUUCUGUGUGGCAC | | | | 621 |
| 221510 | | | | | |
| mmu_piRNA_ | GAGGCUACGUUCAGAUUCUGUGUGGCACA | | | | 622 |
| 195214 | G | | | | |
| mmu_piRNA_ | GAAUCAGAUGAUGAAUUCUCCUCUGAAGA | | | | 623 |
| 182187 | | | | | |
| mmu_piRNA_ | CUUGAAUCAUCUGCCGCCUGAACCUGGC | | | | 624 |
| 172710 | UAGCAACAGA | | | | |
| mmu_piRNA_ | CUGAAUCAGAUGAUGAAUUCUCCUCUGAA | | | | 625 |
| 157386 | | | | | |
| mmu_piRNA_ | CUGAAGAUUGGCUCUAGGCUUGAAUCA | | | | 626 |
| 157053 | UCUGCCGCCUGAACCUGGCUAGC | | | | |
| mmu_piRNA_ | CAGAUGAUGAAUUCUCCUCUGAAGAAAC | | | | 627 |
| 110231 | | | | | |
| mmu_piRNA_ | AUCAGAUGAUGAAUUCUCCUCUGAAGAA | | | | 628 |
| 76906 | | | | | |
| mmu_piRNA_ | AGGCUACGUUCAGAUUCUGUGUGGCAC | | | | 629 |
| 65827 | | | | | |
| mmu_piRNA_ | AGAUGAUGAAUUCUCCUCUGAAGAAAC | | | | 630 |
| 53046 | | | | | |
| mmu_piRNA_ | ACAAGUGAGGCUACGUUCAGAUUCUGUGU | | | | 631 |
| 26773 | | | | | |
| mmu_piRNA_ | ACAAACCCACCCAGGAGAUGAGGUGAGC | | | | 632 |
| 25960 | AGACAAAGCCUCUGGCAAUGUC | | | | |
| mmu_piRNA_ | AAUCAGAUGAUGAAUUCUCCUCUGAAGA | | | | 633 |
| 22670 | AACA | | | | |
| mmu_piRNA_ | AAGUGAGGCUACGUUCAGAUUCUGUGU | | | | 634 |
| 21203 | | | | | |
| mmu_piRNA_ | AAGCAGAUGAUGAAUUCUCCUCUGAAGAA | | | | 635 |
| 16189 | | | | | |
| mmu_piRNA_ | UUCCUCGGAUGCUUCAAAUGUAGAAA | | | | 636 |
| 872170 | | | | | |
| mmu_piRNA_ | UUCCAGGUGCAGUAAACCCUCUGGC | | | | 637 |
| 868611 | ACGGA | | | | |
| mmu_piRNA_ | UUCAGAAUCAUCUGCAGCUACAUACUA | | ✓ | | 638 |
| 861086 | | | | | |
| mmu_piRNA_ | UUAUUUCUAAUGAGACAUUCGUCACAGGC | | | | 639 |
| 856363 | | | | | |
| mmu_piRNA_ | AUCAGAUGAUGAAUUCUCCUCUGAGGA | | | | 640 |
| 76907 | | | | | |

piRNA matching structural non-encoding 3 (antisense)

| | | | |
|---|---|---|---|
| mmu_piRNA_ | UCACUUGGCAAGUGUUAUCCCAAUCC | S3-1 | 641 |
| 458407 | | | |
| mmu_piRNA_ | UACAAAUAUUCUCUGUAUAGUCAGCAU | S3-3 | 642 |
| 314894 | | | |
| mmu_piRNA_ | UGGAACUCACUCUGUAGACUAUGCUG | S3-4 | 643 |
| 708099 | | | |

piRNA matching structural non-encoding 3 (sense)

| | | | |
|---|---|---|---|
| mmu_piRNA_ | CUAUCCUCAUGUUAGUCUACAGAGAAUA | S3-5 | 644 |
| 149964 | | | |

piRNA matching Matric protein (antisense)

| | | | | |
|---|---|---|---|---|
| mmu_piRNA_ | UGUGAUCAGAGCACUUCAGAAGCUGG | M1 | | 645 |
| 807260 | | | | |
| mmu_piRNA_ | UGAGCACGAACCUGGAAGCAGCUCC | M2 | | 646 |
| 603506 | UACC | | | |
| mmu_piRNA_ | UGUGCAUCAGAGAGAAGGCCUGUGUU | M3 | | 647 |
| 809894 | | | | |
| mmu_piRNA_ | UGUCACUGGAAGCAGCCUCAUGAGGG | M4 | ✓ | 648 |
| 790998 | | | | |
| mmu_piRNA_ | UGAAUGUUUUGCCUGAUUGUCUCCAU | M7 | | 649 |
| 567941 | | | | |
| mmu_piRNA_ | ACAGUAGAAAGGUACCCAUUGGGAU | M9 | | 650 |
| 30784 | CACA | | | |
| mmu_piRNA_ | UGGUACCAGAGCACUUCGAAUCACAG | | | 651 |
| 762212 | | | | |
| mmu_piRNA_ | UGACCAGAGCACUUCAGUUCCUAAGA | | | 652 |
| 578463 | | | | |
| mmu_piRNA_ | CAGAGAGACUGGAAGCAGCUCCCAGA | | | 653 |
| 107622 | ACUC | | | |
| mmu_piRNA_ | UGUUCCUUAAAGAAGGAGGGUUAUGU | | | 654 |
| 828274 | | | | |
| mmu_piRNA_ | UGGUCAUGAGUCACUGGAAACAUCAU | | | 655 |
| 766530 | | | | |
| mmu_piRNA_ | UGGGAGCAGAAGGCCUUAAUGAUGUCUA | | | 656 |
| 746831 | | | | |
| mmu_piRNA_ | UGACUCUUUACAGUGAAAGAAUAGCC | | | 657 |
| 583641 | | | | |
| mmu_piRNA_ | UUUGGCUUCUGAUUUCAGAGGCUUAG | | | 658 |
| 931384 | | | | |
| mmu_piRNA_ | UUUGGAAGCAGCUCCUCAGGGACCU | | | 659 |
| 930854 | | | | |
| mmu_piRNA_ | UGAUAGCACUCAAUUCUUCCAAAAGGA | | | 660 |
| 628461 | | | | |

piRNA matching matrix protein (sense)

| | | | | | |
|---|---|---|---|---|---|
| mmu_piRNA_ | UUAUCAAGCUCUGCUUCCAGUGACUC | M10 | | | 661 |
| 853934 | | | | | |
| mmu_piRNA_ | UGCUUCCAGUGACUCAGAGCUUAGUAAU | M11 | | ✓ | 662 |
| 699256 | | | | | |
| mmu_piRNA_ | UUUCAGGUGUUUCCCUGCCAUUCGGUA | M12 | | | 663 |
| 922935 | | | | | |
| mmu_piRNA_ | UUGAGAUGACCUUGAUCUGCCUAUGU | M13 | | | 664 |
| 894300 | | | | | |
| mmu_piRNA_ | UUACCAAAAUAGGAUUUGGCCAGCAG | M14 | | | 665 |
| 847352 | | | | | |
| mmu_piRNA_ | UGUAAAACUCUGAAAUCAGAAACUGU | M15 | | | 666 |
| 779649 | | | | | |
| mmu_piRNA_ | UGAGUGGCCUUUAGAUCUAAUUCAAUC | M17 | ✓ | | 667 |
| 621036 | | | | | |
| mmu_piRNA_ | UGGGCCUUCUCUCUGAAGGGAGAUGCUAA | | | | 668 |
| 753840 | GU | | | | |
| mmu_piRNA_ | UCUUUAUGUAAAACUCUGAAAUCAGAAA | | | | 669 |
| 533992 | | | | | |
| mmu_piRNA_ | UCUGGAAUCCAGGACCCACAGAGGUU | | | | 670 |
| 523622 | | | | | |
| mmu_piRNA_ | UCAUUGUGAGCUCAAGGUCAACCUGG | | | | 671 |
| 474715 | | | | | |
| mmu_piRNA_ | UCAGGAUUCCGAUGUACAUGGAAGAC | | | | 672 |
| 466882 | | | | | |
| mmu_piRNA_ | UAGUUACCAAAAUAGGAUUUGGCCAGCA | | | | 673 |
| 416604 | | | | | |
| mmu_piRNA_ | UAGGCAUCUGAGUAUGUUCCCUCCUGC | | | | 674 |
| 400109 | | | | | |
| mmu_piRNA_ | UACCAAAAUAGGAUUUGGCCAGCAGGAGA | | | | 675 |
| 333076 | | | | | |
| mmu_piRNA_ | UAAAUAUGAAUUAGAUUAGAAGAAC | | | | 676 |
| 263124 | AGUUGG | | | | |
| mmu_piRNA_ | UAAAACUCUGAAAUCAGAAACUGUG | | | | 677 |
| 246499 | | | | | |
| mmu_piRNA_ | GACUUGGGCCUUCUCUCUGAAGGGAGAUG | | | | 678 |
| 189111 | CU | | | | |
| mmu_piRNA_ | AAGUUUCCCUGCCAUUCAGAAGGACAA | | | | 679 |
| 22035 | | | | | |
| mmu_piRNA_ | UUUCUGAAAGAAUCCAGGACCACCAGC | | | | 680 |
| 925373 | | | | | |
| mmu_piRNA_ | UGUUACCCACUUCCAGUGACUCACAGCAA | | | | 681 |
| 824596 | | | | | |
| mmu_piRNA_ | UGUGAGCUCAAUCGGAGGAUCAUGGAA | | | | 682 |
| 805225 | | | | | |
| mmu_piRNA_ | UGGUUAUGAAGAAUCCAGAUGCCUUUU | | | | 683 |
| 776013 | | | | | |
| mmu_piRNA_ | UGAGGGAGUAUGGUCAUUGUGAGACUAA | | | | 684 |
| 614225 | GAUCC | | | | |
| mmu_piRNA_ | UGAAGUGCUCUGGUCUAGCUCGUUAC | | | | 685 |
| 563085 | | | | | |
| mmu_piRNA_ | UCCUAGUGGCCUUUAGAUGAAGAUGU | | | | 686 |
| 495354 | | | | | |
| mmu_piRNA_ | UUUAAAAGAUGCCUUUUUCUGCCUGA | | | | 687 |
| 918221 | | | | | |
| mmu_piRNA_ | UGGUUCAGGCAUUCUUUAAGGAACU | | | | 688 |
| 776262 | AAGUC | | | | |
| mmu_piRNA_ | UGGAUUUAAAAGAUGCCUUUUUCUGUCU | | | | 689 |
| 728430 | | | | | |
| mmu_piRNA_ | UGAUUUAAAAGAUGCCUUUUUCUGCCU | | | | 690 |
| 645098 | | | | | |
| mmu_piRNA_ | UGAGAGGUCUGAAGUGCUCUGAGGUCC | | | | 691 |
| 597622 | | | | | |
| mmu_piRNA_ | UGAAAGAUGCCUUUUUCUGUCUCAGA | | | | 692 |
| 541999 | | | | | |

piRNA matching structural non-encoding 4 (antisense)

| | | | | |
|---|---|---|---|---|
| mmu_piRNA_ | UUAGCUAGUCUAACUUCCUUGAUGA | S4-1 | | 693 |
| 851363 | GUUCC | | | |
| mmu_piRNA_ | UAGCUAGUCUAACUUCCUUGAUGAGU | S4-2 | ✓ | 694 |
| 386893 | UCC | | | |
| mmu_piRNA_ | AGCUAGUCUAACUUCCUUGAUGAGUUC | S4-3 | ✓ | 695 |
| 59149 | | | | |
| mmu_piRNA_ | UGGCUAGCUUCUGAACAGCAGGAUGGG | S4-5 | | 696 |
| 737655 | | | | |
| mmu_piRNA_ | UGCCACUUCAAAUGAACAGCAAGAG | S4-6 | ✓ | 697 |
| 667696 | CUUUU | | | |
| mmu_piRNA_ | UGAGUUGUACUGUUGGUAAACUGAGC | S4-7 | | 698 |
| 624205 | | | | |
| mmu_piRNA_ | UCGCUCCUCAUGGUUUACUCAGUCUGC | S4-8 | | 699 |
| 505268 | | | | |
| mmu_piRNA_ | AACUUGCUGGUUAGCUAGUCUAACU | | | 700 |
| 12411 | UCCUUG | | | |
| mmu_piRNA_ | GCUCAAAGAUGCCACUUCAAAUGAACAGC | | | 701 |
| 211679 | | | | |

piRNA matching structural non-encoding 4 (sense)

| | | | | | |
|---|---|---|---|---|---|
| mmu_piRNA_ | UGAGGAGAGACUGAGUAGUGUUGGA | S4-10 | | | 702 |
| 611658 | | | | | |
| mmu_piRNA_ | UUCAUUUGAAGUGGCAUCUUUGAGCU | S4-11 | | ✓ | 703 |
| 865734 | | | | | |
| mmu_piRNA_ | UGAAUAGAAAAGACAGGUGUUGCAG | S4-12 | | | 704 |
| 564528 | CUGUA | | | | |
| mmu_piRNA_ | UGAACCAGGAGCUAGAAGUUAGAGAU | S4-13 | | | 705 |
| 549289 | | | | | |
| mmu_piRNA_ | UCAGAAGCUAGAAGAGAGUGUCCGAU | S4-14 | ✓ | | 706 |
| 460095 | | | | | |
| mmu_piRNA_ | AAUAGAAAAGACAGGUGUUGCAGCU | S4-20 | | ✓ | 707 |
| 22365 | GUAA | | | | |
| mmu_piRNA_ | UAGGUUCAGAAGCUGGAAGUUCAGGA | | | | 708 |
| 406154 | | | | | |
| mmu_piRNA_ | UAGGAGAGAAAGAAAAGACAGGAUCU | | | | 709 |
| 395278 | | | | | |
| mmu_piRNA_ | UAAGAGAGAAAGAAAAGACAGGAUCU | | | | 710 |
| 283616 | | | | | |
| mmu_piRNA_ | UAAAGGCAGUAGGAGAGACUGAGCCU | | | | 711 |
| 259924 | | | | | |
| mmu_piRNA_ | AAGUCAUGAAUAGAAAAGACAGGUGUU | | | | 712 |
| 20817 | | | | | |
| mmu_piRNA_ | CUGGAAUUAGGAGAUAAACCUCUGGU | | | | 713 |
| 163711 | | | | | |

piRNA matching Luciferase (antisense)

| | | | | |
|---|---|---|---|---|
| mmu_piRNA_ | UUUUCUGUGAUAAAGGCCAAGAAGGA | Lu1 | | 714 |
| 936283 | | | | |
| mmu_piRNA_ | UAGAGCACUCUGAUUGACAGCAGUCUG | Lu2 | | 715 |
| 370919 | | | | |
| mmu_piRNA_ | CGUGUGCAGUGAAAACCAGAGCUAGC | Lu3 | ✓ | 716 |
| 147503 | | | | |
| mmu_piRNA_ | UGGAGAAAGGCAGCCCUGGUUCCUGGAUG | | | 717 |
| 718623 | A | | | |
| mmu_piRNA_ | UGAUAAAGGCCAAGAAGUGCAGGCCCAGA | | | 718 |
| 625432 | | | | |
| mmu_piRNA_ | UCAACAAAGGAUGUCAGGACUGGAACU | | | 719 |
| 449164 | | | | |
| mmu_piRNA_ | UAGCUGUGUCACCUAAGGGUGUUGGG | | | 720 |
| 389077 | | | | |
| mmu_piRNA_ | UAGAAGAAAAAUCAAAGAGAUCAGGA | | | 721 |
| 360479 | | | | |
| mmu_piRNA_ | UAAUUUAUGAAGGAUGUCAGGUGGU | | | 722 |
| 311855 | | | | |
| mmu_piRNA_ | UUUGCAGAUUGUGCCCUGGUUCCUGGGC | | | 723 |
| 929947 | | | | |
| mmu_piRNA_ | UUUCUGUCAAAACCAUCUUCCAGGGAGAC | | | 724 |
| 926061 | | | | |
| mmu_piRNA_ | UUUCUAGUUCCAUUUUUUGAUCCUGGA | | | 725 |
| 924646 | | | | |
| mmu_piRNA_ | UUGGCAGUGCGUUGCUAGUCAAUAUG | | | 726 |
| 906469 | | | | |
| mmu_piRNA_ | UCUAAGCUCAUGUUGUUCCAUUCCAC | | | 727 |
| 507368 | | | | |

piRNA matching Luciferase (sense)

| | | | | | |
|---|---|---|---|---|---|
| mmu_piRNA_ | UCUCAAGAUGUUGGGGUGUUGGCCAG | Lu4 | | | 728 |
| 510231 | | | | | |
| mmu_piRNA_ | UAAGAUGGAAGCGUUUUGGGAGUCA | Lu5 | ✓ | | 729 |
| 287933 | | | | | |
| mmu_piRNA_ | UGCCACUUCAAAAAAUGGAACCCCGG | Lu6 | | | 730 |
| 667694 | | | | | |
| mmu_piRNA_ | UGAGCAUAAUUUUUGGAAACAAACAUG | Lu7 | | | 731 |
| 604539 | | | | | |
| mmu_piRNA_ | UGAGAUGUUGGGGUGUUGGCCAGCUC | | | | 732 |
| 601753 | | | | | |
| mmu_piRNA_ | UUGUUCCAGGAACCACGGAGACUGUC | | ✓ | | 733 |
| 916610 | | | | | |
| mmu_piRNA_ | UUCCCAAUGAUUUGAUUGCCCUCCCA | | | | 734 |
| 869808 | | | | | |
| mmu_piRNA_ | UUAUGAAGAAGUGUUCCAGGCUGGAA | | | ✓ | 735 |
| 854628 | | | | | |
| mmu_piRNA_ | UGGAUGAGGGUCCCAGUAAGCUAUUC | | | | 736 |
| 726366 | | | | | |
| mmu_piRNA_ | UGGAUCUCUCUGAUUUCAAGGUCAGCC | | | ✓ | 737 |
| 725846 | | | | | |
| mmu_piRNA_ | UGCUAUGAAGAAGUGUUUAGGAACCA | | | | 738 |
| 685614 | | | | | |
| mmu_piRNA_ | UCCCAAUGAUUUGAUUGCCCUCCCA | | | | 739 |
| 485965 | | | | | |
| mmu_piRNA_ | UAUGAAGAAGUGUUCCAGGCUGGAAU | | | | 740 |
| 430521 | | | | | |
| mmu_piRNA_ | UAACAGUUUCCCAAUGAUUUGAUUGCCC | | | | 741 |
| 271080 | | | | | |
| mmu_piRNA_ | GCUAACAGUUUCCCAAUGAUUUGAUUGCC | | | | 742 |
| 211183 | | | | | |
| mmu_piRNA_ | UUGUUUGAAAUAGGAUCUCUGUGUGU | | | | 743 |
| 917694 | | | | | |
| mmu_piRNA_ | UUGCUGUCCAGGCCUGAGGAUCAGAA | | | | 744 |
| 901756 | UAGCUGAG | | | | |
| mmu_piRNA_ | GCUUUGGUCAUGGUAUCUCUUCAUAGC | | | | 745 |
| 215384 | | | | | |

piRNA matching structural non-encoding 5 (antisense)

| | | | |
|---|---|---|---|
| mmu_piRNA_ | UGUGAUACCAUGCUGGGCUCCAUGCAGUG | S5-1 | 746 |
| 807014 | UUUUUGU | | |
| mmu_piRNA_ | UGAGAGCAGCUCAAAGAGGCCUAUGA | S5-2 | 747 |
| 596001 | | | |
| mmu_piRNA_ | CAUGAUACCAUGCUCAUGUGAACUCUGA | S5-6 | 748 |
| 127168 | | | |
| mmu_piRNA_ | GCAGCUCAAAGAGGCCUACGAACCAG | | 749 |
| 205186 | | | |
| mmu_piRNA_ | CCUGAGAGCAGCUCAAAGAGGCCUAU | | 750 |
| 141070 | | | |

piRNA matching structural non-encoding 5 (sense)

| | | | | |
|---|---|---|---|---|
| mmu_piRNA_ | UGUGGUAUUUUGGUGAAGAAUCUGGCU | S5-7 | | 751 |
| 815423 | | | | |
| mmu_piRNA_ | UGAGCAUGGUAUCAUGGAGUUCCCCAG | S5-10 | | 752 |
| 604923 | | | | |
| mmu_piRNA_ | UCCUGGUUCGUAGGCCUCUUUGAGCU | S5-11 | | 753 |
| 501107 | | | | |
| mmu_piRNA_ | AUGAGCAUGGUAUCAUGGAGUUCCCCA | S5-20 | ✓ | 754 |
| 80811 | | | | |
| mmu_piRNA_ | AGGCCUCUUUGAGCCCUUCAUGGCUU | S5-22 | ✓ | 755 |
| 65656 | | | | |
| mmu_piRNA_ | UGGUGAAGAAUCUGGCUGCUUUUUGC | | | 756 |
| 769226 | | | | |
| mmu_piRNA_ | UCAGAGUUCACAUGAGCAUGGUAUCAU | | | 757 |
| 462994 | | | | |
| mmu_piRNA_ | UAGGCCUCUUUGAGCUGCUCUCAGG | | | 758 |
| 400537 | CGGUA | | | |
| mmu_piRNA_ | UAAAAGCACAAGGCCUCUUUGAGCCCUA | | | 759 |
| 248015 | | | | |
| mmu_piRNA_ | GAGUUCACAUGAGCAUGGUAUCAUG | | | |
| 197551 | GAGU | | | |
| mmu_piRNA_ | CAGCUGGACUGCACAGGCCUCUUUGA | | | 761 |
| 114520 | CGCC | | | |
| mmu_piRNA_ | AGGCCUCUUUGAGCUGCUCUCAGGCGGU | | | 762 |
| 65657 | | | | |
| mmu_piRNA_ | CAGAGUUCACAUGAGCAUGGUAUCAU | | | 763 |
| 109541 | | | | |
| mmu_piRNA_ | AGCUGGACUGCACAGGCCUCUUUGAG | | | 764 |
| 60206 | CCCUU | | | |

piRNA matching RNA-directed RNA polymerase L (antisense)

| | | | | | |
|---|---|---|---|---|---|
| mmu_piRNA_ | UGAGGCAGUAUUAUGGUUAUUCUCAG | R1 | | | 765 |
| 613008 | | | | | |
| mmu_piRNA_ | UAAUAUAGGGAAACUUCAUUUGGCUC | R2 | | | 766 |
| 302059 | | | | | |
| mmu_piRNA_ | CCUUUCACCAUGUGGAUCUGGGAGAU | R3 | | | 767 |
| 143877 | CAGA | | | | |
| mmu_piRNA_ | CAGGGAAUGCAACUUGAAGCUGACGGA | R4 | | | 768 |
| 119094 | | | | | |
| mmu_piRNA_ | UUGGAAAGUCAGAGCACUGCAGCACUG | | | | 769 |
| 902698 | | | | | |
| mmu_piRNA_ | UGGAACGGAAUCAGGUUGGGCAGCGUGA | | | | 770 |
| 707643 | | | | | |
| mmu_piRNA_ | UGCGCAGAUAUGUUGUAUUUGGACCU | | | | 771 |
| 680263 | | | | | |
| mmu_piRNA_ | UGAUUGAUUGUGAGGAGUGGAUUCUU | | | | 772 |
| 644018 | | | | | |
| mmu_piRNA_ | UGAGGAAAUCAGACAUGGAAAGCAUA | | ✓ | | 773 |
| 609974 | | | | | |
| mmu_piRNA_ | UGACCACUGGACUUCUUGAGUUGUUG | | | | 774 |
| 578379 | | | | | |
| mmu_piRNA_ | UGAAGGCAGAAAUAACAUUGAGCAUU | | | | 775 |
| 560276 | | | | | |
| mmu_piRNA_ | UCCUAGGUACACUGGGACAUUUGCUAGA | | | | 776 |
| 495193 | | | | | |
| mmu_piRNA_ | UCACGUGCCAGCAGCAUUGACCAGGUA | | | | 777 |
| 455962 | | | | | |
| mmu_piRNA_ | UAGGUACAGGAAGACAUACAUGUGC | | | | 778 |
| 403614 | | | | | |
| mmu_piRNA_ | UAGGCAAAGUCAGAAGACAGAUGUGA | | ✓ | | 779 |
| 399365 | | | | | |
| mmu_piRNA_ | UAGGAAUGAGGAAAUCAGACAUGGA | | | | 780 |
| 392918 | | | | | |
| mmu_piRNA_ | UAAGAUGAGGAGUGGGGUGGGAAGGC | | | | 781 |
| 287629 | | | | | |
| mmu_piRNA_ | CGUCCUGACAGAUGCUUCUAACUGCA | | | | 782 |
| 147255 | GUCAUUU | | | | |
| mmu_piRNA_ | UUUCAAUGAAGAUGAGAAGUAUUUCU | | | ✓ | 783 |
| 922108 | GAGAGC | | | | |
| mmu_piRNA_ | UUGGUGCCUAAAUGUAAAGGUUUGAAC | | | | 784 |
| 909482 | | | | | |
| mmu_piRNA_ | UUGGAGUAGUUCUUAUGACUUGAUGGUCC | | | | 785 |
| 905139 | U | | | | |

piRNA matching RNA-directed RNA polymerase L (sense)

| | | | | | |
|---|---|---|---|---|---|
| mmu_piRNA_ | CUGCGCUGUCAGGAACAUCUUGACAAC | R5 | | | 786 |
| 161774 | AGGUUGUCUAUUCUGUUUUG | | | | |
| mmu_piRNA_ | UUCUGACUUUGCCUUUCUGCCAUAGA | | | ✓ | 787 |
| 881038 | | | | | |
| mmu_piRNA_ | UGGCUAACAGACUAUUGAAACAAUGGUU | | | | 788 |
| 737172 | | | | | |
| mmu_piRNA_ | UGGAUUCCACCUCAGAGACCUCGGA | | | | 789 |
| 727902 | | | | | |
| mmu_piRNA_ | UGAUCCCAGGGGUAACCAGUAGAUUGA | | | | 790 |
| 631864 | GUCUC | | | | |
| mmu_piRNA_ | UCCCAUGCUGCCUGUUGGUCCUCCUCUGC | | ✓ | | 791 |
| 488350 | | | | | |
| mmu_piRNA_ | UAGCUUCUGUUUGUAUCCUCCUUCCC | | | | 792 |
| 389592 | | | | | |
| mmu_piRNA_ | GACAAAGAGGUUUCCUCAUGUAGGGA | | | | 793 |
| 183655 | | | | | |
| mmu_piRNA_ | CUGCCUCCCAAGUCCUGGGGUCACAGGU | | | | 794 |
| 161451 | | | | | |
| mmu_piRNA_ | UUUCUGAACAUGUCCACAGCCGCCCU | | | | 795 |
| 925385 | | | | | |
| mmu_piRNA_ | UUGAUUGCUGGAUAACCUAUUGGGGAA | | | | 796 |
| 897252 | | | | | |

piRNA matching structural non-encoding 6 (antisense)

| | | | | |
|---|---|---|---|---|
| mmu_piRNA_ | UGAGAAGAAGGAGUUGGCUGCUGCCA | S6-2 | | 797 |
| 589679 | | | | |
| mmu_piRNA_ | UUUGUGACAUUCUGGCUGCUAACAAAGCU | S6-3 | | 798 |
| 932621 | | | | |
| mmu_piRNA_ | UGAGGAGACUCCAAAGAGGAGACAAU | S6-4 | ✓ | 799 |
| 611643 | | | | |
| mmu_piRNA_ | UAGAUGGUUUUGUGGUCUUAAUUACACCU | S6-5 | | 800 |
| 376792 | | | | |
| mmu_piRNA_ | UACACUAGCAAAAUUUUGCUGAAAGGAGC | S6-6 | | 801 |
| 320338 | | | | |
| mmu_piRNA_ | UUUUGCUGAAAGGAUCCAGAUAUAGC | | | 802 |
| 937100 | | | | |
| mmu_piRNA_ | UUUUGCUGAAAGGACCCAGAUAUGGCU | | | 803 |
| 937098 | | | | |
| mmu_piRNA_ | UUUUGCUGAAAGGACCCAGAUAUAGCUUG | | | 804 |
| 937097 | | | | |
| mmu_piRNA_ | UUUUGCUGAAAGGACCCAGAUAUAGCUG | | | 805 |
| 937096 | | | | |
| mmu_piRNA_ | UUUUGCUGAAAGGACCCAGAUAGAAC | | | 806 |
| 937095 | | | | |
| mmu_piRNA_ | UUUUGCUGAAAGGACCCAGAUAAAG | | | 807 |
| 937094 | | | | |
| mmu_piRNA_ | UUUUGCUGAAAGGACCCAGAGAUAGCUGU | | | 808 |
| 937093 | | | | |
| mmu_piRNA_ | UUUGCUGAAAGGAGCCAGAUAUAGCUGU | | | 809 |
| 930524 | | | | |
| mmu_piRNA_ | UGUGAAUUUUUUUGGUCUUCGUGGGC | | | 810 |
| 802421 | | | | |
| mmu_piRNA_ | UGUCACAGGGUGGCUGCUUGGGGCCUCUA | | | 811 |
| 790476 | AUUGAACCCA | | | |
| mmu_piRNA_ | UGGCUGAAAGGAGGAAGACUAUGCGU | | | 812 |
| 738672 | | | | |
| mmu_piRNA_ | UGGAAAAGUAGAUGGCUAAGGGAGA | | | 813 |
| 703766 | | | | |
| mmu_piRNA_ | UGGCUUUGAACUCUUGGAGAGAGAGU | | | 814 |
| 740986 | | | | |

piRNA matching structural non-encoding 6 (sense)

| | | | |
|---|---|---|---|
| mmu_piRNA_ | UAGUGAGAUGAGAGGGUCUUAAGGAG | S6-7 | 815 |
| 413050 | | | |

This table contains piRNA species having at least 15 nt matches with VSV genome-based pseudotyped SARS-CoV-2 sense or antisense RNA sequence; among them, piRNAs which meet criteria 1 or 2 are indicated by a check. SEQ ID NOs. 522, 523, 529-535, 553-560, 589-591, 597-601, 607-613, 641-650, 661-667, 693-699, 702-707, 714-716, 728-741, 746-748, 751-755, 765-768, 786, 797-801, and 81 are piRNAs for qPCR.

### Example 4: Enrichment of NSC Ex/Mv piRNAS by Pseudotyped SARS-CoV-2 Initial Exposure

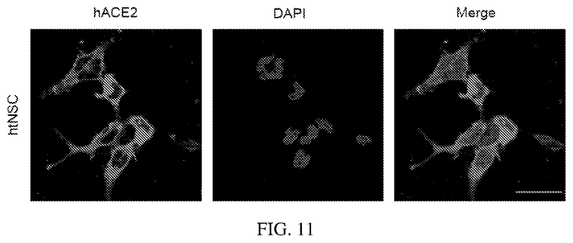

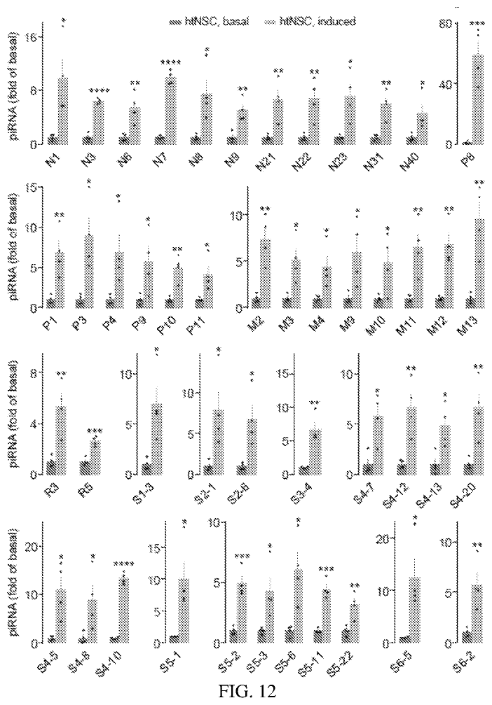

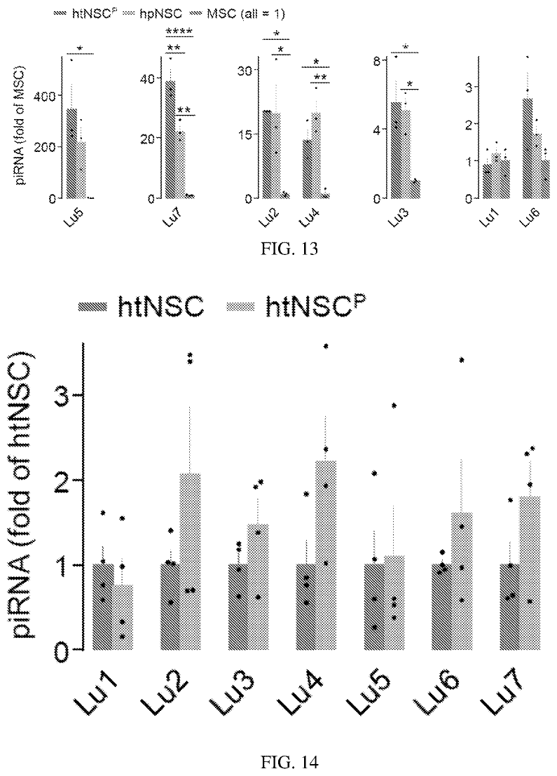

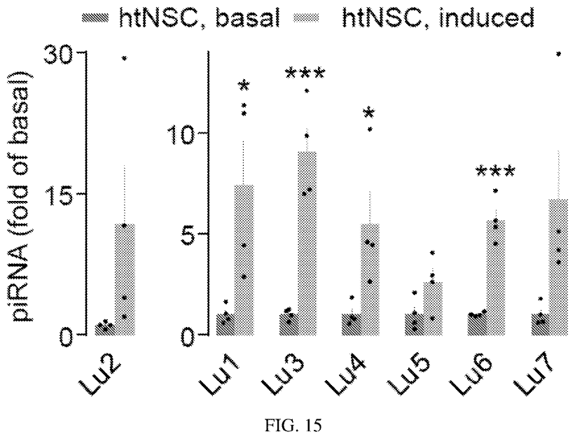

While NSC Ex/Mv contain antiviral piRNA libraries, it was further determined if an initial exposure of a specific virus to NSC could lead to an enhancement or enrichment of certain specific piRNAs in NSC Ex/Mv. To provide an answer to this question, a pseudotyped SARS-CoV-2 virus was employed to provide an initial stimulation to cultured htNSC. Because infection of pseudotyped SARS-CoV-2 requires human angiotensin-converting enzyme 2 (hACE2) as the receptor, an hACE2-htNSC cell line was generated with expression of hACE2 through a lentiviral induction (FIG. 11). After these cell lines were stably maintained for several generations in culture, these cells were treated with pseudotyped SARS-CoV-2 virus for 2 generations, and then maintained under normal culture for about 5 generations. After that, Ex/Mv were isolated and purified from these virally stimulated NSC vs. vehicle stimulated NSC as controls and then measured their piRNA expression levels via qPCR. Among about 80 piRNAs which were examined, about half of them were significantly upregulated due to the exposure to this virus, typically 5 to 10 fold higher than the basal levels (FIG. 12). The enrichment of these piRNAs was more due to negative selection of other irrelevant small RNAs in these Ex/Mv leading to their relatively higher levels of these specific piRNAs. We also evaluated this effect according to luciferase encoding sequence in this viral genome, since we surprisingly found some piRNA species which matched the sense or antisense sequence of luciferase RNA (Table 2), although luciferase is a non-viral protein. Seven of these piRNA species were measured and 5 of them were present in NSC Ex/Mv but negligible in MSC Ex/Mv (FIG. 13, 14), indicating that murine NSC Ex/Mv have piRNA libraries against various types of foreign genomic sequence. It was then examined if these piRNAs could be upregulated due to the viral exposure, and results revealed that 4 of them showed significant upregulation (FIG. 15). Thus, NSC Ex/Mv can be subject to piRNA enhancement and enrichment in response to an initial viral exposure to potentially better counteract against this virus subsequently.

### Example 5: Antiviral Effects of Murine NSC Ex/Mv Through Testing an HIV-Based Lentivirus

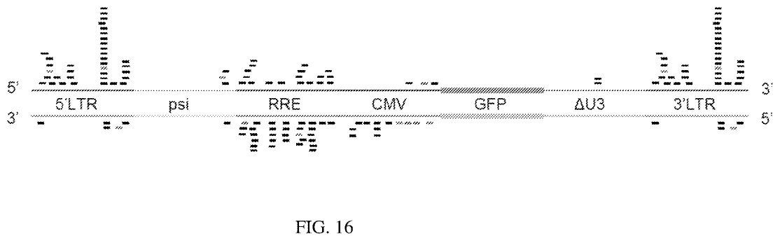

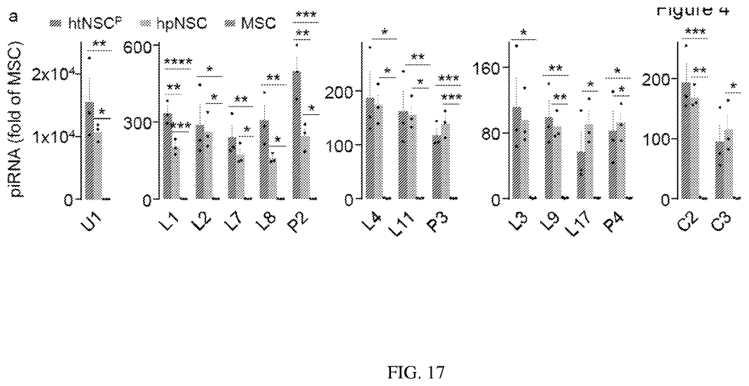

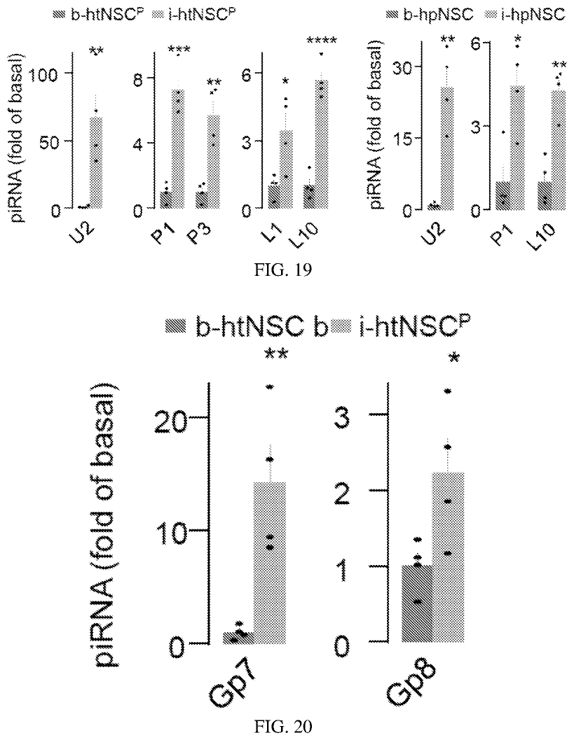

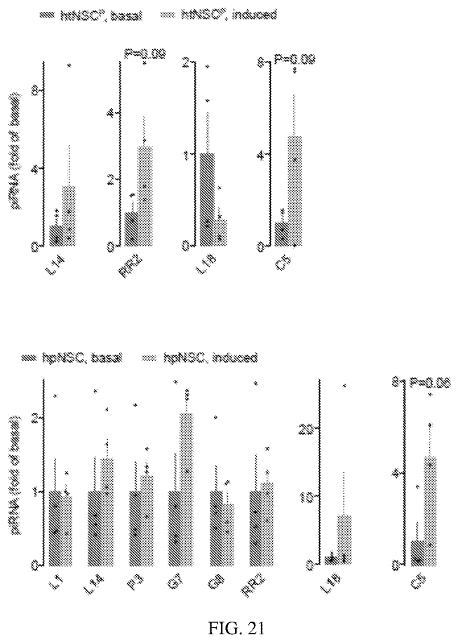

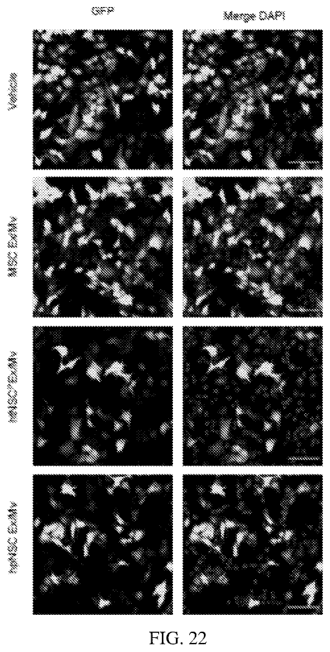

To further assess the predicted antiviral role of murine NSC Ex/Mv piRNAs, an RNA virus that is phenotypically unrelated to SARS-CoV-2 was examined. A VSV glycoprotein (VSVG)-enveloped laboratory recombinant lentivirus which contains a few HIV genomic RNA segments including HIV long terminal repeats (LTR), psi, RRE, and ΔU3 sequences was examined In addition, this viral genome recombinantly contained a different virus-derived promoter (CMV), and a non-viral sequence encoding GFP, as shown in FIG. 16. As similarly performed for SARS-CoV-2, we applied each segment of these sense and antisense sequences to search for mouse piRNA database against this lentivirus. Despite that this recombinant lentiviral RNA sequence is rather short, we identified a list of piRNAs with at least 15 nucleotides matching with the viral sequence and found quite a few which met Criteria 1 or 2 (Table 3). Through qPCR, we examined some of these piRNAs and found that all of them were present in NSC Ex/Mv but absent or negligible in MSC Ex/Mv (FIG. 17, 18). Further, some of these Ex/Mv piRNAs were induced to increase expression levels after an initial exposure of lentivirus to these NSCs, although these effects were slightly stronger in htNSC^{PGHM} than hpNSC (FIG. 19, 20). In addition, just like the strategy of analyzing luciferase piRNAs in FIG. 12, it was determined if GFP encoding sequence could have piRNAs and if so, whether they could be induced through this lentiviral exposure. As shown in Table 3, several mouse piRNAs were found that matched against either the sense or antisense sequence of GFP RNA. qPCR was used to measure 8 of them and all of them were detectable in NSC Ex/Mv but absent in MSC Ex/Mv. Two of these GFP piRNAs were significantly upregulated in NSC Ex/Mv due to a recent exposure of NSC to the lentivirus—(FIG. 21). Thus, these results further support the notion that viral exposure-induced Ex/Mv piRNA reaction in NSCs can apply to a newly incorporated sequence in a viral genome.

TABLE 3
INFORMATION ON MOUSE PIRNAS POTENTIALLY AGAINST RECOMBINANT LENTIVIRUS

| | | | | | SEQ |
|---|---|---|---|---|---|
| PIRNA ID | | | Criteria | Criteria | ID |
| (piRNAQuest) | piRNA sequence | Label | 1 | 2 | NO. |

piRNA matching 5′ LTR/3′ LTR (sense)

| | | | | |
|---|---|---|---|---|
| mmu_piRNA_ | UGUGCCUGGCUAACUAGGGAUGUGUUU | L1 | | 816 |
| 810807 | | | | |
| mmu_piRNA_ | UGUCUCACUGAGCCUGGGAGCUUGCUG | L2 | | 817 |
| 796943 | | | | |
| mmu_piRNA_ | UGGGUGCAGAUCUGAGCCUGGAAAGU | L3 | | 818 |
| 758535 | | | | |
| mmu_piRNA_ | UGAUCUGAGCCUGGGAUGGGGAAGAC | L4 | ✓ | 819 |
| 632877 | | | | |
| mmu_piRNA_ | UUUGAAUUAGUCAGUGUGGAGAGGAGU | L5 | | 820 |
| 927637 | | | | |
| mmu_piRNA_ | UUGUUGAAGAUGCUUAAGCCUCAAAGA | L6 | | 821 |
| 916882 | | | | |
| mmu_piRNA_ | UUGGAUCUGAGCCUGGGUAUGAGCUG | L7 | | 822 |
| 905594 | | | | |
| mmu_piRNA_ | UUAUCCAUGAGCCUGGGAGCUUCUCU | L8 | | 823 |
| 854250 | | | | |
| mmu_piRNA_ | UUAAAGAACCCACUGCUUAUGGCCUAGAC | L9 | | 824 |
| 840907 | | | | |
| mmu_piRNA_ | UGUGUGACUCUGGUGUCCCCUGCUGCU | L10 | | 825 |
| 818932 | | | | |
| mmu_piRNA_ | UGCAAGAGCUCUCUGGCUAGGUGGUU | L11 | | 826 |
| 649949 | | | | |
| mmu_piRNA_ | UGAUGUCUGUCAGUGUGGAAAAAAA | L12 | | 827 |
| 640578 | | | | |
| mmu_piRNA_ | UGAAGACCAGAUCUGAGUUCAGAGAG | L13 | | 828 |
| 555080 | CUU | | | |
| mmu_piRNA_ | UCCAGGCUUACAUUAAUUGGUUAGA | L14 | | 829 |
| 482420 | CCAGAU | | | |
| mmu_piRNA_ | GUGUGUGACUCUGGUGUCCCCUGCUGU | L21 | ✓ | 830 |
| 238327 | | | | |
| mmu_piRNA_ | UGUCUGUCAGUGUGGAAAAAAAAAAA | | | 831 |
| 798725 | | | | |
| mmu_piRNA_ | UGCUGAUGUCUGUCAGUGUGGAAAAA | | | 832 |
| 691993 | AAAAA | | | |
| mmu_piRNA_ | UAGUGUGUGACUCUGGUGUCCCCUGCU | | | 833 |
| 416030 | | | | |
| mmu_piRNA_ | UACAUCUUAAAGAACCCACUGCUUAUGGC | | | 834 |
| 331085 | | | | |
| mmu_piRNA_ | UACAUCAUCCUGAUCCCUCAGACCCA | | | 835 |
| 330766 | | | | |
| mmu_piRNA_ | UACAGAAGUAGUGUGUGACUCUGGUGU | | | 836 |
| 321750 | | | | |
| mmu_piRNA_ | UAAAGAACCCACUGCUUAUGGCCUAGAC | | | 837 |
| 256154 | | | | |
| mmu_piRNA_ | GUAGUGUGUGACUCUGGUGUCCCCUG | | | 838 |
| 228888 | CUGC | | | |
| mmu_piRNA_ | GUACAGAAGUAGUGUGUGACUCUGGUG | | | 839 |
| 228218 | | | | |
| mmu_piRNA_ | GGUACAGAAGUAGUGUGUGACUCUGGU | | | 840 |
| 224710 | | | | |
| mmu_piRNA_ | GAAGUAGUGUGUGACUCUGGUGUCCCC | | | 841 |
| 181446 | | | | |
| mmu_piRNA_ | CUGAUGUCUGUCAGUGUGGAAAAAAAA | | | 842 |
| 159923 | | | | |
| mmu_piRNA_ | CNGAAGUAGUGUGUGACUCUGGUGUCCCC | | | 843 |
| 147685 | | | | |
| mmu_piRNA_ | CAGAAGUAGUGUGUGACUCUGGUGUCC | | | 844 |
| 105042 | | | | |
| mmu_piRNA_ | AGUAGUGUGUGACUCUGGUGUCCCCUGC | | | 845 |
| 70192 | | | | |
| mmu_piRNA_ | AGAAGUAGUGUGUGACUCUGGUGUCCC | | | 846 |
| 46170 | | | | |
| mmu_piRNA_ | ACGUGCUUGCUGUGUGACUCUGGUGACC | | | 847 |
| 37975 | | | | |
| mmu_piRNA_ | ACAGAAGUAGUGUGUGACUCUGGUGU | | | 848 |
| 28124 | | | | |
| mmu_piRNA_ | AAGUAGUGUGUGACUCUGGUGUCCCC | | | 849 |
| 20623 | UGCUGC | | | |
| mmu_piRNA_ | ACAGAAGUAGUGUGUGACUCUGGUGU | | | 850 |
| 28124 | | | | |

piRNA matching 5′ LTR/3′ LTR (antisense)

| | | | | |
|---|---|---|---|---|
| mmu_piRNA_ | UUUUGUUCCCUUUCCACACUGACUGU | L15 | | 851 |
| 937913 | | | | |
| mmu_piRNA_ | UAGCUCAGAUCUGGUCACUCCUCCAA | L17 | | 852 |
| 387174 | | | | |
| mmu_piRNA_ | UAACUUCAGUUACCAGAGUCAAAGGU | L18 | | 853 |
| 277437 | | | | |
| mmu_piRNA_ | UAAAAGGGUCUGAGCUCCCUAAUGGCA | L19 | ✓ | 854 |
| 249106 | | | | |
| mmu_piRNA_ | GAUAACUUCAGUUACCAGAGUCAAAG | L20 | | 855 |
| 198158 | | | | |

piRNA matching psi packing signal (sense)
mmu_piRNA_

| | | | | |
|---|---|---|---|---|
| mmu_piRNA_ | UAGAAGGAGAGAGAUCUUGCCCCACC | P1 | ✓ | 856 |
| 362636 | | | | |
| mmu_piRNA_ | UCUAAUGAAGGAGAGAGAUGCUCUCA | P2 | | 867 |
| 507681 | | | | |
| mmu_piRNA_ | UCCCAGAGAGAUGGGUGCUCGGUCUACA | P3 | | 858 |
| 486685 | | | | |

piRNA matching psi packing signal (antisense)

| | | | |
|---|---|---|---|
| mmu_piRNA_ | UGUAAUCUGGUGACCCAUCUCUCUCC | P4 | 859 |
| 783243 | | | |

piRNA matching RRE (sense)

| | | | |
|---|---|---|---|
| mmu_piRNA_ | UCUGAUGCAACUCACAGUAGGGUCUU | PR1 | 860 |
| 520714 | | | |
| mmu_piRNA_ | UAACAGCAGCAGGAAGCACUUCACAC | | 861 |
| 269624 | | | |
| mmu_piRNA_ | UUCAAUCGCCAGCAGCAGAACAAUA | | 862 |
| 859095 | | | |
| mmu_piRNA_ | UCUUUAGUUCUUGGGAGCAGCCAGCC | | 863 |
| 533940 | | | |
| mmu_piRNA_ | UAAGAGAGUUCCUGGGAGCAGCAGGA | | 864 |
| 283886 | | | |
| mmu_piRNA_ | UAAAGCCUUUGCAACUCACAGUCAUCU | | 865 |
| 258836 | | | |
| mmu_piRNA_ | UGCCCUAAAGGAUCAACUGAGUCAGG | | 866 |
| 672607 | | | |
| mmu_piRNA_ | UGAUGGAGCAGCAGGAAGGAUUACAG | | 867 |
| 638314 | | | |
| mmu_piRNA_ | UGAGGCUAGAGCAGCUCCAGGCAG | | 868 |
| 613519 | CAUGG | | |
| mmu_piRNA_ | UAAUACAGUUCCUUGGGUUCUAGUU | | 869 |
| 300430 | | | |
| mmu_piRNA_ | UAAACCUGGCUGUGGAAACGGAGCAA | | 870 |
| 254330 | | | |
| mmu_piRNA_ | GUCUCUGUUAGCUCUGGCUGUGGAAA | | 871 |
| 231816 | GGCA | | |
| mmu_piRNA_ | CUUACAGGCCAGACAAAUUUUCAGUUG | | 872 |
| 170887 | | | |
| mmu_piRNA_ | CCAGUGAAAUUUCCUCACAGUCUGGG | | 873 |
| 133418 | GAACAGUGU | | |
| mmu_piRNA_ | CACUGGGGCAUCAAGCCUUCACAGG | | 874 |
| 101405 | ACCAAGGGAC | | |
| mmu_piRNA_ | UUCAAAGGAUCAACAGCUGGAAUACA | | 875 |
| 857300 | | | |
| mmu_piRNA_ | UCAAAGGAUCAACAGCUGGAAUACACUU | | 876 |
| 448256 | | | |
| mmu_piRNA_ | UGAAGAAGAGCAGCAGGAAGCUGGGGU | | 877 |
| 553636 | | | |

piRNA matching RRE (antisense)

| | | | | | |
|---|---|---|---|---|---|
| mmu_piRNA_ | UCCAAGAACCCAAGAAACAAGAGAGGA | PR2 | ✓ | | 878 |
| 476685 | | | | | |
| mmu_piRNA_ | GACAGAUGCUGUUGCCUGUAUGAAAC | PR3 | | ✓ | 879 |
| 184866 | | | | | |
| mmu_piRNA_ | UGUCUGCUGCUGCACUAGUGGCCAUU | | | | 880 |
| 798278 | CUUCUAAUAU | | | | |
| mmu_piRNA_ | UGAAAUCGUGUCUGCUGCUGCACUAGU | | | | 881 |
| 545469 | GGCCAU | | | | |
| mmu_piRNA_ | CAGAGCCUGCUGCUCCCAAGGAUGCA | | | | 882 |
| 108215 | GAGGCCUCAGCCGAAGAUUCACCA | | | | |
| mmu_piRNA_ | AAGAGGUCACCAUGUCAUAUGGUCUU | | | | 883 |
| 14954 | CCCUGUGCUCCCU | | | | |
| mmu_piRNA_ | UUGUACUUCCUGCUGCUCCUGGACUU | | | | 884 |
| 911282 | | | | | |
| mmu_piRNA_ | UUCCACAGCCAGGAGAGUACAUUAGG | | | | 885 |
| 866737 | UGCCU | | | | |
| mmu_piRNA_ | UGUAUGUAUGGCUGCCUGGAGCUGCU | | | | 886 |
| 788543 | | | | | |
| mmu_piRNA_ | UGUAUGGCUGCCUGGAGCUGCUCGAA | | | | 887 |
| 788443 | | | | | |
| mmu_piRNA_ | UGGUGAAGCCUAGUUGCAACAGAUGA | | | | 888 |
| 769284 | | | | | |
| mmu_piRNA_ | UGCGCCUGGGUGUCUGGCCUGUACUC | | | | 889 |
| 680363 | | | | | |
| mmu_piRNA_ | UCUUUGUCUGGCCUGUAGGUUAUCGUC | | | | 890 |
| 534796 | | | | | |
| mmu_piRNA_ | UCUGUAUGUAUGGCUGCCUGGAGCUGCU | | | | 891 |
| 526335 | | | | | |
| mmu_piRNA_ | UCACAUCUUUGUCUGGCCUGUAGGU | | | | 892 |
| 454685 | | | | | |
| mmu_piRNA_ | UAUCAUCGGGGUGCUGCUUGAUGCCC | | | | 893 |
| 426852 | | | | | |
| mmu_piRNA_ | UAGUGCUUCCUGCUUUCUGAUUCAG | | | | 894 |
| 414424 | UCAG | | | | |
| mmu_piRNA_ | UAGUAGAGGUCCAGGAACAAAGCUCC | | | | 895 |
| 408415 | | | | | |
| mmu_piRNA_ | UAAUUUGCUGCUGCACUAUGUACC | | | | 896 |
| 312280 | CCUGGCUACCCAGCCUGU | | | | |
| mmu_piRNA_ | UAAGGGAACAGAUGCUGUUGGCUAGC | | | | 897 |
| 293964 | | | | | |
| mmu_piRNA_ | GAAGACCAUUGUAUUGCCUGGAGCUGC | | | | 898 |
| 179348 | | | | | |
| mmu_piRNA_ | CCUGCUGUGAGUUGCAUCAGAUCGGC | | | | 899 |
| 141877 | | | | | |
| mmu_piRNA_ | CCAUCACAUCUUUGUCUGGCCUGUA | | | | 900 |
| 133900 | GGUU | | | | |
| mmu_piRNA_ | CACUGACCCUGACUGCUGCUGCACUA | | | | 901 |
| 100897 | | | | | |
| mmu_piRNA_ | AUCCACUAUGUCUGGCCUGUACAGUCU | | | | 902 |
| 77489 | | | | | |
| mmu_piRNA_ | AGAUAGCCAAGGUUGUACUUCCUGCUGCUC | | | | 903 |
| 52259 | CUGGACUUGGU | | | | |
| mmu_piRNA_ | UGGGCUGAGGGAACUGGAGCUGCUUGA | | | | 904 |
| 754565 | | | | | |
| mmu_piRNA_ | UCAUAGUGCUUCCUGGCAGAGAGUGU | | | | 905 |
| 471141 | | | | | |
| mmu_piRNA_ | UAGUGAUUUCCCCCAAGGAACAAAGGC | | | | 906 |
| 413676 | | | | | |
| mmu_piRNA_ | UAGCCGAGGCUGGCCAUGAGCUGUUGA | | | | 907 |
| 384924 | UCCUCCU | | | | |
| mmu_piRNA_ | AGAAGUUGCAACAGAUGUCAGACUAUU | | | | 908 |
| 46361 | GCCU | | | | |

piRNA matching CMV (sense)

| | | | | |
|---|---|---|---|---|
| mmu_piRNA_ | UAGGUCUAUAUAAGCAGCACCAGUUC | C1 | ✓ | 909 |
| 404508 | | | | |
| mmu_piRNA_ | UAACAGAGUGGGGAUUUCCAAGUCU | C2 | | 910 |
| 269354 | | | | |
| mmu_piRNA_ | UGGAACACUACUACUGUCCAUAGAAGA | | | 911 |
| 706802 | CACA | | | |

piRNA matching CMV (antisense)

| | | | | | |
|---|---|---|---|---|---|
| mmu_piRNA_ | UGUUGAUUUUGGUGCCAUUCUGACCU | C3 | ✓ | | 912 |
| 831015 | | | | | |
| mmu_piRNA_ | UGCUUAUAUAGACCUAGGAGUGACGU | C4 | | ✓ | 913 |
| 698594 | | | | | |
| mmu_piRNA_ | UAGAGAUGUUGAUGUACUGCCAACUU | C5 | | | 914 |
| 370701 | | | | | |
| mmu_piRNA_ | GGCUGAGGUUGUACUGCCAAGUGGACC | C6 | | | 915 |
| 221882 | | | | | |
| mmu_piRNA_ | UGGUGGAGACUUGGAUUUAAAAGGCUG | | | | 916 |
| 772346 | CGUCCAGUGUGAUGACCCA | | | | |
| mmu_piRNA_ | UGCUAAGUGAUGUACUGCAAAAACCAG | | | | 917 |
| 682488 | GCAGU | | | | |
| mmu_piRNA_ | UGAUGUACUGGGCAUAUUCUGACUUUC | | ✓ | | 918 |
| 639859 | | | | | |
| mmu_piRNA_ | UGAGACUUGGAAAUCUAGGGAGGUGG | | | ✓ | 919 |
| 594569 | | | | | |
| mmu_piRNA_ | UAUGCUAAGUGAUGUACUGCAAAAACC | | | | 920 |
| 434361 | | | | | |
| mmu_piRNA_ | UAGCUGAUGUACUGCAAAGUGGAAAA | | | | 921 |
| 388128 | | | | | |
| mmu_piRNA_ | UAAGUGAUGUACUGCAAAAACCAGGCA | | | | 922 |
| 297011 | GUUGU | | | | |

piRNA matching GFP (sense)

| | | | | | |
|---|---|---|---|---|---|
| mmu_piRNA_ | UAGCAGAACACCCCCAGGUCUAUGGG | Gp1 | | ✓ | 923 |
| 380304 | | | | | |
| mmu_piRNA_ | ACCAGCAGAACACCCUUGCUGCUCAUU | Gp2 | | ✓ | 924 |
| 33295 | GAAUCCAG | | | | |
| mmu_piRNA_ | UGUUAGAUGACGUCAUCGAGCUGAAGG | | | | 925 |
| 825325 | | | | | |
| mmu_piRNA_ | UGGGCUGUGGUCCUGCUGGAGGGCUGGUU | | | | 926 |
| 754879 | GCUAAAACCAGCACCG | | | | |
| mmu_piRNA_ | UGCUGCUCCCUGACAACCACUAAGUG | | | | 927 |
| 693185 | | | | | |
| mmu_piRNA_ | UGACACAAGCUGGAGUAAUCACUGAG | | | | 928 |
| 571687 | | | | | |
| mmu_piRNA_ | UGAAGCUGCACGACUUCUCCUCUCC | | | | 929 |
| 559111 | | | | | |
| mmu_piRNA_ | UCUCUGAAACCCUGGUGAACCGAGC | | | | 930 |
| 514448 | | | | | |
| mmu_piRNA_ | UCCGACCACAUGAAGGUGCACAGCCA | | ✓ | | 931 |
| 491839 | GGGCC | | | | |
| mmu_piRNA_ | UCAAGUGUGCAGUGCUUCAGUCACAC | | | | 932 |
| 451468 | AGU | | | | |
| mmu_piRNA_ | UACCUGAGCACCCUGUCCCAGGUCAC | | ✓ | | 933 |
| 339834 | AGAAGAC | | | | |
| mmu_piRNA_ | UACAAAGUGUACUGAAGCUGCACGACU | | | | 934 |
| 314751 | UCU | | | | |
| mmu_piRNA_ | GGCCGUGCUGCUGCCCAAGAAGACGG | | | | 935 |
| 221094 | | | | | |
| mmu_piRNA_ | GCUGCCCUGAGCUGGCAGUACCUGAG | | | | 936 |
| 213175 | CACCCUGUCCCAGGUCACAGAAGA | | | | |
| mmu_piRNA_ | GCGUCCUGCCCAACAUCCAGGCCGUG | | | | 937 |
| 211055 | CUGCUGCCCAAGAAGACGGAGAGC | | | | |
| mmu_piRNA_ | GCCGUGCUGCUGCCCAAGAAGACGGAG | | ✓ | | 938 |
| 208986 | | | | | |
| mmu_piRNA_ | CAGUACCUGAGCACCCUGUCCCAGGU | | | | 939 |
| 121534 | CACAGAAGACUA | | | | |
| mmu_piRNA_ | CAGGGCGGCGUCCUGCCCAACAUCCA | | | | 940 |
| 119484 | GGCCGUGCUGCUGCCCAAGAAGAC | | | | |
| mmu_piRNA_ | CAGGCCGUGCUGCUGCCCAAGAAGACG | | | | 941 |
| 118532 | GAGA | | | | |
| mmu_piRNA_ | ACCAUCUGUAACUCCAGCUGAAGGGCA | | | | 942 |
| 33784 | UC | | | | |
| mmu_piRNA_ | AAGUGUACUGAAGCUGCACGACUUCUC | | | | 943 |
| 21558 | | | | | |
| mmu_piRNA_ | AACAUCCAGGCCGUGCUGCUGCCCAA | | | | 944 |
| 8125 | | | | | |
| mmu_piRNA_ | UGGGAUACUUCAAGGAGGACUUCCUG | | | | 945 |
| 748153 | | | | | |
| mmu_piRNA_ | UGCUGAACAUCCUGGGGCAGAGUGCC | | | | 946 |
| 689909 | | | | | |

piRNA matching GFP (antisense)

| | | | | | |
|---|---|---|---|---|---|
| mmu_piRNA_ | UUCUCUGUGAUAACUCCAGCUUGUGCCA | Gp3 | | | 947 |
| 879841 | | | | | |
| mmu_piRNA_ | UGUGCUGCUUCAUGUGUAGAAUUCAG | Gp4 | | ✓ | 948 |
| 811932 | | | | | |
| mmu_piRNA_ | UAACUACUGUUGCUGCUUCAUGUGGCGC | Gp5 | | | 949 |
| 274911 | | | | | |
| mmu_piRNA_ | AGAAACUGCUGAUGAACUUCAGGGUGA | Gp6 | | | 950 |
| 43765 | AGUA | | | | |
| mmu_piRNA_ | ACUGCUGAUGAACUUCAGGGUGAAGUA | Gp7 | | | 951 |
| 40839 | UCU | | | | |
| mmu_piRNA_ | AAGAAACUGCUGAUGAACUUCAGGGUGAA | Gp8 | | | 952 |
| 12778 | | | | | |
| mmu_piRNA_ | UUUCUCAGUGAUUACUCCAGCUUGUGUC | | | | 953 |
| 924759 | | | | | |
| mmu_piRNA_ | UUUCAUCAGUCUUGAAGAAGAUGGAA | | | | 954 |
| 923238 | AUAC | | | | |
| mmu_piRNA_ | UUCAUCAGUCUUGAAGAAGAUGGAAAU | | | | 955 |
| 864464 | ACAG | | | | |
| mmu_piRNA_ | UGAGGGAACGCUGCUUCAUGUGGAGU | | | | 956 |
| 613973 | | | | | |
| mmu_piRNA_ | UGAAGAAGAUGGUGUCUGUCCAAACA | | | | 957 |
| 553666 | | | | | |
| mmu_piRNA_ | UGAACUCCAGCAGGAAGAGGAGAUAG | | ✓ | | 958 |
| 551452 | CUCU | | | | |
| mmu_piRNA_ | UCAAACAGAUGGUGCGCUCCGAGCGA | | | | 959 |
| 447173 | CGAGC | | | | |
| mmu_piRNA_ | UAUCAAACAGAUGGUGCGCUCCGAGCG | | | | 960 |
| 424692 | AC | | | | |
| mmu_piRNA_ | CUGAACUCCAGCAGGAAGAGGAGAUAG | | | | 961 |
| 156778 | | | | | |
| mmu_piRNA_ | CCUUGAAGAAGAUGUUGGUAUUCUUGC | | | | 962 |
| 143602 | | | | | |
| mmu_piRNA_ | CAUCAGUCUUGAAGAAGAUGGAAAUAC | | | | 963 |
| 126275 | AGGA | | | | |
| mmu_piRNA_ | CAGUCUUGAAGAAGAUGGAAAUACAGG | | | | 964 |
| 122971 | AUA | | | | |
| mmu_piRNA_ | CAGGACAGGAACUCCAGCAGGAGCUGA | | | | 965 |
| 116238 | | | | | |
| mmu_piRNA_ | AUCAAACAGAUGGUGCGCUCCGAGCGA | | | | 966 |
| 76329 | CGAGC | | | | |
| mmu_piRNA_ | UAUGAAGAAGAUGGUGGGAAAGAUCU | | | | 967 |
| 430513 | | | | | |
| mmu_piRNA_ | UAGGACUGGGUGCUCACUGCUGAGGC | | | | 968 |
| 394655 | UA | | | | |

piRNA matching AU3 (sense)

| | | | |
|---|---|---|---|
| mmu_piRNA_ | UUUGGCUCUGAAGACAAGAUCUGUAG | U1 | 969 |
| 931333 | | | |
| mmu_piRNA_ | UGAAGACAAGAUCUGUAGCCAUCCC | U2 | 970 |
| 554204 | | | |

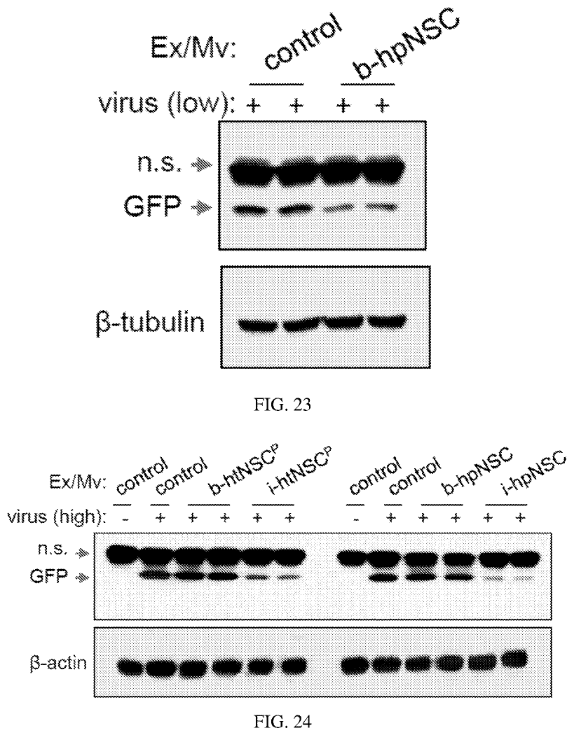

It was then determined if treatment with NSC Ex/Mv could provide a protective effect against lentiviral infection. It was previously experienced that lentiviral infection of mouse brain or mouse primary NSC lines was less efficient compared to other tissue or cell types. A549 cells, a human lung epithelial cell line, was virally infected to reflect lung virus infection. A standard dose of lentivirus was used, lentiviral infection was examined through GFP fluorescence imaging. As shown in FIG. 22, the number of GFP-positive cells clearly reduced due to the treatment of basal NSC Ex/Mv, while MSC Ex/Mv did not have such an effect. Then, experiments were done to analyze the effect of NSC Ex/Mv treatment further quantitatively via western blot. To increase the detection sensitivity, a reduced dose of lentivirus was applied to infect these cells in the presence or absence of basal NSC Ex/Mv. The western blot for lentiviral GFP revealed that this treatment substantially reduced the lentiviral infection (FIG. 23). Subsequently, given that the additional piRNA feature of induced NSC Ex/Mv, basal and induced NSC Ex/Mv were compared for the antiviral effects in A549 cells. To discern the difference, a stronger infection condition was employed by using the double dose of lentivirus compared to the dosage in FIG. 23. Under this condition, basal NSC Ex/Mv failed to suppress viral infection; however, induced NSC Ex/Mv were still effectively antiviral (FIG. 24). Thus, using an HIV-based lentivirus as example of RNA virus, it was effectively shown that murine NSC Ex/Mv have the feature of producing antiviral piRNAs as well as antiviral effects.

### Example 6: Antiviral Innate Immunity of NSC Ex/Mv in Cell-Free Ambient Environment

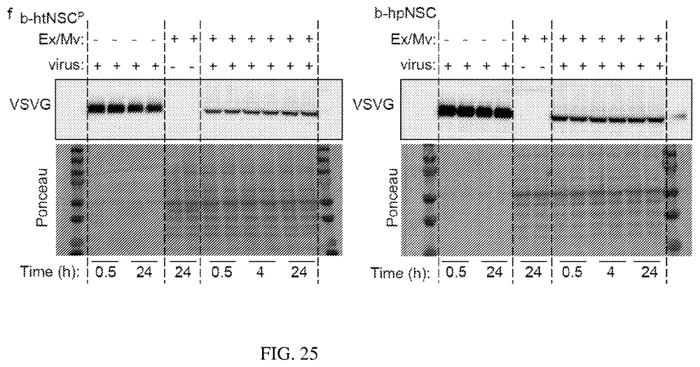

Ex/Mv and RNA viruses are similarly small-sized membranous particles, and both consist of RNA sequences. Because of this close relationship, it provoked the question if NSC Ex/Mv might have an innate immunity-like action to directly interact with viruses in cell-free environment. To test this question, NSC Ex/Mv were directly incubated with VSVG-enveloped lentivirus, and then it was examined how this incubation could affect the virus through western blot for VSVG. Also, to provide a time course profile, several time points including 0.5, 4 and 24 hours were used for Ex/Mv-virus reaction, compared to virus or Ex/Mv alone in the same reaction condition. As shown in FIG. 25, the mixture with basal NSC Ex/Mv whether from the hypothalamus or hippocampus both rapidly led to lentiviral degradation which occurred at 0.5 hour at room temperature since being mixed, but interestingly longer time of incubation although at 4° C. did not further increase viral degradation. In parallel, the induced NSC Ex/Mv from htNSC or hpNSC were observed and both were comparable to basal NSC Ex/Mv in leading to degradation of VSVG-lentivirus. This similarity between basal and induced NSC Ex/Mv in cell-free environment suggested that these NSC Ex/Mv could also provide an innate immunity-like action against viruses. Altogether, besides the potential function by piRNAs which might need to work intracellularly, murine NSC Ex/Mv might provide a cell-free, innate immunity-like mechanism to fight against viruses.

### Example 7: Antiviral Effect of Murine NSC Ex/Mv Against Pseudotyped SARS-CoV-2

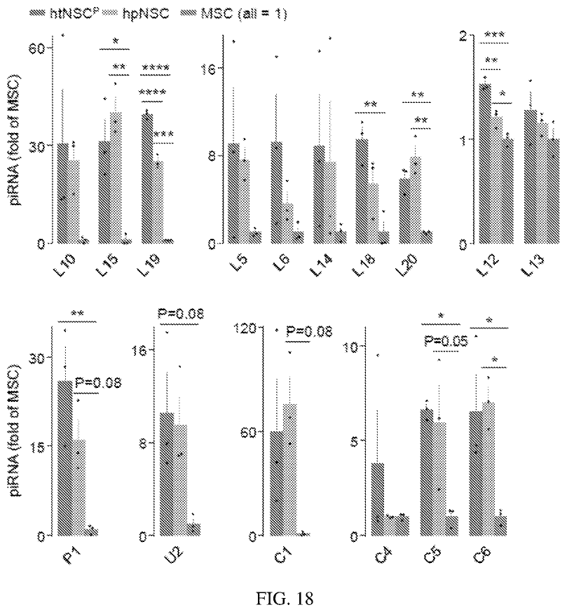

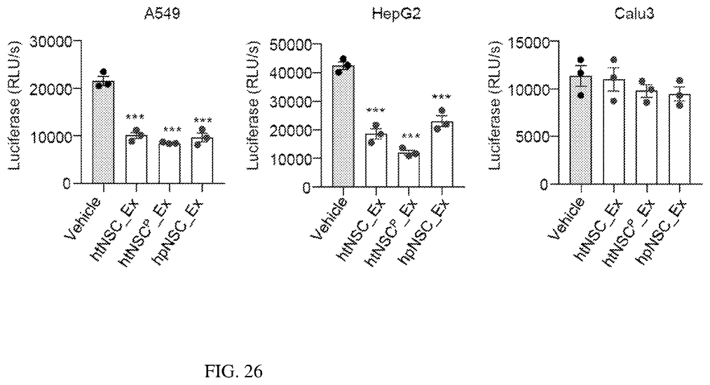

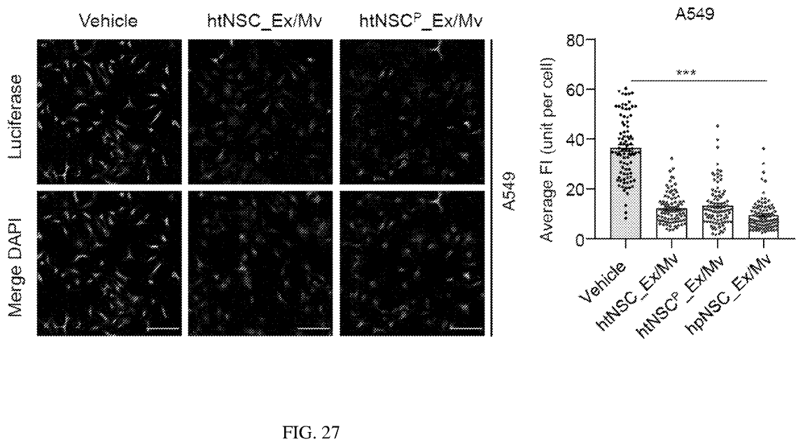

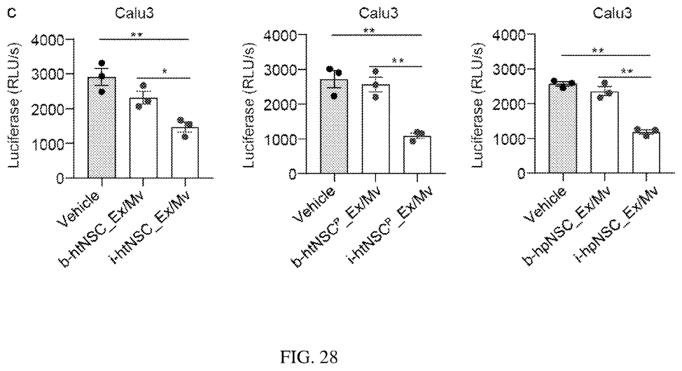

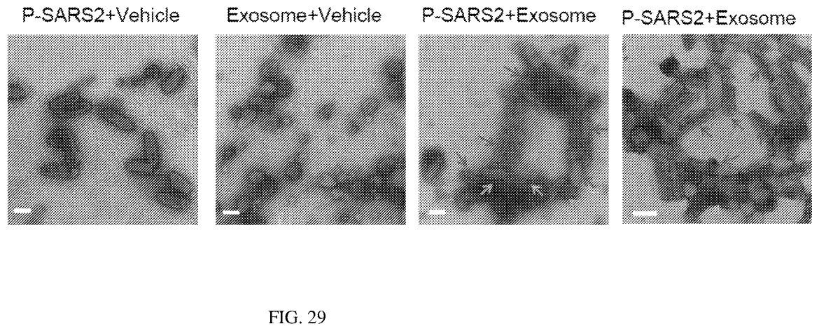

The pseudotyped SARS-CoV-2 in FIG. 4 was used to study the potential effect of murine NSC Ex/Mv against this virus. This pseudotyped SARS-CoV-2 expressed luciferase, and because this recombinant virus is replication-deficient, the viral infection is quantitatively reported by the levels of its luciferase activity. To mimic its infection in various organs, two human respiratory cell models were employed, human alveolar basal epithelial cell A549 and human bronchial epithelial cell Calu3, and human hepatocyte cell model HepG2, all of which express hACE2 and have been used to study SARS-CoV-2. These cells were infected with pseudotyped SARS-CoV-2 virus and in the meanwhile treated with mouse NSC Ex/Mv vs. vehicle control. As shown in FIG. 26, treatment of basal NSC Ex/Mv regardless of NSC types provided a strong antiviral effect in A549 and HepG2. This observation was further verified through immunostaining of luciferase protein levels in these cells (FIG. 27). Of note, compared to A549 and HepG2, Calu3 cells were insensitive to the treatment of basal NSC Ex/Mv (FIG. 26). Based on the observation that inducible NSC Ex/Mv have additional antiviral features, we then focused on treating Calu3 with induced NSC Ex/Mv. To generate these induced NSC Ex/Mv, NSC types, including htNSC^{PGHM}, htNSC and hpNSC, were each made to stably express hACE2, as described in FIG. 18, and exposed to this pseudotyped SARS-CoV-2 for 2 generations, and then maintained under virus-free culture for 5 generations, and finally Ex/Mv that were released by these NSC were isolated and purified for treatment experiments. As shown in FIG. 27, while basal NSC Ex/Mv were ineffective, induced NSC Ex/Mv, regardless of NSC types, were consistently effective in reducing the infection of pseudotyped SARS-CoV-2 in Calu3 cells. Thus, in agreement with the test for HIV-based lentivirus in FIG. 18, the effects of NSC Ex/Mv against VSV-based pseudotyped SARS-CoV-2 in this test further support the antiviral actions of these special extracellular vesicles.

### Example 8: NSC Ex/Mv Combat Against Pseudotyped SARS-CoV-2 in Ambient Environment

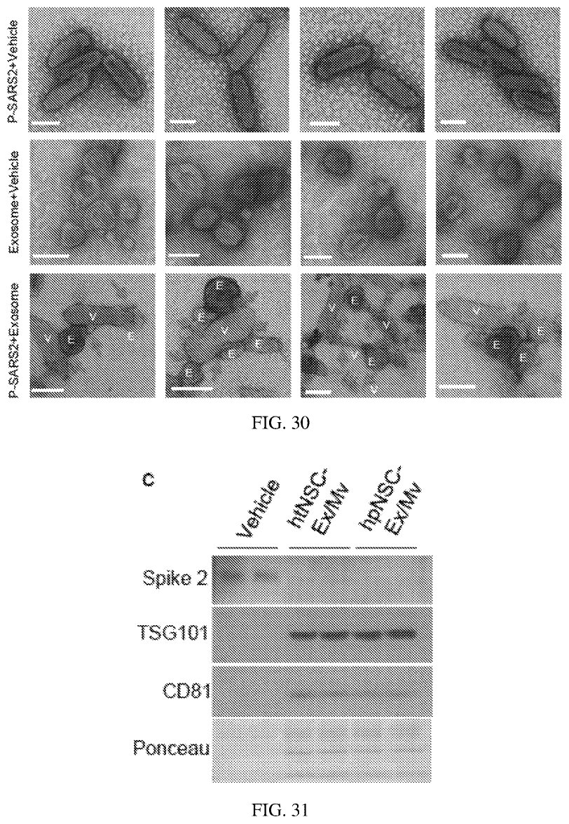

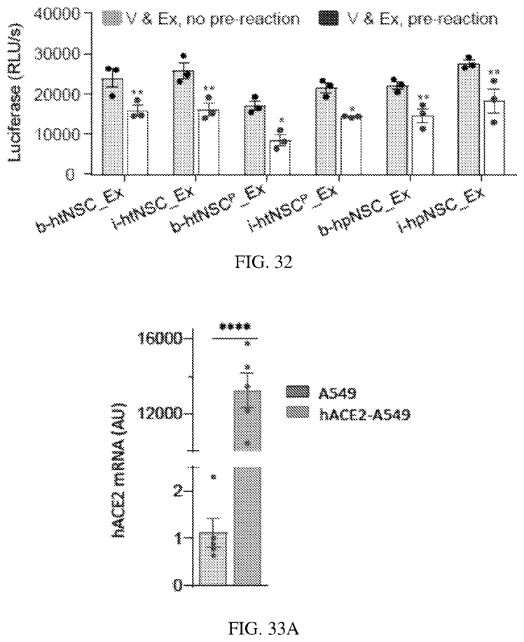

The results in FIG. 24 suggested that NSC Ex/Mv have an ability to break viruses in cell-free ambient environment. It was examined if this observation might also apply to pseudotyped SARS-CoV-2. To test this idea more directly, transmission electron microscopy (TEM) was employed to appreciate any physical interactions between exosomes and viruses. This experiment was designed to focus on exosomes (below 200 nm in diameter) from htNSC, and thus exosomes were purified through a filter and mixed with pseudotyped SARS-CoV-2 in a buffer for 0.5 hour at room temperature and then overnight at 4° C. Exosomes or viruses alone in the same reaction condition were included to provide two technical controls. These samples were then fixed and processed with negative staining and examined under TEM. Clearly, exosomes and viruses had different morphologies distinctly, as viruses were rod-shaped while exosomes were round and cup-shaped. It was observed that while viruses and exosomes usually spread out in the fields, mixture with exosomes morphological evidence suggested that exosomes physically attached, surrounded, and engulfed viruses, and some of these behaviors were apparently associated with breakdown and degradation of viruses (FIG. 30). Independently, experiments in which pseudotyped SARS-CoV-2 viruses were mixed with NSC Ex/Mv versus vehicle in a buffer and then subjected to Western blotting for spike protein S2. The results showed that the mixture with htNSC or hpNSC Ex/Mv both led to degradation of this spike protein (FIG. 31). Functionally, an experiment in which these viruses after being incubated with NSC Ex/Mv or vehicle were used to infect Calu3 cells was performed. As shown in FIG. 32, pre-mixture with NSC Ex/Mv strongly reduced the infection ability of these viruses, regardless of NSC types (FIG. 32). Also, these antiviral effects of NSC Ex/Mv due to the pre-incubation with viruses were comparable between basal and induced NSC particles (FIG. 32). Altogether, NSC Ex/Mv have an innate immunity-like antiviral effect in cell-free environment in addition to the cell-dependent adaptive immunity-like antiviral action.

### Example 9: Antiviral Effects of htNSC Ex/Mv Against SARS-CoV-2 Infection

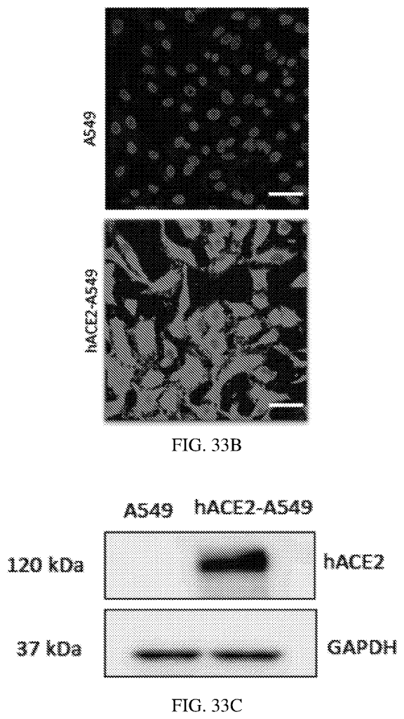

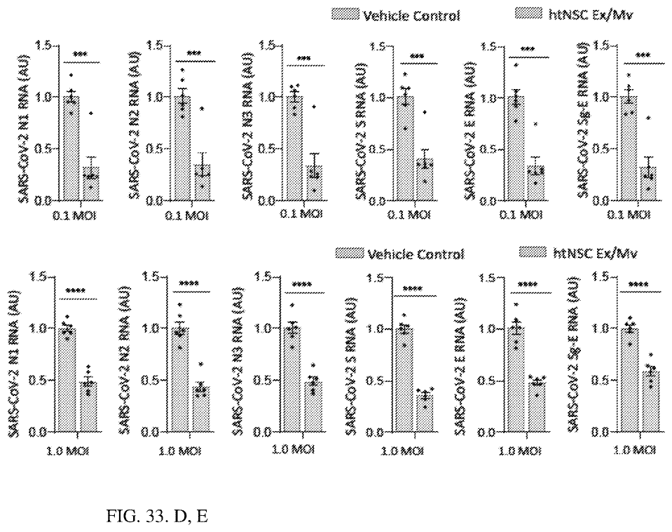

In order to further investigate if htNSC Ex/Mv could inhibit SARS-CoV-2 infection, we employed human alveolar basal epithelial cell line A549, a cell model that has been often used to study SARS-CoV-2 infection. Although A549 cells contain human (hACE2), a receptor that crucially mediates the entry of SARS-CoV-2 into cells, the protein level of hACE2 on the surface of A549 cells is relatively low, and we confirmed that the infection rate of SARS-CoV-2 in A549 cells is modest. Therefore, we optimized this cell model by establishing an A549 cell line which stably overexpressed hACE2, namely hACE2-A549 cells. As shown in FIG. 33A, hACE2 mRNA levels increased about 10,000 folds in hACE2-A549 cells compared to A549 cells. Through immunostaining, we verified that hACE2 was strongly expressed in hACE2-A549 cells while not appreciable in A549 cells (FIG. 33B). We also examined hACE2 protein by Western blot, showing that compared to the strong signal in hACE2-A549 cells, the signal from A549 cells was barely detectable (FIG. 33C).

Viruses of SARS-CoV-2 USA-WA1/2020 were generated and purified in the specially designated BSL-3 facility and quantitated for multiplicity of infection (MOI) according to the method as recently published. MOI 0.1 of SARS-CoV-2 represented a standard dose for its infection, as established in research. Thus, we adopted a loading dose of SARS-CoV-2 virus at MOI 0.1 and 1.0 to indicate a regular and a high dose, respectively. We generated and purified Ex/Mv from cultured mouse htNSC. Cultured hACE2-A549 cells in plates at an appropriate density were added with MOI 0.1 or 1.0 of SARS-CoV-2 USA-WA1/2020 and treated with htNSC Ex/Mv vs. vehicle control. After 2 days of incubation, these hACE2-A549 cells were lysed and analyzed for the quantity of SARS-CoV-2 RNA genomes through quantitative PCR (qPCR). We employed 5 different qPCR conditions to cover 5 different regions of SARS-CoV-2 genomic RNA, including different N, S and E regions which are responsible for encoding nucleocapsid, spike and envelop proteins, respectively. We found that treatment with htNSC Ex/Mv provided a strong effect against SARS-CoV-2 when standard infection dose (0.1 MOI) was tested. When infection dose was increased 10 times (1.0 MOI), the antiviral effect of htNSC Ex/Mv was still highly significant (FIG. 33E), although slightly less pronounced compared to regular dose infection.

Theoretically, the antiviral actions of a treatment could occur through reducing viral entry into cells or inhibiting viral replication in cells or both. We employed a method based on qPCR measurement of sub-genomic E region (Sg-E) which can reflect replication of viruses as established in recent research. As shown in FIG. 33D and E, we found that treatment of htNSC Ex/Mv led to a reduction in the expression levels in Sg-E for both MOI 0.1 and MOI 1.0 infection conditions. Reductions in this sub-genomic sequence were relatively comparable to the reductions in other genomic sequences of the virus, suggesting that htNSC Ex/Mv have a strong effect in suppressing SARS-CoV-2 replication in infected cells.

### Example 10: Reduction of htNSC Ex/Mv piRNAS by PIWIL2 Knockout

Based on piRNA database disposition, the numbers of piRNA species in mammals are large, for example, a mouse could have about 68 million of different piRNA species. In recent research, piRNAs have been related to control of transposon invasion or viral infection. As previously mentioned, we analyzed a mouse piRNAQuest database for piRNA species with sequences that could potentially target SARS-CoV-2 RNA genome, including different segments of SARS-CoV-2 genome, including the sequences that encode spike protein (S), envelope protein (N), membrane protein (M) and nucleocapsid protein (N), open reading frame (Orf) sequences Orf1ab, 3a, 6, 7a, 7b, 8 and 10, untranslated region (UTR) sequences at 5′ end and 3′ end, and gap sequences between some of these segments. This search led to identification of a list of piRNAs (Tables 1-3) some of which possibly target SARS-CoV-2 genome, and we further confirmed that many of these piRNAs were detectable in NSC Ex/Mv.

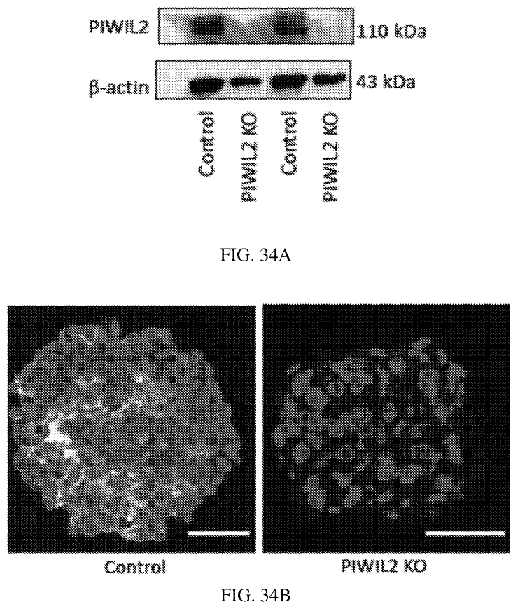

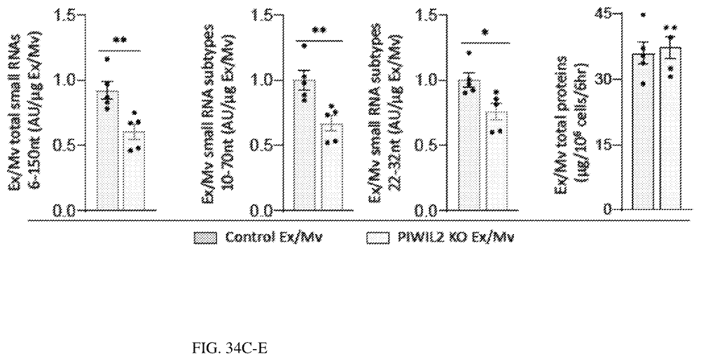

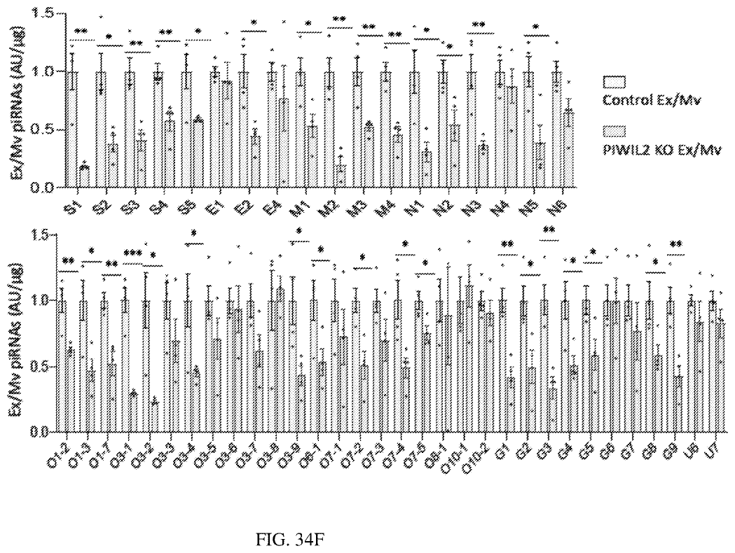

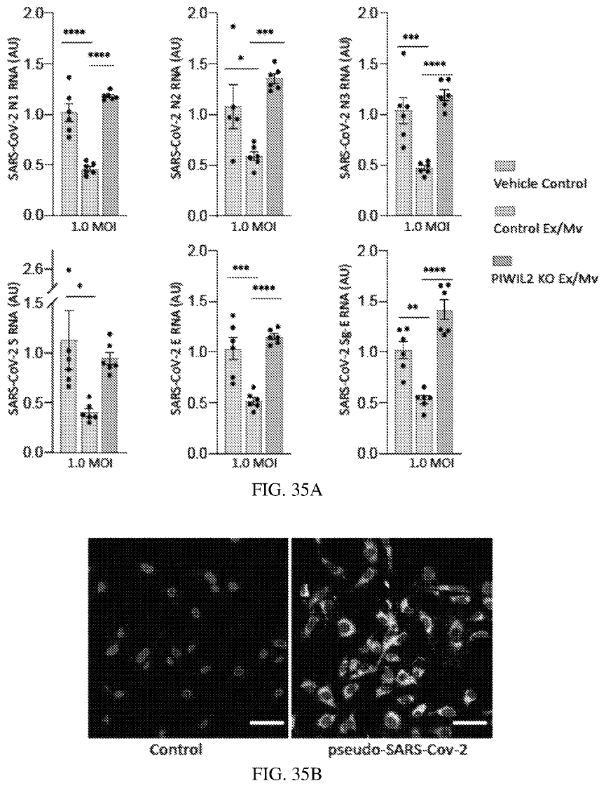

In order to investigate if piRNAs could be important for the antiviral effects of NSC Ex/Mv and because it was not feasible experimentally to directly target individual piRNAs, we developed a strategy by targeting PIWI protein which is specifically required for biogenesis and gene silencing of piRNAs. As mentioned above, our results revealed that PIWI proteins are present in the nervous system, and that PIWIL2 was strongly present in various types of NSCs, including htNSC. Using CRISPR/Cas9 knockout technology, we deleted a genomic sequence for encoding PIWIL2 in htNSC, leading to the establishment of htNSC-PIWIL2 KO cell line. As verified through Western blot in FIG. 34A, PIWIL2 protein was absent in htNSC-PIWIL2 KO, while it was strongly expressed in control htNSC. Immunostaining also confirmed that PIWIL2 protein was absent in the neurospheres of htNSC-PIWIL2 KO, while it was expressed in the neurospheres of control htNSC (FIG. 34B). Then, we collected Ex/Mv from cultured htNSC-PIWIL2 KO vs. control htNSC and isolated Ex/Mv small RNAs for a comparison. As shown in FIG. 34C, PIWIL2 knockout led to about 29% reduction in total small RNAs with size less than 150 bp. This result also suggested that piRNAs represented a significant portion in the total small RNAs of htNSC Ex/Mv. Furthermore, we subjected these small RNAs to bioanalyzer tracing for the size of 10-40 bp, a range that comprised piRNAs and miRNAs. The result showed that there was about 39% reduction in the populations of these small RNAs in Ex/Mv from htNSC-PIWIL2 KO compared to control htNSC (FIG. 34D). We also measured Ex/Mv protein levels and did not find significant difference for Ex/Mv from htNSC-PIWIL2 KO and control htNSC (FIG. 34E). Thus, ablation of PIWIL2 led to a reduction in piRNA content without affecting protein content in htNSC Ex/Mv.

We further analyzed individual piRNAs for their expression levels in Ex/Mv from htNSC-PIWIL2 KO compared to Ex/Mv from control htNSC. As presented above in Tables 1-3, mouse piRNA library has a collection of piRNAs with lead sequences matched against the sense or antisense sequence of SARS-CoV-2 RNA genome. We narrowed down to a list of these piRNAs which are readily detectable in htNSC Ex/Mv, as summarized in Table 4. We found that majority of these piRNAs decreased their expression levels in in Ex/Mv from htNSC-PIWIL2 KO compared to Ex/Mv from control htNSC (FIG. 34F). Hence, we generated htNSC Ex/Mv in which piRNAs were reduced through ablation of PIWIL2 in htNSC.

TABLE 4
piRNA SEQUENCES

| | | | SEQ |
|---|---|---|---|
| | | | ID |
| piRNA ID (piRNAQuest) | piRNA sequence | Label | NO: |
| mmu_piRNA_553635 | UGAAGAAGAGCAAGAAGGAAAGUGAA | O1-2 | 18 |
| mmu_piRNA_443751 | UAUUUAAACUGUCUUAUGUGUCUCCA | O1-3 | 19 |
| mmu_piRNA_923720 | UUUCCAGAGUUGUUGUACCAAUUUCCAAU | O1-7 | 198 |
| mmu_piRNA_555154 | UGAAGACCCAGUCCCUACCUUAGCCUA | S1 | 287 |
| mmu_piRNA_801725 | UGUGAAGGUGUCUUUGUCACUAAUAGAUG | S2 | 288 |
| mmu_piRNA_562740 | UGAAGUCUGCCUGUGAAGUCUGCCUGUGA | S3 | 312 |
| mmu_piRNA_498273 | UCCUGAAGAAGAAUCACAAUCGUUCACAGU | S4 | 313 |
| mmu_piRNA_233057 | GUGAAGUCUGCCUGUGAAGUCUGCCU | S5 | 314 |
| mmu_piRNA_858506 | UUCAAGGCCAGCAGCUACAGAGUGAG | O3-1 | 359 |
| mmu_piRNA_561925 | UGAAGUAACUGUGUAUACUGGGUAUA | O3-2 | 360 |
| mmu_piRNA_443463 | UAUUGUGUGAAUUUGGUUUUGUGGUG | O3-3 | 361 |
| mmu_piRNA_443462 | UAUUGUGUGAAUUUGGUUUUGUCAUU | O3-4 | 362 |
| mmu_piRNA_443460 | UAUUGUGUGAAUUUGGUUUUGUCAGG | O3-5 | 363 |
| mmu_piRNA_88635 | AUUGUGUGAAUUUGGUUUUGUCCUGG | O3-6 | 364 |
| mmu_piRNA_88634 | AUUGUGUGAAUUUGGUUUUGUCAUGG | O3-7 | 365 |
| mmu_piRNA_88633 | AUUGUGUGAAUUUGGUUGUGUCAUGG | O3-8 | 366 |
| mmu_piRNA_86326 | AUGUUCUUCAGGCUCCCCUGCAGGUUUGUUUUUG | O3-9 | 367 |
| mmu_piRNA_104250 | CAGAAGATCAGGAACTAACAGGCAAA | E1 | 381 |
| mmu_piRNA_181390 | GAAGGUUUUACAAGAUAAGGGGCUUC | E2 | 382 |
| mmu_piRNA_466370 | UCAGGACCUCUAGAAGAACAAUCAGU | E4 | 384 |
| mmu_piRNA_419319 | UAGUUUUUCUGUUCAAUGGUUCAUGA | G1 | 393 |
| mmu_piRNA_419318 | UAGUUUUUCUGUUAAGUGAAGAGGGG | G2 | 394 |
| mmu_piRNA_865863 | UUCCAAACAGAAAAUGCAGCUUUCGA | G3 | 397 |
| mmu_piRNA_475430 | UCCAAACAGAAGAACUAGCAAAGCAA | G4 | 398 |
| mmu_piRNA_475429 | UCCAAACAGAAAAGCUUAAAGUUAAG | G5 | 399 |
| mmu_piRNA_700126 | UGCUUCUUUCAGACUUCCCUUCUGUCU | M1 | 400 |
| mmu_piRNA_379382 | UAGCAAUUCCACCGGUGGAAACAGUA | M2 | 408 |
| mmu_piRNA_167046 | CUGUACAAGCAAAGCUCUUGGGAGGU | M3 | 409 |
| mmu_piRNA_142622 | CCUGUAUGCAGCAAAAUGUUGGGUCC | M4 | 410 |
| mmu_piRNA_432244 | UAUGAGGACUUUGAAAGUUGGACUAA | O6-1 | 422 |
| mmu_piRNA_25718 | AAUUUGCUUUUGCUUUAAUCCCAGGU | O7-1 | 430 |
| mmu_piRNA_867226 | UUCCAGAAGAGCCAGGUUCAGUUCCC | O7-2 | 432 |
| mmu_piRNA_320536 | UACACUCUUGGUAGUGGGGAGCCAUGGGAUC | O7-3 | 433 |
| mmu_piRNA_934618 | UUUUAGCCUUUCUGCCGUUCUGACA | G6 | 434 |
| mmu_piRNA_292200 | UAAGGAAUAGCAGAAUGCUUUAAUGC | G7 | 439 |
| mmu_piRNA_934618 | UUUUAGCCUUUCUGCCGUUCUGACA | O7-4 | 442 |
| mmu_piRNA_292200 | UAAGGAAUAGCAGAAUGCUUUAAUGC | O7-5 | 447 |
| mmu_piRNA_658285 | UGCAGCUACAGUUGUGUGCUACUCUC | O8-1 | 454 |
| mmu_piRNA_718264 | UGGACUUCCCUAUGGUCGUGACCUUUCCCGCC | N1 | 458 |
| mmu_piRNA_153125 | CUCCAUGAGCAGUGCUGGGAACAGUAGCAGGAAC | N2 | 459 |
| mmu_piRNA_288153 | UAAGAUGGUAUUUCUAGCUGUUAGGU | N3 | 460 |
| mmu_piRNA_312079 | UAAUUUCCUUGGGUUUGUUUUUGGUC | N4 | 481 |
| mmu_piRNA_657627 | UGCAGCAGAUUUCUUAUUUGGGUUUU | N5 | 482 |
| mmu_piRNA_521705 | UCUGCAGCAGGAAGAGUCUUAUUGUCC | N6 | 483 |
| mmu_piRNA_647225 | UGCAAACCACACAAGGCUUUAUUCCG | G8 | 499 |
| mmu_piRNA_679242 | UGCCUUGUGUGGUGAAGGGUCUGCAC | G9 | 500 |
| mmu_piRNA_865467 | UUCAUUCUGCACAAUGUUAUUCCUGUGAGG | O10-1 | 504 |
| mmu_piRNA_811305 | UGUGCUAUGUAGUUCUGACUGGUGGA | O10-2 | 505 |
| mmu_piRNA_402005 | UAGGGAGAGCUGCCCCUCCAGUUGUCUGU | U6 | 513 |
| mmu_piRNA_43308 | AGAAAAAGUGGUGGCUCUUUUGAAGG | U7 | 517 |

### Example 11: Reduced Effects by piRNA-Impaired htNSC Ex/Mv Against Coronaviruses

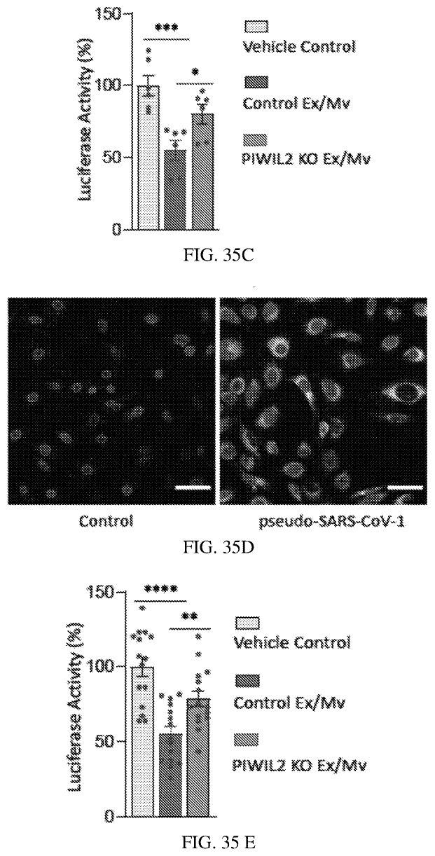

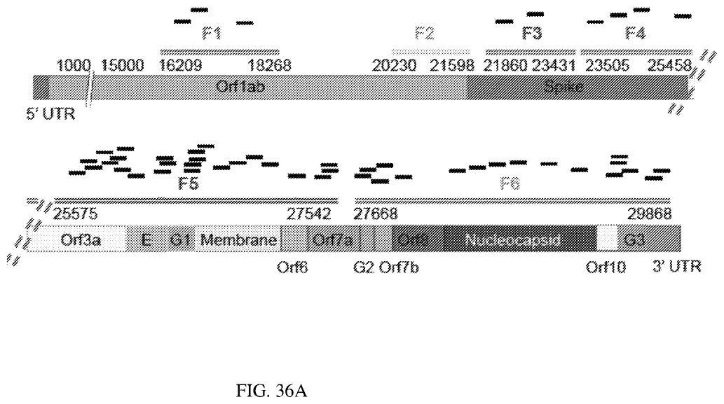

We subsequently examined if htNSC Ex/Mv with reduced piRNAs could still provide a strong effect against SARS-CoV-2. To do so, we employed SARS-CoV-2 USA-WA1/2020, as established in FIG. 33. Cultured hACE2-A549 cells in plates at an appropriate density were infected with MOI 1.0 SARS-CoV-2 and treated with the same amount of Ex/Mv that were derived from htNSC-PIWIL2 KO versus control htNSC. Two days later, hACE2-A549 cells were harvested and lysed for measuring SARS-CoV-2 RNA genomic copies. We found that, while Ex/Mv from control htNSC significantly reduced SARS-CoV-2 infection, the same amount of Ex/Mv from htNSC-PIWIL2 KO failed to provide this antiviral effect (FIG. 35A). We further employed sub-genomic qPCR to evaluate how viral replication could be affected. As shown in FIG. 35A, treatment with Ex/Mv from control htNSC significantly decreased replication of SARS-CoV-2, but treatment with Ex/Mv from htNSC-PIWIL2 KO failed to do so. Thus, the PIWIL2-piRNA system is important for the effect of htNSC Ex/Mv against SARS-CoV-2.

Since PIWI-dependent piRNAs are widely ranged, we predicted that the antiviral mechanism of NSC Ex/Mv potentially through piRNAs should not be limited to SARS-CoV-2 genome. Indeed, our results show that NSC Ex/Mv have antiviral effects against a line of pseudotyped SARS-CoV-2 based on the genome of glycoprotein-deficient vesicular stomatitis virus (ΔG-VSV), and we identified a list of mouse piRNAs with sequences putatively against ΔG-VSV genomic backbone as discussed above. Now given that we developed htNSC-PIWIL2 KO model, we designed to test if the PIWIL2-piRNA system could be necessary for the effect of NSC Ex/Mv against ΔG-VSV-based pseudotyped virus. To be consistent with experiments in this study, we employed hACE2-A549 cell infection model and confirmed that pseudotyped SARS-CoV-2 (10⁵ pfu/10⁶ cells) efficiently infected these cells as reveled by immunostaining for luciferase which was present in ΔG-VSV (FIG. 35B). Using this infection model, we treated these cells with the same amount of Ex/Mv derived from htNSC-PIWIL2 KO versus control htNSC for 3 days. Then, these cells were lysed and measured for luciferase activity which accurately reflected the infection levels. As shown in FIG. 35C, Ex/Mv from control htNSC significantly inhibited the infection, but ablation of PIWIL2 led to a significant reduction in this antiviral effect.

For comparison, we also employed the pseudotyped system to examine SARS-CoV-1, since it employs a different spike protein for infection. As discussed previously, we generated this pseudotyped virus by incorporating SARS-CoV-1 spike protein into ΔG-VSV. Although regular A549 cells were sufficient for infection of pseudotyped SARS-CoV-1, we continued to use hACE2-A549 infection model so that the study could be comparable to other experiments in this work. As shown in FIG. 35D, pseudotyped SARS-CoV-1 showed a strong level of infection of these cells. Therefore, we treated these infected cells (10⁵ pfu/10⁶ cells) with the same amount of Ex/Mv derived from htNSC-PIWIL2 KO versus control htNSC for 3 days. As shown in FIG. 35E, through luciferase activity measurement, htNSC Ex/Mv clearly had a significant antiviral action, but this effect was reduced when PIWIL2 was ablated. Taken together, the PIWIL2-piRNA system is widely important for htNSC Ex/Mv to generate antiviral effects.

### Example 12: Induced htNSC Ex/Mv piRNAs Through Viral RNA Fragment Stimulation

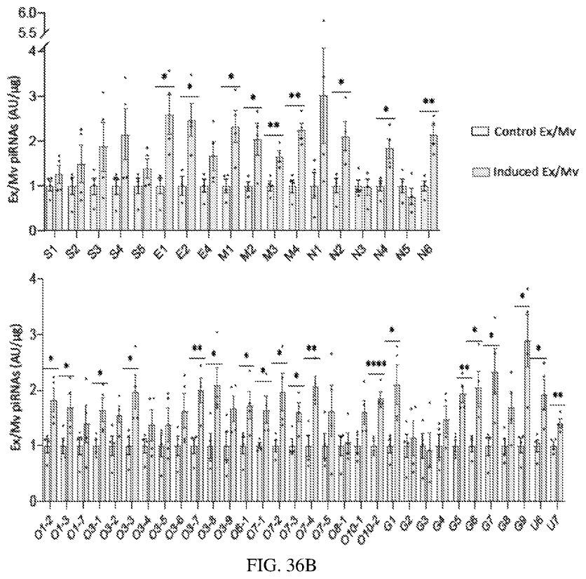

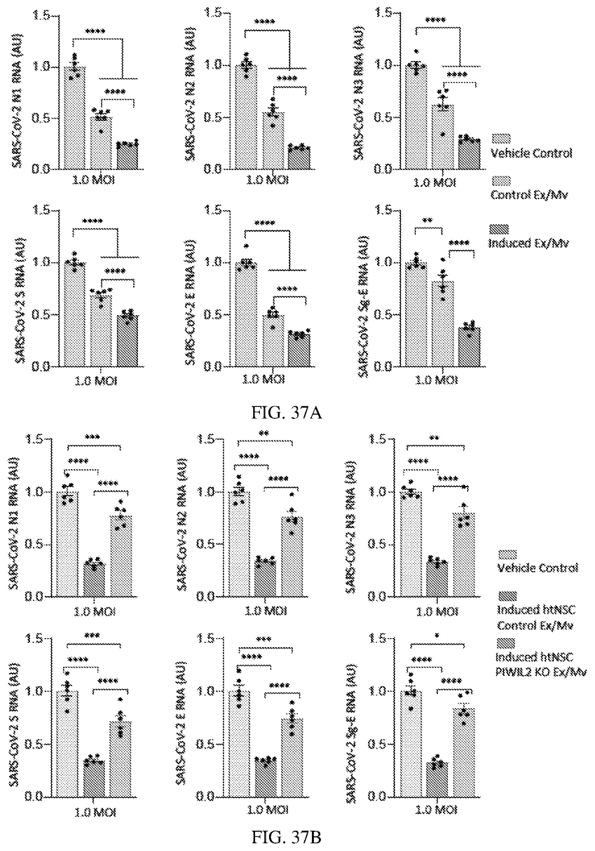

Our results showed that exposure to a pseudotyped virus to mouse NSC led to increased production of Ex/Mv piRNA species with sequences against this viral genome, suggesting that piRNA-based adaptive immunity could exist. In this background, we decided to study if exposure of RNA fragments of SARS-CoV-2 genome to NSC could lead to an enhancement in Ex/Mv piRNAs which matched against this viral genome. As elucidated in FIG. 36A, we designed 6 RNA fragments, namely F1-6 (Table 6), which corresponded to a fragment of encoding sequence for spike (S), membrane (M), nucleocapsid (N) or envelop (E) protein, or a fragment of open reading frame Orf1ab, Orf3a, Orf6, Orf7a, Orf7b and Orf8, 3′ untranslated terminal region (3′ UTR) or gap sequences (G1-3) in SARS-CoV-2 genome. F1-6 were designed to cover all 50 mouse piRNA species in Table 4 and the relative position of these piRNAs are elucidated in FIG. 36A. Using SARS-CoV-2 genome as the template, we produced these RNA fragments through an in vitro transcription system using primers shown in Table 5 below, and purified and quantified these fragments. Subsequently, htNSC cells were exposed to the mixture of F1-6 through transfection, and 2 weeks later, a second exposure of F1-6 was introduced to these cells through transfection. F1-6 RNA fragments did not contain elements for protein translation, so the experiment did not involve protein synthesis from these sequences. Following two exposures with F1-6, these induced htNSC were maintained and expanded in culture for at least 3-4 generations before Ex/Mv were collected from these cells. Small RNAs from Ex/Mv that were released by these induced htNSC versus control htNSC for analyzing piRNAs. Compared to htNSC Ex/Mv, induced htNSC Ex/Mv showed increased levels in many of these piRNAs which corresponded to F1-6 sequences. Among 50 piRNA species in our assay, 28 of them showed significant increases in induced htNSC Ex/Mv compared to htNSC Ex/Mv, and these piRNAs could target various fragments of SARS-CoV-2 RNA genome (FIG. 36B). Of note, among 28 piRNA species which were significantly enhanced, 17 of them matched against the sequences of F1-6 while 11 of them matched against the complementary sequences of F1-6. Thus, RNA stimulation can lead to enhancement of piRNAs with not only antisense but also sense sequences, which were likely processed through different mechanisms. Altogether, SARS-CoV-2 RNA fragments can stimulate htNSC to enhance the production of the potentially antiviral piRNAs and assemble them into Ex/Mv for release.

TABLE 5
PRIMERS FOR GENERATING SARS-COV-2 FRAGMENTS

| Fragment | Primer | Primer sequence | SEQ ID NO: |
|---|---|---|---|
| F1 | Forward | TATGTACACACCGCATACAG | 971 |
| | Reverse | CGCGGGTGATAAACATGTTA | 972 |
| F2 | Forward | ACCCAGGAGTCAAATGGAAA | 973 |
| | Reverse | ACTGACTAGAGACTAGTGGC | 974 |
| F3 | Forward | GAGGCTGGATTTTTGGTACT | 975 |
| | Reverse | CATGAATAGCAACAGGGACT | 976 |
| F4 | Forward | AATAGGGGCTGAACATGTCA | 977 |
| | Reverse | ATCTGAAGGAGTAGCATCCT | 978 |
| F5 | Forward | AAAGAGATGGCAACTAGCAC | 979 |
| | Reverse | AGGACAAGCAAAAGCAAATT | 980 |

TABLE 6
F1-F6 SARS-COV-2 RNA FRAGMENTS

| RNA | | SEQ ID |
|---|---|---|
| Fragment | Sequences | NO: |
| F1 | UAUGUACACACCGCAUACAGUCUUACAGGC | 983 |
| | UGUUGGGGCUUGUGUUCUUUGCAAUUCACA | |
| | GACUUCAUUAAGAUGUGGUGCUUGCAUACG | |
| | UAGACCAUUCUUAUGUUGUAAAUGCUGUUA | |
| | CGACCAUGUCAUAUCAACAUCACAUAAAUU | |
| | AGUCUUGUCUGUUAAUCCGUAUGUUUGCAA | |
| | UGCUCCAGGUUGUGAUGUCACAGAUGUGAC | |
| | UCAACUUUACUUAGGAGGUAUGAGCUAUUA | |
| | UUGUAAAUCACAUAAACCACCCAUUAGUUU | |
| | UCCAUUGUGUGCUAAUGGACAAGUUUUUGG | |
| | UUUAUAUAAAAAUACAUGUGUUGGUAGCGA | |
| | UAAUGUUACUGACUUUAAUGCAAUUGCAAC | |
| | AUGUGACUGGACAAAUGCUGGUGAUUACAU | |
| | UUUAGCUAACACCUGUACUGAAAGACUCAA | |
| | GCUUUUUGCAGCAGAAACGCUCAAAGCUAC | |
| | UGAGGAGACAUUUAAACUGUCUUAUGGUAU | |
| | UGCUACUGUACGUGAAGUGCUGUCUGACAG | |
| | AGAAUUACAUCUUUCAUGGGAAGUUGGUAA | |
| | ACCUAGACCACCACUUAACCGAAAUUAUGU | |
| | CUUUACUGGUUAUCGUGUAACUAAAAACAG | |
| | UAAAGUACAAAUAGGAGAGUACACCUUUGA | |
| | AAAAGGUGACUAUGGUGAUGCUGUUGUUUA | |
| | CCGAGGUACAACAACUUACAAAUUAAAUGU | |
| | UGGUGAUUAUUUUGUGCUGACAUCACAUAC | |
| | AGUAAUGCCAUUAAGUGCACCUACACUAGU | |
| | GCCACAAGAGCACUAUGUUAGAAUUACUGG | |
| | CUUAUACCCAACACUCAAUAUCUCAGAUGA | |
| | GUUUUCUAGCAAUGUUGCAAAUUAUCAAAA | |
| | GGUUGGUAUGCAAAAGUAUUCUACACUCCA | |
| | GGGACCACCUGGUACUGGUAAGAGUCAUUU | |
| | UGCUAUUGGCCUAGCUCUCUACUACCCUUC | |
| | UGCUCGCAUAGUGUAUACAGCUUGCUCUCA | |
| | UGCCGCUGUUGAUGCACUAUGUGAGAAGGC | |
| | AUUAAAAUAUUUGCCUAUAGAUAAAUGUAG | |
| | UAGAAUUAUACCUGCACGUGCUCGUGUAGA | |
| | GUGUUUUGAUAAAUUCAAAGUGAAUUCAAC | |
| | AUUAGAACAGUAUGUCUUUUGUACUGUAAA | |
| | UGCAUUGCCUGAGACGACAGCAGAUAUAGU | |
| | UGUCUUUGAUGAAAUUUCAAUGGCCACAAA | |
| | UUAUGAUUUGAGUGUUGUCAAUGCCAGAUU | |
| | ACGUGCUAAGCACUAUGUGUACAUUGGCGA | |
| | CCCUGCUCAAUUACCUGCACCACGCACAUU | |
| | GCUAACUAAGGGCACACUAGAACCAGAAUA | |
| | UUUCAAUUCAGUGUGUAGACUUAUGAAAAC | |
| | UAUAGGUCCAGACAUGUUCCUCGGAACUUG | |
| | UCGGCGUUGUCCUGCUGAAAUUGUUGACAC | |
| | UGUGAGUGCUUUGGUUUAUGAUAAUAAGCU | |
| | UAAAGCACAUAAAGACAAAUCAGCUCAAUG | |
| | CUUUAAAAUGUUUUAUAAGGGUGUUAUCAC | |
| | GCAUGAUGUUUCAUCUGCAAUUAACAGGCC | |
| | ACAAAUAGGCGUGGUAAGAGAAUUCCUUAC | |
| | ACGUAACCCUGCUUGGAGAAAAGCUGUCUU | |
| | UAUUUCACCUUAUAAUUCACAGAAUGCUGU | |
| | AGCCUCAAAGAUUUUGGGACUACCAACUCA | |
| | AACUGUUGAUUCAUCACAGGGCUCAGAAUA | |
| | UGACUAUGUCAUAUUCACUCAAACCACUGA | |
| | AACAGCUCACUCUUGUAAUGUAAACAGAUU | |
| | UAAUGUUGCUAUUACCAGAGCAAAAGUAGG | |
| | CAUACUUUGCAUAAUGUCUGAUAGAGACCU | |
| | UUAUGACAAGUUGCAAUUUACAAGUCUUGA | |
| | AAUUCCACGUAGGAAUGUGGCAACUUUACA | |
| | AGCUGAAAAUGUAACAGGACUCUUUAAAGA | |
| | UUGUAGUAAGGUAAUCACUGGGUUACAUCC | |
| | UACACAGGCACCUACACACCUCAGUGUUGA | |
| | CACUAAAUUCAAAACUGAAGGUUUAUGUGU | |
| | UGACAUACCUGGCAUACCUAAGGACAUGAC | |
| | CUAUAGAAGACUCAUCUCUAUGAUGGGUUU | |
| | UAAAAUGAAUUAUCAAGUUAAUGGUUACCC | |
| | UAACAUGUUUAUCACCCGCG | |
| F2 | ACCCAGGAGUCAAAUGGAAAUUGAUUUCUU | 984 |
| | AGAAUUAGCUAUGGAUGAAUUCAUUGAACG | |
| | GUAUAAAUUAGAAGGCUAUGCCUUCGAACA | |
| | UAUCGUUUAUGGAGAUUUUAGUCAUAGUCA | |
| | GUUAGGUGGUUUACAUCUACUGAUUGGACU | |
| | AGCUAAACGUUUUAAGGAAUCACCUUUUGA | |
| | AUUAGAAGAUUUUAUUCCUAUGGACAGUAC | |
| | AGUUAAAAACUAUUUCAUAACAGAUGCGCA | |
| | AACAGGUUCAUCUAAGUGUGUGUGUUCUGU | |
| | UAUUGAUUUAUUACUUGAUGAUUUUGUUGA | |
| | AAUAAUAAAAUCCCAAGAUUUAUCUGUAGU | |
| | UUCUAAGGUUGUCAAAGUGACUAUUGACUA | |
| | UACAGAAAUUUCAUUUAUGCUUUGGUGUAA | |
| | AGAUGGCCAUGUAGAAACAUUUUACCCAAA | |
| | AUUACAAUCUAGUCAAGCGUGGCAACCGGG | |
| | UGUUGCUAUGCCUAAUCUUUACAAAAUGCA | |
| | AAGAAUGCUAUUAGAAAAGUGUGACCUUCA | |
| | AAAUUAUGGUGAUAGUGCAACAUUACCUAA | |
| | AGGCAUAAUGAUGAAUGUCGCAAAAUAUAC | |
| | UCAACUGUGUCAAUAUUUAAACACAUUAAC | |
| | AUUAGCUGUACCCUAUAAUAUGAGAGUUAU | |
| | ACAUUUUGGUGCUGGUUCUGAUAAAGGAGU | |
| | UGCACCAGGUACAGCUGUUUUAAGACAGUG | |
| | GUUGCCUACGGGUACGCUGCUUGUCGAUUC | |
| | AGAUCUUAAUGACUUUGUCUCUGAUGCAGA | |
| | UUCAACUUUGAUUGGUGAUUGUGCAACUGU | |
| | ACAUACAGCUAAUAAAUGGGAUCUCAUUAU | |
| | UAGUGAUAUGUACGACCCUAAGACUAAAAA | |
| | UGUUACAAAAGAAAAUGACUCUAAAGAGGG | |
| | UUUUUUCACUUACAUUUGUGGGUUUAUACA | |
| | ACAAAAGCUAGCUCUUGGAGGUUCCGUGGC | |
| | UAUAAAGAUAACAGAACAUUCUUGGAAUGC | |
| | UGAUCUUUAUAAGCUCAUGGGACACUUCGC | |
| | AUGGUGGACAGCCUUUGUUACUAAUGUGAA | |
| | UGCGUCAUCAUCUGAAGCAUUUUUAAUUGG | |
| | AUGUAAUUAUCUUGGCAAACCACGCGAACA | |
| | AAUAGAUGGUUAUGUCAUGCAUGCAAAUUA | |
| | CAUAUUUUGGAGGAAUACAAAUCCAAUUCA | |
| | GUUGUCUUCCUAUUCUUUAUUUGACAUGAG | |
| | UAAAUUUCCCCUUAAAUUAAGGGGUACUGC | |
| | UGUUAUGUCUUUAAAAGAAGGUCAAAUCAA | |
| | UGAUAUGAUUUUAUCUCUUCUUAGUAAAGG | |
| | UAGACUUAUAAUUAGAGAAAACAACAGAGU | |
| | UGUUAUUUCUAGUGAUGUUCUUGUUAACAA | |
| | CUAAAUGUUUGUUUUUCUUGUUUUAUUGCC | |
| | ACUAGUCUCUAGUCAGU | |
| F3 | GAGGCUGGAUUUUUGGUACUACUUUAGAUU | 985 |
| | CGAAGACCCAGUCCCUACUUAUUGUUAAUA | |
| | ACGCUACUAAUGUUGUUAUUAAAGUCUGUG | |
| | AAUUUCAAUUUUGUAAUGAUCCAUUUUUGG | |
| | GUGUUUAUUACCACAAAAACAACAAAAGUU | |
| | GGAUGGAAAGUGAGUUCAGAGUUUAUUCUA | |
| | GUGCGAAUAAUUGCACUUUUGAAUAUGUCU | |
| | CUCAGCCUUUUCUUAUGGACCUUGAAGGAA | |
| | AACAGGGUAAUUUCAAAAAUCUUAGGGAAU | |
| | UUGUGUUUAAGAAUAUUGAUGGUUAUUUUA | |
| | AAAUAUAUUCUAAGCACACGCCUAUUAAUU | |
| | UAGUGCGUGAUCUCCCUCAGGGUUUUUCGG | |
| | CUUUAGAACCAUUGGUAGAUUUGCCAAUAG | |
| | GUAUUAACAUCACUAGGUUUCAAACUUUAC | |
| | UUGCUUUACAUAGAAGUUAUUUGACUCCUG | |
| | GUGAUUCUUCUUCAGGUUGGACAGCUGGUG | |
| | CUGCAGCUUAUUAUGUGGGUUAUCUUCAAC | |
| | CUAGGACUUUUCUAUUAAAAUAUAAUGAAA | |
| | AUGGAACCAUUACAGAUGCUGUAGACUGUG | |
| | CACUUGACCCUCUCUCAGAAACAAAGUGUA | |
| | CGUUGAAAUCCUUCACUGUAGAAAAAGGAA | |
| | UCUAUCAAACUUCUAACUUUAGAGUCCAAC | |
| | CAACAGAAUCUAUUGUUAGAUUUCCUAAUA | |
| | UUACAAACUUGUGCCCUUUUGGUGAAGUUU | |
| | UUAACGCCACCAGAUUUGCAUCUGUUUAUG | |
| | CUUGGAACAGGAAGAGAAUCAGCAACUGUG | |
| | UUGCUGAUUAUUCUGUCCUAUAUAAUUCCG | |
| | CAUCAUUUUCCACUUUUAAGUGUUAUGGAG | |
| | UGUCUCCUACUAAAUUAAAUGAUCUCUGCU | |
| | UUACUAAUGUCUAUGCAGAUUCAUUUGUAA | |
| | UUAGAGGUGAUGAAGUCAGACAAAUCGCUC | |
| | CAGGGCAAACUGGAAAGAUUGCUGAUUAUA | |
| | AUUAUAAAUUACCAGAUGAUUUUACAGGCU | |
| | GCGUUAUAGCUUGGAAUUCUAACAAUCUUG | |
| | AUUCUAAGGUUGGUGGUAAUUAUAAUUACC | |
| | UGUAUAGAUUGUUUAGGAAGUCUAAUCUCA | |
| | AACCUUUUGAGAGAGAUAUUUCAACUGAAA | |
| | UCUAUCAGGCCGGUAGCACACCUUGUAAUG | |
| | GUGUUGAAGGUUUUAAUUGUUACUUUCCUU | |
| | UACAAUCAUAUGGUUUCCAACCCACUAAUG | |
| | GUGUUGGUUACCAACCAUACAGAGUAGUAG | |
| | UACUUUCUUUUGAACUUCUACAUGCACCAG | |
| | CAACUGUUUGUGGACCUAAAAAGUCUACUA | |
| | AUUUGGUUAAAAACAAAUGUGUCAAUUUCA | |
| | ACUUCAAUGGUUUAACAGGCACAGGUGUUC | |
| | UUACUGAGUCUAACAAAAAGUUUCUGCCUU | |
| | UCCAACAAUUUGGCAGAGACAUUGCUGACA | |
| | CUACUGAUGCUGUCCGUGAUCCACAGACAC | |
| | UUGAGAUUCUUGACAUUACACCAUGUUCUU | |
| | UUGGUGGUGUCAGUGUUAUAACACCAGGAA | |
| | CAAAUACUUCUAACCAGGUUGCUGUUCUUU | |
| | AUCAGGAUGUUAACUGCACAGAAGUCCCUG | |
| | UUGCUAUUCAUG | |
| F4 | AAUAGGGGCUGAACAUGUCAACAACUCAUA | 986 |
| | UGAGUGUGACAUACCCAUUGGUGCAGGUAU | |
| | AUGCGCUAGUUAUCAGACUCAGACUAAUUC | |
| | UCCUCGGCGGGCACGUAGUGUAGCUAGUCA | |
| | AUCCAUCAUUGCCUACACUAUGUCACUUGG | |
| | UGCAGAAAAUUCAGUUGCUUACUCUAAUAA | |
| | CUCUAUUGCCAUACCCACAAAUUUUACUAU | |
| | UAGUGUUACCACAGAAAUUCUACCAGUGUC | |
| | UAUGACCAAGACAUCAGUAGAUUGUACAAU | |
| | GUACAUUUGUGGUGAUUCAACUGAAUGCAG | |
| | CAAUCUUUUGUUGCAAUAUGGCAGUUUUUG | |
| | UACACAAUUAAACCGUGCUUUAACUGGAAU | |
| | AGCUGUUGAACAAGACAAAAACACCCAAGA | |
| | AGUUUUUGCACAAGUCAAACAAAUUUACAA | |
| | AACACCACCAAUUAAAGAUUUUGGUGGUUU | |
| | UAAUUUUUCACAAAUAUUACCAGAUCCAUC | |
| | AAAACCAAGCAAGAGGUCAUUUAUUGAAGA | |
| | UCUACUUUUCAACAAAGUGACACUUGCAGA | |
| | UGCUGGCUUCAUCAAACAAUAUGGUGAUUG | |
| | CCUUGGUGAUAUUGCUGCUAGAGACCUCAU | |
| | UUGUGCACAAAAGUUUAACGGCCUUACUGU | |
| | UUUGCCACCUUUGCUCACAGAUGAAAUGAU | |
| | UGCUCAAUACACUUCUGCACUGUUAGCGGG | |
| | UACAAUCACUUCUGGUUGGACCUUUGGUGC | |
| | AGGUGCUGCAUUACAAAUACCAUUUGCUAU | |
| | GCAAAUGGCUUAUAGGUUUAAUGGUAUUGG | |
| | AGUUACACAGAAUGUUCUCUAUGAGAACCA | |
| | AAAAUUGAUUGCCAACCAAUUUAAUAGUGC | |
| | UAUUGGCAAAAUUCAAGACUCACUUUCUUC | |
| | CACAGCAAGUGCACUUGGAAAACUUCAAGA | |
| | UGUGGUCAACCAAAAUGCACAAGCUUUAAA | |
| | CACGCUUGUUAAACAACUUAGCUCCAAUUU | |
| | UGGUGCAAUUUCAAGUGUUUUAAAUGAUAU | |
| | CCUUUCACGUCUUGACAAAGUUGAGGCUGA | |
| | AGUGCAAAUUGAUAGGUUGAUCACAGGCAG | |
| | ACUUCAAAGUUUGCAGACAUAUGUGACUCA | |
| | ACAAUUAAUUAGAGCUGCAGAAAUCAGAGC | |
| | UUCUGCUAAUCUUGCUGCUACUAAAAUGUC | |
| | AGAGUGUGUACUUGGACAAUCAAAAAGAGU | |
| | UGAUUUUUGUGGAAAGGGCUAUCAUCUUAU | |
| | GUCCUUCCCUCAGUCAGCACCUCAUGGUGU | |
| | AGUCUUCUUGCAUGUGACUUAUGUCCCUGC | |
| | ACAAGAAAAGAACUUCACAACUGCUCCUGC | |
| | CAUUUGUCAUGAUGGAAAAGCACACUUUCC | |
| | UCGUGAAGGUGUCUUUGUUUCAAAUGGCAC | |
| | ACACUGGUUUGUAACACAAAGGAAUUUUUA | |
| | UGAACCACAAAUCAUUACUACAGACAACAC | |
| | AUUUGUGUCUGGUAACUGUGAUGUUGUAAU | |
| | AGGAAUUGUCAACAACACAGUUUAUGAUCC | |
| | UUUGCAACCUGAAUUAGACUCAUUCAAGGA | |
| | GGAGUUAGAUAAAUAUUUUAAGAAUCAUAC | |
| | AUCACCAGAUGUUGAUUUAGGUGACAUCUC | |
| | UGGCAUUAAUGCUUCAGUUGUAAACAUUCA | |
| | AAAAGAAAUUGACCGCCUCAAUGAGGUUGC | |
| | CAAGAAUUUAAAUGAAUCUCUCAUCGAUCU | |
| | CCAAGAACUUGGAAAGUAUGAGCAGUAUAU | |
| | AAAAUGGCCAUGGUACAUUUGGCUAGGUUU | |
| | UAUAGCUGGCUUGAUUGCCAUAGUAAUGGU | |
| | GACAAUUAUGCUUUGCUGUAUGACCAGUUG | |
| | CUGUAGUUGUCUCAAGGGCUGUUGUUCUUG | |
| | UGGAUCCUGCUGCAAAUUUGAUGAAGACGA | |
| | CUCUGAGCCAGUGCUCAAAGGAGUCAAAUU | |
| | ACAUUACACAUAAAUGGAUUUGUUUAUGAG | |
| | AAUCUUCACAAUUGGAACUGUAACUUUGAA | |
| | GCAAGGUGAAAUCAAGGAUGCUACUCCUUC | |
| | AGAU | |
| F5 | AAAGAGAUGGCAACUAGCACUCUCCAAGGG | 987 |
| | UGUUCACUUUGUUUGCAACUUGCUGUUGUU | |
| | GUUUGUAACAGUUUACUCACACCUUUUGCU | |
| | CGUUGCUGCUGGCCUUGAAGCCCCUUUUCU | |
| | CUAUCUUUAUGCUUUAGUCUACUUCUUGCA | |
| | GAGUAUAAACUUUGUAAGAAUAAUAAUGAG | |
| | GCUUUGGCUUUGCUGGAAAUGCCGUUCCAA | |
| | AAACCCAUUACUUUAUGAUGCCAACUAUUU | |
| | UCUUUGCUGGCAUACUAAUUGUUACGACUA | |
| | UUGUAUACCUUACAAUAGUGUAACUUCUUC | |
| | AAUUGUCAUUACUUCAGGUGAUGGCACAAC | |
| | AAGUCCUAUUUCUGAACAUGACUACCAGAU | |
| | UGGUGGUUAUACUGAAAAAUGGGAAUCUGG | |
| | AGUAAAAGACUGUGUUGUAUUACACAGUUA | |
| | CUUCACUUCAGACUAUUACCAGCUGUACUC | |
| | AACUCAAUUGAGUACAGACACUGGUGUUGA | |
| | ACAUGUUACCUUCUUCAUCUACAAUAAAAU | |
| | UGUUGAUGAGCCUGAAGAACAUGUCCAAAU | |
| | UCACACAAUCGACGGUUCAUCCGGAGUUGU | |
| | UAAUCCAGUAAUGGAACCAAUUUAUGAUGA | |
| | ACCGACGACGACUACUAGCGUGCCUUUGUA | |
| | AAUGUACUCAUUCGUUUCGGAAGAGACAGG | |
| | UACGUUAAUAGUUAAUAGCGUACUUCUUUU | |
| | UCUUGCUUUCGUGGUAUUCUUGCUAGUUAC | |
| | ACUAGCCAUCCUUACUGCGCUUCGAUUGUG | |
| | UGCGUACUGCUGCAAUAUUGUUAACGUGAG | |
| | UCUUGUAAAACCUUCUUUUUACGUUUACUC | |
| | UCGUGUUAAAAAUCUGAAUUCUUCUAGAGU | |
| | UCCUGAUCUUCUGGUCUAAACGAACUAAAU | |
| | AUUAUAUUAGUUUUUCUGUUUGGAACUUUA | |
| | AUUUUAGCCAUGGCAGAUUCCAACGGUACU | |
| | AUUACCGUUGAAGAGCUUAAAAAGCUCCUU | |
| | GAACAAUGGAACCUAGUAAUAGGUUUCCUA | |
| | UUCCUUACAUGGAUUUGUCUUCUACAAUUU | |
| | GCCUAUGCCAACAGGAAUAGGUUUUUGUAU | |
| | AUAAUUAAGUUAAUUUUCCUCUGGCUGUUA | |
| | UGGCCAGUAACUUUAGCUUGUUUUGUGCUU | |
| | GCUGCUGUUUACAGAAUAAAUUGGAUCACC | |
| | GGUGGAAUUGCUAUCGCAAUGGCUUGUCUU | |
| | GUAGGCUUGAUGUGGCUCAGCUACUUCAUU | |
| | GCUUCUUUCAGACUGUUUGCGCGUACGCGU | |
| | UCCAUGUGGUCAUUCAAUCCAGAAACUAAC | |
| | AUUCUUCUCAACGUGCCACUCCAUGGCACU | |
| | AUUCUGACCAGACCGCUUCUAGAAAGUGAA | |
| | CUCGUAAUCGGAGCUGUGAUCCUUCGUGGA | |
| | CAUCUUCGUAUUGCUGGACACCAUCUAGGA | |
| | CGCUGUGACAUCAAGGACCUGCCUAAAGAA | |
| | AUCACUGUUGCUACAUCACGAACGCUUUCU | |
| | UAUUACAAAUUGGGAGCUUCGCAGCGUGUA | |
| | GCAGGUGACUCAGGUUUUGCUGCAUACAGU | |
| | CGCUACAGGAUUGGCAACUAUAAAUUAAAC | |
| | ACAGACCAUUCCAGUAGCAGUGACAAUAUU | |
| | GCUUUGCUUGUACAGUAAAUGUUUCAUCUC | |
| | GUUGACUUUCAGGUUACUAUAGCAGAGAUA | |
| | UUACUAAUUAUUAUGAGGACUUUUAAAGUU | |
| | UCCAUUUGGAAUCUUGAUUACAUCAUAAAC | |
| | CUCAUAAUUAAAAAUUUAUCUAAGUCACUA | |
| | ACUGAGAAUAAAUAUUCUCAAUUAGAUGAA | |
| | GAGCAACCAAUGGAGAUUGAUUAAAUGAAA | |
| | AUUAUUCUUUUCUUGGCACUGAUAACACUC | |
| | GCUACUUGUGAGCUUUAUCACUACCAAGAG | |
| | UGUGUUAGAGGUACAACAGUACUUUUAAAA | |
| | GAACCUUGCUCUUCUGGAACAUACGAGGGC | |
| | AAUUCACCAUUUCAUCCUCUAGCUGAUAAC | |
| | AAAUUUGCACUGACUUGCUUUAGCACUCAA | |
| | UUUGCUUUUGCUUGUCCU | |
| F6 | AACACUUUGCUUCACACUCAAAAGAAAGAC | 988 |
| | AGAAUGAUUGAACUUUCAUUAAUUGACUUC | |
| | UAUUUGUGCUUUUUAGCCUUUCUGCUAUUC | |
| | CUUGUUUUAAUUAUGCUUAUUAUCUUUUGG | |
| | UUCUCACUUGAACAUGAUUGAACUUUCAUU | |
| | AAUUGACUUCUAUUUGUGCUUUUUAGCCUU | |
| | UCUGCUAUUCCUUGUUUUAAUUAUGCUUAU | |
| | UAUCUUUUGGUUCUCACUUGAACUGCAAGA | |
| | UCAUAAUGAAACUUGUCACGCCUAAAUGAA | |
| | AUUUCUUGUUUUCUUAGGAAUCAUCACAAC | |
| | UGUAGCUGCAUUUCACCAAGAAUGUAGUUU | |
| | ACAGUCAUGUACUCAACAUCAACCAUAUGU | |
| | AGUUGAUGACCCGUGUCCUAUUCACUUCUA | |
| | UUCUAAAUGGUAUAUUAGAGUAGGAGCUAG | |
| | AAAAUCAGCACCUUUAAUUGAAUUGUGCGU | |
| | GGAUGAGGCUGGUUCUAAAUCACCCAUUCA | |
| | GUACAUCGAUAUCGGUAAUUAUACAGUUUC | |
| | CUGUUUACCUUUUACAAUUAAUUGCCAGGA | |
| | ACCUAAAUUGGGUAGUCUUGUAGUGCGUUG | |
| | UUCGUUCUAUGAAGACUUUUUAGAGUAUCA | |
| | UGACGUUCGUGUUGUUUUAGAUUUCAUCUA | |
| | AAUGUCUGAUAAUGGACCCCAAAAUCAGCG | |
| | AAAUGCACCCCGCAUUACGUUUGGUGGACC | |
| | CUCAGAUUCAACUGGCAGUAACCAGAAUGG | |
| | AGAACGCAGUGGGGCGCGAUCAAAACAACG | |
| | UCGGCCCCAAGGUUUACCCAAUAAUACUGC | |
| | GUCUUGGUUCACCGCUCUCACUCAACAUGG | |
| | CAAGGAAGACCUUAAAUUCCCUCGAGGACA | |
| | AGGCGUUCCAAUUAACACCAAUAGCAGUCC | |
| | AGAUGACCAAAUUGGCUACUACCGAAGAGC | |
| | UACCAGACGAAUUCGUGGUGGUGACGGUAA | |
| | AAUGAAAGAUCUCAGUCCAAGAUGGUAUUU | |
| | CUACUACCUAGGAACUGGGCCAGAAGCUGG | |
| | ACUUCCCUAUGGUGCUAACAAAGACGGCAU | |
| | CAUAUGGGUUGCAACUGAGGGAGCCUUGAA | |
| | UACACCAAAAGAUCACAUUGGCACCCGCAA | |
| | UCCUGCUAACAAUGCUGCAAUCGUGCUACA | |
| | ACUUCCUCAAGGAACAACAUUGCCAAAAGG | |
| | CUUCUACGCAGAAGGGAGCAGAGGCGGCAG | |
| | UCAAGCCUCUUCUCGUUCCUCAUCACGUAG | |
| | UCGCAACAGUUCAAGAAAUUCAACUCCAGG | |
| | CAGCAGUAGGGGAACUUCUCCUGCUAGAAU | |
| | GGCUGGCAAUGGCGGUGAUGCUGCUCUUGC | |
| | UUUGCUGCUGCUUGACAGAUUGAACCAGCU | |
| | UGAGAGCAAAAUGUCUGGUAAAGGCCAACA | |
| | ACAACAAGGCCAAACUGUCACUAAGAAAUC | |
| | UGCUGCUGAGGCUUCUAAGAAGCCUCGGCA | |
| | AAAACGUACUGCCACUAAAGCAUACAAUGU | |
| | AACACAAGCUUUCGGCAGACGUGGUCCAGA | |
| | ACAAACCCAAGGAAAUUUUGGGGACCAGGA | |
| | ACUAAUCAGACAAGGAACUGAUUACAAACA | |
| | UUGGCCGCAAAUUGCACAAUUUGCCCCCAG | |
| | CGCUUCAGCGUUCUUCGGAAUGUCGCGCAU | |
| | UGGCAUGGAAGUCACACCUUCGGGAACGUG | |
| | GUUGACCUACACAGGUGCCAUCAAAUUGGA | |
| | UGACAAAGAUCCAAAUUUCAAAGAUCAAGU | |
| | CAUUUUGCUGAAUAAGCAUAUUGACGCAUA | |
| | CAAAACAUUCCCACCAACAGAGCCUAAAAA | |
| | GGACAAAAAGAAGAAGGCUGAUGAAACUCA | |
| | AGCCUUACCGCAGAGACAGAAGAAACAGCA | |
| | AACUGUGACUCUUCUUCCUGCUGCAGAUUU | |
| | GGAUGAUUUCUCCAAACAAUUGCAACAAUC | |
| | CAUGAGCAGUGCUGACUCAACUCAGGCCUA | |
| | AACUCAUGCAGACCACACAAGGCAGAUGGG | |
| | CUAUAUAAACGUUUUCGCUUUUCCGUUUAC | |
| | GAUAUAUAGUCUACUCUUGUGCAGAAUGAA | |
| | UUCUCGUAACUACAUAGCACAAGUAGAUGU | |
| | AGUUAACUUUAAUCUCACAUAGCAAUCUUU | |
| | AAUCAGUGUGUAACAUUAGGGAGGACUUGA | |
| | AAGAGCCACCACAUUUUCACCGAGGCCACG | |
| | CGGAGUACGAUCGAGUGUACAGUGAACAAU | |
| | GCUAGGGAGAGCUGCCUAUAUGGAAGAGCC | |
| | CUAAUGUGUAAAAUUAAUUUUAGUAGUGCU | |
| | AUCCCCAUGUG | |

### Example 13: Enhanced Antiviral Action of Induced htNSC Ex/Mv for SARS-CoV-2

Given that viral RNA fragment-stimulated htNSC lead to Ex/Mv with higher levels of piRNAs which putatively target SARS-CoV-2, we then studied if this induction could help the antiviral action of htNSC Ex/Mv. Cultured hACE2-A549 cells in plates at an appropriate density were infected with SARS-CoV-2 at the dose of MOI 1.0. These cells were treated with induced htNSC Ex/Mv versus control htNSC Ex/Mv, while vehicle treatment was included to provide as a positive control. After 2-day infection and treatment, we collected these cells for determining the harvested viruses by qPCR measurement of viral genomic copies. We found that while treatment of htNSC Ex/Mv led to a significant effect against SARS-CoV-2, this antiviral effect was further significantly enhanced through treatment with induced htNSC Ex/Mv (FIG. 37A). Sub-genomic qPCR analysis further revealed that compared to control htNSC Ex/Mv, induced htNSC Ex/Mv showed a significantly stronger effect to suppress SARS-CoV-2 replication in infected cells (FIG. 37A). To summarize, htNSC can be induced through viral RNA fragment stimulation to generate Ex/Mv with increased piRNAs for enhanced effects against SARS-CoV-2.

### Example 14: Antiviral Action of Induced htNSC Ex/Mv Requires PIWI-piRNA System

Finally, we studied if the PIWI-piRNA system could be important for the enhanced antiviral effect of induced htNSC Ex/Mv. To do so, htNSC-PIWIL2 KO versus control htNSC were twice exposed to viral RNA fragments F1-6 for stimulation, as similarly described in FIG. 36. Then, Ex/Mv were isolated and purified from htNSC-PIWIL2 KO versus control htNSC to treat hACE2-A549 cells upon infection of MOI 1.0 SARS-CoV-2. After 2 days of incubation, these cells were harvested, lysed and analyzed for genomic copies of SARS-CoV-2. As shown in FIG. 37B, while induced htNSC Ex/Mv showed an enhanced antiviral effect as similarly observed in FIG. 37A, ablation of PIWIL2 led to abrogation of not only the enhanced effect but also the basal effect of Ex/Mv against SARS-CoV-2. Sub-genomic qPCR assay further showed that replication of SARS-CoV-2 in infected hACE2-A549 cells was greatly suppressed by induced htNSC Ex/Mv but only weakly by induced htNSC-PIWIL2 KO Ex/Mv (FIG. 37B). Taken together, the PIWIL2-piRNA system is important for not only the basal antiviral effect but also RNA-stimulated antiviral effect of htNSC Ex/Mv.

### Discussion

Murine NSC Ex/Mv abundantly produce piRNAs that can target viral RNAs including SARS-CoV-2 virus, and while some of these exosomal piRNAs can be further enhanced through an initial viral exposure, treatment of these NSC Ex/Mv provided strong antiviral effects through testing a recombinant lentivirus as well as a pseudotyped SARS-CoV-2 virus. Based on these findings, the role of piRNAs could be much expanded from the best appreciated functions of them in regulating germ cell development and preventing against transposon invasion. It is clear that NSC are special in that they have much stronger abilities to produce and release Ex/Mv, compared to other cells including non-neural stem cells such as MSC. In this work, it was shown that these NSC Ex/Mv contain piRNA species not only abundantly but with a huge collection of diverse species. Without being held to theory, it is believed that this level of diversity might not entirely come from a single cell but could be related to the heterogeneity of individual cells in NSC population, which remains to be studied. The antiviral effects observed in this study lend support to an interventional strategy, which can complement vaccine development, since the latter is challenged by the hurdle of targeting an RNA virus, partially because variants and mutations of RNA viruses are high, for instance, systematic-level mutational and protein profile analyses revealed a large number of amino acid substitutions in SARS-CoV-2 indicating that the viral proteins are heterogeneous. In clinical studies, largely due to the limited access of human NSCs, the option of using human MSC has been considered for exosomal therapeutics, and recent studies of using MSC or their derived exosomes to target COVID19 showed positive outcomes; on the other hand, there is a great lack of understandings regarding the therapeutic potentials of NSC exosomes. In this work, it was demonstrated that NSC Ex/Mv have both the abundance and diversity of antiviral piRNAs, which are absent or weak in MSC Ex/Mv. In addition, the abilities of NSC in producing exosomal small RNAs are stronger compared to MSC, which further supports that NSCs are important for exosomal therapeutics. The application of NSC Ex/Mv will increase the width and effectiveness of exosomal therapeutics. Because murine NSC Ex/Mv can fight against pseudotyped SARS-CoV-2 in an ambient environment, despite the cross-species issues, murine NSC Ex/Mv could be used for humans perhaps such as in a nasal spray to help aggregate and break SARS-CoV-2, which currently represents an urgent need.

Additionally, we demonstrated that murine piRNAs-containing Ex/Mv from htNSC can target SARS-CoV-2 through innate immunity, and the antiviral effects of these extracellular vesicles can be induced to increase through an adaptive immunity-like mechanism following twice exposure to viral RNA fragments. Further, our loss-of-function experiments by targeting PIWIL2 revealed that the PIWI-piRNA system is required for both innate and induced antiviral actions of htNSC Ex/Mv. Overall, despite that this study was based on in vitro infection models, this work is significant and informative, as it provides a novel working model and direction for exploring new options to combat SARS-CoV-2.

Murine NSCs have great capabilities of making Ex/Mv that are enriched with small RNAs. Our results indicate that targeted pandemic SARS-CoV-2 showed a strong treatment effect by htNSC Ex/Mv against this virus. Thus, based on an infectious wildtype virus, our results provide another support to the notion that the immunity of NSC Ex/Mv against viruses is important for protecting the brain from viral infection, especially considering that compared to peripheral tissues, the brain is in general separated from peripheral immune system particularly due to the blood-brain barrier. However, although our study analyzed only htNSC, we speculate that some peripheral cells can release Ex/Mv with similar antiviral actions and piRNA properties. To identify and study these peripheral cells should be very valuable for developing Ex/Mv-based antiviral options.

In this work, we demonstrated results showing viral RNA fragments can stimulate NSC to induce Ex/Mv with increased expression levels of piRNAs which could potentially target these RNA sequences. The success of this induction indicates that RNA vaccine strategy can be extended to in vitro model, but notably viral RNA fragments in our experiment did not involve protein translation, and our in vitro model did not involve classical adaptive immune cells such as lymphocytes. Currently, RNA vaccines have been developed to effectively combat SARS-CoV-2 pandemic, and the antiviral effects of RNA vaccines are believed to rely on the production of neuralization antibody against SARS-CoV-2 spike protein. The findings in our study would call for an investigation regarding if piRNA-dependent immunity could be induced by these vaccines. If so, it would be interesting to discern the contributions from induced neutralization antibody versus induced piRNAs.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of treating an individual in need of treatment or prevention of infection with a virus, comprising
administering to the individual a therapeutically effective amount of extracellular vesicles released from neural stem cells and/or neural progenitor cells.

2. The method of claim 1, wherein the extracellular vesicles are isolated from mouse, human, rat, hamster, guinea pig, rabbit, pig, goat, cow, dog, cat, non-human primates, and combinations thereof.

3. The method of claim 1, wherein the neural cell vesicles comprise exosomes, microvesicles, or a combination thereof.

4. The method of claim 1, wherein the neural stem cells and/or neural progenitor cells originate from the hypothalamus or hippocampus.

5. The method of claim 1, wherein administering is parenteral, intravenous, subcutaneous, intranasal, oral, pulmonary, ocular, vaginal, rectal, or intrathecal.

6. The method of claim 1, wherein the virus is-_of the family Arbovirus, Arenaviridae, Arterivirus, Astroviridae, Birnaviridae, Bromoviridae, Bunyaviridae, Caliciviridae, Circoviridae, Closteroviridae, Comoviridae, Coronaviridae, Cystoviridae, Filoviridae, Flaviviridae, Flexiviridae, Hepadnaviridae, Hepevirus, Herpesviridae, Leviviridae, Luteoviridae, Mesoniviridae, Mononegavirales, Mosaic Viruses, Nidovirales, Nodaviridae, Orthomyxoviridae, Papillomaviridae, Papovaviridae, Parvoviridae, Paramyxoviridae, Picobirnaviridae, Picobirnavirus, Picornaviridae, Potyviridae, Poxviridae, Reoviridae, Retroviridae, Roniviridae, Sequiviridae, Tenuivirus, Togaviridae, Tombusviridae, Totiviridae, Tymoviridae; or Alfuy virus, Banzi virus, bovine diarrhea virus, Chikungunya virus, Dengue virus (DNV), Epstein Barr Virus (EBV), Hepatitis B virus (HBV), Hepatitis C virus (HCV), herpes simplex virus type 1 (HSV-1), herpes simplex virus type 2 (HSV-2), human cytomegalovirus (hCMV), human immunodeficiency virus (HIV), Ilheus virus, influenza virus (including avian and swine isolates), rhinovirus, norovirus, adenovirus, Japanese encephalitis virus, Kaposi's sarcoma associated herpesvirus (KSHV), Kokobera virus, Kunjin virus, Kyasanur forest disease virus, louping-ill virus, measles virus, MERS-coronavirus (MERS), metapneumovirus, any of the Mosaic Viruses, Murray Valley virus, parainfluenza virus, poliovirus, Powassan virus, respiratory syncytial virus (RSV), Rocio virus, SARS-coronavirus (SARS), St. Louis encephalitis virus, tick-borne encephalitis virus, West Nile virus (WNV), Ebola virus, Nipah virus, Lassa virus, Tacaribe virus, Junin virus, yellow fever virus, Varicella zoster virus (VZV), or vesicular stomatitis virus (VSV).

7. (canceled)

8. The method of claim 1, wherein the virus is HIV, VSV, or SARS-CoV-2.

9. The method of claim 1, wherein the extracellular vesicles comprise piRNAs, wherein the piRNAs have a perfect match with a sense or antisense sequence of the genome of the virus in a primary lead sequence at positions 2-8.

10. The method of claim 9,
i) wherein the virus is HIV, and wherein the piRNA sequences are homologous with a sense or antisense sequence of long-terminal repeat (LTR), Rev response element (RRE), psi, U3, or a combination thereof, and wherein the piRNAs comprise any of SEQ ID NOs: 816-970; or
ii) wherein the virus is VSV, and wherein the piRNA sequences are homologous with a sense or antisense sequence of the RNA sequences encoding nucleocapsid protein (N sequence), phosphoprotein (P sequence), matrix protein (M sequence), RNA polymerase (R sequence), a non-encoding structural sequence, or a combination thereof, and wherein the piRNAs comprise any of SEQ ID NOs: 522-815.

11. (canceled)

12. (canceled)

13. (canceled)

14. The method of claim 9, wherein the viral infection is caused by SARS-CoV-2, and wherein the piRNA sequences are homologous with a sense or antisense sequence of the spike protein, envelope protein, membrane protein, nucleocapsid protein, open reading frame sequence 1ab (Orf1ab), Orf 3a, Orf 6, Orf 7a, Orf 7b, Orf 8, Orf 10, 5′ end untranslated region (UTR) sequence, 3′ end UTR sequence, a non-encoding sequences, or a combination thereof, wherein the piRNAs comprise any of SEQ ID NOs: 1-521.

15. (canceled)

16. The method of claim 9, wherein the piRNAs have a perfect match with a sense or antisense sequence of the genome of the virus in a primary lead sequence at positions 2-11, and no more than 5 mismatches in a secondary lead sequence at nucleotides 12-21.

17. A method of treating an individual in need of treatment or prevention of infection with a virus, comprising
administering to the individual a therapeutically effective amount of a composition comprising one or more piRNAs, wherein the piRNAs have a perfect match with a sense or antisense sequence of the genome of the virus in a primary lead sequence at positions 2-8,
wherein the one or more piRNAs comprises a population of 1, 5, 10, 20, 50, 100, 200, 500, 1000 or more distinct piRNA sequences,
wherein the composition comprising piRNAs is in the form of polymeric particles, liposomes, micelles, lipid-polymer particles, dendrimers, inorganic nanoparticles, hydrogels, or a combination thereof,
wherein administering is parenteral, intravenous, subcutaneous, oral, intranasal, pulmonary, ocular, vaginal, rectal, or intrathecal.

18. (canceled)

19. (canceled)

20. (canceled)

21. The method of claim 17, wherein the virus is HIV, and wherein the piRNA sequences are homologous with a sense or antisense sequence of long-terminal repeat (LTR), Rev response element (RRE), psi, U3, or a combination thereof, wherein the piRNAs comprise any of SEQ ID NOs: 816-970.

22. (canceled)

23. The method of claim 17, wherein the virus is VSV, and wherein the piRNA sequences are homologous with a sense or antisense sequence of the RNA sequences encoding nucleocapsid protein (N sequence), phosphoprotein (P sequence), matrix protein (M sequence), RNA polymerase (R sequence), a non-encoding structural sequence, or a combination thereof, wherein the piRNAs comprise any of SEQ ID NOs: 522-815.

24. (canceled)

25. The method of claim 17, wherein the virus is SARS-CoV-2, and wherein the piRNA sequences are homologous with a sense or antisense sequence of the spike protein, envelope protein, membrane protein, nucleocapsid protein, open reading frame sequence 1ab (Orf1ab), Orf 3a, Orf 6, Orf 7a, Orf 7b, Orf 8, Orf 10, 5′ end untranslated region (UTR) sequence, 3′ end UTR sequence, a non-encoding sequence, or a combination thereof, wherein the piRNAs comprise any of SEQ ID NOs: 1-521.

26. (canceled)

27. The method of claim 17, wherein the piRNAs have a perfect match with a sense or antisense sequence of the genome of the RNA virus in a primary lead sequence at positions 2-11, and no more than 5 mismatches in a secondary lead sequence at nucleotides 12-21, wherein the virus is of the family Arbovirus, Arenaviridae, Arterivirus, Astroviridae, Birnaviridae, Bromoviridae, Bunyaviridae, Caliciviridae, Circoviridae, Closteroviridae, Comoviridae, Coronaviridae, Cystoviridae, Filoviridae, Flaviviridae, Flexiviridae, Hepadnaviridae, Hepevirus, Herpesviridae, Leviviridae, Luteoviridae, Mesoniviridae, Mononegavirales, Mosaic Viruses, Nidovirales, Nodaviridae, Orthomyxoviridae, Papillomaviridae, Papovaviridae, Parvoviridae, Paramyxoviridae, Picobirnaviridae, Picobirnavirus, Picornaviridae, Potyviridae, Poxviridae, Reoviridae, Retroviridae, Roniviridae, Sequiviridae, Tenuivirus, Togaviridae, Tombusviridae, Totiviridae, Tymoviridae, or Alfuy virus, Banzi virus, bovine diarrhea virus, Chikungunya virus, Dengue virus (DNV), Epstein Barr Virus (EBV), Hepatitis B virus (HBV), Hepatitis C virus (HCV), herpes simplex virus type 1 (HSV-1), herpes simplex virus type 2 (HSV-2), human cytomegalovirus (hCMV), human immunodeficiency virus (HIV), Ilheus virus, influenza virus (including avian and swine isolates), rhinovirus, norovirus, adenovirus, Japanese encephalitis virus, Kaposi's sarcoma associated herpesvirus (KSHV), Kokobera virus, Kunjin virus, Kyasanur forest disease virus, louping-ill virus, measles virus, MERS-coronavirus (MERS), metapneumovirus, any of the Mosaic Viruses, Murray Valley virus, parainfluenza virus, poliovirus, Powassan virus, respiratory syncytial virus (RSV), Rocio virus, SARS-coronavirus (SARS), St. Louis encephalitis virus, tick-borne encephalitis virus, West Nile virus (WNV), Ebola virus, Nipah virus, Lassa virus, Tacaribe virus, Junin virus, yellow fever virus, Varicella zoster virus (VZV), or vesicular stomatitis virus.

28. (canceled)

29. (canceled)

30. A composition comprising a polymeric particle, liposome, micelle, lipid-polymer particle, dendrimer, inorganic nanoparticle, hydrogel, or a combination thereof, and a population of piRNAs, wherein the population of piRNAs have a perfect match with a sense or antisense sequence of the genome of a virus in a primary lead sequence at positions 2-8.

31. The composition of claim 30, wherein the virus is HIV, and wherein the piRNA sequences are homologous with a sense or antisense sequence of long-terminal repeat (LTR), Rev response element (RRE), psi, U3, or a combination thereof, wherein the piRNAs comprise any of SEQ ID NOs: 816-970.

32. (canceled)

33. The composition of claim 30, wherein the virus is SARS-CoV-2, and wherein the piRNA sequences are homologous with a sense or antisense sequence of the spike protein, envelope protein, membrane protein, nucleocapsid protein, open reading frame sequence 1ab (Orf1ab), Orf 3a, Orf 6, Orf 7a, Orf 7b, Orf 8, Orf 10, 5′ end untranslated region (UTR) sequence, 3′ end UTR sequence, a non-encoding sequence, or a combination thereof wherein the piRNAs comprise any of SEQ ID NOs: 1-815.

34. (canceled)

35. The composition of claim 30, wherein the piRNAs have a perfect match with a sense or antisense sequence of the genome of the RNA virus in a primary lead sequence at positions 2-11, and no more than 5 mismatches in a secondary lead sequence at nucleotides 12-21,
wherein the virus is of the family Arbovirus, Arenaviridae, Arterivirus, Astroviridae, Birnaviridae, Bromoviridae, Bunyaviridae, Caliciviridae, Circoviridae, Closteroviridae, Comoviridae, Coronaviridae, Cystoviridae, Filoviridae, Flaviviridae, Flexiviridae, Hepadnaviridae, Hepevirus, Herpesviridae, Leviviridae, Luteoviridae, Mesoniviridae, Mononegavirales, Mosaic Viruses, Nidovirales, Nodaviridae, Orthomyxoviridae, Papillomaviridae, Papovaviridae, Parvoviridae, Paramyxoviridae, Picobirnaviridae, Picobirnavirus, Picornaviridae, Potyviridae, Poxviridae, Reoviridae, Retroviridae, Roniviridae, Sequiviridae, Tenuivirus, Togaviridae, Tombusviridae, Totiviridae, Tymoviridae; or a Alfuy virus, Banzi virus, bovine diarrhea virus, Chikungunya virus, Dengue virus (DNV), Epstein Barr Virus (EBV), Hepatitis B virus (HBV), Hepatitis C virus (HCV), herpes simplex virus type 1 (HSV-1), herpes simplex virus type 2 (HSV-2), human cytomegalovirus (hCMV), human immunodeficiency virus (HIV), Ilheus virus, influenza virus (including avian and swine isolates), rhinovirus, norovirus, adenovirus, Japanese encephalitis virus, Kaposi's sarcoma associated herpesvirus (KSHV), Kokobera virus, Kunjin virus, Kyasanur forest disease virus, louping-ill virus, measles virus, MERS-coronavirus (MERS), metapneumovirus, any of the Mosaic Viruses, Murray Valley virus, parainfluenza virus, poliovirus, Powassan virus, respiratory syncytial virus (RSV), Rocio virus, SARS-coronavirus (SARS), St. Louis encephalitis virus, tick-borne encephalitis virus, West Nile virus (WNV), Ebola virus, Nipah virus, Lassa virus, Tacaribe virus, Junin virus, yellow fever virus, Varicella zoster virus (VZV), or vesicular stomatitis virus.

36. (canceled)

37. (canceled)
